(19) United States
(12) Patent Application Publication
(10) Pub. No.: US 2014/0011236 A1
(43) Pub. Date: Jan. 9, 2014

(54) PROMOTERS FOR HIGH LEVEL RECOMBINANT EXPRESSION IN FUNGAL HOST CELLS

(71) Applicant: MERCH SHARP & DOHME CORP., Rahway, NJ (US)

(72) Inventors: Robert Davidson, Enfield, NH (US); Bianka Prinz, Lebanon, NH (US); I-Ming Wang, Furlong, PA (US); Stephen Hamilton, Enfield, NH (US); Ming-Tang Chen, Lebanon, NH (US); Brian Mickus, Souderton, PA (US)

(21) Appl. No.: 14/005,340

(22) PCT Filed: Mar. 15, 2012
(86) PCT No.: PCT/US2012/029146

§ 371 Date: Sep. 16, 2013

Related U.S. Application Data
(60) Provisional application No. 61/466,220, filed on Mar. 22, 2011.
(60) Provisional application No. 61/473,426, filed on Apr. 8, 2011.

(51) Int. Cl. C12P 21/00

(52) U.S. Cl. CPC C12P 21/00
USPC 435/69.1; 536/23.1; 536/23.53; 536/23.52; 435/320.1; 435/254.11; 435/254.23; 435/254.21; 435/254.22; 435/254.3; 435/254.6; 435/254.7; 435/254.4; 435/247

(57) ABSTRACT

The present invention provides, in part, promoters for recombinant expression of polypeptides in host cells such as Pichia pastoris as well as methods of use thereof.

The present application claims the benefit of U.S. provisional patent application No. 61/466,220, filed Mar. 22, 2011; and U.S. provisional patent application No. 61/473,426, filed Apr. 8, 2011; each of which is herein incorporated by reference in its entirety.

### FIELD OF THE INVENTION

The field of the invention relates to promoters from fungal cells such as Pichia pastoris and methods of use thereof.

### BACKGROUND OF THE INVENTION

The methylotrophic yeast Pichia pastoris is one of the most widely used expression hosts for genetic engineering. This ascomycetous single-celled budding yeast has been used for the heterologous expression of hundreds of proteins (Lin-Cereghino, Curr Opin Biotech, 2002; Macauley-Patrick, Yeast, 2005). As a protein expression system, P. pastoris provides the advantages of a microbial system with facile genetics, shorter cycle times and the capability of achieving high cell densities. Secreted protein productivities have routinely been reported in the multi-gram per liter ranges. Several promoter systems are available for expression of proteins, for example, the methanol-inducible AOX1 promoter. The AOX1 promoter is a desirable aspects of the P. pastoris system because it is tightly regulated and highly induced on methanol (Cregg, Biotechnology, 1993, 11:905-910). The native Aox1p can be expressed up to 30% of total cellular protein when cells are grown on methanol. One drawback to this system is that cultivation on methanol during large scale fermentation can be complicated.

Constitutive promoter systems have been developed using the GAPDH promoter and more recently the TEF promoter (Waterham, Gene 1997, 186: 37-44; Ahn, Appl Microb Biotech, 2007, 74:601-608). These promoters are not as strong as AOX1, but, in some instances have proven to yield higher levels of secreted product than expression by AOX1, probably due to cultivation on a more energetically rich carbon source such as glycerol or glucose.

Importantly, P. pastoris is a eukaryote which provides the further advantage of having basic machinery for protein folding and post-translational modifications. Recent progress in the field, including humanization of the P. pastoris N-glycosylation pathway and a better understanding of the yeast secretory pathway, has resulted in the need to express multiple heterologous genes in the same strain, in some cases up to a dozen or more (Hamilton, Science, 2006, 313: 1441-1443; Wildt, Nat Rev Microbiol, 2005, 3: 119-128). Consequently, bottlenecks in strain engineering can arise with the availability of expression tools such as gene regulatory elements (i.e., promoters) and selection markers to introduce them. Several selectable markers have been developed for gene expression in P. pastoris including the recyclable URA5 system and multiple gene cassettes can be linked to the same marker to alleviate the problem of introduction of large numbers of genes. Moreover, a number of useful promoters have been identified aside from those named above including several methanol-inducible promoters such as AOX2 (the isogene of AOX1), Dihydroxyacetone Synthase (DAS), Formaldehyde Synthase (FLD1), and PEX8, other genes in core metabolism such as Isocitrate Lyase (ICL1), phosphate inducible PHO89, as well as the copper inducible heterologous S. cerevisiae CUP1 (Kobayashi, J. Biosci. & Bioeng., 2000, 89:479-484; Tschopp, Nuc. Acids. Res., 1987, 15; 3859-3876; Resina, J. Biotech., 2004, 109: 103-113; Menendez, Yeast, 2003, 20: 1097-1108; Ahn, AEM, 2009; Koller, Yeast, 2000, 96:651-656; U.S. Pat. No. 4,855,231). However, many of these promoter systems are not compatible with each other or would require starving for multiple nutrients, which can complicate bioprocess development. Moreover, many promoters that are considered to be constitutive, such as core metabolism and glycolysis genes, are significantly up- or down-regulated when carbon source conditions vary. For example, GAPDH is significantly down-regulated during cultivation on methanol, which can impact the expression of the desired gene of interest (Zhang, J. Ind. Micro. & Biotech., 2007, 34: 117-122). Therefore, additional useful promoters would be of value and interest to the field. Here, we present the identification of several novel P. pastoris promoters under relevant bioprocess conditions, and provide examples to demonstrate the utility of these promoters for heterologous gene expression.

### SUMMARY OF THE INVENTION

The present invention provides an isolated hybrid polynucleotide comprising a promoter selected from the group consisting of: Pichia pastoris GAPDH promoter; Pichia pastoris Pp02g05010 (PpPIR1) promoter; Pichia pastoris Pp05g08520 (ScCCW12) promoter; Pichia pastoris Pp01g10900 (ScCHT2) promoter; Pichia pastoris Pp05g07900 (ScAAC2/PET9) promoter; Pichia pastoris Pp02g01530 (ScPST1) promoter; Pichia pastoris Pp05g00700 (unknown) promoter; Pichia pastoris Pp02g04110 (ScPOR1) promoter; Pichia pastoris Pp01g03600 (ScBGL2) promoter; Pichia pastoris Pp01g14410 (ScACO1) promoter; Pichia pastoris Pp01g09650 (ScYHR021C) promoter; Pichia pastoris Pp01g02780 (ScYLR388W) promoter; Pichia pastoris Pp03g09940 (ScPIL1) promoter; Pichia pastoris Pp02g10710 (ScMDH1) promoter; Pichia pastoris 01g09290 (ScFBA1) promoter; Pichia pastoris Pp03g03520 (PpDAS2) promoter; Pichia pastoris Pp03g08760 (ScCWP1) promoter; Pichia pastoris Pp03g00990 (ScYGR201c) promoter; Pichia pastoris Pp02g05270 (AN2948.2) promoter; Pichia pastoris Pp02g12310 (ScDUR3) promoter; Pichia pastoris Pp03g05430 (ScTHI4) promoter; Pichia pastoris Pp03g03490 (AN2957.2) promoter; Pichia pastoris Pp05g09410 (ScTHI13) promoter; Pichia pastoris Pp02g07970 (ScPEX11/PMP27) promoter; Pichia pastoris Pp01g12200 (AN7917.2) promoter; Pichia pastoris Pp03g11380 (ScPMP47) promoter; Pichia pastoris Pp03g08340 (unknown) promoter; Pichia pastoris Pp05g04390 (ScTIR3) promoter; Pichia pastoris Pp01g08380 (ScYIL057c) promoter; Pichia pastoris Pp01g05090 (ScSAY1) promoter; Pichia pastoris Pp01g13950 (ScTPN1) promoter; Pichia pastoris Pp03g11420 (ScARO10) promoter; Pichia pastoris Pp02g11560 (ScMET6) promoter; Pichia pastoris Pp01g08650 (ScYNL067W) promoter; Pichia pastoris Pp01g01850 (PpPDHbeta1) promoter; Pichia pastoris Pp03g03020 (ScSAM2) promoter; and Pichia pastoris Pp03g02860 (PpSAHH) promoter (e.g., any of: nucleotides 1-1000 of SEQ ID NO: 14; nucleotides 1-1000 of SEQ ID NO: 15; nucleotides 1-1000 of SEQ ID NO: 16; nucleotides 1-1000 of SEQ ID NO: 17; nucleotides 1-1000 of SEQ ID NO: 18; nucleotides 1-1001 of SEQ ID NO: 19; nucleotides 1-1000 of SEQ ID NO: 20; nucleotides 1-1000 of SEQ ID NO: 21; nucleotides 1-1000 of SEQ ID NO: 22; nucleotides 1-1000 of SEQ ID NO: 23; nucleotides 1-1000 of SEQ ID NO: 24; nucleotides 1-1000 of SEQ ID NO: 25; nucleotides 1-1000 of SEQ ID NO: 26; nucleotides 1-1000 of SEQ ID NO: 27; nucleotides 1-1000 of SEQ ID NO: 28; nucleotides 1-1000 of SEQ ID NO: 29; and SEQ ID NOs: 47-63 and 70-76); operably linked to a heterologous polynucleotide (e.g., encoding an interferon or an immunoglobulin, for example, an immunoglobulin chain of an antibody or antigen-binding fragment thereof that binds specifically to VEGF, HER1, HER2, HER3, glycoprotein IIb/IIIa, CD52, IL-2R alpha receptor (CD25), epidermal growth factor receptor (EGFR), Complement system protein C5, CD11a, TNF alpha, CD33, IGF1R, CD20, T cell CD3 Receptor, alpha-4 (alpha 4) integrin, PCSK9, immunoglobulin E (IgE), RSV F protein or ErbB2; or, VEGF, HER1, HER2, HER3, glycoprotein IIb/IIIa, CD52, IL-2R alpha receptor (CD25), epidermal growth factor receptor (EGFR), Complement system protein C5, CD11a, TNF alpha, CD33, IGF1R, CD20, T cell CD3 Receptor, alpha-4 (alpha 4) integrin, PCSK9, immunoglobulin E (IgE), RSV F protein or ErbB2 polypeptide; or an immunogenic polypeptide fragment thereof; or a detectable reporter such as green fluorescent protein, Aequorea victoria GFP mutant 3, luciferase, Renilla luciferase, Photinus pyralis luciferase, Photinus pyralis luciferase slk mutant, Vibrio fischeri luxA, Vibrio fischeri luxB, Vibrio fischeri luxC, Vibrio fischeri luxD, Vibrio fischeri luxE, Vibrio fischeri luxAB, Vibrio fischeri luxCDABE, Vibrio harveyi luxA, Vibrio harveyi luxB, Vibrio harveyi luxC, Vibrio harveyi luxD, Vibrio harveyi luxE, Vibrio harveyi luxAB, Vibrio harveyi luxCDABE, Photorhabdus luminscens LuxA, Photorhabdus luminscens LuxB, Photorhabdus luminscens LuxC, Photorhabdus luminscens LuxD, Photorhabdus luminscens LuxE, Photorhabdus luminscens LuxCDABE, E. coli lacZ, the Aequorea victoria Aequorin, KanMX, pat1, nat1, hph, CAT, Sh Ble, GUS, CYH2 or CAN1. In an embodiment of the invention, a hybrid polynucleotide of the present invention is in an isolated vector and/or an isolated host cell (e.g., wherein the host comprises a vector that comprise the hybrid polynucleotide). Examples of host cells include fungal cells such as a Pichia cell, Pichia pastoris, Pichia flnlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia, Saccharomyces cerevisiae, Saccharomyces, Hansenula polymorpha, Kluyveromyces, Kluyveromyces lactis, Candida albicans, Aspergillus nidulans, Aspergillus niger, Aspergillus oryzae, Trichoderma reesei, Chrysosporium lucknowense, Fusarium, Fusańum gramineum, Fusarium venenatum and Neuraspora crassa. The present invention further comprises a composition comprising the host cell and growth culture medium (e.g., wherein the medium also includes methanol and/or the polypeptide encoded by the heterologous polynucleotide, for example, wherein the polypeptide is secreted from the host cell).

The present invention also provides a method for making a polypeptide comprising introducing, into an isolated fungal host cell (e.g., a Pichia cell, Pichia pastoris, Pichia flnlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia, Saccharomyces cerevisiae, Saccharomyces, Hansenula polymorpha, Kluyveromyces, Kluyveromyces lactic, Candida albicans, Aspergillus nidulans, Aspergillus niger, Aspergillus oryzae, Trichoderma reesei, Chrysosporium lucknowense, Fusarium, Fusańum gramineum, Fusarium venenatum and Neuraspora crassa), an isolated hybrid polynucleotide comprising a promoter selected from the group consisting of Pichia pastoris GAPDH promoter; Pichia pastoris Pp02g05010 (PpPIR1) promoter; Pichia pastoris Pp05g08520 (ScCCW12) promoter; Pichia pastoris Pp01g10900 (ScCHT2) promoter; Pichia pastoris Pp05g07900 (ScAAC2/PET9) promoter; Pichia pastoris Pp02g01530 (ScPST1) promoter; Pichia pastoris 01g09290 (ScFBA1) promoter; Pichia pastoris Pp03g03520 (PpDAS2) promoter; Pichia pastoris Pp03g08760 (ScCWP1) promoter; Pichia pastoris Pp03g00990 (ScYGR201c) promoter; Pichia pastoris Pp02g05270 (AN2948.2) promoter; Pichia pastoris Pp02g12310 (ScDUR3) promoter; Pichia pastoris Pp03g05430 (ScTHI4) promoter; Pichia pastoris Pp03g03490 (AN2957.2) promoter; Pichia pastoris Pp05g09410 (ScTHI13) promoter; Pichia pastoris Pp02g07970 (ScPEX11/PMP27) promoter; Pichia pastoris Pp01g12200 (AN7917.2) promoter; Pichia pastoris Pp03g11380 (ScPMP47) promoter; Pichia pastoris Pp03g08340 (unknown) promoter; Pichia pastoris Pp05g04390 (ScTIR3) promoter; Pichia pastoris Pp01g08380 (ScYIL057c) promoter; Pichia pastoris Pp01g05090 (ScSAY1) promoter; Pichia pastoris Pp01g13950 (ScTPN1) promoter; Pichia pastoris Pp03g11420 (ScARO10) promoter; Pichia pastoris Pp02g11560 (ScMET6) promoter; Pichia pastoris Pp01g08650 (ScYNL067W) promoter; Pichia pastoris Pp01g01850 (PpPDHbeta1) promoter; Pichia pastoris Pp03g03020 (ScSAM2) promoter; and Pichia pastoris Pp03g02860 (PpSAHH) promoter; operably linked to a heterologous polynucleotide; and culturing the host cell under conditions wherein said polynucleotide is expressed; optionally wherein said host cell is cultured in the presence of methanol.

The present invention further comprises a method for inducing expression of a heterologous polynucleotide in a fungal host cell, wherein said host cell comprises a promoter selected from the group consisting of: Pichia pastoris 01g09290 (ScFBA1) promoter; Pichia pastoris Pp03g03520 (PpDAS2) promoter; Pichia pastoris Pp03g08760 (ScCWP1) promoter; Pichia pastoris Pp03g00990 (ScYGR201c) promoter; Pichia pastoris Pp02g05270 (AN2948.2) promoter; Pichia pastoris Pp02g12310 (ScDUR3) promoter; Pichia pastoris Pp03g05430 (ScTHI4) promoter; Pichia pastoris Pp03g03490 (AN2957.2) promoter; Pichia pastoris Pp05g09410 (ScTHI13) promoter; Pichia pastoris Pp02g07970 (ScPEX11/PMP27) promoter; Pichia pastoris Pp01g12200 (AN7917.2) promoter; Pichia pastoris Pp03g11380 (ScPMP47) promoter; Pichia pastoris Pp03g08340 (unknown) promoter; Pichia pastoris Pp05g04390 (ScTIR3) promoter; Pichia pastoris Pp01g08380 (ScYIL057c) promoter; Pichia pastoris Pp01g05090 (ScSAY1) promoter; Pichia pastoris Pp01g13950 (ScTPN1) promoter; operably linked to the heterologous polynucleotide, comprising culturing the fungal host cell in a growth medium comprising methanol.

The present invention further comprises a method for repressing expression of a heterologous polynucleotide in a fungal host cell, wherein said host cell comprises a promoter selected from the group consisting of: Pichia pastoris Pp03g11420 (ScARO10) promoter; Pichia pastoris Pp02g11560 (ScMET6) promoter; Pichia pastoris Pp01g08650 (ScYNL067W) promoter; Pichia pastoris Pp01g01850 (PpPDHbeta1) promoter; Pichia pastoris Pp03g03020 (ScSAM2) promoter; and Pichia pastoris Pp03g02860 (PpSAHH) promoter; operably linked to the heterologous polynucleotide, comprising culturing the fungal host cell in a growth medium comprising methanol.

### BRIEF DESCRIPTION OF THE FIGURES

FIG. 1: Schematic representation of the two molecular profiling experiments. A) The wild type/glycoengineered strain comparison study was performed in 0.5 L Sixfors reactors and samples were taken during and at the end of glycerol batch phase (dark gray) and twice during methanol feeding (black). B) A) The mAb comparison study was performed in 1 L Sartorius Q's reactors and samples were taken during glycerol batch phase (dark gray), during glycerol fed-batch phase (light gray), and at five timepoints during methanol feeding (black).

FIG. 2: K-means cluster of wild type/glycoengineered strain comparison study glycerol-to-methanol gene signature. Gene expression data intensity profiles from the wild type/glycoengineered strains study were analyzed by first ratioing strain-specific, individual sample data to the Batch (50 mg/ml of wet cell weight; glycerol) timepoint. Three individual ANOVA analyses were then performed using 3 factors (Batch, 4 h MeOH, and 24 h MeOH), one for each of the strains with individual replicates with a cutoff of P<=0.005. These genes were then clustered by K-means with K=6 using a 2 fold-change cutoff in at least 4 samples, resulting in a total of 2,882 sequences clustered. Black indicates upregulated while white indicates downregulated with full saturation reached at +/−3.16 fold change.

FIG. 3: K-means cluster of mAb comparison study glycerol-to-methanol gene signature. Gene expression data intensity profiles from the mAb comparison study were analyzed by first ratioing strain-specific, individual sample data to the Batch (glycerol) timepoint. A two factor ANOVA was were performed comparing the two glycerol vs. 5 methanol timepoints with a significance cutoff of P<=0.01. These genes were then clustered by K-means with K=using a 1.2 fold-change cutoff in at least 3 samples, resulting in a total of 2,882 sequences clustered. Black indicates upregulated while white indicates downregulated with full saturation reached at +/−3.16 fold change.

FIG. 4: Dotplot depiction of intensity profiles of methanol inducible genes in the wild type/glycoengineered strain comparison study. The raw gene intensity profiles for the four-replicate-combined samples from A) y11430, B) YGLY8316, and C) YGLY8323 were plotted linearly by intensity on glycerol/batch (Intensity 1) vs. methanol induction/24 hrs MeOH (Intensity 2). The genes for the 17 newly identified methanol-inducible promoters are marked inclusive and exclusive of the entire 5150 P. pastoris geneset.

FIG. 5: Dotplot depiction of intensity profiles of methanol inducible genes in the mAb comparison study. The raw gene intensity profiles for the three-replicate-combined samples from YGLY13992 at A) 48 hrs induction and B) 96 hrs induction were plotted linearly by intensity on glycerol/batch (Intensity 1) vs. methanol induction/48 MeOH or 96 MeOH (Intensity 2). The genes for the 17 newly identified methanol-inducible promoters are marked inclusive and exclusive of the entire 5150 P. pastoris geneset.

FIG. 6: Relative comparison of expression profiling intensities of the genes of identified methanol-inducible promoters from the mAb comparison study. The triplicate-combined raw intensity values (referenced to batch) of the 17 newly identified methanol-inducible genes were plotted linearly as samples from batch (glycerol) and 48 hrs induction (methanol) for four different strains, A) YGLY8316 parental (no mAb), B) YGLY13992 (anti-HER2), C) YGLY12501 (anti-HER2), and YGLY10360 (VEGF). The previously known AOX1 (Pp05g01320) and GPD (Pp02g08660) genes are plotted similarly.

FIG. 7: Restriction maps of plasmids containing exemplary inducible promoters. The E. coli/P. pastoris shuttle vectors are depicted circularly as they are maintained in E. coli. For introduction into P. pastoris the plasmids are digested with SfiI to release the pUC19 portion, allowing integration at the TRP1 locus and selection with the P. pastoris URA5 gene. The promoters A) GAPDH (GPD) in pGLY580, B) CWP1 in pGLY8529, C) Pp03g03520/DAS2 in pGLY8530, D) FBA1 in pGLY8531, E) YGR201C in pGLY8532, and F) Pp03g03500/DAS1 in pGLY8533 and transcriptional terminators (TT) flank NotI/PacI sites that can be used for cloning open reading frames in front of the promoters.

FIG. 8: Dotplot depiction of intensity profiles of constitutive genes in the wild type/glycoengineered strain comparison study. The raw gene intensity profiles for the four-replicate-combined samples from A) y11430, B) YGLY8316, and C) YGLY8323 were plotted linearly by intensity on glycerol/batch (Intensity 1) vs. methanol induction/24 hrs MeOH (Intensity 2). The genes for the 13 newly identified constitutive promoters are marked inclusive and exclusive of the entire 5150 P. pastoris geneset.

FIG. 9: Dotplot depiction of intensity profiles of constitutive genes in the mAb comparison study. The raw gene intensity profiles for the three-replicate-combined samples from YGLY13992 at A) 48 hrs induction and B) 96 hrs induction were plotted linearly by intensity on glycerol/batch (Intensity 1) vs. methanol induction/48 MeOH or 96 MeOH (Intensity 2). The genes for the 13 newly identified constitutive promoters are marked inclusive and exclusive of the entire 5150 P. pastoris geneset.

FIG. 10: Relative comparison of expression profiling intensities of the genes of identified constitutive promoters from the mAb comparison study. The triplicate-combined raw intensity values (referenced to batch) of 12 newly identified constitutive genes were plotted linearly as samples from batch (glycerol) and 48 hrs induction (methanol) for four different strains, A) YGLY8316 parental (no mAb), B) YGLY13992 (anti-HER2), C) YGLY12501 (anti-HER2), and YGLY10360 (VEGF). The previously known AOX1 (Pp05g01320) and GPD (Pp02g08660) genes are plotted similarly.

FIG. 11: Restriction maps of plasmids containing exemplary constitutive promoters. The E. coli/P. pastoris shuttle vectors are depicted circularly as they are maintained in E. coli. For introduction into P. pastoris the plasmids are digested with SfiI to release the pUC19 portion, allowing integration at the TRP1 locus and selection with the P. pastoris URA5 gene. The promoters A) PIR1 in pGLY8620, B) CCW12 in pGLY8621, C) CHT2 in pGLY8622, D) PET9 in pGLY8623, E) PST1 in pGLY8624, F) TEF1/PpTEF in pGLY8625, G) GAPDH/PpGPD in pGLY8626, and H) PMA1 in pGLY8627 and transcriptional terminators (TT) flank NotI/PacI sites that can be used for cloning open reading frames in front of the promoters.

FIG. 12: Dotplot depiction of intensity profiles of methanol-repressible genes in the wild type/glycoengineered strain comparison study. The raw gene intensity profiles for the four-replicate-combined samples from A) y11430, B) YGLY8316, and C) YGLY8323 were plotted linearly by intensity on glycerol/batch (Intensity 1) vs. methanol induction/24 hrs MeOH (Intensity 2). The genes for the 6 newly identified methanol-repressible promoters are marked inclusive and exclusive of the entire 5150 P. pastoris geneset.

FIG. 13: Dotplot depiction of intensity profiles of methanol-repressible genes in the mAb comparison study. The raw gene intensity profiles for the three-replicate-combined samples from YGLY13992 at A) 48 hrs induction and B) 96 hrs induction were plotted linearly by intensity on glycerol/batch (Intensity 1) vs. methanol induction/48 MeOH or 96 MeOH (Intensity 2). The genes for the 13 newly identified methanol-repressible promoters are marked inclusive and exclusive of the entire 5150 P. pastoris geneset.

FIG. 14: Relative comparison of expression profiling intensities of the genes of identified methanol-repressible promoters from the mAb comparison study. The triplicate-combined raw intensity values (referenced to batch) of 6 newly identified methanol-repressible genes were plotted linearly as samples from batch (glycerol) and 48 hrs induction (methanol) for four different strains, A) YGLY8316 parental (no mAb), B) YGLY13992 (anti-HER2), C) YGLY12501 (anti-HER2), and YGLY10360 (VEGF). The previously known AOX1 (Pp05g01320) and GPD (Pp02g08660) genes are plotted similarly.

FIG. 15: Relative activity of constitutive promoters by beta-galactosidase reporter gene assay. Four putative strong constitutive promoters from the P. pastoris genes PIR1 (Pp02g05010), CCW12 (Pp05g08520), CHT2 (Pp01g10900), PET9 (Pp05g07900), and PST1 (Pp02g01530), along the TEF (Pp01g00550), GPD (Pp02g08660), and PMA1 (Pp02g12610) promoters as controls were introduced into a GFI5.0 glycoengineered P. pastoris strain (Bobrowicz et al., Glycobiol 2004; Davidson U.S. Pat. No. 7,795,002). Resulting transformants were cultivated in 96 deep well plate format in liquid medium with glycerol for 72 hrs, pellets harvested and then cultivated for 24 hrs in medium with methanol. The pellets were harvested and subjected to standard beta-galactosidase assays (Guarente Methods Emzymol 1983, 101: 181-191).

FIG. 16: Secreted production of the human Fc fragment by P. pastoris methanol-inducible promoters. Four new inducible promoters were fused to the Human Fc gene: CWP1 (Pp03g08760), PpDAS2 (Pp03g03520), FBA1 (Pp01g09290), YGR201C (Pp03g00990), as well as PpDAS1 (Pp03g03500), PpAOX1 (Pp05g01320) as controls, and introduced into a GFI5.0 glycoengineered P. pastoris strain (Bobrowicz et al., Glycobiol 2004; Davidson U.S. Pat. No. 7,795,002). Resulting transformants were cultivated in Applikon micro 24 5 ml fermenters liquid medium with glycerol for 72 hrs, supernatants harvested and then cultivated for 72 hrs in medium with methanol and the supernatants again harvested. The harvested supernatants were subjected to Protein A purification and HPLC separation for Fc titer determination. None of the glycerol samples yielded any detectable Fc.

FIG. 17: Cartoon depiction of the Protein A-ScSED1 display strategy. Previous attempts to co-secrete the Protein A-ScSED1 anchor and the secreted full length mAb resulted in no detectable cell surface display of the mAb. Introduction of the repressible promoters in front of the Protein A-ScSED1 anchor drives production only during glycerol phase and represses production during the methanol phase when the mAb production is initiated results in successful mAb capture and cell surface display.

FIG. 18: Restriction map of plasmid pGLY4136 containing the Protein A-ScSED1 anchor fusion. The E. coli/P. pastoris shuttle vector is depicted circularly as it is maintained in E. coli. The AMU promoter can be replaced using the flanking BglII/EcoRI restriction sites. For introduction into P. pastoris the resulting plasmids are digested with SfiI to release the pUC19 portion, allowing integration at the TRP1 locus and selection with the P. pastoris URA5 gene.

FIG. 19: Protein A display with methanol-repressible promoters detected by FACS with a labeled Ab. Four methanol repressible promoters were fused to the protein-A/SED1 anchor: Pp03g11420 (ARO10), Pp02g11560 (MET6), Pp01g08650 (ScYNL067W), and Pp03g03020 (SAM2) and the resulting constructs introduced into strains YGLY17108 (A, no secreted mAb) and YGLY13979 (B, secreted anti-HER2 mAb). Transformants (as well as YGLY17108 expressing neither cell surface anchor nor secreted mAb) were cultivated for 48 h in glycerol and subjected to FACS analysis using fluorescent rabbit IgG1-Alexa Fluor 488 conjugated Ab. Yeast cells capable of binding the conjugated Ab are visible via increased FITC-A channel fluorescence intensity displayed and are shifted to the right.

FIG. 20: Repressible promoter driven Protein A-ScSED1 anchor is capable of cell surface display of an anti-HER2 mAb. (A) The YGLY17108 control and clones transformed with plasmids containing the protein A-SED1 anchor driven by the repressible promoters Pp03g11420 (ARO10), Pp02g11560 (MET6), Pp01g08650 (ScYNL067W), and Pp03g03020 (SAM2). (B) Strain YGLY13979 (expressing secreted anti-HER2 mAb) was then transformed with the same plasmids containing the protein A-SED1 anchor driven by Pp03g11420 (ARO10), Pp02911560 (MET6), Pp01g08650 (ScYNL067W), and Pp03g03020 (SAM2). The resulting transformants from each were cultivated in glycerol-containing medium and induced in methanol containing medium, then were subjected to FACS analysis by labeling with fluorescent Fab anti-Fc DyLight-488 conjugated to detect the heavy chain of the secreted displayed antibody. YGLY17108 is used as a negative control for both groups of strains.

FIG. 21: Repressible promoter driven Protein A-ScSED1 anchor is capable of cell surface display of two different anti-PCSK9 mAbs. Two anti-PCSK9 mAb expressing strains, YGLY18483 and YGLY18281, were transformed with a plasmid containing a protein A-SED1 anchor driven by the repressible promoter Pp03g03020 (SAM2). The resulting transformants were cultivated in glycerol-containing medium and induced in methanol containing medium, then were subjected to FACS analysis by labeling with fluorescent Fab anti-Fc DyLight-488 to detect the antibody heavy chain and with biotinylated PCSK9 antigen and further labeled with streptavidin-Alexa Fluor 635 conjugate to detect the biotinylated PCSK9.

FIG. 22: Relative activity of constitutive promoters at 40 L fermentation scale by beta-galactosidase reporter gene assay. Six constitutive promoters were fused to the E. coli lacZ gene and the gene fusions introduced into a P. pastoris glycoengineered strain. The strong constitutive promoters included the previously undescribed P. pastoris genes PIR1 (Pp02g05010) and CHT2 (Pp01g10900), along the TEF (Pp01g00550) and PMA1 (Pp02g12610) as controls as well as the traditional short 500 bp version of the GPD (Pp02g08660) and the novel 1 kb long version with the native transcriptional terminator. Clones expressing the lacZ gene under control of these promoters were cultivated in a 40 liter stainless steel bioreactor in a standard methanol-induced, carbon-limited fedbatch process. At the timepoints indicated, cells were harvested, subjected to centrifugation and beta-galactosidase assay in duplicate.

### DETAILED DESCRIPTION OF THE INVENTION

A hybrid polynucleotide of the present invention refers to a polynucleotide comprising a promoter of the present invention operably linked a heterologous polynucleotide.

A heterologous polynucleotide e.g., that is operably linked to a promoter of the present invention, refers to a polynucleotide encoding a polypeptide that is not naturally contiguous with or operably linked to the nucleotide sequence of the promoter of the present invention. Heterologous polynucleotides encoding a heterologous polypeptide (e.g., an immunogenic polypeptide or oligopeptide) include for example, polynucleotides encoding a detectable reporter, interferon (interferon alpha 2a or interferon alpha 2b) or an immunoglobulin (e.g., a heavy chain and/or light chain, e.g., linked to an immunoglobulin light chain constant domain such as kappa or lambda; or heavy chain constant domain such as gamma, e.g., gamma, gamma-1, gamma-2, gamma-3 or gamma-4) which can form part of an antibody or antigen-binding fragment thereof such as, anti-VEGF, anti-HER1, anti-HER2, anti-HER3, anti-glycoprotein IIb/IIIa, anti-CD52, anti-IL-2R alpha receptor (CD25), anti-epidermal growth factor receptor (EGFR), anti-Complement system protein C5, anti-CD20, anti-CD11a, anti-TNF alpha, anti-CD33, anti-IGF1R, anti-CD20, anti-T cell CD3 Receptor, anti-alpha-4 (alpha 4) integrin, anti-PCSK9, anti-immunoglobulin E (IgE), anti-RSV F protein or anti-ErbB2.

In an embodiment of the invention, a detectable reporter is green fluorescent protein, such as Aequorea victoria GFP mutant 3, luciferase, Renilla luciferase, Photinus pyralis luciferase, Photinus pyralis luciferase slk mutant, Vibrio fischeri luxA, Vibrio fischeri luxB, Vibrio fischeri luxC, Vibrio fischeri luxD, Vibrio fischeri luxE, Vibrio fischeri luxAB, Vibrio fischeri luxCDABE, Vibrio harveyi luxA, Vibrio harveyi luxB, Vibrio harveyi luxC, Vibrio harveyi luxD, Vibrio harveyi luxE, Vibrio harveyi luxAB, Vibrio harveyi luxCDABE, Photorhabdus luminscens LuxA, Photorhabdus luminscens LuxB, Photorhabdus luminscens LuxC, Photorhabdus luminscens LuxD, Photorhabdus luminscens LuxE, Photorhabdus luminscens LuxCDABE, E. coli lacZ, the Aequorea victoria Aequorin gene, KanMX, pat1, nat1, hph, CAT, Sh Ble, GUS, CYH2 or CAN1.

“MeOH” is methanol.

### Molecular Biology

In accordance with the present invention there may be employed conventional molecular biology, microbiology, and recombinant DNA techniques within the skill of the art. Such techniques are explained in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, at al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

A “polynucleotide”, “nucleic acid” includes DNA and RNA in single stranded form, double-stranded form or otherwise.

A “polynucleotide sequence” or “nucleotide sequence” is a series of nucleotide bases (also called “nucleotides”) in a nucleic acid, such as DNA or RNA, and means a series of two or more nucleotides. Any polynucleotide comprising a nucleotide sequence set forth herein (e.g., promoters of the present invention) forms part of the present invention.

A “coding sequence” or a sequence “encoding” an expression product, such as an RNA or polypeptide is a nucleotide sequence (e.g., heterologous polynucleotide) that, when expressed, results in production of the product (e.g., a heterologous polypeptide such as an immunoglobulin heavy chain and/or light chain).

As used herein, the term “oligonucleotide” refers to a nucleic acid, generally of no more than about 100 nucleotides (e.g., 30, 40, 50, 60, 70, 80, or 90), that may be hybridizable to a polynucleotide molecule. Oligonucleotides can be labeled, e.g., by incorporation of ³²P-nucleotides, ³H-nucleotides, ¹⁴C-nucleotides, ³⁵S-nucleotides or nucleotides to which a label, such as biotin, has been covalently conjugated.

A “protein”, “peptide” or “polypeptide” (e.g., a heterologous polypeptide such as an immunoglobulin heavy chain and/or light chain) includes a contiguous string of two or more amino acids.

A “protein sequence”, “peptide sequence” or “polypeptide sequence” or “amino acid sequence” refers to a series of two or more amino acids in a protein, peptide or polypeptide.

The term “isolated polynucleotide” or “isolated polypeptide” includes a polynucleotide or polypeptide, respectively, which is partially or fully separated from other components that are normally found in cells or in recombinant DNA expression systems or any other contaminant. These components include, but are not limited to, cell membranes, cell walls, ribosomes, polymerases, serum components and extraneous genomic sequences. The scope of the present invention includes the isolated polynucleotides set forth herein, e.g., the promoters set forth herein; and methods related thereto, e.g., as discussed herein.

An isolated polynucleotide or polypeptide will, preferably, be an essentially homogeneous composition of molecules but may contain some heterogeneity.

“Amplification” of DNA as used includes the use of polymerase chain reaction (PCR) to increase the concentration of a particular DNA sequence within a mixture of DNA sequences. For a description of PCR see Saiki, et al., Science (1988) 239:487.

In general, a “promoter” or “promoter sequence” is a DNA regulatory region capable of binding an RNA polymerase in a cell (e.g., directly or through other promoter-bound proteins or substances) and initiating transcription of a coding sequence to which it operably links. A “promoter of the present invention” includes any of the following promoters:

Pichia pastoris GAPDH promoter (e.g., wherein any sequence operably linked to the promoter is also operably linked to a downstream CYC1 terminator);
Pichia pastoris Pp02g05010 (PpPIR1) promoter;
Pichia pastoris Pp05g08520 (ScCCW12) promoter;
Pichia pastoris Pp01g10900 (ScCHT2) promoter;
Pichia pastoris Pp05g07900 (ScAAC2/PET9) promoter;
Pichia pastoris Pp02g01530 (ScPST1) promoter;
Pichia pastoris Pp05g00700 (unknown) promoter;
Pichia pastoris Pp02g04110 (ScPOR1) promoter;
Pichia pastoris Pp01g03600 (ScBGL2) promoter;
Pichia pastoris Pp01g14410 (ScACO1) promoter;
Pichia pastoris Pp01g09650 (ScYHR021C) promoter;
Pichia pastoris Pp01g02780 (ScYLR388W) promoter;
Pichia pastoris Pp03g09940 (ScPIL1) promoter;
Pichia pastoris Pp02g10710 (ScMDH1) promoter;
Pichia pastoris Pp01g09290 (ScFBA1) promoter;
Pichia pastoris Pp03g03520 (PpDAS2) promoter;
Pichia pastoris Pp03g08760 (ScCWP1) promoter;
Pichia pastoris Pp03g00990 (ScYGR201c) promoter;
Pichia pastoris Pp02g05270 (AN2948.2) promoter;
Pichia pastoris Pp02g12310 (ScDUR3) promoter;
Pichia pastoris Pp03g05430 (ScTHI4) promoter;
Pichia pastoris Pp03g03490 (AN2957.2) promoter;
Pichia pastoris Pp05g09410 (ScTHI13) promoter;
Pichia pastoris Pp02g07970 (ScPEX11/PMP27) promoter;
Pichia pastoris Pp01g12200 (AN7917.2) promoter;
Pichia pastoris Pp03g11380 (ScPMP47) promoter;
Pichia pastoris Pp03g08340 (unknown) promoter;
Pichia pastoris Pp05g04390 (ScTIR3) promoter;
Pichia pastoris Pp01g08380 (ScYIL057c) promoter;
Pichia pastoris Pp01g05090 (ScSAY1) promoter;
Pichia pastoris Pp01g13950 (ScTPN1) promoter;
Pichia pastoris Pp03g11420 (ScARO10) promoter;
Pichia pastoris Pp02g11560 (ScMET6) promoter;
Pichia pastoris Pp01g08650 (ScYNL067W) promoter;
Pichia pastoris Pp01g01850 (PpPDHbeta1) promoter;
Pichia pastoris Pp03g03020 (ScSAM2) promoter; or
Pichia pastoris Pp03g02860 (PpSAHH) promoter;
(e.g., nucleotides 1-1000 of SEQ ID NO: 14; nucleotides 1-1000 of SEQ ID NO: 15; nucleotides 1-1000 of SEQ ID NO: 16; nucleotides 1-1000 of SEQ ID NO: 17; nucleotides 1-1000 of SEQ ID NO: 18; nucleotides 1-1001 of SEQ ID NO: 19; nucleotides 1-1000 of SEQ ID NO: 20; nucleotides 1-1000 of SEQ ID NO: 21; nucleotides 1-1000 of SEQ ID NO: 22; nucleotides 1-1000 of SEQ ID NO: 23; nucleotides 1-1000 of SEQ ID NO: 24; nucleotides 1-1000 of SEQ ID NO: 25; nucleotides 1-1000 of SEQ ID NO: 26; nucleotides 1-1000 of SEQ ID NO: 27; nucleotides 1-1000 of SEQ ID NO: 28; nucleotides 1-1000 of SEQ ID NO: 29; and SEQ ID NOs: 47-63 and 70-75) and/or functional variants thereof. Promoter functional variants are discussed in greater detail below.

A coding sequence (e.g., of a heterologous polynucleotide, e.g., reporter gene or immunoglobulin heavy and/or light chain) is “operably linked to”, “under the control of”, “functionally associated with” or “operably associated with” a transcriptional and translational control sequence (e.g., a promoter of the present invention) when the sequence directs RNA polymerase mediated transcription of the coding sequence into RNA, preferably mRNA, which then may be RNA spliced (if it contains introns) and, optionally, translated into a protein encoded by the coding sequence. A promoter of the present invention operably linked to a coding sequence forms part of the present invention. In an embodiment of the invention, a polynucleotide is operably linked to a transcriptional terminator sequence, e.g., any of those that are included in SEQ ID NOs: 14-29.

The scope of the present invention includes cassettes comprising any of the promoters of the present invention upstream of a polylinker sequence into which a polynucleotide (e.g., a heterologous polynucleotide) can be inserted if desired, optionally, operably linked to a transcriptional terminator sequence (e.g., any of SEQ ID NOs: 14-29). Methods for recombining a cassette (e.g., any of SEQ ID NOs: 14-29) with a polynucleotide (e.g., a heterologous polynucleotide) comprising cleaving the polylinker (e.g., with a restriction endonuclease) and inserting the polynucleotide into the cassette at the cleaved polylinker and religating the recombined polynucleotides together, form part of the present invention as does any such recombined cassette, e.g., formed by such a method. Host cells and uses of such recombined cassettes for expressing a polypeptide (e.g., heterologous polypeptide) discussed herein form part of the present invention as well.

The present invention includes vectors which comprise promoters of the invention optionally operably linked to a heterologous polynucleotide. The term “vector” includes a vehicle (e.g., a plasmid) by which a DNA or RNA sequence can be introduced into a host cell, so as to transform the host and, optionally, promote expression and/or replication of the introduced sequence. In general, a plasmid is circular, includes an origin (e.g., 2 μm origin) and, preferably includes a selectable marker. In plasmids which can be maintained in yeast, commonly used yeast markers include URA3, HIS3, LEU2, TRP1 and LYS2, which complement specific auxotrophic mutations in a yeast host cell, such as ura3-52, his3-D1, leu2-D1, trp1-D1 and lys2-201, respectively. If the plasmid can be maintained in E. coli, it may include a bacterial origin (ori) and/or a selectable market such as the β-lactamase gene (bla or AMP^{r}). Commonly used yeast/E. coli shuttle vectors are the Yip (see Myers et al., Gene 45: 299-310, (1986)), YEp (see Myers et al., Gene 45: 299-310, (1986)), YCp and YRp plasmids. The YIp integrative vectors do not replicate autonomously, but integrate into the genome at low frequencies by homologous recombination. The YEp yeast episomal plasmid vectors replicate autonomously because of the presence of a segment of the yeast 2 μm plasmid that serves as an origin of replication (2 μm ori). The 2 μm ori is responsible for the high copy-number and high frequency of transformation of YEp vectors. The YCp yeast centromere plasmid vectors are autonomously replicating vectors containing centromere sequences, CEN, and autonomously replicating sequences, ARS. The YCp vectors are typically present at very low copy numbers, from 1 to 3 per cell. Autonomously replicating plasmids (YRp) which carry a yeast origin of replication (ARS sequence; but not centromere) that allows the transformed plasmids to be propagated several hundred-fold. YIp, YEp, YCp and YRp are commonly known in the art and widely available. Another acceptable yeast vector is a yeast artificial chromosome (MAC). A yeast artificial chromosome is a biological vector. It is an artificially constructed chromosome and contains the telomeric, centromeric, and replication origin sequences needed for replication in yeast cells (see Marchuk et al., Nucleic Acids Res. 16(15):7743 (1988); Rech et al., Nucleic Acids Res. 18(5):1313 (1990)).

Vectors that could be used in this invention include plasmids, viruses, bacteriophage, integratable DNA fragments, and other vehicles that may facilitate introduction of the nucleic acids into the genome of a host cell (e.g., Pichia pastoris). Plasmids are the most commonly used form of vector but all other forms of vectors which serve a similar function and which are, or become, known in the art are suitable for use herein. See, e.g., Pouwels, et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

A polynucleotide (e.g., a heterologous polynucleotide, e.g., encoding an immunoglobulin heavy chain and/or light chain), operably linked to a promoter of the present invention, may be expressed in an expression system. The term “expression system” means a host cell and compatible vector which, under suitable conditions, can express a protein or nucleic acid which is carried by the vector and introduced to the host cell. Common expression systems include fungal host cells (e.g., Pichia pastoris) and plasmid vectors, insect host cells and Baculovirus vectors, and mammalian host cells and vectors.

The term methanol-induction refers to increasing expression of a polynucleotide (e.g., a heterologous polynucleotide) operably linked to a methanol-inducible promoter of the present invention in a host cell by exposing the host cells to methanol.

The term methanol-repression refers to decreasing expression of a polynucleotide (e.g., a heterologous polynucleotide) operably linked to a methanol-repressible promoter of the present invention in a host cell by exposing the host cells to methanol.

The present invention also contemplates any superficial or slight modification to a promoter of the present invention. For example, the present invention includes any “functional variant” of any of: Pichia pastoris Pp02g05010 (PpPIR1) promoter; Pichia pastoris Pp05g08520 (ScCCW12) promoter; Pichia pastoris Pp01g10900 (ScCHT2) promoter; Pichia pastoris Pp05g07900 (ScAAC2/PET9) promoter; Pichia pastoris Pp02g01530 (ScPST1) promoter; Pichia pastoris Pp05g00700 (unknown) promoter; Pichia pastoris Pp02g04110 (ScPOR1) promoter; Pichia pastoris Pp01g03600 (ScBGL2) promoter; Pichia pastoris Pp01g14410 (ScACO1) promoter; Pichia pastoris Pp01g09650 (ScYHR021C) promoter; Pichia pastoris Pp01g02780 (ScYLR388W) promoter; Pichia pastoris Pp03g09940 (ScPIL1) promoter; Pichia pastoris Pp02g10710 (ScMDH1) promoter; Pichia pastoris 01g09290 (ScFBA1) promoter; Pichia pastoris Pp03g03520 (PpDAS2) promoter; Pichia pastoris Pp03g08760 (ScCWP1) promoter; Pichia pastoris Pp03g00990 (ScYGR201c) promoter; Pichia pastoris Pp02g05270 (AN2948.2) promoter; Pichia pastoris Pp02g12310 (ScDUR3) promoter; Pichia pastoris Pp03g05430 (ScTHI4) promoter; Pichia pastoris Pp03g03490 (AN2957.2) promoter; Pichia pastoris Pp05g09410 (ScTHI13) promoter; Pichia pastoris Pp02g07970 (ScPEX11/PMP27) promoter; Pichia pastoris Pp01g12200 (AN7917.2) promoter; Pichia pastoris Pp03g11380 (ScPMP47) promoter; Pichia pastoris Pp03g08340 (unknown) promoter; Pichia pastoris Pp05g04390 (ScTIR3) promoter; Pichia pastoris Pp01g08380 (ScYIL057c) promoter; Pichia pastoris Pp01g05090 (ScSAY1) promoter; Pichia pastoris Pp01g13950 (ScTPN1) promoter; Pichia pastoris Pp03g11420 (ScARO10) promoter; Pichia pastoris Pp02g11560 (ScMET6) promoter; Pichia pastoris Pp01g08650 (ScYNL067W) promoter; Pichia pastoris Pp01g01850 (PpPDHbeta1) promoter; Pichia pastoris Pp03g03020 (ScSAM2) promoter; or Pichia pastoris Pp03g02860 (PpSAHH) promoter (e.g., any of nucleotides 1-1000 of SEQ ID NO: 14; nucleotides 1-1000 of SEQ ID NO: 15; nucleotides 1-1000 of SEQ ID NO: 16; nucleotides 1-1000 of SEQ ID NO: 17; nucleotides 1-1000 of SEQ ID NO: 18; nucleotides 1-1001 of SEQ ID NO: 19; nucleotides 1-1000 of SEQ ID NO: 20; nucleotides 1-1000 of SEQ ID NO: 21; nucleotides 1-1000 of SEQ ID NO: 22; nucleotides 1-1000 of SEQ ID NO: 23; nucleotides 1-1000 of SEQ ID NO: 24; nucleotides 1-1000 of SEQ ID NO: 25; nucleotides 1-1000 of SEQ ID NO: 26; nucleotides 1-1000 of SEQ ID NO: 27; nucleotides 1-1000 of SEQ ID NO: 28; nucleotides 1-1000 of SEQ ID NO: 29; and SEQ ID NOs: 47-63 and 70-75). A functional variant of a promoter includes any sequence variant (e.g., comprising one or more point mutations and/or deletions) that retains the ability to cause the expression of an operably linked polynucleotide (e.g., of a coding sequence) at any detectable level or at a level at least equal to that of the corresponding non-variant promoter. Methods for determining whether a particular promoter (e.g., comprising one or more point mutations and/or deletions) promotes expression (e.g., transcription) of a sequence to which it is functionally linked are conventional and well known in the art. For example, expression can be determined by Northern blot detection of RNA; or, ELISA or Western blot detection of protein encoded by the operably linked coding sequence.

The present invention includes polynucleotides which hybridize to a promoter of the present invention or a complement thereof (e.g., any of nucleotides 1-1000 of SEQ ID NO: 14; nucleotides 1-1000 of SEQ ID NO: 15; nucleotides 1-1000 of SEQ ID NO: 16; nucleotides 1-1000 of SEQ ID NO: 17; nucleotides 1-1000 of SEQ ID NO: 18; nucleotides 1-1001 of SEQ ID NO: 19; nucleotides 1-1000 of SEQ ID NO: 20; nucleotides 1-1000 of SEQ ID NO: 21; nucleotides 1-1000 of SEQ ID NO: 22; nucleotides 1-1000 of SEQ ID NO: 23; nucleotides 1-1000 of SEQ ID NO: 24; nucleotides 1-1000 of SEQ ID NO: 25; nucleotides 1-1000 of SEQ ID NO: 26; nucleotides 1-1000 of SEQ ID NO: 27; nucleotides 1-1000 of SEQ ID NO: 28; nucleotides 1-1000 of SEQ ID NO: 29; and SEQ ID NOs: 47-63 and 70-75) but which retain the ability to drive expression, e.g., at a detectable level or at a level at least equal to that of the corresponding non-variant promoter. Preferably, the polynucleotides hybridize under low stringency conditions, more preferably under moderate stringency conditions and most preferably under high stringency conditions. A polynucleotide is “hybridizable” to another polynucleotide when a single stranded form of the nucleic acid molecule (e.g., either strand) can anneal to the other nucleic acid molecule under the appropriate conditions of temperature and solution ionic strength (see Sambrook, at al., supra). The conditions of temperature and ionic strength determine the “stringency” of the hybridization. Low stringency hybridization conditions may be 55° C., 5×SSC, 0.1% SDS, 0.25% milk, and no formamide; or 30% formamide, 5×SSC, 0.5% SDS. Moderate stringency hybridization conditions are similar to the low stringency conditions except the hybridization is carried out in 40% formamide, with 5× or 6×SSC. High stringency hybridization conditions are similar to low stringency conditions except the hybridization conditions are carried out in 50% formamide, 5× or 6×SSC and, optionally, at a higher temperature (e.g., 57° C., 59° C., 60° C., 62° C., 63° C., 65° C. or 68° C.). In general, SSC is 0.15M NaCl and 0.015M sodium citrate. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook, et al., supra, 9.50-9.51). For hybridization with shorter nucleic acids, i.e., oligonucleotides, the position of mismatches becomes more important, and the length of the oligonucleotide determines its specificity (see Sambrook, et al., supra, 11.7-11.8).

Also included in the present invention are polynucleotides comprising nucleotide sequences which are at least about 70% identical, preferably at least about 80% identical, more preferably at least about 90% identical and most preferably at least about 95% identical (e.g., 95%, 96%, 97%, 98%, 99%, 100%) to a promoter of the present invention (reference polynucleotide; e.g., any of nucleotides 1-1000 of SEQ ID NO: 14; nucleotides 1-1000 of SEQ ID NO 15; nucleotides 1-1000 of SEQ ID NO: 16; nucleotides 1-1000 of SEQ ID NO: 17; nucleotides 1-1000 of SEQ ID NO: 18; nucleotides 1-1001 of SEQ ID NO: 19; nucleotides 1-1000 of SEQ ID NO: 20; nucleotides 1-1000 of SEQ ID NO: 21; nucleotides 1-1000 of SEQ ID NO: 22; nucleotides 1-1000 of SEQ ID NO: 23; nucleotides 1-1000 of SEQ ID NO: 24; nucleotides 1-1000 of SEQ ID NO: 25; nucleotides 1-1000 of SEQ ID NO: 26; nucleotides 1-1000 of SEQ ID NO: 27; nucleotides 1-1000 of SEQ ID NO: 28; nucleotides 1-1000 of SEQ ID NO: 29; and SEQ ID NOs: 47-63 and 70-75) when the comparison is performed by a BLAST algorithm wherein the parameters of the algorithm are selected to give the largest match between the respective sequences over the entire length of the respective reference sequences; but which retain the ability to drive expression, e.g., at a detectable level or at a level at least equal to that of the corresponding non-variant promoter.

Functional variants of the promoters disclosed herein include truncations of the nucleotide sequences set forth herein (e.g., any of nucleotides 1-1000 of SEQ ID NO: 14; nucleotides 1-1000 of SEQ ID NO: 15; nucleotides 1-1000 of SEQ ID NO: 16; nucleotides 1-1000 of SEQ ID NO: 17; nucleotides 1-1000 of SEQ ID NO: 18; nucleotides 1-1001 of SEQ ID NO: 19; nucleotides 1-1000 of SEQ ID NO: 20; nucleotides 1-1000 of SEQ ID NO: 21; nucleotides 1-1000 of SEQ ID NO: 22; nucleotides 1-1000 of SEQ ID NO: 23; nucleotides 1-1000 of SEQ ID NO: 24; nucleotides 1-1000 of SEQ ID NO: 25; nucleotides 1-1000 of SEQ ID NO: 26; nucleotides 1-1000 of SEQ ID NO: 27; nucleotides 1-1000 of SEQ ID NO: 28; nucleotides 1-1000 of SEQ ID NO: 29; and SEQ ID NOs: 47-63 and 70-75) e.g., wherein the 5′ or 3′ end of the sequence is truncated by 1, 2, 3, 4, 5, 6, 7, 8, 9, 10, 50, 100, 200 or 500 nucleotides; but which retain the ability to drive expression, e.g., at a detectable level or at a level at least equal to that of the corresponding non-variant promoter.

The following references regarding the BLAST algorithm are herein incorporated by reference: BLAST ALGORITHMS: Altschul, S. F., et al., J. Mol. Biol. (1990) 215:403-410; Gish, W., et al., Nature Genet. (1993) 3:266-272; Madden, T. L., et al., Meth. Enzymol. (1996) 266:131-141; Altschul, S. F., et al., Nucleic Acids Res. (1997) 25:3389-3402; Zhang, J., et al., Genome Res. (1997) 7:649-656; Wootton, J. C., et al., Comput. Chem. (1993) 17:149-163; Hancock, J. M., et al., Comput. Appl. Biosci. (1994) 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., J. Mol. Biol. (1991) 219:555-565; States, D. J., at al., Methods (1991) 3:66-70; Henikoff, S., et al., Proc. Natl. Acad. Sci. USA (1992)89:10915-10919; Altschul, S. F., at al., J. Mol. Evol. (1993) 36:290-300; ALIGNMENT STATISTICS: Karlin, S., at al., Proc. Natl. Acad. Sci. USA (1990) 87:2264-2268; Karlin, S., et al., Proc. Natl. Acad. Sci. USA (1993) 90:5873-5877; Dembo, A., et al., Ann. Prob. (1994) 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

### Host Cells

The present invention encompasses any isolated host cell (e.g., fungal, such as Pichia pastoris, bacterial, mammalian) including a promoter of the present invention, e.g., operably linked to a polynucleotide encoding a heterologous polypeptide (e.g., a reporter or immunoglobulin heavy and/or light chain) as well as methods of use thereof, e.g., methods for expressing the heterologous polypeptide in the host cell. Host cells of the present invention, comprising a promoter of the present invention, may be genetically engineered so as to express particular glycosylation patterns on polypeptides that are expressed in such cells. Host cells of the present invention are discussed in detail herein. Any host cell comprising a promoter of the present invention disclosed herein forms part of the present invention.

A “host cell” that may be used in a composition or method of the present invention, as is discussed herein, includes cells comprising a promoter of the present invention in which such a promoter can cause expression of a polynucleotide encoding a heterologous polypeptide to which it is operably linked. Higher eukaryote cells which are host cells include mammalian (e.g., Chinese hamster ovary (CHO) cells), insect, and plant cells. In an embodiment of the invention, the host cell is a lower eukaryote such as a yeast or filamentous fungi cell, which, for example, is selected from the group consisting of any Pichia cell, Pichia pastoris, Pichia flnlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia, Saccharomyces cerevisiae, Saccharomyces, Hansenula polymorpha, Kluyveromyces, Kluyveromyces lactis, Candida albicans, Aspergillus nidulans, Aspergillus niger, Aspergillus oryzae, Trichoderma reesei, Chrysosporium lucknowense, Fusarium, Fusańum gramineum, Fusarium venenatum and Neuraspora crassa.

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. Predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)).

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.” “FNGase”, or “glycanase” or “glucosidase” refer to peptide N-glycosidase F (EC 3.2.2.18).

In an embodiment of the invention, O-glycosylation of glycoproteins in a host cell is controlled. The scope of the present invention includes isolated host cells (e.g., fungal cells such as Pichia pastoris) comprising a promoter of the present invention (e.g., operably linked to a heterologous polynucleotide encoding a heterologous polypeptide) wherein O-glycosylation is controlled (as discussed herein) and methods of use thereof. For example, host cells are part of the present invention wherein O-glycan occupancy and mannose chain length are reduced. In lower eukaryote host cells such as yeast, O-glycosylation can be controlled by deleting the genes encoding one or more protein O-mannosyltransferases (Dol-PMan: Protein (Ser/Thr) Mannosyl Transferase genes) (PMTs) or by growing the host in a medium containing one or more Pmtp inhibitors. Thus, the present invention includes isolated host cells comprising a promoter of the present invention (e.g., operably linked to a heterologous polynucleotide encoding a heterologous polypeptide) e.g., comprising a deletion of one or more of the genes encoding PMTs, and/or, e.g., wherein the host cell can be cultivated in a medium that includes one or more Pmtp inhibitors. Pmtp inhibitors include but are not limited to a benzylidene thiazolidinedione. Examples of benzylidene thiazolidinediones are 5-[[3,4bis(phenylmethoxy)phenyl]methylene]-4-oxo-2-thioxo-3-thiazolidineacetic Acid; 5-[[(3-(1-25 Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo3-thiazolidineacetic acid.

In an embodiment of the invention, a host cell (e.g., a fungal cell such as Pichia pastoris) includes a nucleic acid that encodes an alpha-1,2-mannosidase that has a signal peptide that directs it for secretion. For example, in an embodiment of the invention, the host cell is engineered to express an exogenous alpha-1,2-mannosidase enzyme having an optimal pH between 5.1 and 8.0, preferably between 5.9 and 7.5. In an embodiment of the invention, the exogenous enzyme is targeted to the endoplasmic reticulum or Golgi apparatus of the host cell, where it trims N-glycans such as Man₈GlcNAc₂ to yield Man₈GlcNAc₂. See U.S. Pat. No. 7,029,872.

Host cells (e.g., a fungal cell such as Pichia pastoris) comprising a promoter of the present invention (e.g., operably linked to a heterologous polynucleotide encoding a heterologous polypeptide) are, in an embodiment of the invention, genetically engineered to eliminate glycoproteins having alpha-mannosidase-resistant N-glycans by deleting or disrupting one or more of the beta-mannosyltransferase genes (e.g., BMT1, BMT2, BMT3, and BMT4)(See, U.S. Published Patent Application No. 2006/0211085) or abrogating translation of RNAs encoding one or more of the beta-mannosyltransferases using interfering RNA, antisense RNA, or the like. The scope of the present invention includes such an isolated fungal host cell (e.g., Pichia pastoris) comprising a promoter of the present invention (e.g., operably linked to a heterologous polynucleotide encoding a heterologous polypeptide).

Host cells (e.g., a fungal cell such as Pichia pastoris) comprising a promoter of the present invention (e.g., operably linked to a heterologous polynucleotide encoding a heterologous polypeptide) also include those that are genetically engineered to eliminate glycoproteins having phosphomannose residues, e.g., by deleting or disrupting one or both of the phosphomannosyl transferase genes PNO1 and MNN4B (See for example, U.S. Pat. Nos. 7,198,921 and 7,259,007), which can include deleting or disrupting the MNN4A gene or abrogating translation of RNAs encoding one or more of the phosphomannosyltransferases using interfering RNA, antisense RNA, or the like. In an embodiment of the invention, a “eukaryotic host cell” has been genetically modified to produce glycoproteins that have predominantly an N-glycan selected from the group consisting of complex N-glycans, hybrid N-glycans, and high mannose N-glycans wherein complex N-glycans are, in an embodiment of the invention, selected from the group consisting of Man₃GlcNAc₂, GlcNAC₍₁₋₄₎Man₃GlcNAc₂, NANA₍₁₋₄₎GlcNAc₍₁₋₄₎Man₃GlcNAc₂, and NANA₍₁₋₄₎Gal₍₁₋₄₎Man₃GlcNAc₂; hybrid N-glycans are, in an embodiment of the invention, selected from the group consisting of Man₉GlcNAc₂, GlcNAcMan₅GlcNAc₂, GalGlcNAcMan₅GlcNAc₂, and NANAGalGlcNAcMan₅GlcNAc₂; and high mannose N-glycans are, in an embodiment of the invention, selected from the group consisting of Man₆GlcNAc₂, Man₇GlcNAc₂, Man₉GlcNAc₂, and Man₉GlcNAc₂. The scope of the present invention includes such an isolated fungal host cell (e.g., Pichia pastoris) comprising a promoter of the present invention (e.g., operably linked to a heterologous polynucleotide encoding a heterologous polypeptide).

As used herein, the term “essentially free of” as it relates to lack of a particular sugar residue, such as fucose, or galactose or the like, on a glycoprotein, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent.

As used herein, a glycoprotein composition “lacks” or “is lacking” a particular sugar residue, such as fucose or galactose, when no detectable amount of such sugar residue is present on the N-glycan structures. For example, in an embodiment of the present invention, glycoprotein compositions are expressed using a promoter of the present invention (e.g., operably linked to a heterologous polynucleotide encoding a heterologous polypeptide), as discussed herein, and will “lack fucose,” because the cells do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

### Promoters and Genes

The present invention encompasses any isolated polynucleotide comprising any of the promoters set forth herein and functional variants thereof, (e.g., operably linked to a heterologous polynucleotide encoding a heterologous polypeptide and/or a terminator from the same or from a different gene). Vectors comprising such polynucleotides as well host cells comprising such vectors and expression methods using such vectors and/or host cells fall within the scope of the present invention.

For example, a promoter of the present invention includes any of the following promoters:

Pichia pastoris Pp02g05010 (PpPIR1) promoter;
Pichia pastoris Pp05g08520 (ScCCW12) promoter;
Pichia pastoris Pp01g10900 (ScCHT2) promoter;
Pichia pastoris Pp05g07900 (ScAAC2/PET9) promoter;
Pichia pastoris Pp02g01530 (ScPST1) promoter;
Pichia pastoris Pp05g00700 (unknown) promoter;
Pichia pastoris Pp02g04110 (ScPOR1) promoter;
Pichia pastoris Pp01g03600 (ScBGL2) promoter;
Pichia pastoris Pp01g14410 (ScACO1) promoter;
Pichia pastoris Pp01g09650 (ScYHR021C) promoter;
Pichia pastoris Pp01g02780 (ScYLR388W) promoter;
Pichia pastoris Pp03g09940 (ScPIL1) promoter;
Pichia pastoris Pp02g10710 (ScMDH1) promoter;
Pichia pastoris Pp01g09290 (ScFBA1) promoter;
Pichia pastoris Pp03g03520 (PpDAS2) promoter;
Pichia pastoris Pp03g08760 (ScCWP1) promoter;
Pichia pastoris Pp03g00990 (ScYGR201c) promoter;
Pichia pastoris Pp02g05270 (AN2948.2) promoter;
Pichia pastoris Pp02g12310 (ScDUR3) promoter;
Pichia pastoris Pp03g05430 (ScTHI4) promoter;
Pichia pastoris Pp03g03490 (AN2957.2) promoter;
Pichia pastoris Pp05g09410 (ScTHI13) promoter;
Pichia pastoris Pp02g07970 (ScPEX11/PMP27) promoter;
Pichia pastoris Pp01g12200 (AN7917.2) promoter;
Pichia pastoris Pp03g11380 (ScPMP47) promoter;
Pichia pastoris Pp03g08340 (unknown) promoter;
Pichia pastoris Pp05g04390 (ScTIR3) promoter;
Pichia pastoris Pp01g08380 (ScYIL057c) promoter;
Pichia pastoris Pp01g05090 (ScSAY1) promoter;
Pichia pastoris Pp01g13950 (ScTPN1) promoter;
Pichia pastoris Pp03g11420 (ScARO10) promoter;
Pichia pastoris Pp02g11560 (ScMET6) promoter;
Pichia pastoris Pp01g08650 (ScYNL067W) promoter;
Pichia pastoris Pp01g01850 (PpPDHbeta1) promoter;
Pichia pastoris Pp03g03020 (ScSAM2) promoter;
Pichia pastoris Pp03g02860 (PpSAHH) promoter; or
Pichia pastoris GAPDH promoter (e.g., operably linked to a terminator, such as the CYC1 terminator; e.g., wherein any sequence operably linked to the promoter is also operably linked to a downstream CYC1 terminator);
or a functional variant of such a promoter; optionally, operably linked to a heterologous polynucleotide, e.g., a reporter or immunoglobulin heavy and/or light chain. For example, specific, non-limiting examples of promoters of the present invention comprising a nucleotide sequence set forth below:
Pp is Pichia pastoris
Sc is Saccharomyces cerevisiae
An is Aspergillus niger

Sequences of Constitutive Genes
SEQ ID NO: 1 - Pp02g05010 (PpPIR1) ORF:
atgatgtacaggaacttaataattgctactgcccttacttgcggtgcatacagtgcctac
gtgccttccgaaccatggagcacactgacacctgatgctagccttgaaagtgccctcaaa
gattactcacaaacttttggaatagctattaagtccttagatgccgacaagattaagaga
gaggctgtttcccagattggagatggacagattcaggcggctacaatcacctcatctgaa
ccgaaagtaactgcccaagtagtttcccaaataggggacggccaaattcaagccacgacc
tccacttcatcaaaatcgaaagaaaccgctcaagttgtttcccaaataggtgacggtcaa
attcaagccacgacctccacttcatcaaaatcgaaagaaaccgctcaagttgtttcccaa
ataggtgacggtcaaattcaagccacgacctccacttcatcaaaatcgaaagaaaccgct
caagttgtttcccaaataggtgacggtcaaattcaagccacgacctccacttcatcaaaa
tcgaaagaaaccgctcaagttgtttcccaaataggtgacggtcaaattcaagctacgacc
tccacttcatcggaagtaaaacaaactacaggagttgtctcccaaataggagatggccag
atccaagccactacagccactacatctgtcgcttctcagattggagacggccaagtgcag
gagtcaaaaccaacggacacatcagaggataaagggacttctgatttagtgtcttgcctt
actgattcttctcttgctttggttcttgaaaagggtgtgctgacagacgctcagggtaga
attggtgcgattgtggccaataggcaatttcaatttgatggaccgccaccccaagctggc
accatatatgcaggaggatggtcgattacagacgatgctaagctggcccttggtaacagt
acaaccttctatcaatgtctttcgggtaccttctacaatctctatgacgaaaaaattggc
gaacagtgtgaaccagtcgagttggacattgtagacctcatagagtgt
SEQ ID NO: 2 - Pp01g10900 (homologous to ScCHT2) ORF:
atgcaatacagatctctctttttaggttccgccttattggccgctgctaacgctgctgtt
tacaacaccaccgtcactgacgttgtttccgagttggagaccaccgttctgactatcacc
tcttgtgctgaggacaagtgtatcaccagtaagtccaccggattgatcactacctccacc
ctcaccaagcacggtgttgtcactgttgtcaccactgtctgtgacttgccaagcaccacc
aagagctacgtcccacctgctaagactactactattcctcctccagagaagactaccacc
actgtcccacctccagccaagactaccaccactgtcccacctccagccaagactactagt
accgtcccacctccagctaagaccagctctcaccatgagtctaccatcactgtgactgtc
ccatcctccacttctaccaagaagattgagactgagtctaccacttaccactttgttacc
cagaccactactgctagaaacattaccccaccagccatcaccacccaatctcacggtgcc
gctggtatgaacgccgccaacttcgtcggattaggtgctgccgctgttgccgccgctgct
ttggtcttg
SEQ ID NO: 3 - Pp05g07900 (homologous to ScAAC2/PET9) ORF:
atggctgacaacaacaagtctaacttcttcgtcgacttcatgatgggtggtgtttccgcc
gccgtctccaagaccgctgctgctccaattgagagagtcaagcttcttatccagaaccaa
gatgagatgcttaagcagggccgtcttgctaagaagtacgatggtatcgctgaatgtttc
aagagaaccgctgctgatgagggtattgcttctttctggagaggtaacactgctaacgtt
attcgttatttcccaactcaagctctgaactttgctttcaaggacaaattcaaggctatg
ttcggtttcaagaaggatgagggatggtggaagtggttggccggtaaccttgcttccggt
ggtttggctggtgccacttctttgttcttcgtttactctttggattacgccagaaccaga
ttggctaacgatgccaaggcttccaagggttccggtgagcgtcaattcaacggtttgatc
gatgtctacaagaagactttggctactgatggtattgctggtttgtacagaggtttcttg
ccttccgttgttggtattgttgtttaccgtggtttgtacttcggtttgtacgactctttg
aagccaatcgttttggttggtcctcttgaaggttctttcttggcttccttcctgttgggt
tggaccgttactactggtgcttctactgcttcttacccattggacactgttagaagaaga
atgatgatgacctctggtcaagccgttaagtacaacggtgctttcgacgctttcagaaag
atcgttgctgctgagggtatcaaatctttgttcaagggttgtggtgctaacatcctgaga
ggtgtcgctggtgccggtgttatctccatgtacgaccaattgcaaatgattatgttcgga
aagaagttcaaa
SEQ ID NO: 4 - Pp05g08520 (homologous to ScCCW12) ORF:
atgctaaccaaggttatttcactcgctattttaactgcttcagcctttgctgattctgga
gagttcactctttggaacttgtcacccggtgacccttacgactcaactttctggggggta
tctgaaggtttaatcgtccctgtagaacctggtgtgacttttgttatcactgatgaccta
cagcttaagactactgatgatcaattcgttactgttggtgaggactccgctctaggttta
ggagctgaaggttcggtagaattcagcatcatcaacgaggatggcattacctctctttac
tacaacggtgagcttgttactgcttacatttgtgagggtgcagaaccccagatttatctc
acaggttcagaggaggaccccgaatgtgtttcttacactgtcgctgtgataggcgtagac
ggcgaagccccaccaactttcccagaggaagacgacgagacaacaacaaccgatgatcca
accgatgagccaaccgatgagccaactgatgagccaactgatgagccaactgatgagcca
accgatgagccaactgatgagccaaccgatgagccaactgatgaaccaactgatgagcca
actgatgagccaactgatgagccaaccgatgagccaactgatgagccaaccgatgagcca
accgatgagccaactgaagagccaactgaagagccaactgaagagccaaccgatgaacca
actccacctcctcctcattggggaaatgaaactgtaactgctactaagactgagtatgaa
actacaaaagttactatcacttcctgtgaggaaaccaaatgctatgagactacttctgat
gcttgggtttctacttgcaccacagaaattggcggaaaggtaactaaaattgttacttgg
tgcccaatcccatctactccaggtcctaaaccacctaagcctaccaagccaaccgaaact
aagccaaccactgttcctgcaccaaccactaagaagccagaaacaccaactactaagaag
ccagaaacgccagcccctgagaagccagaaaagacaaccactgttattcctccaccaacc
actgagaagccatccactttgtctaccagtagtgttactggaagtgttaccattccaact
ataactgccactggcggtgctggttccaatttcaacttgggtggattaaccgtcggagtt
gctggtattgcaatggctttgtttgtg
SEQ ID NO: 5 - Pp02g01530 (homologous to ScPST1) ORF
(including 1 intron):
atgcagtttggaaaggttctatttgctatttctgccctggctgtcacagctctgggagaa
acaacttcttcattgagtaagtattgatcatttgaaattttttcgggagttttttttgat
gttctgcaaaggaatccaagaaacatttatgaggttaccaggtaggtttttgttcagaag
taactaggttgactcaagctttcaactgttcaatctaatagcctgaactgtttgagttta
cctggtagtcctgagtctttcaattacgagagaatccctgtgatccgatccctgtcgcca
aattcaaatcgcccaagacggtcagccaatttgttttctgatggcttgggcgctttatgc
gtcagggacgaggaactacagttttgccatacatgatacgggactatccaatgcagctca
ccctttgtggtgtgaatcttgcccgtttcctaaaacgaatcaaacgggcggagctcaatg
gcagttccacatctgtatggagctatttttagatttttggttcctttcgcgatttgttga
tcatgacactcccctaacatttcattttcttctgagataacgtactaacaatatgccaag
gtgccacattgacttcaaccacacgaatttcaattgcatctggatgtagtcttgaagact
tcactgctactgctcagtctaatcttgatgagttgtccgattgtgaagctgtcagaggtg
atatccacattgccggaagtctgggatcagctgctatcgctaatgtcaaggccatttacg
gttctttaattgtcaagaatgccacctctttggtttcattgaccgctgattctttgacca
ccattaccgaacaattggctctttctgagttgaccattttggacacactctcttttgctc
aattgacttccgttggttctatttacttcgtcaccttgccagctttggaagagaccggtt
tccacactggtgtctctgacactgagtctgtttacatttctgacactgctttaaccaact
tggacggtatcgttgctaccgatgtcgatgttttcaatgtcaacaacaacttcaatttgg
acactgttgactcgcacttggaaactgtttcctctgctttggaggtttccttcaactcag
acgatgttgaagtatcctttgacaatttgttgtgggctaacaacattaccttccgtaacg
ttgcttccgtctctttgaacaacctgaccactgttaacgcttctttgggtttcattgagt
ctggtttccaaaagttgagtttcccaggaatcaccagggttggtggatctttctccattg
ttgacaacgacgatttagaagaaatagacttttctaacgttcagtcgattggtggtggtt
taatcatcgctaacaactctaagttgactgacttcagtgaatgggaggacttgcaaactg
ttggtggtgctcttgttttggagggttctttcgacaacggttccttcccatctttgagag
ctgttagaggtgccttctctttggagtctgatggagatatctcttgtgatgacttctctg
atatcaggggtgacactgccggtgagtatcagtgctctgctgcttcgttctccacttctg
ctagtgctcagtcttcttctagttcaaccagtactggcggatcctctacccacaccggta
gttccactgctaccagctcttctagtgaggatgctggtgtagctttggctcctgcttctc
tcttcactttgttagcctcaattgttctcggattcttg
SEQ ID NO: 6 - Pp05g00700 (unknown) ORF:
atgcaattcaagtctatcgttttaactctagctgccgttaccgttgctcaagctaacaac
ctatcaaacgagagtaatggtactaatcactccaaccatacttcttccgtgccaactgga
gctgccgttcgtgcctctggtatgggagctggcttgttgggagctggtgttgtagccggt
gttgctctattgatt
SEQ ID NO: 7 - Pp02g04110 (homologous to ScPOR1) ORF:
atggccgtaccagcattttccgacatctcaaaagcatctaatgatgtactaggcaaggac
ttttatcacttgaccccagtctctttggacgtgaagactgttgctgccaacggtgtaact
ttcactgccaaaggtaaatctgccggtgacaagctatctggaaacctggagaccaagtac
gccgacaaaaagaacggtttgactttgactcagggctggaacactgccaatgccctagac
accaaggttgagctggctgacactttgactcccggtttgaaagctgaggttgtcggatct
gttgttcccgacaagaagaaggacgctaagctgaatttgacctatgctcaccaagcattc
actgcccgaacattcttggatttgttaaagggccctacagtgaatgctgactttaccgct
ggtaaagacggtgtcactttgggtggaactgcttcctatgacattaatgccgcttccgtc
accaaatatgctttcgctgtcggatacaaggccccagactactccatttctctttctgct
ttggataacgtcaaactgttttctgccggttattaccacaaagtttctcctctcgtcgag
gtaggtggtaaagctacttacgactctaagtcctctattgcaaacccagttgctttggaa
gtagcaactaagtatcaggttgattcgacagctttcgttaaagccaaaatagctgattct
ggtattgcctcatttgcctactcccaagatttaagaaagggtgttaagttaggtttagga
gctgctattgacgttctcaagttgaacgaggccactcataagctaggtgtatctctttct
ttctctgct
SEQ ID NO: 8 - Pp01g03600 (homologous to ScBGL2) ORF:
atgatctttaatcttaaaacactggctgcggttgcaatctccatttcacaagtgtctgca
gtttcctctctgggttttgctctcggaaacaagaacgttgacggaacttgtaagtacttg
gccgactacgaggccgacttggatactattagaggcggctctgaagccgttgctattaga
gcttattccgctgaagactgtaacactttacaatacctcggtcctgctgttgaagagaag
ggcttcaaattagttctatcagtcagaccactggatgagagctactaccaggcagaaaag
aatgcactaagtgaataccttccccaattatctgtttcgactttgcaatttttgtcagtt
ggatccgaagctttgtacagagacgacttgccagcttcagatctggctgataaaattaga
gatatgaaggagtttttggctggcttgactgacaagaatggggactcctactcttccgtc
ccagtcggaaccatcgattcatggaacgtccttgtagacgcctccgctgcaccagctatt
gaagcatctgatgccgtttacgccaacgctttctcatactggcaaggtcaaggtccttcc
aactctacctattccttctttgacgatatcatgcaagcattgcaagtaatccagaccatc
aagggatctactgatatcgatttctgggttggtgagaccggatggcctaccgacggtgac
aactttggtgatgctgttccatctattgagaacgctgacaacttttggaaagaagctatc
tgcggtatcagaggttggggcattaacacattcgttttccaggcatttgacgaagactgg
aaggaagaggacgacgctgttgaaaaccacttcggtgtttgggacagttccagacagtta
aagctggactcattaggttgcgacttttcttct
SEQ ID NO: 9 - Pp01g14410 (homologous to ScACO1) ORF:
atgttatccgccagaagagtacttgctaagataaacagccgtggattggccacggtgtca
gggctcaccaaggattctctcgttgagatgaacctgttggaaaagggcaactacattaat
tacaagcaacaacttgacaacgtcaatattgtcaaggaaagactgggaagacctttgacc
tacgctgaaaagctgttgtacggtcacttggacaagcctcatgaacaagacattgagaga
ggtgtctcttacttgaaattgagaccagacagaatcgcttgtcaggatgctaccgctcag
atggccattttgcaattcatgtccgctggtatgccttctgtcgctactccaactactgtg
cactgtgatcacttgattcaggctcaaaagggtggtgctgccgatttggagcgtgccatc
agactgaacaaggaagtctacgatttcttggcaaccgcttgtgccaaatacaacattggt
ttctggaagccaggttcaggtattattcatcaaattgttctggaaaactatgctttccca
ggtgaattgctgatcggtaccgattcccacactcctaacgctggtggtttgggtcaattg
gccatcggtgttggtggtgctgatgccgtcgatgttatggctggtttgccatgggaattg
aaggccccaaagattatcggtgttaagctgaccggtagaatgaatggatggacttctcca
aaggatatcattctgaagttggctggtatcactaccgtcaagggtggtactggtgctatc
gtcgagtacttcggtgatggtgttgacaccttctcttgtactggtatggctaccatctgt
aatatgggtgctgaaattggtgccactacttctgtgttcccattcaacaactccatggtt
gacttcttggacgctactggaagatctgagattggtgagtttgccaaggtcttccaaaag
gagtacttgtctgccgaccctggttgtgagtacgaccaggttatcgagatagacctgaac
accttagagccacacattaacggtcctttcaccccagatttggccactcctgtctccaag
atgaaggaggttgccgttgccaatgactggcctctcgaggtcaaggttggtttgatcggt
tcttgtactaactcctcttatgaggacatgaccagagccgcttctatcattgaagatgct
gcctcccatggtgtcaaggctaagtctttgtacactgtcactccaggttccgaacaaatt
cgtgctaccattgccagggatggtcaactgaagactttcactgacttcggtggatccgtt
ttggctaacgcttgtggtccatgtattggacaatgggatcgtcaagatatcaagaagggt
gacaagaacactattgtctcttctttcaacagaaacttcacttctagaaatgagggtaac
ccagctactcacgcttttgttgcttccccagagatggtcaccgcttatgctattgcaggt
gatttgagattcaacccattgactgacaagcttaaggacaaggacggaaacgaattcttg
cttaaggaccctgtgggagtcggtcttcctgtccgtggttacgaccctggtgaaaacact
taccaggctcctcctgaagacagagcctccgttgaagttgtcatttctccaagctcagac
cgtctgcaaagactgactccattccagccatgggatggaaaggacgctgagagattgcca
attctgattaagtccgttggtaagaccaccaccgatcatatttctatggctggtccttgg
ttgaagtaccgtggtcacttgcagaacatttccaacaattacatgattggtgccataaac
gctgaaaacggtgaagccaacaacgttaagaaccactacaccggtgtctactccggtgtc
ccagacactgccgccgcgtaccgtgacaatggtgttaagtgggtcgtcattggtggtgag
aacttcggtgaaggttcctccagagaacacgcagccttggagccaagatacttgggtggt
ttcgctattatcaccaagtccttcgctcgtattcacgagaccaacttgaagaaacaaggt
ctgttgccattgaacttcactgatcctgcagcttacgacagaattcaacccgatgatgag
gttgacattttgggattgaccgagttggctccaggtaagaatgtcactttgagagttcac
cctgccgacggctccccaacttgggaaactccattgtctcacacctacaatgccgaacag
atcgaatggttcaagtacggttctgctttgaacaacatggctgccgtcaaggcctctaaa
SEQ ID NO: 10 - Pp01g09650 (homologous to ScYHR021C) ORF:
atggttttagtccaagatttattgaaccccaacccagtctccgaggccaagcaacacaaa
ctaaagactttggtccaagctccaagatccttcttcatggatgtcaagtgcccaggttgt
tttgagatcaccactgtcttctctcatgctcaaaccgctgtaacctgtgattcatgcacc
actgttttgtgtactccaaccggtggtaaggctagattgtcagagggatgtgccttcaga
aggaag
SEQ ID NO: 11 - Pp01g02780 (homologous to ScYLR388W) ORF:
atggctcacgaatcagtttggttttcacacccaagaaactacggaaagggttctagacag
tgccgtgtctgtgcctctcaccaaggtttaatccgtaagtacggcctgaacatctgccgt
caatgctttagagagagagccaacgacattggattccacaagtaccgt
SEQ ID NO: 12 - Pp03g09940 (homologous to ScPIL1) ORF:
atgcatagaacttactccctacgttcaggccgtgctcctacggctgctgatgtcaacaac
cctccaccacctacctctactaccaagagcaagttctttggtaaaaactccattgcttct
tctcttcgaaaaaacgccgctggcgcttttggacctgaattgtccaagaagttggctcaa
ttgatcaaaattgagaagaacttggagcgatccatcgagcttgttgccagagagagaaga
actgttgctaagcagttgtctctgtggggtgaggaaaacgatgatgacgtgtctgatgtt
accgacaaattgggtgttctcatttacgagattggtgaattagaagatcaatttgttgac
aagtacgatcaatacagaatcactctgaaatccatccgtaacatagaatcttctgtccaa
ccatccagagacagaaagcaaaaaatcaccgatgagattgcccacctcaagtacaaagac
cctcaatctccaaagattccagtcttggagcaagagctcgtccgtgctgaggccgaatct
ttggttgctgaggctcaattgtccaacattaccagagagaaactgaaggctgctttcaac
taccaattcgacgcccttgctgaattgtctgagaagctagcattgattgctggttatggt
aaggctttgctagagttgttggatgactctccagtcactcctggtgaaaccagacctgcc
tatgatggttacgaggcttccaaacaaattattatcgatgctgagaatgctctttcctct
tggactcaggacaatgctgctgtcagaccgtcactttctatcagaaatagtgaatatgat
gaggagcccaacgaagagtgggaagaaaaggaagagggtgacgaaactcgtgacgaaact
caagcc
SEQ ID NO: 13 - Pp02g10710 (homologous to ScMDH1)
atgttgtccacaattgccaagcgtcaattctcctcctctgcctctactgcctacaaggtt
gccgttcttggtgccgctggtggaattggtcagcctttgtcgttgctgatgaagttgaac
cacaaggttactgacttagccctgtatgacatccgtttggctccaggtgtagccgctgat
gtatcccacatcccaaccaactccaccgtcaccggttacactcctgaagataatggtttg
gaaaagacactgacgggagctgatctggtcatcattccagctggtgtcccaagaaagcca
ggtatgaccagagacgatctgttcaacaccaatgcttctattgtcagagatttggccaaa
gctgttggtgactacagtcctagtgctgcggttgctattatttctaacccagttaactcc
actgttccaattgttgctgaggtcttgaagtccaagggtgtctacaacccaaagaagcta
ttcggtgtcaccactttggatgttctgagagcctctcgtttcttgtctcaagtgcaaggt
accaacccagccagtgagccagtcactgttgttggtggtcactcaggtgtcactattgtt
cctctgctgtctcaatctaagcacaaggacttgccaaaggacacttacgacgctctggtc
caccgtatccaatttggtggtgatgaggttgtcaaggccaaagatggtgctggatctgct
acgctgtctatggctcaggccggtgctagatttgccagctccgtgttgaacggtttggcc
ggtgagaatgatgtcgttgagccatctttcgtcgactctccattgttcaaggatgagggt
attgaattcttctcctccaaggttactttgggtccagagggtgtcaagaccatccatggt
ttgggagaattgtctgctgctgaagaggagatgatcacaactgccaaggagactttggcc
aagaacatcgctaagggtcaagagtttgttaagcaaaaccca

### Sequences of Constitutive Promoter-Polylinker-Terminator Cassettes

[Unless otherwise described, the polylinker is in bold, and the promoter precedes the polylinker and the terminator follows the polylinker. In some sequences, upstream and/or downstream restriction cloning sites are also bolded at the 5′ and/or 3′ ends of the displayed sequences. The scope of the present invention encompasses embodiments wherein the bolded cloning sites are absent completely or are other than that specifically disclosed herein; as well as wherein sequences not having bolded cloning sites do encompass a cloning site of any sort].

As mentioned, the scope of the present invention encompasses compositions and methods comprising the following whole cassettes or the promoters in said cassettes.

SEQ ID NO: 76; Pichia pastoris GAPDH Promoter and CYC1 terminator
AGATCTTTTTTGTAGAAATGTCTTGGTGTCCTCGTCCAATCAGGTAGCCATCTCTGAAATATCTGGC
TCCGTTGCAACTCCGAACGACCTGCTGGCAACGTAAAATTCTCCGGGGTAAAACTTAAATGTGGAGT
AATGGAACCAGAAACGTCTCTTCCCTTCTCTCTCCTTCCACCGCCCGTTACCGTCCCTAGGAAATTT
TACTCTGCTCGAGAGCTTCTTCTACGGCCCCCTTGCAGCAATGCTCTTCCCAGCATTACGTTGCGGG
TAAAACGGAGGTCGTGTACCCGACCTAGCAGCCCAGGGATGGAAAAGTCCCGGCCGTCGCTGGCAAT
AATAGCGGGCGGACGCATGTCATGAGATTATTGGAAACCACCAGAATCGAATATAAAAGGCGAACAC
CTTTCCCAATTTTGGTTTCTCCTGACCCAAAGACTTTAAATTTAATTTATTTGTCCCTATTTCAATC
AATTGAACAACTATCAAAACACAGCGGCCGCACTAGCTTAATTAAACAGGCCCCTTTTCCTTTGTCG
ATATCATGTAATTAGTTATGTCACGCTTACATTCACGCCCTCCTCCCACATCCGCTCTAACCGAAAA
GGAAGGAGTTAGACAACCTGAAGTCTAGGTCCCTATTTATTTTTTTTAATAGTTATGTTAGTATTAA
GAACGTTATTTATATTTCAAATTTTTCTTTTTTTTCTGTACAAACGCGTGTACGCATGTAACATTAT
ACTGAAAACCTTGCTTGAGAAGGTTTTGGGACGCTCGAAGGCTTTAATTTGCAAGCTGCCGGCTCTT
AAGCGGACCG
SEQ ID NO: 14 - Pp02g05010 (PpPIR1) promoter-polylinker-terminator
cassette (promoter is nucleotides 1-1000 of SEQ ID NO: 14):
GGCAAACTGTTTAGAGTTGTGACGATGATCATGTCCAATAACTATTGATTTACCGCCAGCTTTTGAC
ATATAGTTGGCCAGTCCCTGAGATGCTTGTATTATCGTGAGATCATTCATTAGACTAAAACCAGCAC
CCATTCTACCTCGAAGTCCCGCTGTTCCAAAAGTAATTCTTCTTCGTAAGCGTTCTTCAAGAACTTT
AAACTTTTCATTCTCAAGAAGTTTGAGAATCTCTGATCTAGTGGATGGATTACGATCAATGCTAAGC
CATTGATGCGCTAACGCCTTAATTGATGGGGACATGATGTCAGATGTAATACCTTGTTTACCGAAAT
TTAGCTATCTCACGTAGCCTAGAATGGCCGCTGCTAATCGGCCTATATCTAAAACGGCATATCGAGG
ATCGATGAAGTTTACACGTGACGCATTCAGTGTAAGTAAATTACATGCAGTGCCGGCTACTTGACTA
AAGTAAGAAAAAACTCAGAAATCAAAGAAATAACAGAAGCTGTACGACACAACGAAATAAATACAAA
CTTTCTTAGGATGCAATTTAACAAGAATCGTCTTATTGTCTCTCGCAAACATTCAAGAATTTCAGCC
CATTATCCACATTCAGTCATGGCGCATTATTTAGATTGTGTTGTTTGATAAAAAAAGAATACAGCAA
GTAACAACGAGTAGTCAGTTACTGAGATATTTTTTAGTACCGTCGATCCGGTTACGGGGAATTTAAC
TGCAATATATCAAACGTTAAACTGGATCGAGTTCAAAAAAACAAGCTCTAACTCAGTCAAGCTTTAT
GTATAGTTTCCTAATCAGGTAATTTTTTATGATCTTGTCTGTAGGTTTGATTAAATTTCCATTACAT
CTAAAAATAGTAACCCAGCCATCTGAAAAATGGATCTTTCGGGATTCAATATTCACAGTATAAAAGG
AAGACTTCTCTTAGGATTCTGACCCGTTCATACTCATTGCTAAACATATTAAATACTGAGAAGCGGC
CGCGGCGCGCCTTAATTAAATGTTGGGTTTATGGTCATTTCATGATATTGGCTGTTCTTGTGTAAAA
TTTTAACTTCTTCGTATAAAATGCATCTTATTTTTTGTATCATGAAATCGTAGTGTCGATTGAGTGG
CATCAAACTGGACTCTTTTCGCGTTACTAAGCTTTTGGATGTGCAATTACATTTGGCCTCAATACTA
AGACCCATCTGCAGTAAAAGTTGAGACCTTAGTAAGGAAAACACATAACTTGATTACTTTGGGTCGC
TAAACTTTGCAACATGAATCTGCATGGTCAGAAAACTCGTCTTCGCAGTGTACTGCACACCATAATA
GTTCACATCTTTCGCGCTTCCGTCTAGTGGTATGATTTCCCCAGAAAACACACCACATCAGCCAAAG
ATAGCATGATTTTGAACTAAATGAGATGAAGATTATATAAATTTGACCGCAGTCTGAAGGGCTATAA
ATAGGGCTTAGTTCTTCTCCCCATTTAATAGTCAGAATAACTACTCTACTCGGACCGATTTAAATGG
SEQ ID NO: 15 - Pp05g08520 (homologous to ScCCW12) promoter-
polylinker-terminator cassette (promoter is nucleotides 1-1000 of
SEQ ID NO: 15):
TTTTCGTTTGGGTTCATTAACTGACGTATGACATGGCGTGGACAGCAAGTCGTCCAAAAATAACTAG
GCAGCTGCGACATTTTTTCGTAGAGCTAGTTTCATTCGAACCTGAATTTCGGTATGCATGGCATGGC
GAACGTAAACAAAAACCTTTGCCTCTGTTGAAATTAACCACCAAAAATTGATGGTCTCATGATGTGG
AACTCACGATATTTGTTCTAGCCCCTCCCCCGTGGTAATCACAAGTCGAAGTGTGCCCACTTTTTCA
GTGTAAGTATTGTGCCTAACATATATCATTATTCTATGTGATAAACGCCACAAGTCAAAAAGACTGA
CACGATGATACTCTCCAGGGTCCTGTTGACTCTACTCCTTACAACCCTCAGGGACCAGAGCAGTTCT
CGGTAACTACTGTGACGTGAGGAGAGTCGAACAAGACCGAACGTGGGACCAGTAAAATGCAATCTGC
ATGCACAGAAACGACCGTGATTGTGTCCAATGTAAGAAGCTGGAATTGTGCATGCCATGACTAGGTT
TTGAGGCTAGTACCAAATGGAAAAGTACACACCACCAACTGGGTTGGTGGTGACAGTCAGAGATCCA
GAACCATTGCAGGCAGTTTCCAGACTCTGCTGAGTCACTTTCGTGTTCAATGAAAGTAAATGGAAGT
AAATCCCTTACAAATTGTCCAATTGAGAAATAACATCTTCAAACTGCTGTTCGCATTTGCCGAGAAA
AAGCTTCCCTATTTGGGTTCTATTCAAAGGAAAACATCAATGCCTGATTTACCCTGGGAATGATGTG
TTCTTATGTATGATCTTATGCACACCCCTGTCCAGAAAGATCCACAAAAATCCTATTCCATTCTGGG
TCGAACCTAATCCCAAGCCATCCAAAAATGGCTAATAAGAGATTTAAAAATAGTAGATATCACGGTA
TATAAACAGCCAATTAGTCCTTTAATTGGCTTGAAGATAATTAATATAACTACCGTTCAATAGCGGC
CGCGGCGCGCCTTAATTAAATTGCTTATTATTAACACTGTATTCTATTGTTTCTCATTGTAGCCACA
TTGCACAGCTCTGTTCTTATTATTGCCAATCTGAAAGTTAAGCCTAAGTCCGTTTTTAAGCTATTGA
TGTACAACATCTTTCACGTGATACAGATGCTGTCTGCCCATGCATGCCTAAATCTAATCCCTAATAT
ACTACATTTCGCTTATTTTACCCTCACTACTTCTCCTAGATTATCGGTGAATAAAATGCTGCGGCTA
GATAAAATCCGCCAATCTGCTAACACTGACTATTTTGGCTAGGTTCGTCTGTGTTTATCTTTAACCA
GGTTATTGTTCATCTGTGTTATTTTTGTATTGTGTTTTGCACCATGCGCGTCATTGCGCATACCAAG
CATTGAGTAACAAAGAGAGATTCAATCATGAACAATAAGCAAAACCTAGTTACAATAAGGGAGTAGT
ATAACAGCTTATGCGTTATGCATTAATCGACTAAAAACTCAGCAAACCACGGACCGATTTAAATGG
SEQ ID NO: 16 - Pp01g10900 (homologous to ScCHT2) promoter-
polylinker-terminator cassette (promoter is nucleotides 1-1000 of
SEQ ID NO: 16):
CTTGCCAGGATCTGACCACTACAAGAAGCGTAATTAAGTATTTATTATGTAGCTTGAGGAATTGATT
TCTAAAGGCCAAAGATGATGTAGAGCAATAGTAGAGTGATTAGCAAGTTTCTGAACATGTTTTGTGG
TCGGGTATTAGGCGCCGTACCTCTGTATTTTTTTTTTTTGGCAGATGGGATGACCACTGTGCTTACA
TAATTTTCGATGGGCCCTTTTGGAAACGACCACTCCTTCCGATCACGTAATACTAGGCGAGTATCAC
TACGCGTCGGTATCCGCATTTTAGCCGCTTTAACGACAAGGTCAGGGTAAATACTGTGTTGCATGGG
CGATGCCGTAGGATCTGGCCAACTTGGAACAGAGGAGCCCACCGCGTAGGCAGCGCTTGGGACCAGT
TTGTTAGCCAATCAGTTCCCCCGTCTGAGTCCCTACACGCTCAGGCTTGCATGGATATGCATGAAAA
ATGTCAACTCAGTCGCACACAGGGATGCTGTTGTCGTGGTGAGAAAAAGCTAAAATAGCAAACCCAT
AGCAATCGAGAGACTGTGATTGGAAGCAAAAGAACCAGGCATTGACTAGGAAAGACTGACAAGAAAA
TTAGACGGTGCCAGTGGTATTTCATTGAATTGCACTGAACACGCCTCCCATTTCCTTCCCTCAATCC
ATTCAAAGGGCAATTAAACCTTATCTCGGCCTACATTAGTTGTCATGGAAACGTCTCACTTTCTCTC
TGTGTCCCACAGTAGCGAGTAGGCGGAGAGACCAAAAATGGTCTCTCGGCCCTAATCAAAACATCCT
TTGTCGCGTTGTTTTCAGATCGATTCGCAATCTGAGAGAAAAACAATAACACCAAAATAAAAGCTTG
GAAAAAGAAAACATGATTCACATCCACTAGTAAAATATAAATACTCCTGCCGCCTCCCCCTTTCCTT
TCTTCTTATTCCTCAACTCACTGTTTCAGTTTATTCCAACTACTTTCACTCACTTATCAAAAGCGGC
CGCGGCGCGCCTTAATTAAGTTTGATCACTGAATTACCTCACACGGTTGTATTTTAGCAAAATTTCT
TCGTAATTAACCAATTTTTTTATTTACAAACTTTGTAGAGGTCGCTTTTTCAAGAGATAAAGAGCGA
TCGGCCTTCGGTTTTTTATTCCGCTTTGGGATATCTCCGAAGACTTAAGGTTAAGGCTTATATATTG
CGCGATGCACCGTTTCTATTTCTCTCCCGCTTTTCATCCTTAAGTCCAATCAGTATGGCTTTTTTGT
CGAAGTTCTTGGGGGGCAAATCCGGCGAAGCCCAGGCGGCTGAGGTGATCTCAGAAATAGTTCCTCT
TTCACAGTGCCCCGCTACTTGTTCGGCCGCTGAGTGCTTCACAAGGTATCCAAACAGTTACAAAGAT
GACCCAGGCTCCAAAAACGATCTCCTTTGGAAATCAACTAAACCGTACTCTTTACACATTATCATTC
CAACGGGTAAGTCCGATTGGAAGCGTGACGCCACTGATGTTGATGGCACGCGGACCGATTTAAATGG
SEQ ID NO: 17 - Pp05g07900 (homologous to ScAAC2/PET9) promoter-
polylinker-terminator cassette (promoter is nucleotides 1-1000 of
SEQ ID NO: 17):
TTGTTGTTCCTAAGGCTCTGAGAGTTCTGAGATTGAAGCCAGGTAGAAAATTCACCACTGTCGGAAA
GTTGTCTACTTCCGTCGGTTGGAAATACGAGTCTGTTGTTGAGAAGTTGGAGGAGAAGAGAAAGGCT
GAGGAAGCTGAGTACCAGGAGAAGAAGAGAGCTTACACCCAGAGATTAGACGCAGCTAGTGCCGAGT
TTGCCCAAACCGAGGAGGGAAAGCAGTTGGCTGCCTTTGGTTACTAAATAGTAAAGTAGGGTATCTT
CAAGTAATAGTATACTAACCATCTGAAATAACCACCGTCCTGTAGTTTTTTTTCGATATCGAAGAGC
CTATGCTAGTACTGTGGATTTGCGCTCCATCCAACATCTGTGCGCAAACTAAAACTTCCGAGACTGA
CATCTACCATCGCTAGACCCTAAGTAAAACCAATCTCGCGTCCGAACTTTTAAATTTCAGTCCTTAA
AACTTCAGAGCATTGGTTGTAGTTTCCGGATCTGAGGGGTCGTATTGGAGTCAAGAGACGGAGCTGC
CTCCACAGCGCGAAACGTCAACCCCAACACCAACCTGAATTTGCAATCACCATGGGGACAAGTTTCA
GCAGTCAATGGGCAATTCAGACGTTGATACGGTACCCATTTGCTAAGCTCAATGACGATCCATCCAA
CTTCAGAGAAAGGCCTTTCTCTGGTATGCTCTGGTATTCATTCGTCTTTTATCACTCTCGTTGCACA
ATGCCCGGGTACTCCCGGAACAAGGGAGTCTTCCAGCCAAGCTGTACAGAGTGAAAAATAGAAATAC
ACCTTTGCAATCAAGACGCGCGTTGGCCAATCACAAGACTTAATCGGTGCAAAGAAGGATTACCAAA
TTTTTTTTTCCCAAAATCGCTATATAGAAATAATGGAGGAAAAAGGGTTAATATAAAGGAGAATTCC
CCCGTTTTTCTCCCCTTTTCTTTTCTTCTTCAGGCTTTCTTACAAATCTATAATATTCCAAAGCGGC
CGCGGCGCGCCTTAATTAAGCCAATTAGTTTGAAGTGAGATTTTATTTCATTCCTGTTAATATTATA
TACTAGAGTATATTTTAAATTAATTGTTCATGAACTTGCCAAATTATGTTAGTTTTGTGTAAACAAT
CTTAGGCTATCCAATTTAGTTCTACTTTTGGTAGATTTCCTGTTTTGGTAAATTACAAACAACAATG
ATTTGACTTATATTCTATTCGGAATTTTACTTATCACCTTGTACAGTTTGTGGGGATTTCCGGACAT
GAAGAAGTTTGGACTGGAAAAGGACGGAGATGGAATCTTCAGGCTCAAACGAAGGTCCTCCTCTCCC
AATAAGAAGGGCAAAACAACATTGCCTCCTTTCTCGTCACCTCCAAATCTAGCCCATGACAGTCCAA
CAGGGCCCCCAATATTACCTACTCTCAGAACCAGAGTTTCTGAAACTGACACTTTGGTAGATGGTAT
CAGTGATACGGTCAACATTCCCTCTCCACAAGCTATGAATGCGAGGGCCGCGGACCGATTTAAATGG
SEQ ID NO: 18 - Pp02g01530 (homologous to ScPST1) promoter-
polylinker-terminator cassette (promoter is nucleotides 1-1000 of
SEQ ID NO: 18):
CGTGGGAAGAAGGGTTGGATGGCTTATCCAAATTGATCACACCTGAAGAGATATAGTCCTTCAAGTC
TCTTTTCAAGGGAGAGCACCCGTGAGGGATAGGAGTATAATGCCCACTTCTAACCAACAACTTGTCG
TAGTTCTTCAAGAGCAAAGGCCAGTCCGAAGTATCATTGGAAGGAGTCACGGCCTCTGGCTTAATTG
TGTAATCAGTTTCACCCATGATAAGTTAACGTAAAGAAAATTTTCAGTAGAAAAGAAAAAAAAAACC
TAACCTTTAAGTGTCGTGTACTCAACAAACGATGTTGGATGAACCTTTTCACATAGTCACTGGAGTT
AGGAAGTGCGATGCGGTATTACATTACAGATGGATGAAGGCCTCCCGCAATGCTTTAGTTTGAACAT
CTACGTGGGATTTAAGGTGCAACCAACAACCGGAGTCTGTTAATCTAAATTGGGAACAACGGACATT
GACAAAAAAGAAGCGGCTATTTGTTTATGTTTACCATGAGGATTTCAGCTCAATCCGTCTTATATAA
TCTGTCACCTACTTGAGTTTTGTGGCCTTTTCAACAACGGAATTGAGATACAGTTTGGGATTTTTTT
CATGCTAAGTCGTATTTCTCTTTCACCCTTTGCTGAACTAATCCAAAGTTTATTTTGATCTTTCAAC
GTCTACACATTTTGAGCTACTTCTGATCTTCTCTTCGAGTTTTGGGCTTCGGTACCCGTAACAATAG
TGCAAACTGCTAGTGTGTAAATATTCGCGCATCGTGAATAATCCTCAAACCTACATGTTGATGCAAC
TAGTTTACCCTTTTTAGTATTATTCCCCGATCTGGACAAACAAAAATAACGTCTCACAACAAATCAA
GCCTCGATTTTTTGCTCTGGAAAATATCAACCGACGTACTATATTAGGCCTCCTAAGGCCATTTTTT
TTTTCCATTTTCTCTTCACATCCTTGCGCTTATTTTTTTTTGCTCTAATTAAAAGTCCAAAGGCGGC
CGCGGCGCGCCTTAATTAAACATAGAGAAATAAAAAGAAGAAGCAGAAAAGTTGAATGGAATATTTG
TATTGTTTAGTTTTATTTTAAAATTTAGTTCGTAATGAATGTTTTATAAGTATTTCTATACGTACTT
ACAACCCCTTACCGTAGTTTCCAGACTCAAAAAAGGAAAAGATAATCTTGACAATGTTTTCTAAAGA
GGCCAACCATTCCAAGAGTTGAGCGGTGACCTTTTCTTGGTCATCTCCCAATTTTTTGAAAAAGTCC
GCTCTATATGGACAGGCTTTCATAGCTAACTTGAAAATTGGCTTGATCACGAACGAATGATGCTTAC
TTAAGGTTTTACCATAGGCATTAGTGAAAGCCTTCGTGAGTTCATAGTCTGGATTGTCTACAGTCTC
TCTCATAGCTGCTGCAGTAAATTGAAGGCCTCTAGATAGCCATAATAAACCTTGGGTAGCGGATTTG
CCCTTGATCGATGCGTCTTTCTCATCCAAAACCAAGTCTTGAAGCGTAGCCGGACCGATTTAAATGG
SEQ ID NO: 19 - Pp05g00700 (unknown) promoter-polylinker-terminator
cassette (promoter is nucleotides 1-1001 of SEQ ID NO: 19):
GTCCAGAAAACTTATGAAACGAAAATTTCTCGTTGACAAAAGAATCAAGTAGTTGCAAAATCACTGT
TTACAATTGGACATTTCGAATGTACTATGAGGCGATCGATCTTCCTTGATTGAATAGTCCAATACGG
AAAATGAAATCGTGTCGATTTATTACTTAATCGGTCGAGGTATGTTCGAACGGTTTCAATGAAAGCA
GGAGAAGAAGGTTACTCTCCAAGACCACATTGACCATCCGAGCCCGCCAGGCCCTACTTGACAGCCA
GGCTACTAAAACTGAATGTAACAAGGAAAAGAGAGTTTACTACTTTTCGATCTCCTAAAGCTAAGGA
GACTGGATGGTTTTGGTTGTACCTGCAGGACAGGGGATTCGCTCTGAGTTGAAAGGCCAAATTGAGA
TTCTGTGACAAACAAAAAGAGCTGGTCCTATGGAATTCCAGATTCTTCTATCTTGCATAAATACATG
CTTATTTAGGTAACTCATTTTGCCCAAATAGGAAATATTGGAGTATCCTTTCTATAACAGAATTGTG
TTTAATTCTTATGTCATCCTACGGGCATCGCTCTATATCATTCACCACGTACGTTCCAATGATAGCG
TAATAGCGTGCGAAGAAAAGGGAATAGTAAGCCACTTGTAGAAAAGTGTTGGCTGATCACTACTCAA
AATCAGCTCCATACCCTGAGTTAATCAGTTCACGTTGCTCAAGCGTCTAAAAATAGATTGCTATTGC
CTGCGAATTCTCTTAGGCCTGGAACGTTCTACTGGGCTCGCGGACCGTGTTTATTCCTGATTCTGGA
GGTAAACGGTGGCTTTTGCAGAAAACGCGCAGGCAGTGTTAATTGCAAATTTTTTCTTCCGTCGTAT
TTCTTTTTCCTGCTGGTGAACCTTGATTATTTCCCATATAAATAAGGGTTGAAGCTCCTTGATTTTT
TTTCTTTTCTTGTATCCAAAAAATCACACTTTGAGCAGTCAATATTTATAATATTAGCCGAAGGCGG
CCGCGGCGCGCCTTAATTAAGCTGTTTGTCCTTATACATCATTCCAAGGTTAGAAAAGGCCGGAAAA
CAGACATTTTTATTTCATAGAATATTGCAGTAGGATATATCCGCTACACTCGCTTATCAGCCCATTT
TAATTTAATATTTTAGTTTAATTTTTTAACTGATCAGTTAGCCTTCTTTCAGCAAGGGCTGAAAGAT
GTCGTGCGTAAACTCAAATAGTTACTTACTTTAGCAATGATCATTTGTTTGCTCTTGTTGTAGCCAA
GTTTCTCGGCAGACAAAAGCCGCTCGTTGAAGTCCATTCGGAAAGTCAAAGGTCATCGAAAGTATTT
CGAAGGGGCTGAATCTCGAAGGGGGCTGAATCTGCAGGGACTTTACGTGTCTTATCGATCAAAAGAT
TAATTCTAAAACAGGCAAGAAATGCGCGAATTAATCTTACAACTATAGTTGCTGCTGCTTTTTAGAG
CGGTCAACGTTTCTTACCAGGTTGTTTAATTTATTGATTATTCAATTG
SEQ ID NO: 20 - Pp02g04110 (homologous to ScPOR1) promoter-
polylinker-terminator cassette (promoter is nucleotides 1-1000 of
SEQ ID NO: 20):
TTATGCCATCGTGAACACAAATATCACAGGTGCATTTGGTGCTATTTCCTGGTGTCTATTAGACTGG
CGTTTGGAGCGCCGTTTCAGTACTGTTGCTCTATGCTCCGGTGCCATTTCGGGCCTCGTGGCAGCAA
CTCCAGCCTCAGGTATCATTCCTCTTTGGGCCAGTGTTATTCTTGGTATTGTATCAGGAGTGGTTTG
TAACTACGCAACCAAGATTAAAGTCATTTGTCGAGTCGATGATTCCATGGATGTTCTAGCAGAGCAC
GGTATCGCTGGTGTTATTGGTCTCGTCTTCAACGCATTATTTGGGTCGGCTACTGTCATTGGTTATG
ATGGCCTTACCGAGCACGAAGGTGGTTGGATAGACCACAACTGGAAACAGTTGTACAAACAGATTGC
ATTCATTTTTGCTTGTATTGGATACTCGATGGCCATCACCGCTCTTATCTGTTTCATCCTCAACCGT
ATTCCATTTTTGCAACTGCGAGCTTCAGAAGAGGCTGAGGAGAAAGGTATGGATGAGGATCAGATTG
GAGAGTTCGCTTATGACTACGTGGAAGTACGTCGTGATTTTTTGGCTTGGGGATCAGGCCCAAACAA
TGGCTTCAAGGAGCCGGAAGTTCTGGATCAGGTAGTTCCGGTTAATGATTTCAGCAGTGACCAGAAT
GTGACTAATGAGACCAACGAATCTGAGAAGCAGTAGAGTAAATATAGAGATGATATTTAGTGTATTC
TAATGCTTATGTAATGTATTAAGCAAAAAGTTGTGTTTATGAGTTAGCATTTGTCTTAGCAAACATA
AAATTATGTCGACATTTGCAACCCGCATGTCTAGTGTTTTTAGATCGATCTTCGATGTGTAGAATAA
TGCCTCCACGTGATGCCCCGCGATTTTGTTGGGTCTCAATGCCTCCAACATAAACCCATCACGTATA
AAAAGCCCTCTTAACCCTCCCCCCTGTTTCGTTTGCTTCATCACTTAACCTGAACTATCAAAGCGGC
CGCGGCGCGCCTTAATTAATAAGCGTTCTAGGTAGCAAGTTTTTTAAAGATGAAAAATTAGTAATAT
GATGAGTACTCGTATATTGCTGCTATGTCTAGCGTACTTCTGATTACCCCACTCGGACGAACTCTGG
TTTGGTGTTCTTGTCGATCAGTAAATCGGTTTTGAATGTCTCGATGTACTCCACATCGCCCTCCCCT
TTTCCCCCGGCAAAACGTCCATACTCATCAAATATGCCAGAAATACACCACCCCTGAAGCAGTTTTC
TCATGATACCCACTAATACCCCCACCCTATGTTTGCCCTTATTCGAATGAATTAACATTGGGTAGTT
TTCTTTGTGAACAATTAGCTTCAGAGCCTGCTGAATAACCGAGTCATCCTTGAACATAAACGGCTCT
ACACAGCTTTGCATATTTAGCTAATGAAAATCTATACCTTGATCGCGGAGCCACCGGTAATAGTCAT
AGTTGTCCGTCTTGTCACCCAAATATATGATTGTTTTTAGGTTCAGTTTT
SEQ ID NO: 21 - Pp01g03600 (homologous to ScBGL2) promoter-
polylinker-terminator cassette (promoter is nucleotides 1-1000 of
SEQ ID NO: 21):
TCTAATAATCTGAAAACTCGATTTGTTAATATTGGACGAGACACCCTTTTTGAACTAGAAAAAGGCC
AGGAAGTCCCCATGAACGTAAGGGTTACCGTTGATTTGAAAAAGAAAATAGTTGTATCTCCCTTGGA
TGCTTACGGAATCACAGGTTTAAAATCAAACTTCGGCTACAGTGTCAGAGCTGTCAAGGAATTTCAC
CAAATATATACAGAGAGCGGTAATCCAGATGGTTACTCTCGATCCTGTTTCGTTGAAGCAGGGGATT
TTTTCATAAACCAATCTCACACGCAGGATCACAATATGGAAAAGTTACCAATGATTGATAATTCCGA
GGAATTTAAAGATGGTCAAGAGCATCTTTTACTAGTGATCACAAGATGGAGAGAGCTCCAAGAATTT
TTCAAAGCTGACAATTCCGACATGCTCAAAGAGATAGAGAGCTGCGAGTCAATGTTTGATAACCGTC
TTGAGATAATCAACGGAACCAAACTAGAAGATGCGATTCTGATAGCCCTTGCCAAACTAGAGAGTTA
GTATGTTGATAGCATCAAAGTTCTAGGATGTCAGATGTCTAGAATCGTTCTGATTCGAATTGTTCAT
TTTGAGGCATATCCAAACCATTTTGGGCTTGTTTGGATGCAAGTTTCTTCGCGCGTGTATTGCTCCC
TACGTTATCACCACGACAACTAACCGTCTAGATCCGAAACAGTGAGTCCTTCAATTGGAAGTTCGTC
TACAGGTGACGGGAAAAGAACCATAAAATCATAGTAAATAAATGAAATCAGTATCTTAATTATCCCT
ACTAACCCATCCTTGTTGCTAGGTATCCTCTCGTATAGTGTCTCCTCAAAAACTCACCGAAGCTAAA
AATAGAACCGTATGATGTGGTCGTTCCCCACCCGACAATCTCGATATTTCAAACCCATCTCCCGCCC
TTTCCTTTTCAGTGTTTTCTTTTAGATTAGCTTCTTCTACTGATTACTTCTCTGTTGCAAAGGCGGC
CGCGGCGCGCCTTAATTAATGAGCTCTATCAAGTCATTTTATTATTACCCTCAAATAGGTCATATAG
CTTTTATACCTCAGACTTAAGCGTATTACGTGATTGAGAGAAGGGCGTTAGCAACTATTTTGACATT
TTTTGACTCTGCCACATTTGATTACATAATCAAAGAGGCCACCTCTCCGACGGAGTCCCAAAATTCT
AGTATATGAACCGAGAATCTTATCCACCCACCAAAAACCTTGCACCTTTAGCTGATACGATCAATGC
TTTCTACACGTGTAGTTAGACTACCAAAGGTTATTAGCGGGGGCAATAAGCAACTGGCACGAGGGTT
GGCCTCCCTTTCGGATGAAAAGTCGTTAAACGAAGCAAACCCAACGGATCTTGCAGGACTTAAAAGA
AAGGCCAAAAGAAGACCTACGAAACTTGCTGATGAATTGAAGACAGGTCCATCGTTCGCTGATTTCG
TCACGGGGAAGGCTAAGGACATGCTAGTAGATCCATTGGAACTGGCGAGA
SEQ ID NO: 22 - Pp01g14410 (homologous to ScACO1) promoter-
polylinker-terminator cassette (promoter is nucleotides 1-1000 of
SEQ ID NO: 22):
TTCATCAGTTGCCCATTCACTCTTATCTGGCTTCAAGGCTTTACTGTTTCCCCTTTAGCTATCCTGA
TGCCCTACCAAATCGATTGTTGCCCTTACACCAATGTACGATCACAACAATTCCCATGTTGTTAGTC
CCTACCATTTGCGAAGCTCCGAGGCCTAGGTCGTTGAACGTCACACATTACAGCTGCTCCGAAATTC
GCATAAGGAACACTCGCACCAATCACAAGGTGATGGAAATTGCTTACGGAGTAGAAACTCCCAATCC
CGAAGGTAAATACTTTTCTAGTGCACCCCGACATTCGACTCAAAAGGCTTAAACTAAACTCCTAAAA
TGTCCGTGGTTGACCAATAGAAAGTATCACTCAGCTCCCTGATTGTTCATAGCCTAACTGTTTCTGA
ATCTCTCCAAGTTTATTGCTGTCGGGTGAGCCTATGATTATCCCCTTTCACAATAGGCTCATTGTGT
CTTAGGAAGTACCTGCCCACTTCCCCCTGATAAACTTTCCACCATCCCCGGTCATTCGCTCATGACC
TTGTTATTACCATGCCAAAACATCCCATAATGAAAGGGTATCGGCAACATGGGAGCTAAATTTCAGA
CCCTCGAGATGGAGTCGGTAATCGTTCGAGAATCACATGGCCACCCCACATTTCAATTGTAGATCAG
ACTGTCAATCTTGACATAACCGTCGATAAATGACTTAGATTTCCTTCAGATACTCAGATTATCAGTA
TCTGGACTCTCCTGACCTTTTTCTATTTGCTCCAAAGTCTTCGAATCTTTCCTATCTTGTTGGAGTC
TGACAAACTACTTTAACGTTATTGGCTGAATTCCACCCTCGGATCCAACTTCTCCTTTTCGCTACCG
AGCCAAGGCAAATGTCTGATGCAGCTAGTTTTTACTGGCATACCAGGAGATCGCATTCCGGACATAT
ATTATGGAAGTTCCCTTCTTTTTTTTCCTTCTTCTTTTTCCCTTTTGTTCCCGTTATACAGTGCGGC
CGCGGCGCGCCTTAATTAATAAGTTTTAGCCACCTACAAATTCCAATAATCGGCTGTTTGTTCTGAT
TAGGTAATATCATGAATTATTTATTACATATTTTATTTATTTGTATCCTTATCCAAAACAAGGTTTC
AGATCTGCTTAGTACTACGTATCCGTTGTTGTACCAACCTTGTTACCACTACGACCAAGAATCTCAC
TGCGGCTCATTCTTTTGTCTTCAGATCTCCACAATTTGAGAGATATCACTGCTTTTGTGTTTCCTTG
TTCGAAACATCGACTGAGATATATAAATAGACAATGCTGTCCCTCCTACGGCTTGTTTCCTCAGTTT
CTTTAGCACTTCCAATAGCTTTGTATAAAGATGAATACTGAGGATGTTTACTCGCCAGTCATCGCTG
ACAATGGGTCTGAAGATCTCCTAACTACTACCAGCAAAGAAGTAGTCACCCCAGGAGACCTTGGTGC
TAAGCTTGAAGAGATCAAATTAGAATATTCTGAAAATGAGACACCATCCG
SEQ ID NO: 23 - Pp01g09650 (homologous to ScYHR021C) promoter-
polylinker-terminator cassette (promoter is nucleotides 1-1000 of
SEQ ID NO: 23):
TTGGTATACCACATTACTGAAATAGAGACCAGTCCAGGTACAACGACAGCTTCCAACGCACCCAATA
GAGCTCTGATTACCATCAAACTGGCGTAGGTGTCGCATGCAGCATGGCAAGCCACCACGATTCCCCA
AAGAGTGACACAAGTAGCAATTGTTGTAGCGGGAGGGAATTTCTGTAATGCCCAGCAGACTGGGGCT
TCAAAAGCAACTTGAGCTACGTAGAAGATTGTACTCAGATTGTTGAACTGATTTCCTACCAGATTAT
CTTTGATACCCATCACAGCAGCATAGTTCAATAAGAACTTGTCTATAAACTGCAAGAAATATATTGC
TGACAGGATTGTCATGACTCGAATGTCAATCTTAAACTTCAGTTTGGATGGAAGGTCAGTTATCGAC
TCAGTCAGGTTGATATTCTTTTTCTTGAGATACTTGAAGGCCTCATCTTGTTTAATCAAAGACTCAT
CATCGGATGAGCTGTTGCGTTTCTCGTGGTCCATTTCGCTCGCTGAAGAACGAGCTTAGCTGAAATG
CACCCTCTTATATTAAATCAAGAAAATTATCCAACACCTATCATGGGGTAACCTGAGCCGTAGGCAT
CTCGTATTGCCCTATTTGGACTTAAGGGTGAGTCAGCTACATATAGCCGAAAAAGAAAAAGGGGAAA
AAAGCAAGCATCTGCTACGAATAGCCGGGCAGACTGAATCAAAAATGCATTGAATGGAGCCTACATT
ACTTTTAACTTTTCCAGTCCACACTAGCGATAGATTCAAATTGATGATTGCATTGCTCTTTACTACA
TGATGAATCAAACGACTCAACCCCTTTTAGGCAACTCACCAGTCAGAAACTCTTGGTAGGGAGAGGA
AAAAATGAAAAACCGTTTGTCACGTGATAGGGCTGAAGCCTACTTTGTGCCGGGTAACTTTTTTGGA
TGTTCTCCCACTTAATTCTTTTTCAGAGGCAAAAATCTTTTCTGCATCAAGTATCCTACACCGCGGC
CGCGGCGCGCCTTAATTAATAAAAGAATTATTGAAGATGGTTGAAAGAAGTGGAACGATCAAGAGGA
TAACGTGTAAATGGTCAAAATCACTATCGTTATTTTTGCATAGTCATGTACATTAATGTTCAAGTGA
AGTGTAATATTAGAGATGAACTGTAGGAAAGTTGGACGTAGCCAGACTTTTGTCTGTTCTAGTTGTA
AGAAGTTGCTATTGTAGTTTTCTTTGGTCGTGTGAGTATTAATAGTTGATCCTTTTGTCTGTGGTTT
AGTTGTAAAAATGTCGGATTATGCGTTCGAGTCCAAGATACTGCATTAATTATCAAGCACCGTTTAA
TCCTGCCTCGTTTGTCGTAAAGCTTATCCACCCCTGGATCTCGCCTTTTTTCGCACCAGCCCAATTC
GTTAATGGAACCGTCCGTTCCATTTAGAGACGTCCCGAACCAGGAGTTGGTTATATGAATTCGAACC
CCTGGCATAAATGTCACATGAATTCGTCATCACACTCCGATATCGTAGCT
SEQ ID NO: 24 - Pp01g02780 (homologous to ScYLR388W) promoter-
polylinker-terminator cassette (promoter is nucleotides 1-1000 of
SEQ ID NO: 24):
ACTGAAAGGAAGTTGGACATGTCTGGCAAAACAAATTTAATATTCGATTTAAAACGTTCAATTCTGA
AGGAGTTGATGCTGGGCTGGTCTGTGATTTTCAAATCCTGAGAGTTTGCTGTGGAGTCATTAAGAGA
TACTAACAGCAAATTGGCAATATCGACGCCATCAATATCATCTGCCAATGCCAAGATGGTGACAACT
TTCTCTGCACCATTGGAGCCAGCAAACAATTGTCTTTCCAAAGACGTCTGAAGGATTTTATTTTGTC
GTAACTGTTGAGCAATATTCTTTCTCTCCTGTTTGGTTGTTATCTTGAGTTTCTTATTAGTTCCTGT
CTCCGATTTCTCAACTTTACCCTTGTAACTTGCCTTCAAGGAGCCCTTTGAGGCATGTCTCGACTTG
AAGGGCTTATGGTTATTCTTAAGAGTGGACCTATGGGAATGACCAGCCATTGCTTAGGAGAAATTGT
ATAATTAAAGTACACTGTAGGTCTAAAATTTTCAGATCCTTTAATACATAATTTTTTTTTTCTCATC
TCCCTAAGCTCATCACGTGTAATATACCGAAAGTAAGCCGTATGTCATGTGCACAATTCCACGGAAA
GGATGCCAAATACATCCCATCATCACTAGGTTTGAGCCCTAGAATGACACGTGAGATACAGAGTTTT
TTTTTCCCACTGTTCGCGCAAACTGGACTGGGTTCCTGGAGTCAATTTGTTCGTTCCTATTCACTTC
GTACACCAAAAACATCAAAGAGATCAATTATTGTAAGTTTGAACCACCATGATATTTTAACTGGAGT
GGAGAGGCTGATGTATTGGATGTTTATGTGAAGCCAAACAACAGGTCACACCAAAGAAATTCAAAAT
TCATGTTGAAAGGGAAACTATCAAGACCATCAGAGAGTACCAGTTGAAAAAAAAAAAATCCTGCAAA
CTCAAGCCAGCTCATTCACTTCAGTAAAATCCTAAAGGACGCCTACTAACATTACTAGCATCGCGGC
CGCGGCGCGCCTTAATTAATAAACAAATTTTAGGTCCTTTTCAAAAATTCACATGTATAATTTAATC
TGAAATTATCTATCTTTTATTTCCTCAAATCGAGAGTCGATTTGTGCCTGTTGGCGAATGTATTCCA
GAGTCCGTTGGAAGTGTTTCGTTGGAGTATCGATGCTTGTATTCTCCTCACCGATTGTCAGGATACT
GTTTTGGATGAGTTCAATCAAGCCATTGAGATACTTGATGGTGATGAGTTCGTTGCCATGTATGAAA
AAGTAAAGAGATCTACTTAGAATCTCCACAAACAGTTCCAGTGAGACATTTGTCTCCAAGGAGGAGT
CTGCTATCCGAAGGGACTTTTGCAGGCATTCTAGAACTTTCTTGTTATCAGTTTTAATCACAACCTG
CAGTTCATCCTCACTAGTTTTGGCTGTCTCTGACTCTATCTCGTCTTCCTCATCCAGCTCTTCGATA
ATCCACCACAAATGACTAGCCAGATAAACTGCACGGCATTGATCTGTCTT
SEQ ID NO: 25 - Pp03g09940 (homologous to ScPIL1) promoter-
polylinker-terminator cassette (promoter is nucleotides 1-1000 of
SEQ ID NO: 25):
ATGTGGGAGATTTATGTTGTTTACAAAGAGGACAAGACGTTCATTGGATTTGCCACCGGATACTCGT
ATTGGAAGTATCCTGGCCATGAAATCTTCGACTCCGATGCGAAATACCTTTGGAGAAAGAAGATCTC
ACAGTTTGTTATCTTACCTCCATACCAAGGACAATCTCACGGAAGCCAACTTTACAAGACGATATTT
GAACAATGGTTCAAGGATGACCAAGTGGCAGAAATTACTCTCGAAGACCCTAGTGAGGCATTTGACG
ATCTAAGGGACAGATGTGATCTGGAGAGACTTTATCAAAGAGGGTTGTTGGAAACATTACCGGAACC
AGTAATCTCTCCCGAATGGTTTCAAACACAGCAAGCAAAAGAGAAAATAGAGAAGAGACAGTTCCAG
CGGTGTGTTGAAATGCTCTTATTACACGCCTTGAAATCAAAGAAAAACACCCGTCTTCAAATAAAAA
AGAGACTTTTCATCAAGAACAGAGACGCACTCTTGGACCTCGACGAAGCCACCAGAAAGGACAAACT
GCAAGTTGCTTATGAACGCTTAGAAGAAGACTACCAGAGAATATTGGAAAAAGTTAGATTTGTCAAA
AAACGACCTCATCAGGACACTTAATATATAATTCCGACCTACGTAATAACCTAACAATATGAATAAT
AAGATAGAAACAACCCTCCCGCAAACATAAGCAAGAACAATTGGAAACAATGCCATACTTGCTATAT
TGGGTTATTTTGATTACCTCATTGGGATATCGAATTTCTAGTTCCAATTCGAAATTACGGTCGGAAA
TTTCTCGGTTTGGCTTTTGATGCAGCAATATTCCATCCTCGGAATGGCGTAATGCAGGAAGCACCGA
GGATGACGGCCTCAGTCATGTTCTTTCGTTTTGAATTGCTTCCTTCTCCCAGGAGACTGCTATAAAA
ATCAAACAATCTCTCCCCCATTTCAAGTTCACTTTCATCAATTGAAAACAAATCATCGGAATGCGGC
CGCGGCGCGCCTTAATTAATAAGATGTCCCATTTAGTATTAGCATTGAACGATGTTGATGTTGTCTG
AGTATATGGTTAATACGTAATAGCTCCGGGGAGCTAATTTAAGTCTCCCTTAATAATTGATTCTAGC
CAACAATTAGTGTTATTGTAATATCTTTTCCTTCCAAAATTCCATAGTTCTGTAGAGGCTCATGATC
GATAGCTACCAGGTTATGAGTCGCAAGCGTCAATATGCCATAAAAGACACTAGCTGCGATGCTCTTA
TTACACCCAGCCTCGGTGATATTGCTGGAAACTAGCTCTTGAAAGGTGACTTTTGAAAACACCTTGT
TTGAAATGGGGTTAACTTTTCGTTGACCAACAATTGAAGATTTATTACGCAAAAAATGGTAGAATCG
CTCATCTCTGGGATTCGAAGGAGGTTTGTCTTCCAAATGATTGTACAGAGAGTCATGGGTTGTAAAC
TCAGCATCAAAACTCTGTTCGTAAAGTGCTTCATCAATGATATTGTGTCT
SEQ ID NO: 26 - Pp02g10710 (homologous to ScMDH11) promoter-
polylinker-terminator cassette (promoter is nucleotides 1-1000 of
SEQ ID NO: 26):
TTGACGGTTATGCGGAAGGTTCTTGAACTCATAAATACTCCAGTCAAGTTTGAATCTGGCATTAGAA
AATAGATTTGCTGTGTAGCTCTGAACTCGTTGGTCTGGCTTGATTATTTTTTGGGATAGACTGTGTT
TCGGATTGTATTTCAAGTTGAGGTCATTAGTTAGTGATTTGGGAGAGGCAATAGATTTTTTGGAGGC
ACTTTTAGATTGTTTGGTCGGGCCTTTATAAAGAGGTTTGGTTGTGGACTGCTTTGAAGAGCTCTTA
GCTGGCTGGCTTAAAATACGAGAGATATTGACATTGCCCACATATTTTTCCTTCATGAAGGAGATGA
GGTGAAATATTCTTTAGTAATCTAGGAGGGAATTCTTGGAAGGGTAATTTAGACGGAAGACGTCATC
TTGCGGAAGGGTGAAACATTCCAAGGAGAGTGCTGGTTCCGAGATTCTCCGCTCCTGTAGAAACCTT
CAACCTTCCAACATTAGAACCTTCGCGAATAGACTCCCTATCCAATCACGTTTCTCCGTTTCAAGGA
AGTAGCCTGGGTGTCCGGTACGCCAAAAAAAAAAAAGGCCTCACCGGAACTTGACAATTTCATTCAT
TTTAGCTCAGCCTTACAACAGATTCACCAGTCCATACTGCTCTGCCATCTGCCAATTATAGTCTCCG
AGAAAAAAAAAACATCATGCTTTTCACTGTATTTCTTCCTTTTATCCACACCCCCAACCGATCAACC
CTGCTTCACGTCCTCCTCGGAAGTGGTGCTGCATTGGGCCCGTTTTTAGTTTGTTCCCCCACACTGA
GATGAACAACCCGACTCTCGGTTCTCAATTGCGCATAAACGTCACTTTGAATAGTCTCGCTAAAGTA
CCGGATATAGCTCGGCTACACCTCGTATTACCCCGTAACTATCTGTCGGAGCCGACCCCTTGATTCA
CATTATATATATCGTCGACAACTTCCACTATTCTCCAACCCTCAACCATTATAATCCCTTCAGCGGC
CGCGGCGCGCCTTAATTAATAAGTTTGATTATATGTACTTAGATTATTTTTCAATGAAATGAATGAG
ATTTGGAGATCCTGGGTGACGATATTGAACGTAGTCAAACAGCTAATTTTTTTTCTTTTTTCCCTTC
AAAATGAGCACAGTAACCATAGTTCCTTGCAGAGCACTCACAAAAGTGTTGAGTAGTTGGTTACCAA
GTACATTTCTGGGGCAACCGGTTTAGTATCTAGTGGGAGATTAGCCCACTCGTTAACAGCACTAGGG
TATTTTTAGACCCCCTATCTGGGCTATGATTCGGGTTAGCTCCAGCAGCTTCCCCATAGAGCGCAGT
CTAGCAAGAAGTCTTGTCATGTAGACCCCTCTGCTCCGTGCTGTAATTATGCGGTGAAAGTGCTTTA
TCCGATAGACTCCTCAGTCCTTTCACCCTCACTCATGCTCCTCCTAGTAACAACGAATCTAAAATCC
TCATCTGAAATTTCCCAATCAGGAAATCTTGGTCTCATTTTGCTCTCATC
SEQ ID NO: 27 - Pp01g00550 (PpTEF1) promoter-polylinker-terminator
cassette (promoter is nucleotides 1-1000 of SEQ ID NO: 27):
GTATTTGACAGGTTGGGGAGCAAATAAGTGATGATGTCCCATGAAAGTAGAAAATGGCTAGTAGAAG
GCAAAAATTTGAAATTCTTAGAGTCAAATAGTTAGACTCCAAGTTCTAATCCACATTTGGTCAGTTT
CATAGCATCCAGAGCTTTTGCCACTGGTGAACATATCTACCCATTGCGATGCAACAAGTCACTGAAA
GCCTAAAACGGAGATTCCCCTATCTTACAGCCTCGTTCAAAAAAACTGCTACCGTTTATCTGCTATG
GCCGATGTGAGGATGCGCTCATGCCCAAGAGTCCAACTTTATCAAAAACTTGACCCGTCATACAGGC
TCTAGATCAAGAAGCAAACTTAATCTCAGCATCTGGTTACGTAACTCTGGCAACCAGTAACACGCTT
AAGGTTTGGAACAACACTAAACTACCTTGCGGTACTACCATTGACACTACACATCCTTAATTCCAAT
CCTGTCTGGCCTCCTTCACCTTTTAACCATCTTGCCCATTCCAACTCGTGTCAGATTGCGTATCAAG
TGAAAAAAAAAAAATTTTAAATCTTTAACCCAATCAGGTAATAACTGTCGCCTCTTTTATCTGCCGC
ACTGCATGAGGTGTCCCCTTAGTGGGAAAGAGTACTGAGCCAACCCTGGAGGACAGCAAGGGAAAAA
TACCTACAACTTGCTTCATAATGGTCGTAAAAACAATCCTTGTCGGATATAAGTGTTGTAGACTGTC
CCTTATCCTCTGCGATGTTCTTCCTCTCAAAGTTTGCGATTTCTCTCTATCAGAATTGCCATCAAGA
GACTCAGGACTAATTTCGCAGTCCCACACGCACTCGTACATGATTGGCTGAAATTTCCCTAAAGAAT
TTCTTTTTCACGAAAATTTTTTTTTTACACAAGATTTTCAGCAGATATAAAATGGAGAGCAGGACCT
CCGCTGTGACTCTTCTTTTTTTTCTTTTATTCTCACTACATACATTTTAGTTATTCGCCAACGCGGC
CGCGGCGCGCCTTAATTAAATTGCTTGAAGCTTTAATTTATTTTATTAACATAATAATAATACAAGC
ATGATATATTTGTATTTTGTTCGTTAACATTGATGTTTTCTTCATTTACTGTTATTGTTTGTAACTT
TGATCGATTTATCTTTTCTACTTTACTGTAATATGGCTGGCGGGTGAGCCTTGAACTCCCTGTATTA
CTTTACCTTGCTATTACTTAATCTATTGACTAGCAGCGACCTCTTCAACCGAAGGGCAAGTACACAG
CAAGTTCATGTCTCCGTAAGTGTCATCAACCCTGGAAACAGTGGGCCATGTCTTTTGCTCCTTCAAA
AATGGCAATGGGTAGGCTGCCTCCTCTCTTGTGTATCCTCTCTGGGACCACTCAGCGTCACTTGTGC
TAATAATATCTTTTAGGTTGTGTGGGGAGTTCTGCAAGATTGCACCATCTGTTTCTCCGTTTTCTAC
TTTACGGATTTCTTCTCTAATAGAGATCATAGAGTCAATGAATCTGTCTACGGACCGATTTAAATGG
SEQ ID NO: 28 - Pp02g08660 (PpGAPDH/GPD) promoter-polylinker-
terminator cassette (promoter is nucleotides 1-1000 of SEQ ID NO:
28):
CTGCTACTCTGGTCCCAAGTGAACCACCTTTTGGACCCTATTGACCGGACCTTAACTTGCCAAACCT
AAACGCTTAATGCCTCAGACGTTTTAATGCCTCTCAACACCTCCAAGGTTGCTTTCTTGAGCATGCC
TACTAGGAACTTTAACGAACTGTGGGGTTCCAGACAGTTTCAGGCGTGTCCCGACCAATATGGCCTA
CTAGACTCTCTGAAAAATCACAGTTTTCCAGTAGTTCCGATCAAATTACCATCGAAATGGTCCCATA
AACGGACATTTGACATCCGTTCCTGAATTATAGTCTTCCACCGTGGATCATGGTGTTCCTTTTTTTC
CCAAAGAATATCAGCATCCCTTAACTACGTTAGGTCAGTGATGACAATGGACCAAATTGTTGCAAGG
TTTTTCTTTTTCTTTCATCGGCACATTTCAGCCTCACATGCGACTATTATCGATCAATGAAATCCAT
CAAGATTGAAATCTTAAAATTGCCCCTTTCACTTGACAGGATCCTTTTTTGTAGAAATGTCTTGGTG
TCCTCGTCCAATCAGGTAGCCATCTCTGAAATATCTGGCTCCGTTGCAACTCCGAACGACCTGCTGG
CAACGTAAAATTCTCCGGGGTAAAACTTAAATGTGGAGTAATGGAACCAGAAACGTCTCTTCCCTTC
TCTCTCCTTCCACCGCCCGTTACCGTCCCTAGGAAATTTTACTCTGCTGGAGAGCTTCTTCTACGGC
CCCCTTGCAGCAATGCTCTTCCCAGCATTACGTTGCGGGTAAAACGGAGGTCGTGTACCCGACCTAG
CAGCCCAGGGATGGAAAAGTCCCGGCCGTCGCTGGCAATAATAGCGGGCGGACGCATGTCATGAGAT
TATTGGAAACCACCAGAATCGAATATAAAAGGCGAACACCTTTCCCAATTTTGGTTTCTCCTGACCC
AAAGACTTTAAATTTAATTTATTTGTCCCTATTTCAATCAATTGAACAACTATCAAAACACAGCGGC
CGCGGCGCGCCTTAATTAAATCGATTTGTATGTGAAATAGCTGAAATTCGAAAATTTCATTATGGCT
GTATCTACTTTAGCGTATTAGGCATTTGAGCATTGGCTTGAACAATGCGGGCTGTAGTGTGTCACCA
AAGAAACCATTCGGGTTCGGATCTGGAAGTCCTCATCACGTGATGCCGATCTCGTGTATTTTATTTT
CAGATAACACCTGAAGACTTTTGGGTCGGAGGACTGGCTCTTTCCGATCAAATTGGAATGGAAAATT
GCTCCTCTAAGAAAGGGTGCCAACACTCTTTGTAACACAGGACACCGTTTATTGCTAACTCGATTGC
ATTCTTTCCTTTCCCACACCGGGATCTGGTCTTGGTGAACAATCTCTCCTGTCCTTATCTAAATATA
TCATCGCACTCTAACCTTCCTTATTACTTTTCGAGCGTCCGTCCTGTATTATCTTCAACCTGAAACC
AAACTCTAACCAGGCTTCACTCGTGGATCTATAATTGAACATGAAAAACTCGGACCGATTTAAATGG
SEQ ID NO: 29 - Pp01g12610 (PpPMA1) promoter-polylinker-terminator
cassette (promoter is nucleotides 1-1000 of SEQ ID NO: 29):
AATGAGAATAATGTAATATGCAAGATCAGAAAGAATGAAAGGAGTTGAAAAAAAAAACCGTTGCGTT
TTGACCTTGAATGGGGTGGAGGTTTCCATTCAAAGTAAAGCCTGTGTCTTGCTATTTTCGGCGGCAC
AAGAAATCGTAATTTTCATCTTCTAAACGATGAAGATCGCACCCCAACCTGTATGTAGTTAACCGGT
CGGAATTATAAGAAAGATTTTCGATCAACAAACCCTAGCAAATAGAAAGCAGGGTTACAACTTTAAA
CCGAAGTCACAAACGATAAACCACTCAGCTCCCACCCAAATTCATTCCCACTAGCAGAAAGGAATTA
TTTAATCCCTCAGGAAACCTCGATGATTCTCCCCTTCTTCCATGGGCGGGTATCGCAAAATGAGGAA
TTTTTCAAATTTCTCTATTGTCAAGACTGTTTATTATCTAAGAAATAGCCCAATCCGAAGCTCAGTT
TTGAAAAAATCACTTCCGCGTTTCTTTTTTACAGCCCGATGAATATCCAAATTTGGAATATGGATTA
CTCTATCGGGACTGCAGATAATATGACAACAACGCAGATTACATTTTAGGTAAGGCATAAACACCAG
CCAGAAATGAAACGCCCACTAGCCATGGTCGAATAGTCCAATGAATTCAGATAGCTATGGTCTAAAA
GCTGATGTTTTTTATTGGGTAATGGCGAAGAGTCCAGTACGACTTCCAGCAGAGCTGAGATGGCCAT
TTTTGGGGGTATTAGTAACTTTTTGAGCTCTTTTCACTTCGATGAAGTGTCCCATTCGGGATATAAT
CGGATCGCGTCGTTTTCTCGAAAATACAGCTTAGCGTCGTCCCCTTGTTGTAAAAGCAGCACCACAT
TCCTAATCTCTTATATAAACAAAACAACCCAAATTATCAGTGCTGTTTTCCCACCAGATATAAGTTT
CTTTTCTCTTCCGCTTTTTGATTTTTTATCTCTTTCCTTTAAAAACTTCTTTACCTTAAAGGGCGGC
CGCGGCGCGCCTTAATTAAGCTTCACGATTTGTGTTCCAGTTTATCCCCCCTTTATATACCGTTAAC
CCTTTCCCTGTTGAGCTGACTCTTGTTGTATTACCGCAATTTTTCCAAGTTTGCCATGCTTTTCGTG
TTATTTGACCGATGTCTTTTTTCCCAAATCAAACTATATTTGTTACCATTTAAACCAAGTTATCTTT
TGTATTAAGAGTCTAAGTTTGTTCCCAGGCTTCATGTGAGAGTGATAACCATCCAGACTATGATTCT
TGTTTTTTATTGGCTTTGTTTGTGTGATACATCTGAGTTGTGATTCGTAAAGTATGTCAGTCTATCT
AGATTTTTAATAGTTAATTGGTAATCAATGACTTGTTTGTTTTAACTTTTAAATTGTGCGTCGTATC
CACGCGTTTAGTTTAGCTGTTCATGGCTGTTAGAGGAGGGCGATGTTTATATACAGAGGACAAGAAT
GAGGAGGCGGCGTGTATTTTTAAAATGGAGACGCGACTCCTGTACACCTTCGGACCGATTTAAATGG
Sequences of inducible genes
SEQ ID NO: 30; Pp01g09290 (homologous to ScFBA1) gDNA ORF
atgtctacatttgatttcctttccagaaaaagcggtgtcatcgttggagatgacgttaga
aagctattcgaatatgcaagagaaaggaaatttgccattccttccatcaatgtaacgtcc
tcttctacagcggttgctgtattggaggctgctagagacaacaaatcaccagttatgctg
caagtatctcagggaggtgctgcttttttccagggaaaaggcgtcaataataaagacctt
agcgcttcagtgactggatctattgctgctgctctattgatcagaactattgcaccttct
tatggcatacctgtcattctgcacactgaccactgtcaaaaaaaatggctcccttggttt
gacggaatgttagatgcagatgaagaatatttcaagacccatggagagcctttgttctcc
tcccacatgttagacctgtcggaggaaacagacgatgaaaacattgctatttgtgtgaaa
tatttcaagagaatgacaaaaatgaaccagtggttagaaatggagattgggataactggt
ggggaggaagatggagtgaataacgaaaatgctgataaagactcactctataccagtccc
gaaactgtttttgcagttcataaagcactggctcctatttctccaaacttcgccattgct
gcagcctttggcaatgttcatggggtctacaaacctggcaatgtggagttgagaccatca
attttgggtgaacatcaagcttacgcagctcaacagttaggccttaaaaatggatcaaaa
ccacttttcctagttttccatggcggttcgggatcttctcaacaagagttcaacactgct
attaaccatggagttgttaaggttaatttagacactgattgtcaatatgcatacaccatt
ggttcaagagactatatcctgaaaaacaaagactatcttcaatccatggttggaaatccg
cagggagctgataaacccaacaagaaatactttgatccaagagtgtggattagagagagt
gagaaaaccatgagtggccgtgttaaagaggctctggaggtgttccatgctgctggtacc
ttcaagtctgagtcaaaactg
SEQ ID NO: 31; Pp03g03520 (PpDAS2) gDNA ORF
atggctagaattcccaaagcagtttcttacaatgatgacatccatgacttggtcatcaaa
accttccgttgttacgttctcgacttagtcgaacagtatggtggtggtcaccctggttct
gccatgggtatggtcgccattggtatcgctctgtggaagtaccagatgaagtacgctcca
aatgatccagactacttcaacagagatcgttttgtcttgtcaaacggtcacgtctgtttg
ttccaatacttgttccagcacttaactggtttgaaggagatgactgtcaagcaacttcaa
tcttaccactcttccgattatcactcattgactcctggacaccctgaaattgagaaccct
gctgttgaggttaccactggtcccctgggacaaggtatctctaacgctgtcggtatggcc
attggttcaaagaacctggccgctacttacaacagacctggcttccctgtcgttgacaac
actatctatgctattgttggtgatgcttgtttgcaagagggacctgctttggaatcgatt
tccttagccggtcacttggccttggacaaccttattgtgatctacgacaacaaccaggtt
tgttgtgatggttccgtcgatgttaacaacaccgaagacatctccgcaaagttcagagct
cagaactggaatgttatcgacattgtagacggttctagagatgtcgctaccattgtcaag
gctatcgattgggccaaggctgagactgagagaccaactctgatcaacgttagaactgaa
attggacaggattctgctttcggtaaccaccacgctgctcacggttctgctctaggtgag
gaaggtatccgggagttgaagactaagtacggttttaaccctgcccaaaagttctggttc
cctaaagaagtatacgacttctttgctgagaaaccagctaaaggtgacgagttagtaaag
aactggaaaaagttagttgatagctatgtcaaagagtaccctcgtgagggacaagagttc
ctttctcgtgttagaggtgagcttccaaagaactggagaacttacattcctcaagacaag
cctaccgaaccaaccgccaccagaacctctgctagagaaattgttagggcccttggaaag
aaccttcctcaagttattgccggttccggtgacttatctgtctcaattcttttgaactgg
gacggagtgaagtacttcttcaaccctaagttacagactttctgtggattaggtggtgac
tactctggtagatatattgagtttggtatcagagaacactctatgtgtgctattgccaac
ggtttggctgcatacaacaagggtactttcttgcctattacctctaccttctacatgttc
tacctgtatgcagcacctgccttgcgtatggctgctcttcaagagttgaaagcgattcac
attgctacacacgactctattggagctggtgaagatggtccaacccaccagcctattgct
ttgtcttcattattcagagctatgcccaacttctactacatgagaccagccgatgctacc
gaagttgcagctctgtttgaagtggctgttgagcttgaacactccacattgctttctctg
tccagacacgaggttgaccaatacccaggtaagacttctgcccaaggagccaaaagaggt
ggttacgttgttgaagactgcgaaggaaagccagatgtgcaactgatcggaactggttcc
gagttggaattcgctattaagactgctcgtttgctaagacaacagaagggatggaaggtc
agagttctgtcattcccatgtcagagattgtttgacgagcagtctattacttacagacgt
tccgtccttagaagaggagaagttccaactgtcgttgttgaggcctatgtcgcatacgga
tgggagagatacgccactgctggttacaccatgaacaccttcggtaagtctcttcctgtt
gaggatgtctacaaatacttcggatacactcctgagaagattggtgagagagtggttcaa
tatgtcaactctatcaaggctagtcctcaaatcctttacgaattccacgacttgaaggga
aaaccaaagcatgacaagttg
SEQ ID NO: 32; Pp03g08760 (homologous to ScCWP1) gDNA ORF
atgttcaacctgaaaactattctcatctcaacacttgcatcgatcgctgttgccgaccaa
accttcggtgtccttctaatccggagtggatccccatatcactattcgactctcactaat
agagacgaaaagattgttgctggaggtggcaacaaaaaagtgaccctcacagatgaggga
gctctgaagtatgatggtggtaaatggataggtcttgatgatgatggctatgcggtacag
accgacaaaccagttacaggttggagcactaacggtggatacctctattttgaccaaggc
ttaattgtttgcacggaggactatatcggatatgtgaagaaacatggtgaatgcaaaggt
gacagctatggtatggcttggaaggtactcccagccgacgatgacaaggatgatgacaag
gatgatgataaagatgatgacaaggattatgacgatgacaatgaccacggtgatggtgat
tactattgctcgatcacaggaacctatgccatcaaatccaaaggcagtaagcatcaatac
gaggccatcaaaaaagttgatgcacatcctcatgtcttctctgtaggaggagatcaggga
aacgatctgattgtgactttccaaaaggattgttcgctggtagatcaagataacagaggc
gtatatgttgaccctaattctggagaagtcggaaacgttgacccttggggagaactcacg
ccatctgttaaatgggatattgacgacggatacctgatctttaatggtgagtccaatttc
aggtcatgtccatctggtaatggatattcattgtctatcaaggattgtgttgggggaact
gacattggccttaaagtatgggagaaa
SEQ ID NO: 33; Pp03g00990 (homologous to ScYGR201c) gDNA ORF
atgtcacaaggaacaatttacttagtactcgcctccccaagatcatctttattcaaggat
ctgattgaatattacggtctcgatatcaagatctctgacacttccgacccagagtttgcg
aagacatttcccttgaagagaactccctccttcaaaggtcctgatttcgtactacatgaa
gctttggcaatatttgtgtatatcacttcgttgattcctcaaaaccatgggctgtacggt
tcttccaacttggattacgcccaaaccatcaagtggctatccttcactgcgtctgagatc
atatctggcttagttcaagcactgtatccgctcatcggaaacctgccatactccaaagat
ggcgttgaccaggcactcaaagatttggagtcgtatgttgctgtttatgaaacccagtta
aagcaaacgaagtatcttgttggcgataagattactttggctgatctgtttgctgttcag
tctatcaggtggggattacaatatatttgggacgtcaattggttgaaacaacatccattg
attgatgcctggttcaaggatgttactcaacatccaatcatcgtcaaatctcttggagat
ttcaagccgttgcagaaggctcttcctaatgctcctcctaagggcaat
SEQ ID NO: 34; Pp02g05270 (homologous to AN2948.2) gDNA ORF
atgagcaaacaaactccgtcaggaatgacttttatttccctagtggatgattcaaatgat
tctcctgtaactcaatctacttcagccgataaggacgagaagcatacagctgtgtcgcag
gagcagataaacgctgctggaaagaagcagaaccaagttgatgacgaggaagaagaggag
cagaaggaggaatacaaacgttctcttgaagaaattgaaatcaaagatgctgcttattac
aagatgacagatcctgttctcctgtatgatattccggcccaggttaattcttctgatgag
tacatcaaatggtctcccaatatcgtacagacgagaatgttgctcaaccatttgcaaatt
ccatttgaagtacaatggttaacgtaccctcaaataaagccaaccttacaaaagttaggt
gtcaagccttgggattcagatcctgagtatactcttccagctatttcatataaggggact
actgtgatgggcagatccgagattgaggaatttgttaagcaaaactttgaccccttatca
actccaaattttacagattattttctggacatcgaagaccgtaaatttaaccgagctatt
tccaactattcagatcagattcttatggggaagctttctttcccactgttggctttttcg
gaaagtattgtggtaaaagatgagaatgaccctgaatatttccaaagaacaaaaaatgaa
agattcggtgttgactgccaggctttggtaaaggataaagaaaagattcatgaaatggtg
gaagccatttgcaatgaattgcctggatttatggagctatatgattactcggaagctgag
aacgctgatttactatttctgaaagtaaacaagtatttgatgggctttcacattactcgt
gctgatcttattattgcatcatatgttttctggatcaagactgctgttaaggctggaatg
gaagaacactcatttcacaattattggtttgatgcttggtataacagaatctccaaactt
cttgactcccccttcgatggctcaaaggttgaacaagaagcgcttgatgatttgaacaag
aggatagagggctctcatcagaatcgtgtcagagagtctggggagcaaattgatgaaaat
gacaagaccgagaatgtcaatgaactcactgaacctgccagttcgactgttgagcagccc
cccgcg
SEQ ID NO: 35; Pp02g12310 (homologous to ScDUR3) gDNA ORF
atgactctcagctcacaggcttctaatgccatcatttatgtgacttatgggctgacttta
atattctgtgtggggctagcttggtatcataatgacaagtcaaaattcttatcgtcaaac
cagacaaaaacagggataccactggcccttaacttcatagcctcggcgatggggtgtggt
attcttacaacatacacacaagtggctaacttggcaggtatacatggattaatgacttat
actattgttggagctttgcccatattctttttctccttctggggacctcttatcagaaga
aaatgtccagaaggatttgttctgactgaatggactttccaaagatttggaagtgtcaca
ggatattatctgtctcttgccacaattttgacaatgttcttgttcatggtggcagaactt
agtgctattaagttcgctgtcgaagctttgacaggtttggacggccttcctgtggtcatc
gttgagtgtattgtcacgaccatttatacttctattggaggcttccaggttagtttttct
acagataattatcaggcctgtgttgttctagtcttggcagttattggtgtcattggcttt
gctctgaacgtcaacattgacccagaacttaaaagagaaactgaaagctacctattgggt
gccaacaagttaggatggcaattactttacatcttgtttattgctattgctacttgcgat
tgtttcatatcaggattttggttaagaacatttgcagcaaaaacaaacaaggaccttatg
attggaaccggtattgcgtctattgttgccatgaccatctgcactttggtaggtttgcca
ggcatttttggtgtgtggacaggagatgtggttatcggaagtcccgaaggttacctgtct
tttttcatcatggtatcaaccatgggcaactggatgataggattaatcctgatcttttcc
attgttctaagtacttgtacctttgactcgcttcaaagtggattgacctccactattgtc
aacgattttggaagaggacgtatgcctctttgggcagctagagtaatcactattttggtt
atggtcccttccattgtcgttgctgtcaaagttgcagatgatgttttaaagatatacttc
attgccgatttgatttcttcctcggtcattccttctctgttcatcggtctctccactcgc
ttctacttctggtctggttgggaagtagtcggtggtggatttgctggtctttttttcgtg
tgggtcttcggaaccgtttactacggtgatgccgctgaaggaggcaagttgttactgatt
tggaatggtatctacgattctgaagattggggtccatttggtgcgtttgttgttgcccca
ggtgtgtctttggtaggtggtttgatcatctgtggtgtacgattagcagttctcaaggta
tacagcaacattaagggtactccatttactgctcttgatagacctgaaaaacttggcttt
ggtggtgtcgcaattggtgactcttccgatattgaagaggtctacgaagagactttaaac
gaatctcaatcaaaaaaatctactgattacgtcaaggaagaagataatttccgtgct
SEQ ID NO: 36; Pp03g05430 (homologous to ScTHI4) gDNA ORF
atgacgctttccttattattgagaagtactaacatttttagggctcctaccgcaattgaa
actcaaactcaaaccactccagctttcaccgaaccccaggttctgaagctcaagcagaat
gttagaaacccagactctctggttgctaacgctgttaccccagcttttgactggaacacc
tttgagttcgctcccatccgtgagtcaaccgtttctcgcgctatgaccaagcgttacttt
gctgatttggacaagtacgctgaatcggatgttgtgattgtcggtgctggttctgctggt
ttgtctgctgcttatactttgggtaaggctagacctgatttgaagattgccatcattgaa
tcaaacgtcgccgttggtggtggatgcttccttggtggtcagttgttctctgctatggtc
ttgagaaagcctgctcatttgttcttaaacgatcttggattggagtacgaggacgaggga
gattatgttgttgttaagcacgcagcttactttatcactactctttgttccaaggttctt
gctcttcctaacgtcaagctgttcaatgccaccgctgttgaggacttgcttaccagaaag
gacgagaacggccagattcgtattgctggtgttgttaccaactggactttggtcacaatg
caccaccacgaccaatcttgtatggaccccaacactatcaacgctaatgttgttttgtca
gctactggccacgatggtccgttcggtgctttctgcatcaagcgtggtgtcgagattggt
gccgttaaaaaaatggacggtatgcgtggtcttgacatgaacaaagctgaggatgctgtt
gttaagggtgccagtgaaattgctccaggattggttgttgccggtatggaggttgctgag
cactctggttccaacagaatgggtcccactttcggtgccatggctctttctggagtcaag
gccgcagaggaagttctgaaggtcttcgatgagagaaagaagcagaaccagcaatgctat
ggtggactttccgct
SEQ ID NO: 37; Pp03g03490 (homologous to AN2957.2) gDNA ORF
atgacccttagttcgtctcatctgaatagtcaacactccgacactttggcaaatggcact
aacggtaactattctagcaccgtttccaacaacttgagcttaagtttgaactccttctct
ttctctgacaagttctcattgagtccaccaacaatcactgacgccgaaaagttttcattg
atgagaaacttcattgacaacatctcgccatggtttgacacttttgacaataccaaacag
tttggaacaaaaattccagttctggccaaaaaatgttcttcattgtactatgccattctg
gctatatcttctcgtcaaagagaaaggataaagaaagagcacaatgaaaaaacattgcaa
tgctaccaatactcactacaacagctcatccctactgttcaaagctcaaataatattgag
tacattatcacatgtattctcctgagtgtgttccacatcatgtctagtgaaccttcaacc
cagagggacatcattgtgtcattggcaaaatacattcaagcatgcaacataaacggattt
acatctaatgacaaactggaaaagagtattttctggaactatgtcaatttggatttggct
acttgtgcaatcggtgaagagtcaatggtcattccttttagctactgggttaaagagaca
actgactacaagaccattcaagatgtgaagccatttttcaccaagaagactagcacgaca
actgacgatgacttggacgatatgtatgccatctacatgctgtacattagtggtagaatc
attaacctgttgaactgcagagatgcgaagctcaattttgagcccaagtgggagtttttg
tggaatgaactcaatgaatgggaattgaacaaacccttgacctttcaaagtattgttcag
ttcaaggccaatgacgaatcgcagggcggatcaacttttccaactgttctattctccaac
tctcgaagctgttacagtaaccagctgtatcatatgagctacatcatcttagtgcagaat
aaaccacgattatacaaaatcccctttactacagtttctgcttcaatgtcatctccatcg
gacaacaaagctgggatgtctgcttccagcacacctgcttcagaccaccacgcttctggt
gatcatttgtctccaagaagtgtagagccctctctttcgacaacgttgagccctccgcct
aatgcaaacggtgcaggtaacaagttccgctctacgctctggcatgccaagcagatctgt
gggatttctatcaacaacaaccacaacagcaatctagcagccaaagtgaactcattgcaa
ccattgtggcacgctggaaagctaattagttccaagtctgaacatacacagttgctgaaa
ctgttgaacaaccttgagtgtgcaacaggctggcctatgaactggaagggcaaggagtta
attgactactggaatgttgaagaa
SEQ ID NO: 38; Pp05g09410 (homologous to ScTHI13) gDNA ORF
atgtctactaacaagatcactttctgtcttaactggcaagctgccccataccatgcccca
atctacctggcccaaaaattgggctacttcaaggatgagggtcttgatattgctatcttg
gaaccaggtaacccatctgatgtcactgaactaattggctctggaaaggttgacatgggt
cttaaggctatgattcatactttggctgccaaggctcgtggtttccccgttacatctgtc
ggttctcttttggatgagcccttcaccggaattttgtatctcgagagctccggtatcact
gatttccaatccctcaagggaaagagaattggttacgttggtgaattcggaaaaattcaa
ttagatgagctgaccaagcattatggaatgactcctgatgactacactgctgttagaagc
gggatgaacgttgctagagagataatcaacggtaacattgatgctggtattggtattgaa
tgtgttcagcaagttgagttggaggagtatctgagatcccaaggaaaggacgttgatggg
gctaaaatgctcagaattgacaagctggccgagctaggatgctgctgtttctgtaccgtc
ttgtacattgtcaacgacaagttcttggctgctaacccagataaggtcagaaagtttatg
agtgctgttaagagagctactgactatgttattcaaaagccagctgaggcatacgctgat
ttcattgagattaagccacttatgggaactcctctgaactacaagattttccaaagaagt
tatgcctacttttctgagtctctttacaacgtccacagagactggaacaaggtcaatgcc
tacggtaagagattgatggttttgccagaagactttaaggccaattataccaacgagtac
ctatcttggccagaacccaaggaagtctcggatccactggaggctcaaagaagaatgaac
atccaccaagaacaatgcaagtgtaatccatctttcaaaagactggctctcactggtctt
SEQ ID NO: 39; Pp02g07970 (homologous to ScPEX11/PMP27) gDNA ORF
atggttttagacactgttgtttaccaccccacactggataaggttatccagtatctggac
agctcggcaggtagagacaaattgctccgtctgttgcaatatttaaccaagtttgtctct
ttctacctgatcaagaatggacattcaattgttactgcccagacagtacgccgaatagag
gctattgcaaccttgaacaggaaggctctgagattcctcaagcccctgaaccacctcaaa
tctgcttcggcaacttttgacaataaactaaccgacaaggtcaccagatattcacaggtg
ttgcgagatctcggctacgctgtctacctggcattggactctgtttcctggttcaagcag
ctcggtatctcctccactaaaagactgcctcaagttcaaaaactggcctcattattttgg
ttcgtcgccgttgtcggtggagcagtcaatgatctgagaaagatcagattgtctcaacag
aaagtggcctctctcaaacaagagctggttgtcacttcagacaaggagggagaacaaaca
gtctccaaggagaccatcaacttaatcgagtcagaatccaaactgattggtagcacgaca
ataactcttatcagggatctgttggatggttatatcgctcttaacggctttgccttacaa
aacaaagatgagaaggttggattggccggtgtcatttcctccttgataggaattagggat
gtctggcagggaaaatacatcaatgaa
SEQ ID NO: 40; Pp01g12200 (homologous to AN7917.2) gDNA ORF
atgacatcaaatataaatggttctctgcctcaaagcgctaatgtggtggttattggttct
ggatttgctggaaccgctgtttcttactatttacagaatgaaattgctggtgaccaatcg
attgtgatgctagaagcaagaggagccgtctctggagccacttcccgaaattctggactt
ataaaacctgagtaccatagaaatcatggtgaatatgttgacaagtttgggtcgaaagtt
gcggggcaattggtcaattttgaggttgacaacatgaaggagctggctagaattctaact
cttgattccgatcttaatgaaaaggcagacttccaagagcggatacatcttgatgcttac
tctaaccctcgttcgtcggagactgccatcaatgatttctatgcttttatggaaaatgaa
gaagtctccgtagacctgaaaagaaaagtgcaaatattatttggggatttggccaagcaa
ctgagtaatgtgcctactaccccttttgtcattcattatcctaatggatctgttaattcg
tatgatttcgtcactgctatgttaaccagagctattcaaaaaggcctagccctatatacc
aatactctagtggaagaagtgcagcaattggattctggtgtatggaaagtatctacttct
agaggtgaattatttgcggacaaagtggtttttaccaccaacgcctacacacaaggactc
ctgcctgagttttcgaaatcaattatacctatacgaggcgtttcatcacaagttaacgtt
cgtaatacgggtaatgagcactctctgtccattggctccgacatttatgttccacaatcc
aagaatctgctctacagttcactgggtgagctggaaaaggagaccaatttcatggctaat
tacaacactgtggacgattcaactgtatcgtcccaatcgttagaatatttgcaaaaaaat
ttaggtaaagtgaactcacttgctactgccactgctacttcatcatcgtctggaatcatg
gcctacactgatgatcattttccttacgttgggcaattgagcgagctgggaaaactaaat
gcatatattttggctggttgcaactgttcgggtctttccagaatgctcctttgtgctaaa
gaacttgccaagtccgttgcattggggtcggagttaagcgacagagtcccagctccctac
aaggtaactagggaaagaatgggactcgttgacaaattgatgcaatcggtattgaatgaa
agagaaattagagagactagagcaagactc
SEQ ID NO: 41; Pp03g11380 (homologous to ScPMP47) gDNA ORF
atgtcaaaaaacgctcaggttgacgatcttgcccatgggcttgcaggtgccggaggggga
attctctcaatgattatcacttatccccttctgaccctatcaacccatgcccaatcttca
aaaacccagaaaccactagatggctctgtagatgagaaggaattggagcccaagaagtct
tcaacttacggtactctaaaacggattttgaaaaagcagggagttcggggtctctacaac
gggctggaaagtgctatccttgggatagcagtcaacaacttcatttattactacttctat
gagctaactggaaacactttggaaggcttgtcccgtggtagaaagagaggttccagagtt
ggtggtctctctgcattccaaagtattgttgctggagctattgctggtgtgatttcccgt
attgccacaaatcctatctgggtagcaaataccagaatgactgttctttccagagaacag
agagatctgaagagggtaaacacattgcaggcaattttgtacatcttcaagactgaggga
ttcaaaacgctgttcagtggcctcattccggcattgtttttggtcctgaacccgatcatt
cattacacgatttttgagcaactgaaaacactactggtaaagacaagaaagagagcgttg
actcccttggatgctttactgttgggtgcttttggaaaacttatttcaacggtgatcacc
tacccctacgtcaccctgagaactagaatgcatttgcaaaacgctgagaatgctaggaat
tcttccggtgagagctcagtcagcaattccgctgttctttcggccacgtcagacgaagac
ttgggcaaagagggcgacaacgagaaaaagattgcccaggaacaacctaccaacactatt
tggggcctttccacgaaaatgttaaaagaagaaggaatatcaagtttctacagtggaatg
tctgtcaagttatcgcagtcaatcttgtcagctgcctttttgttctttttcaaagaagag
ttggtatcagccagtgatgtggcaattaaggcggtgaaaaaggtcgactacaagtctttc
ttgaataaccccgctcaagtagtgcctgatttgaaccctgcaccatctcccaatgctgac
ggctctccagtggatatt
SEQ ID NO: 42; Pp03g08340 (unknown) gDNA ORF
atgacgacattatcgttgaagcaaactgcatatgaattcgtcgaaggcatgacaggtgac
taccactacttaaagaaccttgatgcttcagattcaaatctacgataccttaaagattat
agttttccaaccttgcaagagttgaatctttccaatttatcctacctgaagaaacttgtg
tctgatggagatcaattgagcgggttggaaaagttaactgctgatgggtccaatatcgaa
cttatcgatattaagggagatgtcctggcaagtatctcctgttcgaacacaaaagagcta
aaagcaatcaaaggtgaactgccggcgattaactatatctatgcagacaactccagcttg
gaatcggtatccttattgccatatgaagacgaaaaatttcagttcaagagaatgttcttt
ctatatttggtcaactgtcccaatctcatatcctttcaaagagggcagtatcccaagctt
cggactgtcgatttaagtaattccacgatacaagttcttcattcatctcttataaagcat
cttggtgctttatacttgggcaacgcaactattaaagagtttgtaattgacgacaacgac
aatgaagatgagctcacccttcggcaactaaaaactttgaatttatcaaattccaggatc
ccaacaaatatgacagattttcttttaagacatatttttagaaagaagagcaatccctgg
gataaagaatggtcgtcattgatttggcaatcagtcaatacagatcaagaggtgcaagag
gtccagagttttattaagagaattccacagctcaaaaaactggatatatccaattcatca
aaagcaaactcattgctgtccaatctagtgtctgacattccagagttttggaacagctta
gaagatttggatgtattcaattccaacactgagggttttaacttttcaaacagcaaatca
ttgattcacctgggttgcagcgatcccgatgcaactgttttgaaagttgacgacatgccc
gccttgaaggcgctggatgtcttgggaccctctaacattgaaactgtaaagttatccaac
tgcattagactcgaaaaggttgacttcactggctgctacgctttgaaacgctttgagtca
gactctttgagtcttctgcaattgaagttttcaaataactcattggaaacatggtccctt
aatgactcctctatcagaagcatcacgttagagaacaatacaggtttcagaaggctagaa
ggaaatttcccgaatttggaaacgttagatatcagtaatagcgctgttgaagttatcaat
attcatgatggtagcaaattgaagaagctcagcattttgagaactcgaagtgttgaagag
attcaaattgataatctttcaacactacaagaattgcgctatgacaccaaagacgaaaag
aatacgaaagcagtaaaggagtttgcagataagattttatcaaagagggaaccaagtttt
acagccaaggtactccaaagagtccgctttgaacacaggggcttattcacaggtgggttc
aggttttttgatagaagaactgaggaactggtttgctgctttaaagatatcaacatttcc
gaaaataagatcaatgcaaaatgtggcatctgtgaggaaaactacgaggaaggtcagaaa
tgtcagattctctattgcaagcatgcttttcatactgattgcgtagaacagatgctacaa
atgggtaacgataggtgcaggcattgcaacgatgaaattgacattgcctcagtaggtcta
agtagcgatccattgccctttagatggttg
SEQ ID NO: 43; Pp05g04390 (homologous to ScTIR3) gDNA ORF
atgagattttctaacgtcgttttaactgcaattgccgctgccggcgtacaggcagatgaa
gccctttacactgtgttctacaatgatgtcactgagaacgcccaagagtatctgtcttac
atccaggccaatactgcggctggtttcactgacctcttgagtctgtacactgaactggcc
acttacaccgacgattcttacacaagtatctttactgaggaggatttccctgcgagcgaa
ctttcatcgttcgttgttaacctgccatggtattcctccagaattgagccacaagttgcg
gctgctgaaactggtgaaagtgaggaggaatcagagactggtgaaagtgaggaagaatca
gagactggtgaggagacagaaactgagactggatctgagtctgaatctgagtctgaatcg
gagacctccgctactggcactggcactggcacctccgcctctgagagcgcggagactgaa
acttctaccgacgctgctgtgtctatcgatcacccaaagtccaccttattgatgggtttg
actgccgcagttgtcagtatcactttcggagtctttgccttg
SEQ ID NO: 44; Pp01g08380 (homologous to ScYIL057c) gDNA ORF
atgggtagaagaaagtcacaagctgctgcagagagaaatcttgaaccaattaaaattagt
accgactcaattaagaaaaggcctcgtcgagattccaatgaacctccattcaaaaagttt
gatgatctagaaatgtttgaaacttacttgaagggtgaatcttgggataacgattttgat
ttcctccacgctcgtttggattattatcctccatttattcgtaatgaaattcatgacgat
ccggaaaagattaaaccaacaatgaacaataagtccaagaagtttgtgagaaacttgcat
catcacgttgacaaacatctgttaaagcaaattaatgacatggttggaatcgagtacaaa
ttcaaacgggaggaagagaagttaccagatggccgactaatctggcgctacaaagacgaa
tcagatcatggatttgaaggtcttgaccggaaatggacagtcgaagttgatgtcgagtgt
agtcccaacgatccaactgtagttgtcgatatgaggtccattcctattgac
SEQ ID NO: 45; Pp01g05090 (homologous to ScSAY1) gDNA ORF
atgaccttgacgattaacccgagtttaaagaacgtcttagaaaaccagaagggcggcgca
cctgtggagaatgatcttaaggcgctgagaaagcatgctgacaagatagaggaacactac
tacaaagaggtgataaccaaaccatctgacgttgcaggcatgcaatttactatcactgct
catgagggagatcaaattgagtgccgtttctactctaaagatgctgatcctaaccaagta
aggaccagtaagctaccgtgcattattcacttccatgggggtggatacataaccggtgac
gttagccgttacagtcatttgacctctcaatatgtctcagcgactggcgtacctgttctg
agtgtggactatagactggctccagaatatccagctccaattcctcaagaagatggcttt
agtgcgctacagtacctttatgaacattgtgataacctaatgattgatcccaacaagatt
attctaatgggagattcatcaggcgctgggttggctttatctgtggctgcattggcagca
gaaaggggcattccaatagccaagcagattctcatttatccaatgttggactcaaacaat
accgaagaacccgattctgaagaccttaaggagaaaaaaccttatctaacatggactcat
agaatgaacaagctggcctttgattctcttctatcagaaaaagaagaatatgagccatca
ttattcccattcaacgatcctaatgttgattggtccaaatttcccaaaacttatttggaa
accgtgacagttgacatcttaaacgatgacggccggcagctgcatagaaatttgactgag
aacaatgtacaagttgaatttcatgagtgggaaggactttgccatggcttcgaacctctt
ctttggaccgaggaaaatgatctacttcaaaacattttagagaagcgttaccaagcaatc
ctggatgta
SEQ ID NO: 46; Pp01g13950 (homologous to ScTPN1) gDNA ORF
atggaaaaaaaagcttccagtgatctggagttagaaacggatagcaaaatctcggagaca
gagaaaaggggtccagtatcaaagttactgaacggcctcaactatgtctccaagaagcta
gattcctttggtggtgaatccaccggtatcgaaagagtttctccagatcaaaggcggacc
aatatgacgaggattatcattcacgttatgggtctgtggttatcaggatgtggtggtata
acttctatgtccagtttttttttaggacctttgatttttggactggggttcaaagatagt
atgataagtggattagtatcttgcacattggggtgtctcttagccgcctattgtagtacg
atgggaccaaggtcaggactacgtcaaatcgtcagtgctaggttattttttggtccttgg
gctgtcaggttcccagctttaatcagtgctatcggtttcgttggttggagtgtgactaat
tgcgtccttgggggacaaatactgtacagcgtttccaatgacaaactgcccatagaaata
ggcattgtgattatctcaatgatatcgttggtgattgccattttcggtatcagaattctc
ttgcatactgagacgcttatctccattccagtgttcacggtgttaattctcctgtacatc
attggctccaatgaataccccaattacttgaacactgtatccattggggacactatgacg
atcagaggaaacttcttatcatttttcgctttaggtttttctgtgacagctacttgggga
ggatgcgctagtgattattatacactattaccagaaaacaccaaccagctattcgtattt
ttcatgaccttctttgccatttggataccttcgtttactggagcggtgctgtcgatctta
cttggcaatgcggccactgttcatgagccgtggttggaagcttatacgaacaactcactg
ggtggattattgcatgaaattttttcccgttggaatgggtttggaatgttccttttggtg
atattcttcctttcacttatcacaaacaatattattaatacctactctggagcattggaa
ctgcaactgataggtggaccactttcatattttccaagatggttcttgagtgtggtcatg
gttgtaatattcatggtttgttctctggctggtagagaccaatttgccacaattctttcc
aatttcttgccaatgctggggtattggatttccatatacttcactttactgttagaagag
aatgtcatcttccggtccaacaaaactctaattaaactttatcaatatgagttctctacc
attcctggagagaagcaagatcttctggagggcaaatccagaccatattacaactttgac
atctatctttctcgggataggcttactcacggatttgcttcttcattggcgttttgtttt
ggtgtagtcggagcaatttgtggaatgtgccaagtctactacattggacctatagcatcc
aagattggaagccatggagctgatcttggtatgtggttagctattggatttactgctgtt
acatatccggtattcagatatattgaacta
Sequences of methanol-inducible promoters
SEQ ID NO: 47; Pp01g09290 (homologous to ScFBA1) Promoter
ACTCTACCCAGGATTATTTTTCTTCTGCGAATACAAAACTGCTTATATGT
CACACGGATAACTCCTCTTTTAACGAGATAGTTGACTTCTATTAAAAAGT
CCGCATAGTTAGATTTACCTCCATCTTGAGTTAGAAGATGAACCTTTTCA
TTATAGGGTGGATCATACCAATTCCAACCATCTGGGGCCAAGGTTGAAAA
ACTGGGGGTTCTTGGAAAGTTATAGAAGAACAAAGTTCCTATTCTGGCAA
AATTATGAAGGTCATTGGTGACATTGGTAACACTTGTCAGATCCATAAAT
TAATCCATAAGATAAGGCAAATGTGCTTAAGTAATTGAAAACAGTGTTGT
GATTATATAAGCATGGTATTTGAATAGAACTACTGGGGTTAACTTATCTA
GTAGGATGGAAGTTGAGGGAGATCAAGATGCTTAAAGAAAAGGATTGGCC
AATATGAAAGCCATAATTAGCAATACTTATTTAATCAGATAATTGTGGGG
CATTGTGACTTGACTTTTACCAGGACTTCAAACCTCAACCATTTAAAGAG
TTATAGAAGACGTACCGTCACTTTTGCTTTTAATGTGATCTAAATGTGAT
CACATGAACTCAAACTAAAATGATATCTTTTACTGGACAAAAATGTTATC
CTGCAAACAGAAAGCTTTCTTCTATTCTAAGAAGAACATTTACATTGGTG
GGAAACCTGAAAACAGAAAATAAATACTCCCCAGTGACCCTATGAGCAGG
ATTTTTGCATCCCTATTGTAGGCCTTTCAAACTCACACCTAATATTTCCC
GCCACTCACACTATCAATGATCACTTCCCAGTTCTCTTCTTCCCCTATTC
GTACCATGCAACCCTTACACGCCTTTTCCATTTCGGTTCGGATGCGACTT
CCAGTCTGTGGGGTACGTAGCCTATTCTCTTAGCCGGTATTTAAACATAC
AAATTCACCCAAATTCTACCTTGATAAGGTAATTGATTAATTTCATAAAT
SEQ ID NO: 48; Pp03g03520 (PpDAS2) Promoter
AATAAAAAAACGTTATAGAAAGAAATTGGACTACGATATGCTCCAATCCA
AATTGTCAAAATTGACCACCGAAAAAGAACAATTGGAATTTGACAAGAGG
AACAACTCACTAGATTCTCAAACGGAGCGTCACCTAGAGTCAGTTTCCAA
GTCAATTACAGAAAGTTTGGAAACAGAAGAGGAGTATCTACAATTGAATT
CCAAACTTAAAGTCGAGCTGTCCGAATTCATGTCGCTAAGGCTTTCTTAC
TTGGACCCCATTTTTGAAAGTTTCATTAAAGTTCAGTCAAAAATTTTCAT
GGACATTTATGACACATTAAAGAGCGGACTACCTTATGTTGATTCTCTAT
CCAAAGAGGATTATCAGTCCAAGATCTTGGACTCTAGAATAGATAACATT
CTGTCGAAAATGGAAGCGCTGAACCTTCAAGCTTACATTGATGATTAGAG
CAATGATATAAACAACAATTGAGTGACAGGTCTACTTTGTTCTCAAAAGG
CCATAACCATCTGTTTGCATCTCTTATCACCACACCATCCTCCTCATCTG
GCCTTCAATTGTGGGGAACAACTAGCATCCCAACACCAGACTAACTCCAC
CCAGATGAAACCAGTTGTCGCTTACCAGTCAATGAATGTTGAGCTAACGT
TCCTTGAAACTCGAATGATCCCAGCCTTGCTGCGTATCATCCCTCCGCTA
TTCCGCCGCTTGCTCCAACCATGTTTCCGCCTTTTTCGAACAAGTTCAAA
TACCTATCTTTGGCAGGACTTTTCCTCCTGCCTTTTTTAGCCTCAGGTCT
CGGTTAGCCTCTAGGCAAATTCTGGTCTTCATACCTATATCAACTTTTCA
TCAGATAGCCTTTGGGTTCAAAAAAGAACTAAAGCAGGATGCCTGATATA
TAAATCCCAGATGATCTGCTTTTGAAACTATTTTCAGTATCTTGATTCGT
TTACTTACAAACAACTATTGTTGATTTTATCTGGAGAATAATCGAACAAA
SEQ ID NO: 49; Pp03g08760 (homologous to ScCWP1) Promoter
AATTGTCACCTTGAACAGCACAATCTTAAACAACTAGATCTACACCATGA
TAACATTCAACAAACGAATAGCAACATTAGCGGCAACGTTATTTTCATTC
ATTGTGCTTTATACTCTCTTTAACAGTGGAGCTCAATTTTCCAACCAACT
AGATCAGCCTGTTCCCCTCAAAACTCCAGAGCTCATCATACCGAATCAGA
GTACTGAGAATGATCCCCCTCTTCCATTCATGCCAAAAATGGCTAACGAA
ACTTTGAAAGCAGAACTTGGAAATGCTTCCTGGAAACTCTTTCACACTAT
TCTTGCTAGATATCCTGAATCCCCATCGGAGAATCAAAAATCAACCTTAA
ATGACTACATTTATTTGTTTGCACAGGTTTATCCATGTGGAGACTGTGCA
AGACATTTCAATTTATTGCTGCAGAAATACCCTCCACAATTGTCCTCAAG
ACAGGTGGCTGCAGTGTGGGGATGTCATATTCACAATCAGGTCAATAAGA
GATTGGAGAAACCACAATACGACTGCTCCAATATTCTAGAGGATTACGAT
TGTGGATGTGGCTCTGATGAAAAGGAAGTAGATGACACTCTGAATAACGA
AACAATAGAACACTTGCAAAGTATCAAAATTACTGAAAAAGAGAGTGAAC
AATTTGGTCGATGATTACCTGATTAGGGCAAATGGCCAAGACCAAATTAT
GGAGAATCCTCAACATACCACACTTTAACAATGCTATTTTAATCTTTTAA
GCTCAAACCGATGCTCATCGCCCTTTCAAGGTCATACTTAAACTCCTGTG
GGTTATACTTAGAACTCATCTCTCAAATGCGTGACGTCATCGCGCATGTC
AGGTACCCTCAAACGGTCTTTGGGGTAAACTGGTAGACCCTTGGAGGGTA
CCTATATTTGGTATCACTGGCATTGATGAGTTAGATTTGGGTATAAATAG
GTCATTGAATATCCCTTCATGAAGTATCAATTAAGAAATCACAAACACAA
SEQ ID NO: 50; Pp03g00990 (homologous to ScYGR201c) Promoter
ATTGTTGTGAATACTCTCCTTCATTTGGATTTCTTGGACTTCGGACTCTC
TTGATCTCTCTTCGAAAGTTTTAACTCTGTTCATGTATAATTTTACCCGC
TGTAGGTCGCTCATAATACCATGAGTATGCACATCTTTTACTCCATTAAC
TTTCAGGTATGCAAAATACAATGAAGATAGTATATAGCTCAAAGAATTTA
GCATTTTGCATTGATCTAATTGTGACATTTTCTCTATGATATCATCTAGC
TTCTTAAACTCGAGAATCTCGTCCAACGAGGCAGAAACATTGTCCAGTCT
TACGTCAAGATTATTCACGAGTTTCTGGACCGTATCAACGTTTTCCATCT
TAAGATTACAGTAAGTATCGTCCTTTTGAACTGCAAAGGTAGAAAAGTTA
ATTTTTGATTTGGTAGTACACTATGAAACTTGCTCACCCCAATCTTTCCT
CCTGACAGGTTGATCTTTATCCCTCTACTAAATTGCCCCAAGTGTATCAA
GTAGACTAGATCTCGCGAAAGAACAGCCTAATAAACTCCGAAGCATGATG
GCCTCTATCCGGAAAACGTTAAGAGATGTGGCAACAGGAGGGCACATAGA
ATTTTTAAAGACGCTGAAGAATGCTATCATAGTCCGTAAAAATGTGATAG
TACTTTGTTTAGTGCGTACGCCACTTATTCGGGGCCAATAGCTAAACCCA
GGTTTGCTGGCAGCAAATTCAACTGTAGATTGAATCTCTCTAACAATAAT
GGTGTTCAATCCCCTGGCTGGTCACGGGGAGGACTATCTTGCGTGATCCG
CTTGGAAAATGTTGTGTATCCCTTTCTCAATTGCGGAAAGCATCTGCTAC
TTCCCATAGGCACCAGTTACCCAATTGATATTTCCAAAAAAGATTACCAT
ATGTTCATCTAGAAGTATAAATACAAGTGGACATTCAATGAATATTTCAT
TCAATTAGTCATTGACACTTTCATCAACTTACTACGTCTTATTCAACAAT
SEQ ID NO: 51; Pp02g05270 (homologous to AN2948.2) Promoter
CCTGATTTGATCTAATCCTGGCTCATAGTTACCTTGTCTCGCTTGCTGAT
GTTGTTTATGTTCAAACTTGGACTCAACTCCAAATTCAGCTTGCACTTCA
TTTGTGTCGACCAACGAAACTGCAGCCGGTTCAGAAAGGCTGAAACCCTC
TCTTCTCAATAGTCCTAAGATCTGGCTAACTCCAGCTTCTAAAGTCTTGA
ATTTTCTGTCTATATTCAAGTTCATTTTTGCTATCGGTGACAGCTTGAAC
ACTTGGCTTTCTGGTTGAATGGTTTGCTTGATATCAAACGAACCGTTTTG
GTCCAAATTGGGTAAGCATCCAATTATATTCTCCTTTGAATTATCAGGAT
CTGTCGCCCGTTTGAGCAAATCTTCTTCAAAGGAACAGACTTGGCTCATC
GAGGCACATCGAAGGCATGCGATAGAATCTGAAGAAGGAAAACAACGAGT
CTTCTGTCTCCGACAGGATGAGCAAGCCTTTGAAGCTCTAGAATACAAAG
GTTTCTTGGGTTTGGTCACTTTGTTTGACTTTGTGGAGTCATTTCCTGGA
AGCACAGGAGCCATTTCTCAGCTTCTCTCTCAGTTTTTAAAGTTTGTCGA
AATATTTTGATAAATGCTCTGTACTTGATAAGAACTGCTATCTTTGATTT
CGCGACAGAGGTGAAAGAGAACAGAATCCATCACCTCCTTACATCAGAAA
TGAGAGTGAGAATTCATAGCCTACCAAAACAAGGACTCAGGGAATTGATA
AGTTTACCCCACAATTTCGCTTACTCCGCGGTCATGTATGATCGATGATC
CAGTGGTATAGTCCAAACGAAACATTCGACAAAGTTCTTTTTGCTTCTAC
CTAGAGGGGTCTCAATTTACGTTAAAGCCAGTCGGGCATATACTCGATCG
TATAAATAGAAACGTTTTAGCCCTCGTTTTCACCATACCATTCCTTTTTC
CTGGGTATTCTACTAAGACCAAAAGAGTACCTCTCCACACTACCAAAACC
SEQ ID NO: 52; Pp02g12310 (homologous to ScDUR3) Promoter
TATTTAGATATCAAGCATGTGAATAGCGTAGCTGGTATACTAACAATCAT
TTAGGCCCCAAAAGTTAAGAACTCAGACACTATCAACCAGAAGCTGGCCC
TGACCATCAAGTCAGGAAAGTACACCTTGGGATTCAAGTCCACCGTCAAG
GCTATCAGACAGGGTAAGGCCAAGTTGATTATTATCTCTTCCAACACCCC
AGTCTTGAGAAAGTCCGAGTTGGAGTACTACGCCATGTTGTCCAAGACCA
ACATCTACTACTTCCAAGGTGGTAACAACGAGTTAGGAACTGCTTGTGGT
AAGCTCTTCAGAGTTGGTACCCTGGCTATTCTTGATGCTGGTGACTCTGA
CCTGCTTTCCGTTGTTGGAAACTAAACTAATTAGTCGAAGAATTTTTTTT
CAACCAACTTATTTCCTGTAATAGTTAATAGAGATTTTTCATTGGACATT
GCGACCTTAATATTTGCGGGGAGCATCTACTTCCTCATTGGTAACCCTGC
TATTTCTTGAAAAACGATTTTTCGTTTGTAGACTCCACCATATGAACGCA
GATCTGTCGCCCATGGTCTTGCTTGAAATGATTGCGCGAAAGATTTGTAC
CTGTCAACACTAGCCCATATCTCATCACCTCGTAGGACTAGAAATAGGTA
AATGGGTCGACCTTGGGCATGGCCCTAGCATCTGCAAAAGGTGCATCCTT
CAACACTCTTCTGCCCAAGAAAAGTTAGCTCAATTTAAAGCTTCCTGCTT
ACAATTGCTGCCATTTTCCGTTTTCCAATGAGCCTGGATTGTTGATGAGC
ATCTTAGAAATGCCGCATTGGGAAGTCTCAGTCGACTAGCGACTCGAGCT
CATGAGCCATTTGAATCTCCCATGCAGCCTAAGAATTAGTCGTTTCTTTT
CTTTTTCATTTTTCCCCTTCATTTTTTTTGGTCACTGACCAATACTATAT
AAACTTGTGTTCACCCACTTATAGTTGGCGATTATTTCCAGGCACAAGTA
SEQ ID NO: 53; Pp03g05430 (homologous to ScTHI4) Promoter
TGCTGTTTTGGGCTCGTACGGATGTTTCTTAGGTCCGATGATTGGTGTTA
TGACTTGTGACTACTACTTCGTACGTCATCAGAAACTCAAGCTGACAGAC
CTCTACAAAGCCGACAAGAGTTCTATTTACTGGTTCTACAAAGGATTCAA
CTGGAGAGGTTTCGTTGCCTGGATTTGCGGTTTTACTCCAGGTATTACAG
GGTTTCCTAGCGTCAACCCCAACTTGACTGGGGTTCCTACAGCCTGTATC
AAGATGTTCTACATTTCGTTTATCATTGGTTACCCGATCGGATTCTTAGT
TCATCTGGCACTCAATAAGCTATTCCCTCCACCAGGTCTTGGTGAAGTCG
ATGAGTATGACTACTACCACTCTTTCACCGAAAAGGAAGCACTGAAATTA
GGAATGGCCCCTAGTTCCGAGTTGGACAGAGTCAGCACCGATGACCCGAT
CAATATTCCTTACGACGAGAAGTCTTTAGGCTAATGTAGTTAAATAGTTA
ATCGAAACAATCGTGTATCCTCTTTATCGTACCAGCGGGATTCGCTGCTT
GGATGGGTGACTCCTGTCCAGTTGACTCAAAGTAGTCAAAATAGGCCTGG
AGACCCTTAACAGGTCGATGAGTAGCCTACTATGAGAAAACCCCTCACCA
CAACTGGACTATAAAAGGGCACGTCAATCCCCAAAGCAACTCTTTTCTTT
CATCCCTACTTTATTACTTTATCCTTTGATCTTCATTGAAGAAAATCTGA
AACAATTGTAAGGAGCAATCCACACCTCCCCAGCAATGACTCAATTTACT
AACCCCATTGACAGAAAATGTGAGCATCTTTTTTAGATGTCATGATGATA
GGTGGAGTATTCTTAATTATTGCTTTCAGCAAACCGGTGCCCATAAAGTG
TTTCCCATTAAATCAATGAGAGGCATTAAGGCTGAGATTAAACGGTTGAA
CTTGAACTAGATAATTCTAGCGGAAAGAATTGCTCTTTTTATTACGTCGT
SEQ ID NO: 54; Pp03g03490 (homologous to AN2957.2) Promoter
CTGCCGAAAGAAGCACAAGAAATGTGACGAGAACAGAAACCCAAAATGTG
ACTTTTGCACTTTGAAAGGCTTGGAATGTGTCTGGCCAGAGAACAATAAG
AAGAATATCTTCGTTAACAACTCCATGAAGGATTTCTTAGGCAAGAAAAC
GGTGGATGGAGCTGATAGTCTCAATTTGGCCGTGAATCTGCAACAACAGC
AGAGTTCAAACACAATTGCCAATCAATCGCTTTCCTCAATTGGATTGGAA
AGTTTTGGTTACGGCTCTGGTATCAAAAACGAGTTTAACTTCCAAGACTT
GATAGGTTCAAACTCTGGCAGTTCAGATCCGACATTTTCAGTAGACGCTG
ACGAGGCCCAAAAACTCGACATTTCCAACAAGAACAGTCGTAAGAGACAG
AAACTAGGTTTGCTGCCGGTCAGCAATGCAACTTCCCATTTGAACGGTTT
CAATGGAATGTCCAATGGAAAGTCACACTCTTTCTCTTCACCGTCTGGGA
CTAATGACGATCAACTAAGTGGCTTGATGTTCAACTCACCAAGCTTCAAC
CCCCTCACAGTTAACGATTCTACCAACAACAGCAACCACAATATAGGTTT
GTCTCCGATGTCATGCTTATTTTCTACAGTTCAAGAAGCATCTCAAAAAA
AGCATGGAAATTCCAGTAGACACTTTTCATACCCATCTGGGCCGGAGGAC
CTTTGGTTCAATGAGTTCCAAAAACAGGCCCTCACAGCCAATGGAGAAAA
TGCTGTCCAACAGGGAGATGATGCTTCTAAGAACAACACAGCCATTCCTA
AGGACCAGTCTTCGAACTCATCGATTTTCAGTTCACGTTCTAGTGCAGCT
TCTAGCAACTCAGGAGACGATATTGGAAGGATGGGCCCATTCTCCAAAGG
ACCAGAGATTGAGTTCAACTACGATTCTTTTTTGGAATCGTTGAAGGCAG
AGTCACCCTCTTCTTCAAAGTACAATCTGCCGGAAACTTTGAAAGAGTAC
SEQ ID NO: 55; Pp05g09410 (homologous to ScTHI13) Promoter
ATCTTTTCAGCTTCATCGTCAGTGATATTTCTCAGCCCACAGACCAAGTC
AACTTTGGAATCTAACAACCTTGTTCTTACAATGTTAGAACTCTTAAGTC
GCATGCCATGATCTTCAAGCTGAATTTTGTGAAGGAGGTCAAACCCCACA
ATGGCATCTAGTTGTTTAGAATACATGCCTTCGACAAGTGTTTGAGTGTC
CAAAATCAAGAGCTCAAAATTATTGAATTTGTCTGCCAATAACGCCGTAA
ATTGATTAGTGTCCAGCCCACCAACAATAGGAGCACCTATAGTTAATTTT
TCAGATAAATTTAAGTTATCAAGGTAAAGGAGCTCTAAGTTTACCCCTTC
CAACAGGGTTATTTGAGAACTCAATAAATTGTTGAATTCAAAACCAATTG
TCTTTGAATTCTCCACTGGAGCTTCCTTGCTGAAATTGATTTTGATACCA
TTGGCATCAAAGAGACCCGTATGATAACTCCATAAAAAGGGGAGATGATA
GGCCTTAAATTCATCGTTAATCTGCAAATTTATTCCTGACATGTCTTTGT
AAATAGTTATAGTTCAGAAACTGGAATTGAGCTCAAAAAACTGGAATCGA
GCGGATATTTGAAGATTGATGCCTTACTCATGAATTGATTGATAAGAGCT
CCGTGATTCACTCTGTCAATGATTACCCCTCTCCTACCCGATTTGGGACT
TTTTCTTCAGTCTTGGGGACTTTTTTTCATATGACTTGACCTTGCTTTCC
CAATAGGGAAGGACTCACCCATGGATGATTAAGTTTGGATTACTCGTTTA
GGAAATAGTAGCCATGAATCAATTTGAATCATACCATCATGAAATAGGGT
TAGGCTGTAAATGCCTCAAAAATGGCTCTTGAGGCTGGATTTTTGGGTAT
TGGAATGTTGGTAGCAATTGGTATAAAAGGCCATTTGTATTTCACTTTTT
TGTCCTTCATACTTTACTCTTCTCAACTTTGGAAACTTCAATAAATCATC
SEQ ID NO: 56; Pp02g07970 (homologous to ScPEX11) Promoter
CAAACTTAACCGACCGTTCTTCCATCCGTTTATTAATATACACACCTATG
AACTGAGCCAGGTTTTCAGGTCTCTGTGACTCTCTATACATTGACGGAAC
AACATCCGTTCAGTCTCATCCAATTGCAGCCCAAACTCTGAGTTTAGCAA
TTGCAAATGGTTATTATCTGACGAGTAATCGTTGATGGCACATGCCCTCT
GTTTGAACATCTCTTGAACAATAGCCATCAGTTCTGTGTCATTAAACATG
CTTCCCCATTTCACTGACAGTTTGTAGAAATAGGCCAACAATTGATCCAA
ATCGATTTTCAACGCATTGGTTTTGATAGCATTGATGATCTTGGAGCTGT
AAAAGTCCGGCTGGATAAGCTCAATGAAATAGGTTGGTTGATCTGGATCT
TCTTTTGGGTCATTTTGTTCGCTCTGTATTTCACAAATTGCCAGAATCTC
TGCCAACCACAGTGGTAGGTCCAACTTGGTGTTCTGAATCACAGGCTTCC
CCGGGTTGTTCTCTAAATAACCGAGGCCCGGCACAGAAATCGTAAACCGA
CACGGTATCTTTTGTCCGTCCGCCAGTATCTCATCAAGGTCGTAGTACCC
CATGATGAGTATCAAAGGGGATTTGGTTATGCGATGCAACGAGAGATTGT
TTATCCCAGATGCTGATGTAAAAACCTTAACCAGCGTGACAGTAGAAATA
AGACACGTTAAAATTACCCGCGCTTCCCTAACAATTGGCTCTGCCTTTCG
GCAAGTTTCTAACTGCCCTCCCCTCTCACATGCACCACGAACTTACCGTT
CGCTCCTAGCAGAACCACCCCAAAGTTTAATCAGGACCGCATTTTAGCCT
ATTGCTGTAGAACCCCACAACATAACCTGGTCCAGAGCCAGCCCTTTATA
TATGGTAAATCCCGTTTGAACTTCGAAGTGGAATCGGAATTTTTACATCA
AAGAAACTGATACTGAAACTTTTGGCTTCGACTTGGACTTTCTCTTAATC
SEQ ID NO: 57; Pp01g12200 (homologous to AN7917.2) Promoter
ACAATTGTTTAACGCCTTGTTGGACAGTTGGGATTTCAACTGAAGTTTTC
GAAACAGTTCCGGTGATGAGGATATAATTGAGTTCCATTTTGTTGAGACC
AATAAACAGGCATACATGTCTGAGAAATTCAACAGGCGAAAGATCAGCTT
CACTACCTCTATCGGCAAAAAAATGGAAGGATCTATTGTTCTTGGAGTGA
CTAATCTTAGTTTCTTTGTACAAGGCACTGGTGCGTCAAACTTTAAAACT
TTGAAGTGTAGCTGATTTTTCTTGTCCAATTTGATTTTCAGTGCATTTTT
CAGTTCCTTTAATTGTTCCAAAAGAACCTTGGAATATCTAAATTGATTAT
CCTGATCTCTTCTAGTTTCTATTATGGAGATCCCTTCTCTTGCTGTAGAC
AAAGCCTCACTGATCTTCCCTTGTAGGTCTAAAATTTTGCAAGTTCTCAA
ATAACCCTTGCAGTTGTGGCACTCTCTCTTGATCATTTTAAGACCATCTT
TCAAGGCCATATTCAATTGATCTAGCTTTTCCAAACAGGCTGCCCTACAA
TCTAACAGAGATATCAGTTGAGATACACAGCTGGCGGCCAACTCGGGATC
ATTCTCAATGAAATTGATAGTAGAAGTAAATGCCTTGTAGCAGCGCCTAT
ACTCTTTTTGTTTGAAAAGCTCAATACCTGTCTTGAGTCCCTGATCAACC
AATTGACGGTCCATTTGCTGCCTCCTTCAGGGCCCCACGATAGTATACTC
TTTGCCAGTTCAATGCACAGGCATGTAAAAACGCGCTACCATCAGATACC
ACCGATACCGCCCCAAATGGCCCAACTCGGTAATATCTGGGGAGGCTATC
TTAGACTAAAAAGATTAAAAAAATACCCGGGAACCACAACTAAAATGGTG
TCACCCTCTTGCAAACTTATATAACCTCTTCATGTCGTTTGGGAACCACT
ATTACTTCCCCGTTTCAACTACATCGTTTCCAAAGTGGATAGACCGTATA
SEQ ID NO: 58; Pp03g11380 (homologous to ScPMP47) Promoter
AGCTCAGATTGGAAATGATTTTTGATCCTACCAAGAAGCCTTTGATTTCC
AGAATCTCCGCTAAGTAAGTAACCCCCGCAAACGCATGCATCCATGCAAA
CAAAATACTAACAATTTTAGCCCCGTTGTTGAGAAACCCAGAAAATTGAA
TGTTCAACCAATCCAGACGATCAATAAGAAAAAAGGCCCAAAGGCTACTT
CCAAACCTGCTGCCGCCAAACCTGCTCCTTCAAAAGCCGGTCCCAAGGGA
GGTAAGAAGGTGAGAAAGCCAAAGAAGACAGTTGAAGAATTGGATCAGGA
AATGGCTGACTACTTTGAAAATAAGAATTAGCCCAACAAAATATGTACAA
GTATTATATAAATGAATCTACATGGTGTGTTTTATTTAGATCCTCCAAAC
CAAGGAAAGAAACTAAACTTATCTCCGGACTTACGAGTCAAATAACTATC
CGCAGTTCCTTGGAACTCAGACTTTCTTCCATAAGCGGTCATATCATCTT
TGGACTGTGGGAATCCTGGACGAATCTTTGAAATGTCATAATCTTGCTCT
CTATCTCCAAGCACAGCGTCCGGTAAATGCTCGTTCTTCTTTCTCAGATG
AATCTTGGATTTAACAAATAAAGCCGTGCCTATGGCTAATGTACTCAAAA
ACAAAGTCTGCTTCCAGAATTTCGCAAACGATGGAATGCCATTTCCTGTA
AATGTACTCATTGAACCTATGTTTGATTAAAGTTGGTGTGAAGTCATCAA
ACGAGAGTAAAATCAGATACTCGTGCACCGGCCAAAATTGACTGAGCTAA
TCTCTGCAGGCTTGACATCCGAACACAACAAATAGGCGACAAATCTTAAC
TATCTAATCGTAGGCTATGGTAGAACTTTGTGGGGGTAGAGGAAGACTAC
AACAGCAAGACAAAACAAAAGAGTCATAGTTTGACTCTCTGCTTTTTTCT
TCTTTCTCTTCTTTTTCTTCCTCCATATTCGTTATTTATTTCGAACTGGA
SEQ ID NO: 59; Pp03g08340 (unknown) Promoter
GTAAATAAGTTAAAGTTTTAAAAGGAAAGGATGCAAAAAATATCCTTGAA
GGCAACGAATATTTTGAAATCCCCGATGCCAAATAAATGCTATCACTTAA
AGAGCACAATAGAGGTGGAAAAAGAAAAACTTGGTCAAGCTAAGGGTTAG
CAAGTTTCTGTTTGTGATAATCAGGGAGAAGGTGTCAGAAAAAACATGAT
TATGTAAGTGGTGTTAGGAGCCGTTAAAGCATTCTGTCGGCCAATAGCAA
GCCCGCCTTTTGTCATCTTTTTGCGGTTTCTCTGTGTGAGACACTAATCA
CCTTTGTAAGACATCGGGAAAACCGTTGCGCAAAATGAGATAGAGATTGT
TCTCGATAGAGGAGCGTAGTAGCCTCTCCAGCCTGCTTTAGCAACATAAT
AGAAAAGAAATATGCGTTGCCTAGGGAGGCTACGTATGCCCAGCATAAAC
GAGTGTTTACCTTACTTCGCACGAGCAGTAGCCACTAAGATCATTATAAA
CTCACCTATTGTCTTCATGCTGTGCTCCGCGTATTTCTCTGTTCAGGGTG
TCATTTCTCGTCATGAGAATCTGATTGATGACTATGCGAGATTACCCCTG
GATTTTTTTTGATCCCGTAACGCGAACTTGAACATTGACTTTGATATGGC
AATGGGCCCTAATATGCCCTAATATGCCCTAAGCTTAACAATTGACTTCT
GTTCTCTGGCAGACTCCACAGAAAACTGGTTGACAGGTCTAATTTCTTTT
TGAATCATTTCCGGTGATTCATTTTGATGCTTAGAGTGAGTCATGGGTTC
TTTATCCGCATTCTTCTTCGCGTCTGCTGTGCTTAATAATAGCCTACTAA
AAATGTGGGGAGCCTCTTACCTTATGTCTATAAAAACAAGCACATGACTA
TGCCATCGCCTTCATAGTTGTTCTGCGCGTTTTTGCTTGTTTTATGACCG
TAGAGACCAACCAATTTACATATCTACAGGGTAGCACATTCGATAAGAAA
SEQ ID NO: 60; Pp05g04390 (homologous to ScTIR3) Promoter
GTGAACGATGGCGTATATTTGAGCGGCCATTGAATTATTGTTGAGTTGTA
CAATGATTGCAAATGGTCCATGATAAGGGATGACTACTAGCTGTTGAAAG
GGGGGATGAAAATATAAGCCTAGGGGATTCCGGGCGGAAACGAGGGCGGG
GGTGACGATTATTCTTGAAGGCCGCTGACCGGGGTGAGAAGAACAATCCC
TTGGACGGGCAAAACGCTAGATCGAAGATTTTCAGACTCGAACGATCGGC
CATACCTGAGATCGATCGGTCAAAAAGATTCACTTGCTGGGTGCCTCTGT
CGTATTGGCTTCAGGCCCCACAAACTTAACCTTACCTTAATGGTTCTTTG
GTCCTTTGGTTCTATCCTGAAACATATTGCCACATTTCCCCCACCAAAAC
TCTAATCAAACCGAGAAGCTTTATTGCTATTTTCCCCATAGACTAATCCA
TATCCCCTTTGGAAACGCCAACTGCATCTGCACCCTAATGCCTTCAAACC
CATGACCCTAGCACAAGAACCCTCGGAATTATACCATCCCCATCCCATAT
GACGACCATAGTTCGCGTATTCTTCTCCATCATGTCCTAAAAGGGCAAAT
CAGTTTTGTGGAGAAGTCATAGGGTAGGAAAGAAGGTTGCTTCAAATGGT
TGCGACCGGTCCCACATGGGGCATGCGTGAGGTAAAAGAACTCCATCGCA
CTAGGGATCAACGGTAGCAGATTAGTAATTTACCTGAACAGTAATCAACT
AGTTATCACAGTCAATGCGTAGCTTATTGCACCTATCAATTTGCCTAACC
CAAGGACGGACGACATTACTAAGGTGCACCAACTAAGCCCTCTGCGCTAA
AGGAGAAAATCTATATTTGTCTTGCATGTGCCATGCAGTCGTGATTTAAT
TGACGAAAGCAAGTTTTTCCGTTTAAAAGGGACATAAATTCACTGCAATT
GAGGCCCTTTACATTGCTCAGATTCACCAACTTCCTACCGTTCTAATATA
SEQ ID NO: 61; Pp01g08380 (homologous to ScYIL057c) Promoter
TTATCCGATGCGCTTCAAAGCTGGAATTGTAAATATAGAGAAAAAGAAGG
ATGTTGTTTTATTCTTGAAAGAGTATAATTTTACTTCTAGCAACTCTCCC
ACTTCGCTTGACTTCATTTATTTCTTGGGCACATAGGCGTAGTAATCTAG
ACCAACAGATAATTTGCCGGAATGATATAGCGATTGGAAAATGAACTGAA
ATTTTTTGCTGTCTTTCAATTTGACGGGCAGTTCATCAGTGACCGACCAT
ATAAATACGTTGAGAATGTTATTCTTCCTCGTAGTTGAAGTGGCTTCATA
ATTTCAGAACTCAATAGATAAACTAGGATGTTTTAAAGCAATTAATGCTC
ACAAGTAAGGAGCGACTCTCTTGCTTTTCGAATACTAAAAGTATCGTCCC
AACCCAGAAAAAAAGACCTCTTAACTGCAAAATAAACTCTATATATTTCT
TCTAAAACAGTTTCAGGTTGGATAGTATCGCATTCTCATCACTTCTAACT
AGTAGGCCATGAGATATATTAACGTTTACTTGAGTTCTAAGTTCTCCGAA
TTAGATGCACAGCACAAACAAGATTAGGTTTCACTTGGTACAAAATACGA
ACAGAGTTTAAGGTCGTAATTTCATTTCGTTATTGATCCCCACAATCTAT
TCTTATCACAGTCATCAGATAGTCGCGAAAAAGCATGCAGAAAAGGGGGT
CGTCCCTATCTAAGTTGTAGCATTACAACAAATATGACTACACTCAGTGT
CGCAATCGGTATAGCCAACGCTGCAAAATGGATTCTACTGAGAATGGTAT
GATGATCCCAGGATCAATTTCCCAAAAATTAAAAAAAGTAAAATAAAAAG
CATCAGATATTAGGGAGGTGGTAAGATTGCTCTGCAAGCGATCACGAGAT
TTTAGGTTTTCCTTTATGTACTATATAAAGCGCAGATTGGATGCCGCTTT
TCCCTCCTGGGCTATGATAATATAGCGAACGAAATACACGCCAAAATAAA
SEQ ID NO: 62; Pp01g05090 (homologous to ScSAY1) Promoter
GAGCAGGCTTTTTGGCTGCTAACGGGTCCTCAAATTCATTTCCATCTTCG
TCTTCCTCAATTTGTTCCACTGCCTCAACTTCCTCTATATAATGCTTCAA
CATTGATTCAATTCCATTTTTTAGGGTGATGGAACTTGAAGAGCAACTTC
TGCAAGCTCCTCTTAATCTTAGATATACTGTTCCTGTCTCGTACTCAAAT
CTAACAAATTCGATGTCACCACCATCATCCTGGATGGCAGGTCTTATTCT
GGTAAATATTAGCTCTTTAACCATGCTGACGACTTCATCTTCGTCATCCT
CTTCCAGCAAAGCCTGGTCATTTGCGTCTGACTGGTGCTGTTCGTTCAAG
ACAGGAGTACCGTTATTCAAAGACTCTGTGAGGACTGCAAATATCTCTGG
TTTCAACAAAGACCAATCATCTTGAGTCTTCTTCTCTACAGTGATAAAAT
CGTGACCTATCATGATGGTTTTCACTCCATCTATCCCAAACAACTTGAGA
GCTAAAGGTGATTTGAATGCTTGTCTTCCGTTAAGAAACTCTATAGTGGT
TTGCTCAGGTAAAATCTTCATGGATGGCAAAAACTTGAGGGCATCATCGT
TGGGGGTCGTTTGTGTTTGAATAAATAGGCTCCTCAAAAAAGTCCTGTTG
ATAGGCACTATCTTTTGTCTATTTAATAGTCGTAGCATTGCTGTGTGTAT
TGTGATGGGATTGTAGAGGAAAAGAAAAATGAGAAACTAGCACCCTTTAG
ATAGTGCGCATTGGTTGGCATCTTAGTGGGGGAGGCCACAAGGAGAAAGC
ATCTTCTCCGCCTGGTCGTGGTGCTGACAAATAAATCGATCATTAACGGT
ACAGTCATCTTTATTATTTATAATATAATATCATAGATACTATGTTATAA
TTATAATTTAGAAAAGATAAAGTTTGACTAGCCGTTCCCCAGAGGCTATA
TAAGGAAGGTGGTTAAGTCCTCCAGGTTTTACTGTTTCTTCCTTATTGCT
SEQ ID NO: 63; Pp01g13950 (homologous to ScTPN1) Promoter
CCACTAACATTAACCAACAGCGAAATGCACGACATTATTCAAAATGTCGT
ACCGTCTGAGGGTGCAGAGATGTAGTAGTGTGAAAAGAGGGAGAAAACAG
TGGATGCTGGATGTTCGATGTCGGACCTGTGAGTTTGATCAATAGGTCAA
TTACTGGGAAGATGAAATGGATGCTTGTGTAGCGTTTAGGTGCTCAAAGA
AGATTGATGTTGGGGCGAGAGCCTAATTTCAAACGCCAACGAGATTGTTT
GTTGGTGAGCGGGTCCAACTTCCAATTGGATCATTCCTCTGCTCGAGCCA
AAGAGACGTTATTTTTTGACGCCTGCTAAGCTTTTTTTAAGCTTTTTAAG
GATCTTTCGTAGTGAAGAGTTTTGAGTTTTTTTCCTGAACCAAGCTAGAT
AACCTGACTTCATCTGAGAAAAACAGACCACAACGGTTAATCAAAAGTTG
GGAGATCAAATCAGGAGATTTCTCCAGTTACCATGCGCATACACAACAGA
TAACCGATGATTATGAGTCCCCTTTTTCTTTCTTTGACAGGTCTTTTGAT
CGTAAAATCAAATGGACAGTCATAAGTGAAACTTTCATAGAGTGGTGGCG
TACCTATTGCACTCACTAATTTGCAGTACAGTTTACTCGCAGACACCCGT
AGCCTATCAAGTCCCTTTCCCTTACTTATTTCAAAAAATATGCCTCTTTC
AAACCTACCATGACTCGTTCGTTGTAAAAAGGAACCCCTTTAGTCGGGAA
AAAAGTCTCGCAATGAGTTAGATCGGGGTAGTCATAACCGTAGCCATGAC
TCGCTTTAATCATTGCCTGTTTAATCACCGGGTATTAAGCAAATTGCGTT
CACTAACCAATATTTATCCGTTTTAGTCGCAAGAAAATTTCAAATCCGAT
CTGCAAGGTGAGATGAGTCGTCGCTAGATGCGGTTATATAATAAGAGGTC
TTTCCCCATACTACAATCACTTGACCCAAAAGTAGTAGAATTTCACTATA
Sequences of methanol repressible genes
SEQ ID NO: 64; Pp03g11420 (homologous to ScARo10) gDNA ORF
ATGGCACCAAGTGCCTCAACCATTCCAATGGGTGAATACATTTTCAGAAG
AATCCAATCATTAGGCGTATCCAGTGTGTTTGGGGTTCCCGGAGATTTCA
ATCTGAACCTATTGGAGCATCTCTACTCAGTGGAAGGCATGTCGTGGGTA
GGTTGTGCCAACGAACTAAACTCTGCCTATGCTGCAGACGGTTACTCTAG
AGCTTCAAATAAAATGGGATGTGTGATAACAACTTTTGGTGTTGGAGAGT
TGAGTGCAATCAATGGAATATCAGGTGCTTTCTCAGAGTACGTGCCTATT
CTTCATATTGTTGGAACAACTCCTCTCTCTGCCAAGATTGCTGAAAACAA
TCATACCCATCATTTGGTCCCAAAGTTGTCTGTCTTTGAGCCGTCAGACC
ATTTCACGTACGAAAAAATGGTAGCACCTGTTTCTTGTCATCAGGAAACT
GTTGTAAATGCTAGTGATGCACCAGGACAGATTGACACATTGATAAGACA
GATCTTGAAGTACAAAAGGCCAGGATATCTGTTTTTACCTTCTGATTTGG
CCGATATCAATGTAGATGGGGATTTTTTAATTCAAAGAACTACTCAACAA
TTCTATCAATCCGTCGACACAAATCAATCTCTAACAAGAGAGGTTGCAAC
CAAGGTACTGGATAAGATATACAATTGTTCAAATCCTGCCGTGCTGGGAG
ATATACTTTGCGATCGTTTCCAAGTAACTGAACATGTTAGAGCATTTGTC
AAAAATGCATGTATCAAGAGTTTTTCCACTTTCATGGGTAAATCAGTCCT
GGATGAGAGTGACTCAAGATATATCGGAACCTACAATGGCGTGGAGTCAA
ATAACGAGGTGATTGGATACTTCCAGGCTTCTGATCTCATATTGCATATT
GGAAACTACTACAATGAAATCAACTCTGGACATGACACTTTGTACAACAA
CATCGACGAAGAGCAATTGATCCTTATGCATCCAGAGTACATCAAAATTG
GAACTGAGTTGTTCAGGAACGTCAACTTCGTGCACGTCCTAGACGTAATG
CTTCAAATGATGGATGTGTCTCAAATCCCCCGAGGCATTAGCCCCACTTT
ATCAAAGAAAGAGATTAACCACATCGAACACATTTCAGCTTCCACTCCAA
TTTCTCAAACACACCTTCTGCATAAATTGCAAGACTTCATTAAGGAAGAT
GATTTTGTTGTAGTGGAAACAGGATCTATTATGTTCGGACTTCCGGATTT
AGTCCTTCCAAAGGGTGCCCGTTTGTTTGGACAGCATTTCTACTTGTCCA
TTGGCTACGCTTTACCTGCTGCCCTAGGAGTAGGAGTTGCTATGAAAGAT
GGAAACAGTAAGGGAAGACTCATCCTTTTAGAGGGAGACGGATCCGCCCA
AATGACTATTCAAGAATTCGGAAACTATGTCTACCAGCAAATCACTCCTA
TCATTTTTCTCTTAAACAACAGCGGATACACGGTGGAAAGAATAATTAAG
GGGCCTCAAAGGGAATATAATGATATTTTGCCAAACTGGAATTGGACCGA
AATTTTTAAGACATTTGGAGACAGATATGAATCTAAAAGTGAAACAAAAA
AGATCCAAACTGTCGAGGAGTTGGACCAAGTTATGCTGTACACCAATAAT
AACAATTCCAAGCTGAAGCTTTTTGAAGTAATACTTGATCAAATGGATGT
TCCTTGGAGATTTAGTTATATGACTGCTGCCAGCAAGAACAAAGCCAAAA
TCGTAGGT
SEQ ID NO: 65; Pp02g11560 (homologous to ScMET6) gDNA ORF
AAAATGGTTCAATCATCTGTCTTAGGTTTCCCACGTATCGGTGCCTTTAG
AGAATTAAAGAAGACCACCGAGGCCTACTGGTCTGGTAAGGTCGGAAAAG
ACGAGCTTTTCAAAGTCGGAAAGGAGATCAGAGAGAACAACTGGAAGCTG
CAAAAGGCTGCTGGTGTCGATGTCATTGCTTCCAACGACTTCTCCTACTA
CGACCAAGTTCTTGACCTGTCTCTTCTGTTTAACGCTATTCCAGAGAGAT
ACACTAAGTACGAGTTGGACCCAATTGACACCCTATTCGCCATGGGTAGA
GGTTTACAAAGAAAGGCCACCGACTCCGAGAAGGCTGTTGATGTCACCGC
TTTGGAGATGGTTAAATGGTTTGATTCTAACTACCACTACGTCAGACCCA
CTTTCTCTCACTCCACTGAGTTCAAGCTGAATGGTCAAAAGCCAGTTGAC
GAGTACTTAGAGGCCAAGAAACTTGGAATTGAGACTAGACCAGTTGTTGT
TGGTCCAGTTTCTTACCTGTTCTTGGGTAAGGCTGACAAAGACTCTCTTG
ACTTGGAGCCAATCTCTCTTTTGGAGAAGATTTTGCCTGTCTACGCTGAA
CTACTGGCCAAGCTGTCCGCTGCTGGTGCCACTTCCGTGCAAATCGATGA
GCCAATCCTGGTTTTAGATCTCCCAGAGAAGGTTCAAGCTGCTTTCAAGA
CTGCTTATGAATACCTTGCCAATGCTAAGAACATTCCAAAGTTGGTTGTT
GCCTCCTACTTCGGTGATGTCAGACCAAACTTGGCTTCTATCAAGGGTTT
ACCAGTCCACGGTTTCCACTTTGACTTTGTCAGAGCTCCAGAGCAATTCG
ACGAAGTTGTTGCCGCATTGACAGCTGAGCAAGTTTTGTCCGTCGGTATC
ATTGACGGTAGAAACATCTGGAAAGCTGATTTCTCCGAGGCTGTTGCTTT
CGTTGAAAAGGCTATTGCTGCTTTGGGTAAGGACAGAGTTATTGTTGCCA
CCTCTTCCTCTTTGTTGCACACACCAGTTGACTTGACCAACGAAAAGAAG
CTGGACTCCGAGATCAAGAACTGGTTTTCGTTTGCTACCCAAAAGTTGGA
TGAGGTTGTTGTCGTCGCCAAGGCTGTATCTGGTGAGGATGTCAAGGAGG
CTTTGTCTGTAAATGCCGCTGCCATCAAGTCTAGAAAGGACTCTGCTATC
ACTAACGATGCTGATGTTCAAAAGAAGGTTGACTCCATCAATGAGAAGTT
ATCTTCCAGAGCTGCTGCTTTCCCTGAAAGATTGGCTGCTCAAAAGGGCA
AGTTCAACTTGCCTTTGTTCCCAACCACCACCATTGGTTCTTTCCCACAG
ACTAAGGATATCAGAATCAACAGAAACAAGTTCACCAAGGGTGAAATCAC
TGCTGAGCAATATGACACTTTCATCAAATCTGAGATTGAGAAAGTCGTCA
GATTCCAGGAGGAGATTGGTTTGGATGTTCTTGTCCACGGTGAACCAGAG
AGAAACGATATGGTTCAATACTTTGGTGAGCAGCTGAAGGGTTTTGCCTT
CACCACCAATGGTTGGGTCCAATCTTACGGTTCTCGTTACGTTAGACCAC
CTGTGGTTGTCGGTGACGTTTCTAGACCTCATGCCATGTCTGTCAAGGAG
TCTGTTTACGCTCAGTCCATCACTAAGAAGCCTATGAAGGGTATGTTGAC
TCGTCCTATCACCGTCTTGAGATGGTCTTTCCCAAGAAACGACGTTTCCC
AAAAGGTTCAAGCTCTGCAATTGGGTCTTGCTCTGAGAGATGAAGTTAAC
GACTTAGAGGCCGCAAGTGTCGAAGTTATTCAAGTTGACGAGCCAGCTAT
TAGAGAAGGTTTGCCATTGAGAAGCGGTCAAGAAAGATCTGACTACTTGA
AATACGCTGCTGAATCTTTCAGAATTGCTACTTCCGGTGTCAAGAACACT
ACTCAGATCCACTCTCACTTCTGTTACTCTGATTTGGATCCTAACCATAT
CAAGGCTTTGGACGCTGACGTTGTCTCTATTGAGTTCTCTAAGAAAGATG
ATCCTAACTACATTCAAGAGTTCTCTAACTACCCTAACCACATCGGATTG
GGTTTGTTTGACATCCACTCTCCAAGAATTCCTTCCAAGGAGGAGTTCAT
TGCCAGAATTGGTGAGATTCTTAAGGTGTACCCAGCTGACAAGTTCTGGG
TCAACCCTGACTGTGGTTTGAAGACCAGAGGCTGGGAGGAGGTCAGAGCC
TCTTTGACTAATATGGTTGAAGCTGGTAAGACCTACCGTGAAAAGTACGC
TCAGAAT
SEQ ID NO: 66; Pp01g08650 (homologous to ScYNL067W) gDNA ORF
ATGAAATACGTTTTATCTGAGCAAGTCCTTACAGTCCCAGAAGATGTGTC
TGTGTCTATTAAGGCCAGAATTATCAAGGTGACTGGACCAAGAGGTGAAC
TGACCAAGGATCTGAAGCACATAAACGTTGCTTTTGAGAAATCTGGCGAC
AACGAGATTAAGATCATTGTGCATCACGGTAACAGAAAGCACGTTGCTGC
TTTGAGAACTGTCAAGTCATTAATTTCTAACATGATCACTGGTGTCACCA
AGGGTTACAAGTACAAGATGAGATTGGTTTATGCGCATTTCCCAATTAAT
GTCAACTTCCTCGAGAGAGACGGTAATCAGTACGTTGAGATCAGAAACTT
CTTGGGTGAGAAGAGAGTCAGAGAGGTCAAAGTTTACGAGGGTGTCACTG
CATCCAACTCTTCTGCTTTGAGATGAGCTAATCTTTGAGGGTAACTCC
ATTGAGAACGTCTCCCAAACTTGTGCCGATGTCCAACAGATTTGCCGTGT
TAGAAACAAGGATATTCGTAAATTCTTGGACGGTATCTACGTCTCCGAGA
AAGGAACCATTGTCCAAGACGAA
SEQ ID NO: 67; Pp01g01850 (PpPDHbeta1) gDNA ORF
ATGAGTGTAAACTCATTGAGAGCACCTTCTTCCTCGGCGGGTCCAACAAA
GTTGTCTGTCAGAGACGCTTTGAATTCAGCCATGGCCGAAGAATTGGACA
GAGACCCTGAGGTGTTCTTGATCGGTGAGGAGGTCGCACAGTACAACGGT
GCTTACAAGGTTTCCAGAGGACTGCTAGACAAATACGGGCCCAAACGAAT
CGTTGATACCCCAATTACCGAAATGGGTTTCACTGGTCTTGCTGTGGGTG
CTTCGTTGGCAGGCTTGAAGCCAATCTGCGAATTCATGACATTTAACTTT
GCCATGCAGTCAATCGATCACATTATCAATTCCGCTGCCAAGACCCTCTA
CATGTCTGGTGGTAAGCAACCCTGTAACATCACTTTCCGTGGTCCTAACG
GAGCTGCTGCTGGTGTTGCAGCCCAACATTCCCAGGACTACTCTGCTTGG
TACGGATCTATCCCAGGTCTGAAAGTTATCTCTCCCTACTCTGCCGAGGA
CTATAAGGGTCTGTTCAAGAGCGCCATCAGAGACCCAAACCCTACCATCT
TTTTGGAAAATGAACTGTTGTACAACGAAGAGTTCGAAGTTTCTCCTGAG
GTTCTGTCCCCTGATTTCACTGTTCCAATTGGTAAAGCCAAGATCGAGCG
TGAAGGTACCGATATCACGATTGTATCCCACAGCAGAAATTTGCAGTTCT
GTTTGGAGGCAGCCACCATTTTGAAGGAAAAGTATGGTGTCTCATCTGAG
GTTCTCAACCTTCGTTCCATCAAGCCATTGGATGTTCCTGCCATTGTTGA
ATCTGTCAAGAAGACCAACCATCTGATAACTGTTGAAGCCGGTTTCCCAG
CCTTTGGTGTTGGTTCCGAGATTTGCGCTCAGGTCATGGAATCCGAAGCT
TTTGACTACCTAGATGCCCCTGTTGAAAGAGTGACCGGATGTGAAGTTCC
AACCCCCTACGCCAAGGAATTAGAAGACTTTGCCTTCCCAGACACCCCAA
CTATTATAAGAGCTGTCGAGAAGGTTCTTTCGTTGAAAGAG
SEQ ID NO: 68; Pp03g03020 (homologous to ScSAM2) gDNA ORF
ATGTCTAAAAACGAAACATTCTTTTTCACTTCTGAATCCGTCGGTGAAGG
TCATCCAGACAAGCTTTGTGATCAGGTCTCCGATGCTGTTTTGGATGCTT
GTTTGACCGTCGACCCTCTAAGTAAGGTCGCCTGTGAAACCGCTGCTAAG
ACCGGTATGGTCATGGTTTTCGGTGAAATTACCACCAAAGCTCAACTGGA
CTTCCAAAAAATTATCAGAGACACTGTCAAGCACATTGGTTACGACCACT
CTGACAAGGGTCTGGACTACAAGACCATGAGCGTTCTTGTCGCCATCGAG
CACCAATCTCCTGATATCGCTCAAGGTCTTCACTACGAGAAGGCTTTGGA
GGAGTTGGGAGCCGGTGACCAAGGTATCATGTTTGGTTATGCTACTGATG
AAACTGATGAGAAGTTGCCTTTGACCTTGCTTTTGGCTCATCAACTGAAC
CACGAGCTGGCTTCTTGCAGAAGATCTGGATCTCTTCCATGGTTGAGACC
AGACACCAAGACCGAAGTTACTATTGAGTACAAATACGACAACGGTGCTG
TTATTCCTCTGAGAGTTGACACTGTCGTCATCTCCGCCCAACACTCCGAA
GAGATCACCACTGCTGACATTAGAGTGCAATTGACTGAGCACGTGATCAA
GAAGGTTATTCCAAGCCACCTGCTGGATGAGAAGACAAAGTACCACATTC
AACCATCTGGCAAGTTTATCATTGGTGGTATCGCCGGTGACGCTGGTTTG
ACTGGTAGAAAGATTATTGTCGACACTTACGGTGGATGGGGTGCTCACGG
AGGAGGAGCCTTCTCCGGTAAGGATTTCTCCAAGGTCGACCGTTCCGCTG
CTTACGCTGCCAGATGGGTTGCCAAGTCTCTTGTACACGCCAAGCTGGCC
AGAAGATGTTTGGTCCAATTCTCTTACGCTATTGGTGTTCCAGAGCCTCT
TTCCATCTACGTTGACACATACGGTACCTCTACCTACTCATCTGACGAAT
TGGTCAAGATTATCAACAAGAACTTCGACCTGAGACCTGGTGTTATCGTG
AAGGAGCTAGACCTTGCCAGACCAATCTACTTCAAGACTGCTTCCTACGG
TCACTTCACCAACCAAGAAAACCCATGGGAGCAGCCAAAGGTTCTTAAGC
TT
SEQ ID NO: 69; Pp03g02860 (PpSAHH) gDNA ORF
ATGTCTAACTACAAAGTCGCCGACATTTCACTTGCTGCCTTCGGTAGAAA
GGACATTGAACTCAGTGAGAATGAGATGCCAGGTCTCATTTACATCAGAG
AGAAGTACGGACCTGCCCAACCTTTGAAAGGTGCCAGAATCGCCGGATGT
CTGCACATGACTATTCAAACCGCCGTCCTCATTGAGACTTTCGTCGCCTT
GGGTGCTGAGGTCACCTGGTCCTCATGTAACATTTTCTCCACCCAGGACC
ACGCTGCCGCTGCTATTGCTGCTACCGGTGTTCCAGTCTTTGCCTGGAAG
GGAGAGACCGAGGAGGAGTACTTGTGGTGTATCGAGCAACAATTATTTGC
CTTCAAGGACAACAAGAAGCTGAACTTGATTTTGGACGACGGTGGTGATT
TGACTTCTTTGGTCCACGAGAAGTACCCTGAAATGTTGGATGACTGTTTC
GGTCTGTCCGAGGAGACCACCACTGGTGTCCACCACTTGTACAAGATGGT
CAAGGATGCTACCTTGAAGGTTCCTGCCATCAACGTCAACGACTCCGTCA
CCAAGTCCAAGTTTGACAACTTGTACGGTTGTCGTGAATCTTTGATCGAC
GGTATCAAGCGTGCCACCGATGTTATGATCGCAGGTAAGGTTGCCGTTGT
CGCTGGTTTCGGTGACGTTGGTAAAGGTTGTGCCATGGCTCTTAGAGGTA
TGGGTGCCAGAGTTATCATCAGTGAGATTGACCCTATCAACGCTCTGCAA
GCTGCTGTTGAAGGTTACCAAGTTGCCCCTCTTGATGACGTTGTCTCCAT
TGGTCAAATCTTTGTTAcCACCACTGGTTGCAGAGACATCATCACCGGTA
AGCACTTCGAGCAAATGCCAGAAGATGCCATTGTCTCCAACATTGGTCAC
TTCGACATTGAGATTGACGTTGCTTGGTTGAAGGCCAACGCTCAGGACGT
CAGCAACATCAAGCCTCAAGTTGACAGATACTTAATGAAGAATGGTCGTC
ACGTTATTCTTTTGGCTGACGGTAGATTGGTCAACTTGGGTTGTGCCACT
GGTCACTCTTCTTTCGTCATGTCCTGTTCTTTCTCTAACCAGGTCCTGGC
TCAAATTGCTCTGTTCAAGTCTAACGACAGTGAGTTCAGAAAGCAATTCG
TTGAGTTCGAAAAGTCTGGTCCATTCGATGTTGGTCTCCACGTTTTACCA
AAAATCTTGGATGAAACTGTTGCCAGATGCCATTTGGCTCACTTAGGTGC
TAAGCTGACCAACTTGTCCAGTGTTCAATCTGAGTACTTAGGTATCCCAG
TTGAGGGACCTTTCAAGGTTGATCACTACCGTTAC
Sequences of methanol repressible promoters
SEQ ID NO: 70; Pp03g11420 (homologous to ScARo10) Promoter
AAAGTAATCCGGAAGTTGAAGCTCTGAACAACGACTTAGACAACATGAGC
GACTTTGACCCTGCGGACTTTGATTACTCAGATAGTGACGAGGAAGAAAA
GAAAAAGGACGATAATGTTCCAGTTCAGATACCATCCCATTTGGCTGCTA
TAGCTGCCCAAGAACCCTACCCTGAGGACAGTGACAACGATAATGACAAT
AAATTCTCATCTGATGAAGAGGCCAAGATCTTTGGACCAGACTCTGAAGA
CGAATTGAGCAGTGAAGATGAAAAACCTAAGAAGAAGAGAGCCAGAACCG
ACACGTCGGATGATGTTTCTAAAAAGTCCAAGAGCCTTAAGGGTTTACCG
ATGTTCGCCTCGATAGAAGACTACGCTGATCTCTTGGAGGATGATGGCGA
GGACGAAGAGTGATGGCCTAGAACATTTTCATAGCATTATCTATCTAATT
TCAATCAATCTAAGAAGGTATCTTTTAACCTGGCATTTTTTTATATTACC
GTCTCTTTCCGTCCTTTCGGTTAACCATGGATTGGTGTGGCATTTTTGAA
GTGCCACAAGTGCGGATATATTCGGTTATTCGGAGTCTGCCACAATATTT
TCGCACACTTTTTCCCACAAGCAGTCGCCATACTCCTTCCTGAATAACCG
AAAATTTACAAGGAAATTTCTGGCACGAAAAATTCCCTTTTATAGAGGGA
ACCTACCCACCGGCGTTCCCGAAAGGGATCAGGGCTCCTTCCAACGATCC
CATTTCAAATATCGTAGATTTGTGGACCAGTCTTATCTCGGAAAACGATA
ACCATTGCAGACTACAAAAGACACAACACTGTTGAATATTCTAGTGTAGA
TTCCCTTGCTCGGTGTGTAGGCATATTTTCCTATAAGTACAGTCTCATGT
TACCTAAAATCTCTCTAAACAACTTGTAGAAGCAACCATAATGGCCCCAG
TTCCAGATATAGCATACCATACTTTGAACGACCTGGATCATTCATGGTCC
SEQ ID NO: 71; Pp02g11560 (homologous to ScMET6) Promoter
GATGACGTTGATGCCGGGCATTTGCGATACTGATTCAGAAATTCTGGACG
CAGCATTGTTCAAAATGTGTCGTTGTCTAGAAAATTCGTCCCGAGTTTCG
TACGCCTGTGATAGTAATCTGTCAGCAAACGAGTTAACATTATCCACCCG
ATTGCGCTCTTGTCTCATGTACTCTTCTTCATTCAGGCTATCGACGTTGC
TTGTGTGAGAGCGTTTCTTGTGCTCCTGTATGTCAGACCTAACAGAGAAT
AGAAGGTTTAGTTTGTTCCGCTCCTGTTGAATTGACTCCTGAATGCGGCC
AAAATCACGTTTATGGTCATTAAGTATCTCTTTATGTCTGTGTAATTGCT
GTAGTTTGGAAGCGGCAAGAGTGTCATCCGAATCTGCGATTCGGCTCAAG
GCATTGACCAATTCCTGCCTTCGTTCCAGGATATCCTTTAACGCCTTCTC
CAACGACAATTCTTCGCCTGTGGCAGAACTAGACGATGATTGGCCAAAAG
ACGCATATTGAGACAGTAGCGACTCTGTCTTGTTCTCCAATTGCAACGCT
TGGGACCTTGTTTGGGAGTAGTTCGACATTGGGTTCCTCTGAGATGTTTG
ACAAGTGAGAGCTAAATGATAACGAAATGCCTACCTGGCAGGACGTGTAC
TGATCAAACCTCCCAGGTTCACATCGGTCACTTGCTCGATTCCAGCAAGC
TACGCCCTTTAAGTTTTGTCCACCAGCTTTGCGCACTCTCTTGCCTCTTT
CGAACCCCGAGCGCGCTTCAGATGCAGATCAAAGCACGAGATGCCACGTG
ACAGTCCATGTATTCTTTCGTTTATCTTCGTATAGACAATAATATTTCAT
TGACTCTGTCAATGGTCGATGTTCACGTGCAAAAATTTTCAATTCGTTTG
TTGGGCGACACCTCCACTACGTATATAAAAGGATCCGACCGCCCACTTGT
CCTTGCTTCCTGTAATTGTTTCCCAAACAACTAGTAGTTCAATTATTACT
SEQ ID NO: 72; Pp01g08650 (homologous to ScYNL0G7W) Promoter
ATGCTTACGCGTCTGTTTCAGTCTTTTCAGCTAAGTCTTGTGAACCATGT
GAGTCGTGTGCATATCACTAGAACATATTCTAGTAGTTCTTGTTCATGCT
TTGTGCTGTTGACTACCGTTACCCCTGTTGATTGTTTATGGAAGGTAGTT
CCTGCAGAACGTGCATTTACAAAGAGCTATTGCTTTGAGAATCTTCTATG
TACAACTTACTGCAGGTGTGCATACGCTATTTATGCCAAGCTCTTTACAG
GTTAGAGTAGTCACCTCATGGTAATAATTTCCTGAGCTACAAGTCCACAC
GGGTCTTTACAAACAGGAAAGCTAAACGAAAAATGGGAAGCATTGAAATT
TGGGTTTCTGTCAACCAATGTGGTCTTTACTTCCCATACTCCGTCTAGGG
AATGTCTCTAGGATTGCATAGAAACTTTTCAAAGTTGAACCAGAGGTATT
GCAAAAAAATGTTGGCCAAACAAACACCAAAGGGTGTAAACATGCCTCGT
TCGATTTTACCCCCCCTCCCAATTGCGCATATAAAAGATGGAGCACAGGT
AATCTTTACATTTGGGTACCCTCCCGACATGCGCGTTAAAGGCAGTAGAG
TCTTGGGCAGTACTCGTCAACAGGACCCCAATTGCTGACATTCAACTACA
TACAATTCTTAGTGATTCTTGGAGTACAGTTGATTGATCTGGATAGACAT
CCCTGCGAGTAGTCTTCGGGAATTACAGTGATAATAATAAAGGTGTACTC
CTTTGCCAAGACAGGTAAGCTTCTTCAAGATCTCAAACTTTGTTGTACAT
ATGTACGTTTCCATCAGGGGACTTTCGTTTCGCAGCAACTTCGCTAGTAG
TGGCAAGGAAAAGAAAACTACACTGTGACTGCGATTTCCTGTTTTTACAC
ACTATTAACAAAGTCGTGTATTCAACTGAAAAATTCGCTTTCTATCGCTT
CAGGCCCGTTAGAGTGTGAAAAATTTAGAAACAGTACCAATCAATACGAG
SEQ ID NO: 73; Pp01g01850 (PpPDRbeta1) Promoter
AGTTAAGAACTATAGTTTCTACAGAGCTACTCCAAGTCAACAGCAGTTAC
AGCAGCAGCAGCAATCTCAGCAGGTGCAACAGGCACAGCAAGTTCAACAG
GCTCAACAAGCTCAGCAGGCCCAGCAGGCTCAGCAGGTTCAACAACAGGC
TCAGCAGGCCCAACAGCAGCAGGCACAGCAACAGCAACAACAGCAACAAT
TGCAGCAGCAGCACATCCAACAATTGCAACAAATACAGCAACTACAACTT
GCGCAACAGCAGCAGCAACAGCAACAGCAGCGTCAACAACAGGAACACCT
TCTTCAGCAATTGCAGCAGCAGCAGCAGCAACAATATCACCAACCTCAAA
CACAAGGCCAAAATTTCCCACAACAGTACTTGATGCAACAGGCCCAAGCT
CAGGCCCAAGCTCAAGCTCAAGTTTTAGCCCATGCTCAGCATGACAACTC
ATCCAATCCAATGTTCACTAACATCAGAGAAGGATCTGGGGTCTCAGCTA
CACCTCCACCCCCAGGATTGTTTAGTGGAGTGACCAATCACACTACGGAA
ACTCATAACACTCCATCATCTGAACTATTGAACCAGCTGATGAACGGGGA
GGGTAGAAACTCGATCAATGCTTAGTCGGTGATTATCTGTTATATAAGAA
GTACCTATTAGTTGAATAAAGTAATAATATTGGATGTCTGATGTTCCGAG
GCTTCCCTAGTCCGAGTCGATTGCGCGCGTAAATTGGTGCTTTTCCCCCA
CCGAAACAATAATGAGGGGATCTCCATATCACGTGATGCATTCGGTGTAA
CTTTTAGTGGTATAAACCGCGGTCGGATGCACTCCGCCTAACAAACTTCT
GTGAGGTGCGAAACAAGGAACCCGTAAAGGAAAACCTCATTGATTACCTG
TTAGTTCCTACTTTCTCTTTTACCCACAAGGTTCACTCTCACCACAATGT
TGCCATTTCAAGCAGTAGCCAAAGCAGGCCTCAAGCCCCAATTGACCCGC
SEQ ID NO: 74; Pp03g03020 (homologous to ScSAM2) Promoter
CAGTATGCAACATGGCCGATGTTGGTTTGCGTTTCTGGGCGCACCCTGAT
GGACCACGCTGAGGATGTTTCTCTTCTTCCTGTCTTTGTTGGGCAATGTT
GAGCTGATTACACCGTCAGGAGTTAATATGGGCCGCTCGTGACTTTAGCT
CTATATACGACGCTTGGTATCGCACAAGCTCGTTTTCCCTGCAGAATTGG
AAGCAGTAGACTACAGCTACAGTAATCAGTTGACCGGGGTAATACAGGCA
CTTCCTCAGAAATCCAGATTAAACTCCAAAGGCTGCCTCAAAGATTCCAA
CAACCAGAATGATCAAGTAAACTACGACACGTCCAAAAGAGAAGATGACC
ACGTTCTTTCGAAGAGGCGCATAGAAGTTGGTGACTGGCACGAGGAGTTT
TCATCCAAGGCTGGGAAAAATTCGTTGTTGAATCGTGGGATAATCGCACT
TTAGGCAAACCTTACAGCCTCAAACGCAGCTGTTATCACCGCCAGAGACC
AATTAAGCGCTCTACTTTCATTCCTTCTGGAGCCGCTTGCATGACCCTCT
CCAAAAAAAATATGTCCGAACACTCGCCCTTGGTACTTTCCTTTAAATCC
AGAGATGCATGAAGAAAACCAAGATATTGAGATCACGTGAGTCGTCGCAA
TTGCCTTTAAACGATCCTATACGAATTTTTCATTAAGCAAAGTTTCACAT
GCGACGACGGCTCAGCGTACAAGCACGGCAATTGGGCGAATTGTGCCAAC
ATAAAGCATCAGACTCAGCGTGTCTGTCCTGGCAATACCACCATTGGTGG
TTTGACGCCGAATCATCCACGATTGCCCTTTTAATTGACCCTCTAGCTAA
AGTTTGGCTCAAAAACACGTTGGAGGAATTAATATGTGGCACGTGATGTC
CAACAAATTTTTCAAGACTTACATAAATACTCCATGTCTGCTCCTTCATC
ATTACAACCCACCGTTTCCTTTACCTCAACCTCAAAACACTGTTTATACA
SEQ ID NO: 75; Pp03g02860 (PpSAHH) Promoter
GAGACAAGTATACTGATCTTCTTTCTACCTATTTTGGTTGCTACCCATCC
ACTATACAGGGAGCCCAGCAGACCCCCAAAGGACAACATGGTGGTAATCA
TAGCCCGTTGACTAGATTTCATCGGGATACATTGTTCAAACAACTCCGGA
TGCTGGGCCCACCAAGACATGGGGCCTTCCTGATGCAGCCTACAAGATAT
AATTCTCTGGGGTGCGTTCAACTCACTCAGATGATAGCCAAACAGGAATG
AACCTAAGCAGATCACTAATATGTTGAAGCCCAGCGTGGGTGTAACTTGA
GCTTGGGTGTCGAAATGCATTTTTCAGTTTCAATGTTGCTATAAACAAAC
CAAAGTTGAGGGGAAACAATCTATAAAATAGGATTTTTGAAGTACGTGAA
TTGTTGCTGTCTGCCAGATTCGGGATGCCGATTTGATAAGAGTTTTCAGA
TTTGATAGGGATGTAAGCAAAGCGGGTTGTTTCCAACGGAGAACGAACGA
TAATAACGCAGGAACTCATGACGATATGCAAAGAATCCCCTGATTATTTC
TTCTAAAACGTAGCCCGGTTGTTCTTGTGTCGGGTTTCCTTCAAAAGGGA
CCCTGTGTACTATGTGTTATCCGATCCTTAGAGGGAATCTCCGTGTAAGG
GACATCCAGCATCTTGCCAATTTTTCAAAAGCAATTCTCAGATCTTTGGC
ACCTGACGTCCTAGTCCACGACAAGAGATACGTTCTTCCTGGGGGTGGTT
TCAACGGCCTGAGCTTGCACTTATTACAATTGGCAATCGAGCTGGTATTA
TTTCTCGCATGTAGCAGGATCATACAACATCCAGACACCCCAACCCATAT
CCCTAATTACCACGTTCTCACATGCGCTCAACAAAAAACTCGTAACCCTC
CTTTATTTTTTTAAGGAACCTCAAAAGATTTTCACACCTATAAATATCGT
GAAAAACTATCCTCCATTTTCCTCTCTACTACACTAAACCAAATAATACC

### Expression Methods

The present invention encompasses methods for making a polypeptide (e.g., an immunoglobulin chain or an antibody or antigen-binding fragment thereof) comprising introducing, into an isolated fungal host cell (e.g., Pichia, e.g., Pichia pastoris) or an in vitro expression system, an isolated hybrid polynucleotide comprising a promoter of the present invention, e.g., selected from the group consisting of: Pichia pastoris GAPDH promoter (e.g., wherein any sequence operably linked to the promoter is also operably linked to a downstream CYC1 terminator); Pichia pastoris Pp02g05010 (PpPIR1) promoter; Pichia pastoris Pp05g08520 (ScCCW12) promoter; Pichia pastoris Pp01g10900 (ScCHT2) promoter; Pichia pastoris Pp05g07900 (ScAAC2/PET9) promoter; Pichia pastoris Pp02g01530 (ScPST1) promoter; Pichia pastoris Pp05g00700 (unknown) promoter; Pichia pastoris Pp02g04110 (ScPOR1) promoter; Pichia pastoris Pp01g03600 (ScBGL2) promoter; Pichia pastoris Pp01g14410 (ScACO1) promoter; Pichia pastoris Pp01g09650 (ScYHR021C) promoter; Pichia pastoris Pp01g02780 (ScYLR388W) promoter; Pichia pastoris Pp03g09940 (ScPIL1) promoter; Pichia pastoris Pp02g10710 (ScMDH1) promoter; Pichia pastoris 01g09290 (ScFBA1) promoter; Pichia pastoris Pp03g03520 (PpDAS2) promoter; Pichia pastoris Pp03g08760 (ScCWP1) promoter; Pichia pastoris Pp03g00990 (ScYGR201c) promoter; Pichia pastoris Pp02g05270 (AN2948.2) promoter; Pichia pastoris Pp02g12310 (ScDUR3) promoter; Pichia pastoris Pp03g05430 (ScTHI4) promoter; Pichia pastoris Pp03g03490 (AN2957.2) promoter; Pichia pastoris Pp05g09410 (ScTHI13) promoter; Pichia pastoris Pp02g07970 (ScPEX11/PMP27) promoter; Pichia pastoris Pp01g12200 (AN7917.2) promoter; Pichia pastoris Pp03g11380 (ScPMP47) promoter; Pichia pastoris Pp03g08340 (unknown) promoter; Pichia pastoris Pp05g04390 (ScTIR3) promoter; Pichia pastoris Pp01g08380 (ScYIL057c) promoter; Pichia pastoris Pp01g05090 (ScSAY1) promoter; Pichia pastoris Pp01g13950 (ScTPN1) promoter; Pichia pastoris Pp03g11420 (ScARO10) promoter; Pichia pastoris Pp02g11560 (ScMET6) promoter; Pichia pastoris Pp01g08650 (ScYNL067W) promoter; Pichia pastoris Pp01g01850 (PpPDHbeta1) promoter; Pichia pastoris Pp03g03020 (ScSAM2) promoter; and Pichia pastoris Pp03g02860 (PpSAHH) promoter; or a functional variant thereof, operably linked to a heterologous polynucleotide encoding a heterologous polypeptide and culturing the host cell (e.g., in a liquid culture medium, e.g., YPD medium (e.g., comprising 1% yeast extract, 2% peptone, 2% glucose)), optionally in the presence of methanol, under conditions whereby the polynucleotide encoding the polypeptide is expressed, thereby producing the polypeptide. Expression of the polynucleotide may be induced when the promoter of the present invention is methanol-inducible and the host cells are grown in the presence of methanol.

An expression system, comprising the fungal host cell comprising the promoter of the present invention operably linked to the heterologous polynucleotide, e.g., in an ectopic vector or integrated into the genomic DNA of the host cell, forms part of the present invention. A composition comprising the fungal host cell which includes the promoter of the present invention operably linked to the heterologous polynucleotide in liquid culture medium also forms part of the present invention.

In one embodiment of the invention, a method for expressing a heterologous polypeptide, e.g., as discussed herein, does not comprising starving the fungal host cells of a nutrient such as a carbon source such as glycerol or glucose. Other embodiments include methods wherein the cells are starved. For example, the present invention comprises methods for expressing a polypeptide in a fungal glycosylation mutant strain, e.g., as discussed herein, wherein the host cell comprises a promoter of the present invention (e.g., methanol-inducible) operably linked to a heterologous polynucleotide encoding the polypeptide wherein the host cell is not starved and is cultured in the presence of methanol.

Method for expressing any polypeptide using a promoter of the present invention can be done at any volume including, for example, low volumes and high, industrial volumes. For example, expression is performed, in an embodiment of the invention, in 5 liter or 40 liter volumes. Genes operably linked to CHT2 or PIR1 promoters have been done in 40 liter volumes; and genes operably linked to the DAS promoters have been done in 5 liter volumes.

In an embodiment of the invention, the polynucleotide that is operably linked to the promoter of the present invention is in a vector that comprises a selectable marker. In an embodiment of the invention, the fungal host cells, e.g., Pichia cells, are grown in a liquid culture medium and cells including the vector with the selectable marker are selected for growth; e.g., wherein the selectable marker is a drug resistance gene, such as the zeocin resistance gene, and the cells are grown in the presence of the drug, such as zeocin.

The present invention also encompasses methods for growing cells wherein expression of a polynucleotide is inhibited. For example, such a method comprises, in an embodiment of the invention, introducing, into an isolated host cell (e.g., a fungal cell such as Pichia pastoris) a polynucleotide encoding said polypeptide that is operably linked to a methanol-repressible promoter of the present invention (e.g., SEQ ID NO: 70-75) and culturing the host cell (e.g., in a liquid culture medium, e.g., YPD medium (e.g., comprising 1% yeast extract, 2% peptone, 2% glucose)), in the presence of methanol at a sufficient concentration to inhibit expression, at least partially.

In an embodiment of the invention, polypeptide expression using a methanol-inducible promoter of the present invention includes three phases, the glycerol batch phase, the glycerol fed-batch phase and the methanol fed-batch phase. First, in the glycerol batch phase (GBP), host cells are initially grown on glycerol in a batch mode. In the second phase, the glycerol fed-batch phase (GFP), a limited glycerol feed is initiated following exhaustion of the glycerol in the previous phase, and cell mass is increased to a desired level prior to methanol-induction. Furthermore, the methanol-inducible promoters are de-repressed during this phase due to the absence of excess glycerol. The third phase is the methanol fed-batch phase (MFP), in which methanol is fed at a limited feed rate or maintained at some level to induce the methanol-inducible promoters for protein expression. A limited glycerol feed can be simultaneously performed for promoting production when necessary.

Accordingly, the present invention encompasses methods for making a heterologous polypeptide (e.g., an immunoglobulin chain or an antibody or antigen-binding fragment thereof) comprising introducing, into an isolated host cell (e.g., Pichia, such as Pichia pastoris) a heterologous polynucleotide encoding said polypeptide that is operably linked to a methanol-inducible promoter of the present invention (e.g., SEQ ID NO: 47-63) and culturing the host cells,

(i) in a batch phase (e.g., a glycerol batch phase) wherein the cells are grown with a non-fermentable carbon source, such as glycerol, e.g., until the non-fermentable carbon source is exhausted;
(ii) in a batch-fed phase (e.g., a glycerol batch-fed phase) wherein additional non-fermentable carbon source (e.g., glycerol) is fed, e.g., at a growth limiting rate; and
(iii) in a methanol fed-batch phase wherein the cells are grown in the presence of methanol and, optionally, additional glycerol.

In an embodiment of the invention, prior to the batch phase, an initial seed culture is grown to a high density (e.g., OD₆₀₀ of about 2 or higher) and the cells grown in the seed culture are used to inoculate the initial batch phase culture medium.

In an embodiment of the invention, after the batch-fed phase and before the methanol fed-batch phase, the host cells are grown in a transitional phase wherein cells are grown in the presence of about 2 ml methanol per liter of culture. For example, the cells can be grown in the transitional phase until the methanol concentration reaches about zero.

In an embodiment of the invention, the host cells (e.g., Pichia cells such as Pichia pastoris) are grown under any 1, 2, 3, 4, 5 or 6 of the following conditions:
in a culture medium at a pH of about 5; and/or at a temperature of about 30° C.; and/or
in the presence of any 1 or more trace minerals/nutrients such as copper, iodine, manganese, molybdenum, boron, cobalt, zinc, iron, biotin and/or sulfur, e.g., CuSO₄, NaI, MnSO₄, Na₂MoO₄, H₃BO₃, CoCl₂, ZnCl₂, FeSO₄, biotin and/or H₂SO₄; and/or
in the presence of an anti-foaming agent (e.g., silicone); and/or
at an oxygen concentration of about 20% saturation or higher; and/or
in a glycerol batch phase at a glycerol concentration of about 40 grams/liter; and/or
in the methanol fed-batch phase at a methanol concentration of about 2 grams methanol/liter to about 5 grams methanol/liter (e.g., 2, 2.5, 3, 3.5, 4, 4.5 or 5).

The present invention provides methods for making polypeptides, such as immunoglobulin chains, antibodies or antigen-binding fragments thereof having modified glycosylation patterns, for example, by expressing a polypeptide in a host cell that introduces a given glycosylation pattern and/or by growing the host cell under conditions wherein the glycosylation is introduced. Some of such host cells are discussed herein. For example, the invention provides methods for making a heterologous protein that is a glycoprotein comprising an N-glycan structure that comprises a Man₅GlcNAc₂ glycoform; comprising introducing a polynucleotide encoding the polypeptide wherein the polynucleotide is operably linked to a promoter of the present invention into a host cell and culturing the host cell under conditions wherein the polypeptide is expressed with the Man₅GlcNAc₂ glycoform and/or lacking fucose.

### EXAMPLES

The present invention is intended to exemplify the present invention and not to be a limitation thereof. The methods and compositions disclosed below (including, without limitation, any promoter, terminator, promoter/terminator combination or expression construct, e.g., promoter-gene-terminator) fall within the scope of the present invention.

### Example 1

### Identification of the Putative Complete Set of Protein Coding Genes for P. Pastoris

The complete wild P. pastoris strain NRRL-y11430 genome sequence was determined yielding 9,411,042 bases on 4 large contigs and one smaller contig of 34,728 bp (nucleotide base pairs) that could not be resolved, consistent with the previously published finding that the P. pastoris genome consists of 4 chromosomes. The genome sequence was then annotated using the automated genefinder software FGNESH (Salamov and Solovyev, Genome Res., 2000, 10: 516-522). A total of 5069 protein coding ORFs and 278 non-coding transcripts, were identified. Identified genes were named systematically using the convention Pp (for P. pastoris), the contig number, the letters g (gene) or e (element), and a systematic number. For example, the first gene on Contig 1 is Pp01g00010. Each identified gene was compared to 8 databases using BlastP (Altschul, et al., J. Mol. Biol., 1990, 215: 403-410). The databases were: Aspergillus niger proteins (Pel at al., Nat. Biotechnol., 2007, 25: 221-231), Saccharomyces cerevisiae strain S288C proteins (www.yeastgenome.org), Schizosaccharomyces pombe proteins, Candida albicans proteins, Candida glabrata proteins, Homo sapiens proteins, Pichia stipitis proteins, and the complete UniProtkB protein database (www.uniprot.org). A gene microarray was designed on the Agilent platform in 8×15 format using Agilent earray software using these genes as well as an additional 77 genes that were identified from Genbank as being involved in Glycosylation processes. The 77 non-P. pastoris genes are derived from various species from fungi to human and code for proteins that include glycan transferases, sugar-nucleotide transporters, and enzymes involved in sugar metabolism. Probes were designed for all 5424 genes for 3′ biased hybridization protocol to a density of 2-3 probes per gene (4207 genes with 3 probes/transcript and 1217 genes with 2 probes/transcript). This custom-designed Agilent P. pastoris 15 k 3.0 array (8×15K) gene microarray was used for all whole genome gene-chip RNA expression analyses.

### Example 2

### Cultivation of P. Pastoris Wild Type and Glycoengineered Strains Under Bioprocess Conditions for Gene Expression Analysis

P. pastoris wild type strain NRRL-Y11430 and two N-glycan modified or glycoengineered strains, YGLY8316 and YGLY8323, were chosen for comparative analysis of gene expression. Both N-glycan modified strains have been specifically engineered to produce the galactose terminated human N-glycan intermediate as has been previously reported (Hamilton, Science, 2006; Davidson U.S. Pat. No. 7,795,002). The three strains were each cultivated in quadruplicate in 0.5 L Bioreactors (Sixfors multifermentation system; ATR Biotech, Laurel, Md.) using a standard glycerol-to-methanol fed-batch protocol as described in Barnard at al., 2010 (J. Ind. Microbiol. Biotechnol. 37:961-971). Samples were taken from each bioreactor at the following timepoints:

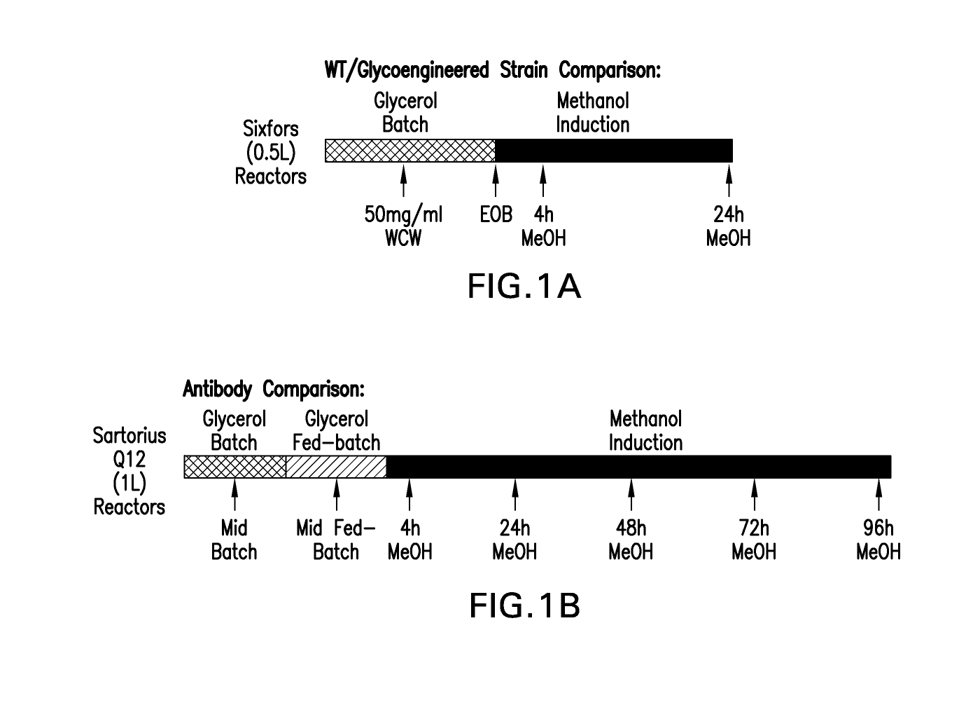

1) during the middle of glycerol batch at 50 mg/ml of wet cell weight (batch),
2) during the starvation period after glycerol exhaustion (End of Batch) as measured by an increase in dissolved oxygen (DO),
3) 4+/−1 hours into methanol-induction, and
4) 24+/−1 hours into methanol-induction (FIG. 1A).

At each timepoint, wet cell weight was measured to determine the amount of cells to harvest and then 1×10⁷ (+/−2×) cells were harvested into 2 ml screwcap microcentrifuge tubes, centrifuged briefly at 5000×g, supernatant was discarded, and the cell pellets were flash frozen using dry ice ethanol. The cell pellets were then used for RNA extraction and microarray hybridization (discussed below). This study is referred to herein as “the wild type/glycoengineered strain comparison study.”

### Example 3

### Cultivation of P. Pastoris Glycoengineered Strains Expressing Secreted Monoclonal Antibodies Under Bioprocess Conditions for Gene Expression Analysis

A P. pastoris glycoengineered strain, YGLY8316, and four highly related glycoengineered strains expressing the monoclonal antibodies MK-HER2 strain A (YGLY12501), MK-HER2 Strain B (YGLY13992), MK-RSV (YGLY14401), and MK-VEGF (YGLY10360) were cultivated in triplicate in Sartorius Q12 1 L bioreactors (Sartorius, Goettingen, Germany) using a standard fed-batch fermentation protocol as described in Barnard at al., 2010 (J. Ind. Microbiol. Biotechnol. 37:961-971). Samples were taken from each bioreactor at the following timepoints: 1) during the middle of glycerol batch at 50 mg/ml of wet cell weight (batch), 2) during the middle of glycerol fed-batch (4+/−1 hours into fed-batch), 3) 4+/−1 hours into methanol induction, 4) 24+/−1 hours into methanol induction, 5) 48+/−1 hours into methanol induction, 6) 72+/−1 hours into methanol induction, 7) 96+/−1 hours into methanol induction (FIG. 1B). At each timepoint, wet cell weight was measured to determine the amount of cells to harvest and then 1×10⁷ (+/−2×) cells were harvested into 2 ml screwcap microcentrifuge tubes, centrifuged briefly at 5000×g, supernatant discarded, and the cell pellets flash frozen using dry ice ethanol. The cell pellets were then used for RNA extraction and microarray hybridization (below). This study is referred to herein as “the mAb comparison study.”

### Example 4

### Gene Expression Analysis Using Agilent P. Pastoris-Specific Microarrays

Following sample collection, samples were processed. Briefly, total RNA was extracted and scrutinized for quality and yield; mRNA was amplified using Ambion MessageAmp II reagents and protocols and then hybridized to a custom-designed Agilent Pichia pastoris 15 k 3.0 array (8×15K) based upon an internal Pichia pastoris genome sequence for strain NRRL Y-11430; subsequent scanning was performed using Agilent Microarray scanners (version B), and output raw image files in .tif format were processed by Agilent Feature Extractor (FE) software. Microarray quality control data were generated from the FE output data and were reviewed for data quality.

Standard Resolver pipelines for the Agilent Single Color Error Model (Weng et al., Bioinformatics 22, 2006, 1111-1121) were used for data summarization and calling using the following parameters: FRACTION=0.12, POISSON=3, and RANDOM=0.05. Briefly, the data was median normalized, and then a background gradient was calculated and subtracted from the normalized data. Next, intensity and ratio error models were constructed which combined replicate measurements and modeled associated error. These models determined whether a particular gene exhibited differential expression for the ratio comparison specified, although such differential expression calls were typically made via ANOVA and t-test statistical tests that were also performed. In addition to these statistical tests, clustering, PCA, and other operations were also performed upon the data using Resolver software, typically utilizing data ratioed to the pool of all other samples within a specific study unless otherwise indicated. In order to determine promoters with desired characteristics (e.g., little gene expression upon glycerol growth but up-regulation upon methanol growth), the Trend tool was utilized to match the 100 closest matching gene expression profiles by distance as described in the Resolver User's Manual and online help sections (Rosetta Resolver User Guide, 2002, Kirkland, Wash.).

### Example 5

### Identification of Strong Methanol-Inducible Promoters by Microarray Gene Expression Analysis

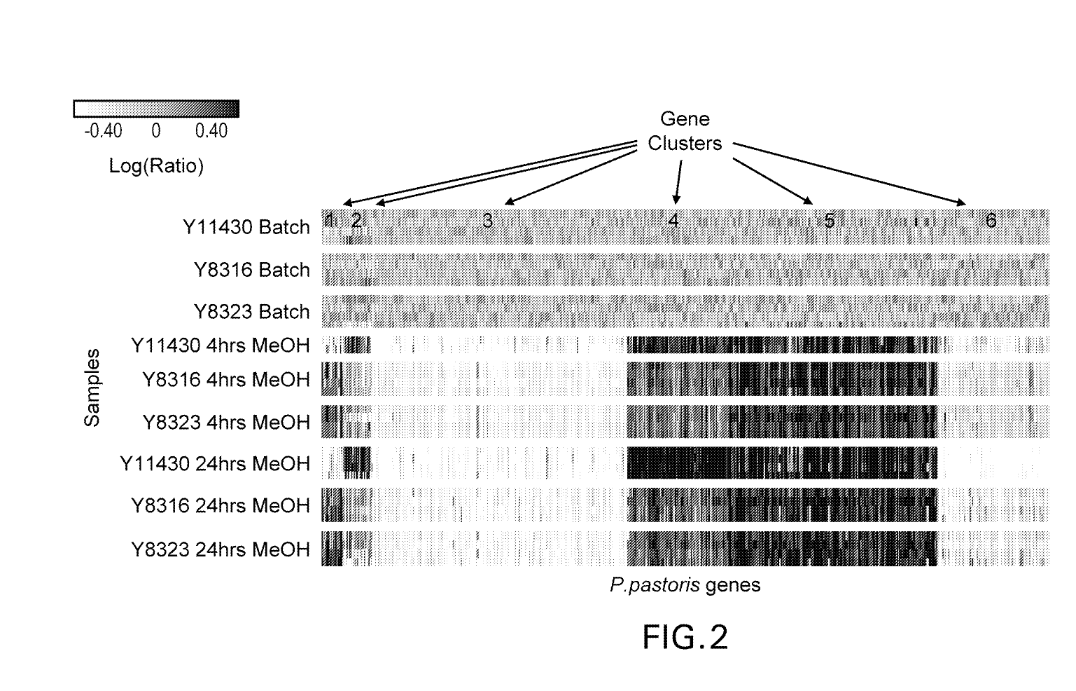

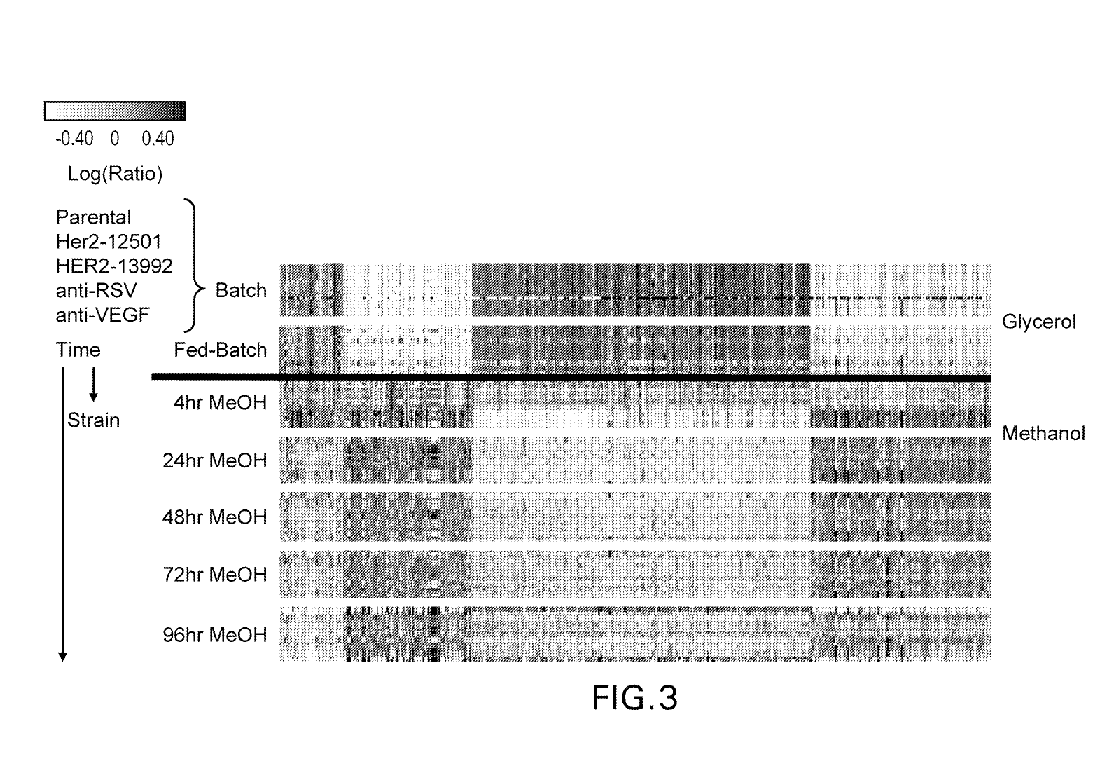

To identify methanol-inducible promoters, gene expression data intensity profiles from the wild type/glycoengineered strains study were analyzed by first ratioing strain-specific, individual sample data to the Batch (50 mg/ml of wet cell weight; glycerol) timepoint. Three individual ANOVA analyses were then performed using 3 factors (Batch, 4 hour MeOH, and 24 hour MeOH), one for each of the strains with individual replicates with a cutoff of P<=0.005. These genes were then clustered by K-means with 6 clusters using a 2 fold change cutoff in at least 4 samples, resulting in a total of 2,882 sequences (FIG. 2). This analysis reveals genes are differentially regulated between strains (FIG. 2, clusters 1 and 2) and genes that are similarly regulated between the wild type and engineered strains but to differing extents (FIG. 2, clusters 4 and 6). But the vast majority of the signature includes genes that are regulated similarly and to relatively similar extents between the wild type and engineered strains (FIG. 2, clusters 3 and 5). A similar analysis was repeated for the mAb expression comparison study data (FIG. 3), performing a two factor ANOVA to observe changes between glycerol and methanol. Here 929 sequences are identified that change between glycerol and methanol in the intersection between the 5 strains p<=0.01. These genes are shown clustered by K-means with 5 clusters using a 1.25 fold change cut-off in at least 3 samples (FIG. 3). Here again, >400 genes are significantly induced in all 5 strains by switching from glycerol to methanol. Therefore, to identify the most interesting methanol-inducible genes, the data were analyzed using intensity values as a factor.

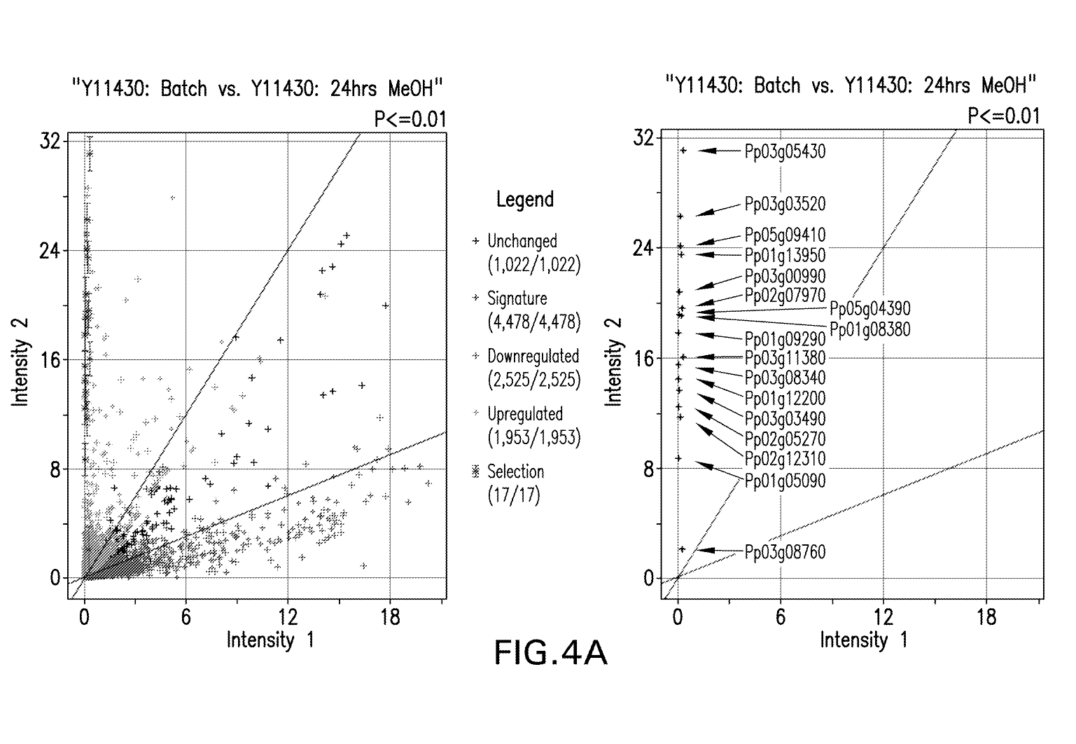

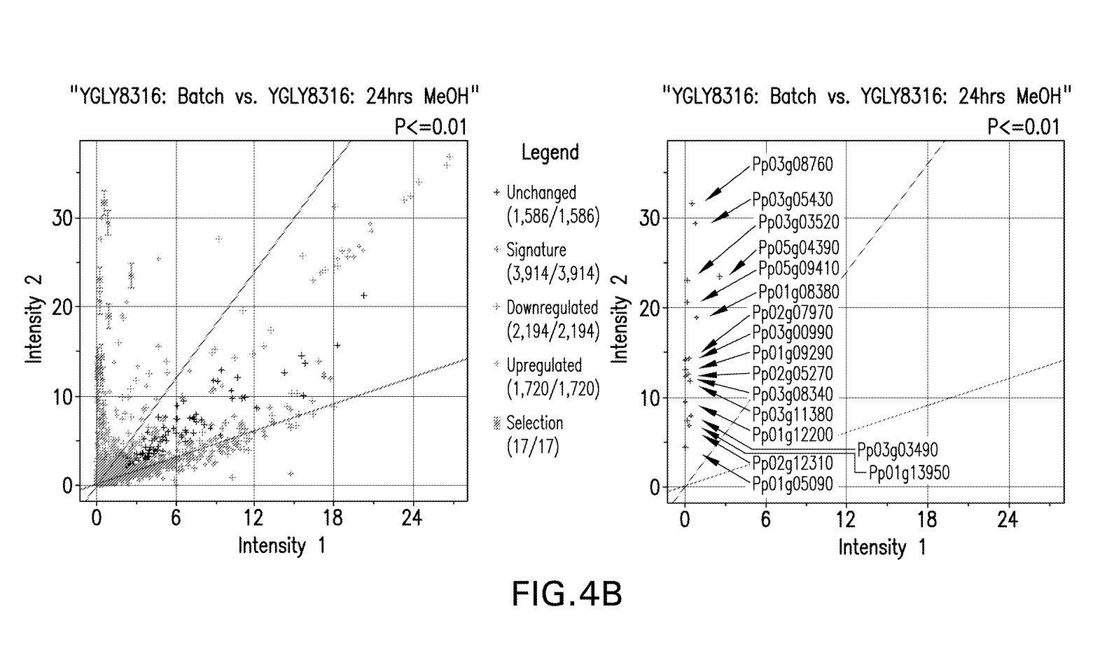

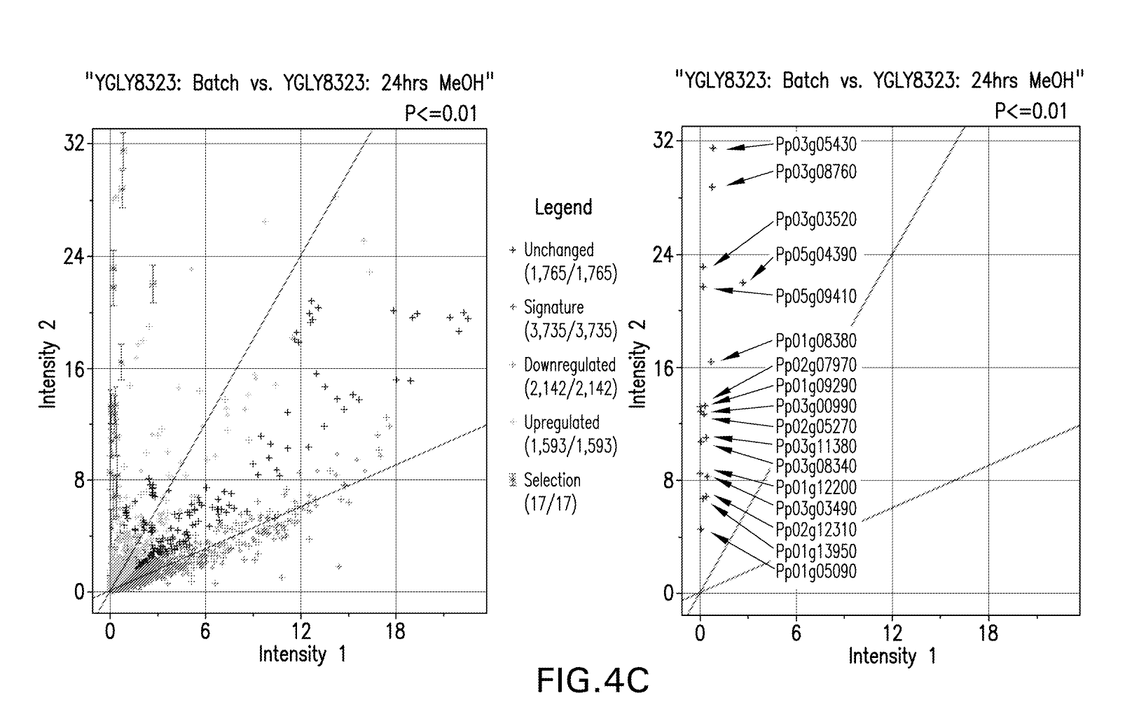

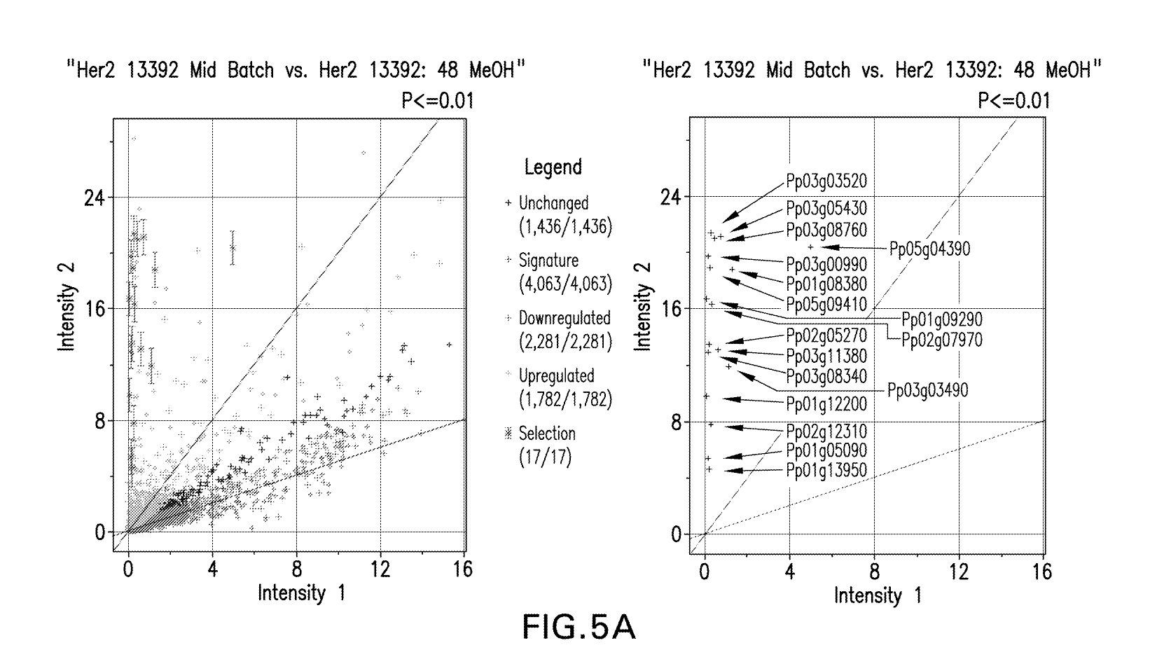

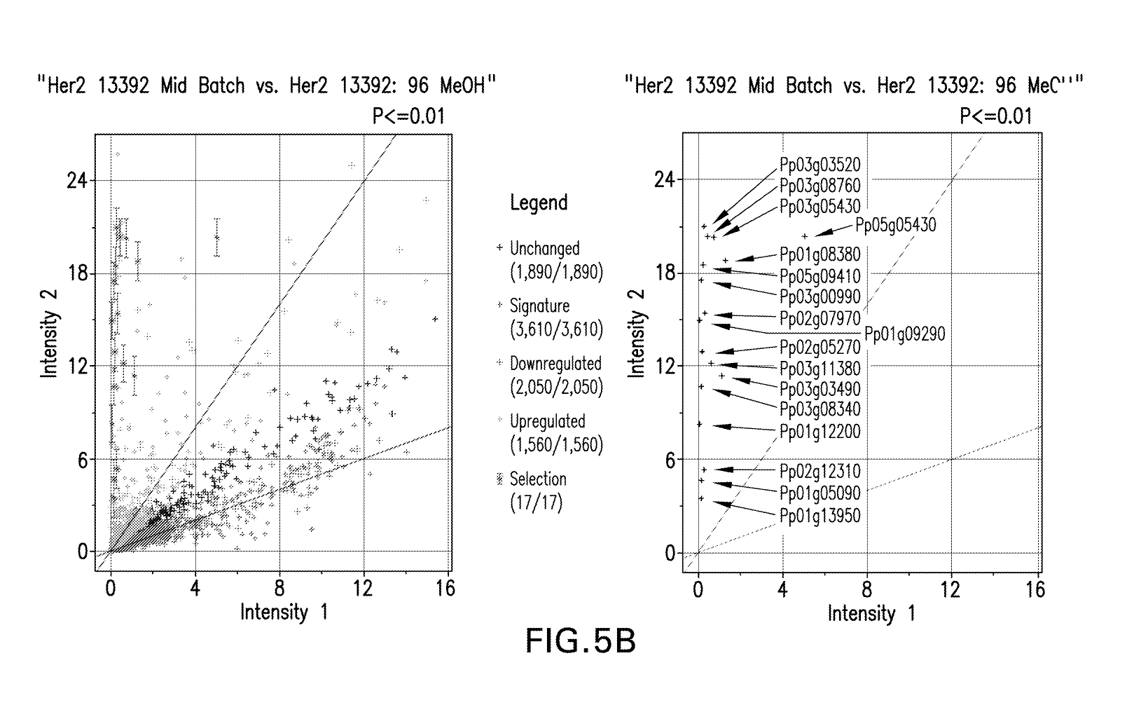

Samples were organized as combined replicates and again referenced strain-specific to the Batch (50 mg/ml of wet cell weight; glycerol) timepoint. Each replicate combined sample for the wild type (y11430) and the glycoengineered strains (YGLY8316 and YGLY8323) was then analyzed individually as an intensity plot comparing the glycerol (Batch) with methanol (24 hrs MeOH) timepoints (FIG. 4A-40). The intersection of those genes with the highest intensity 2 (methanol) but lowest intensity 1 (glycerol) was analyzed by individually comparing intensity profiles at each timepoint as plotted (FIG. 4A-4C). This analysis was repeated using the strains from the mAb comparison experiment with two of the dotplots from strain YGLY13992 (anti-HER2) comparing glycerol (batch) with 48 hour and 96 hour methanol (48 MeOH and 96 MeOH) induction samples shown (FIGS. 5A and 5B). Collectively from these data, 17 genes were identified as having desirable strong methanol-inducible expression that failed to be identified in previous studies that led to previously known AOX1, AOX2, DAS1, FLD1 and several PEX family gene promoters, e.g., PEX5, PEX8, and PEX14 (Ellis, Mol. Cell. Biol., 1985, 5: 1111-1121; Kobayashi, 3. Biosci. & Bioeng., 2000, 89:479-484; Tschopp, Nuc. Acids. Res., 1987, 15; 3859-3876; Resina, J. Biotech., 2004, 109: 103-113; Menendez, Yeast, 2003, 20: 1097-1108; Lin-Cereghino, Mol. Cell. Biol., 2006, 26: 883-897). The methanol-inducible genes identified included (with common gene name or highest homolog in parentheses):

Pp01g09290 (ScFBA1 (one of two identified in P. pastoris, the other FBA1 homolog is not induced by methanol), SEQ ID NO: 30),
Pp03g03520 (DAS2, a second homolog of PpDAS1, SEQ ID NO: 31),
Pp03g08760 (ScCWP1, SEQ ID NO: 32),
Pp03g00990 (Homologous to ScYGR201c, SEQ ID NO: 33),
Pp02g05270 (Homologous to Aspergillus niger AN2948.2, SEQ ID NO: 34),
Pp02g12310 (ScDUR3, SEQ ID NO: 35),
Pp03g05430 (ScTHI4, SEQ ID NO: 36),
Pp03g03490 (homologous to A. niger AN2957.2, SEQ ID NO: 37),
Pp05g09410 (THI13, SEQ ID NO: 38),
Pp02g07970 (ScPEX11, SEQ ID NO: 39),
Pp01g12200 (Homologous to A. niger AN7917.2, SEQ ID NO: 40),
Pp03g11380 (ScPMP47, SEQ ID NO: 41),
Pp03g08340 (unknown, SEQ ID NO: 42),
Pp05g04390 (ScTIR3, SEQ ID NO: 43),
Pp01g08380 (ScYIL057C, SEQ ID NO: 44),
Pp03g11380 (ScPMP47, SEQ ID NO: 45), and
Pp01g13950 (ScTPN1, SEQ ID NO: 46).

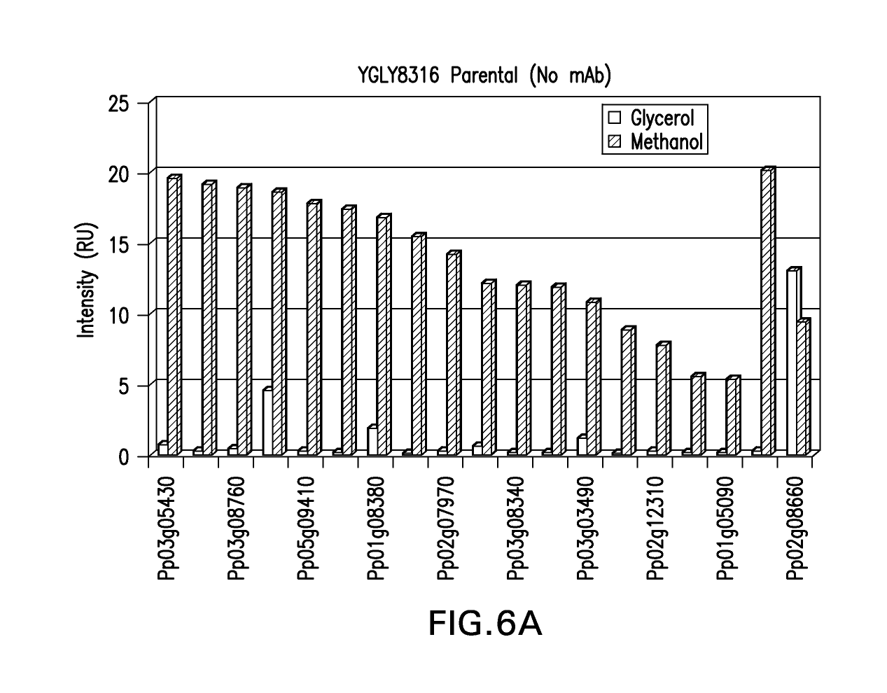

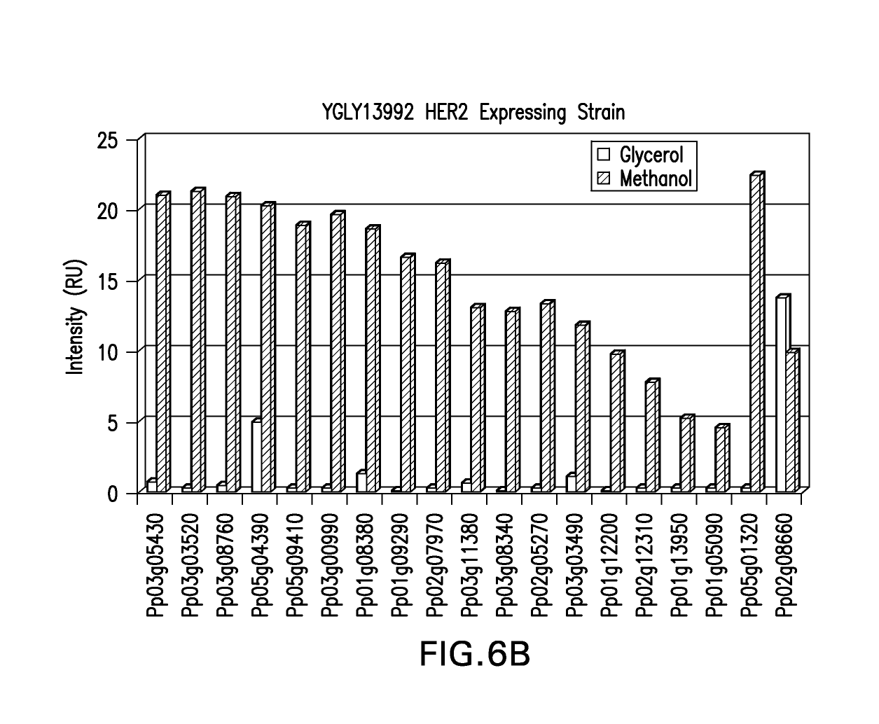

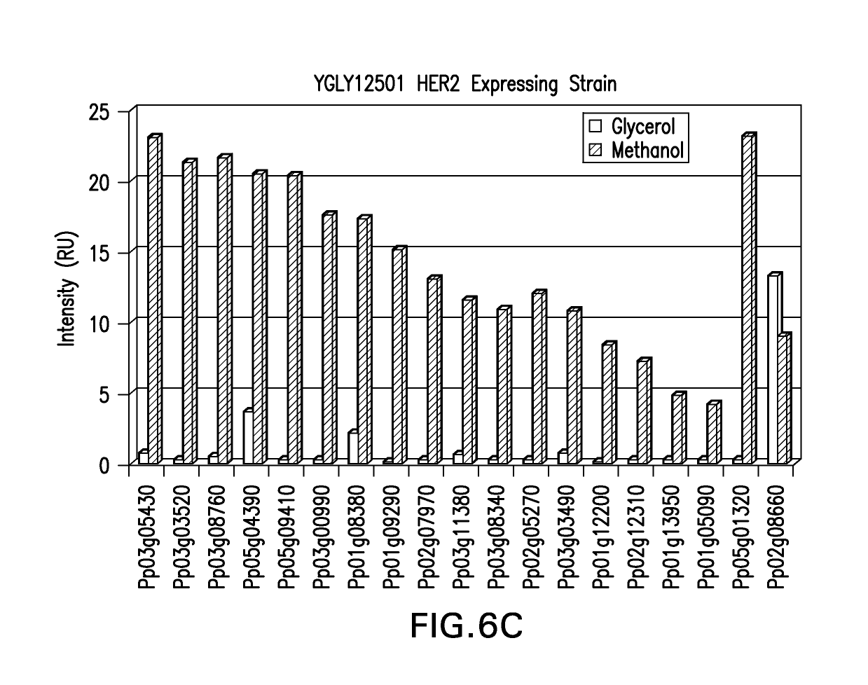

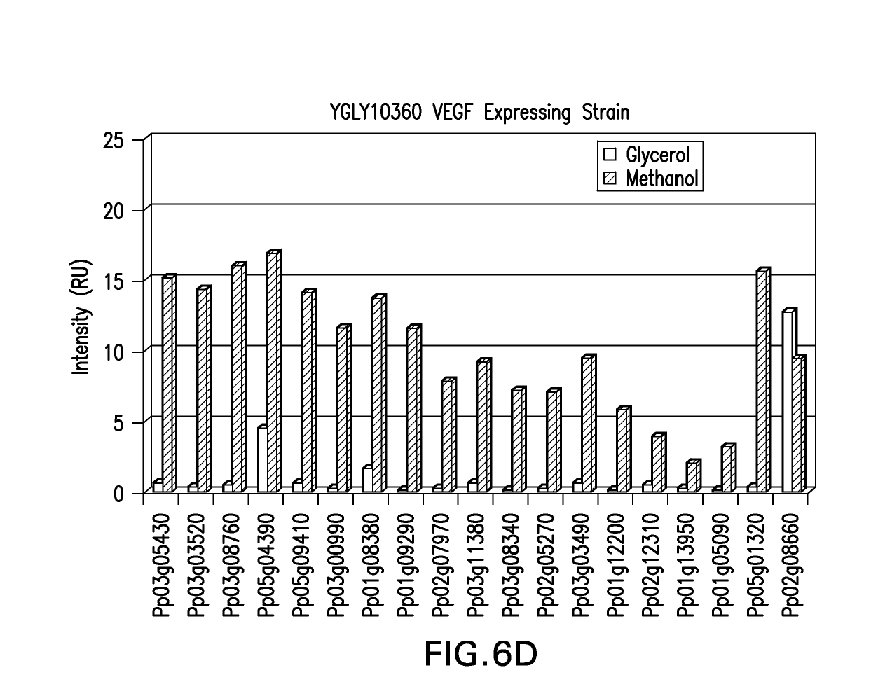

The intensity data for these genes was plotted in comparison to AOX1 (Pp05g01320) and GAPDH (Pp02g08660) as controls (FIG. 6).

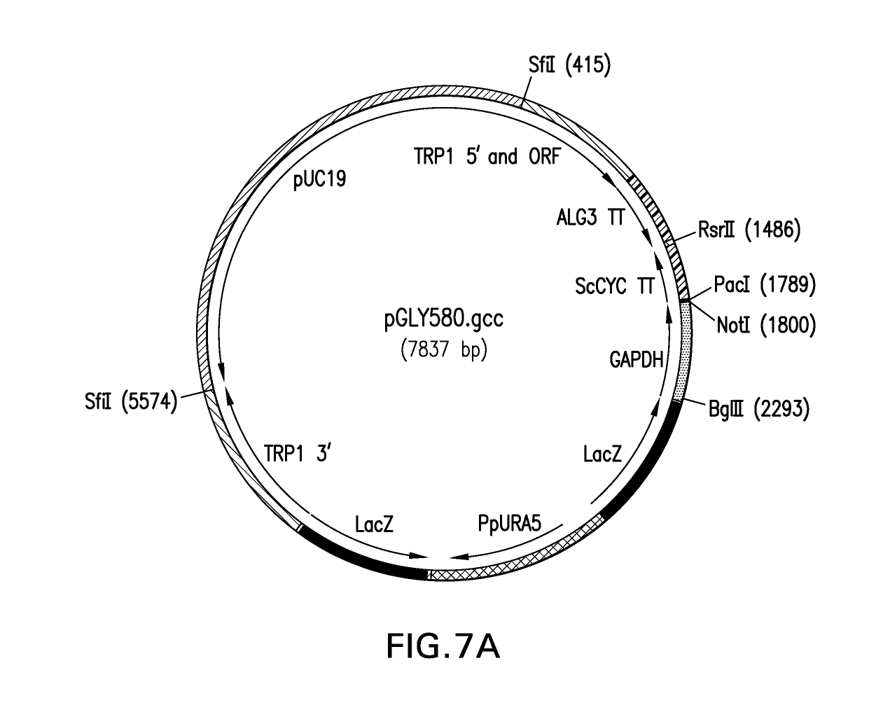

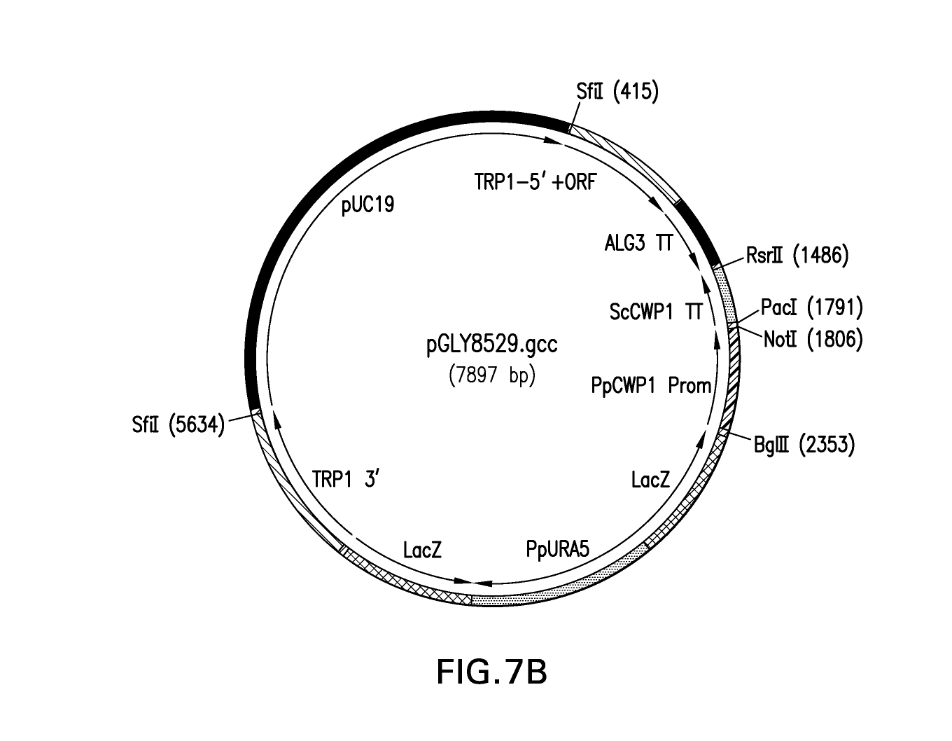

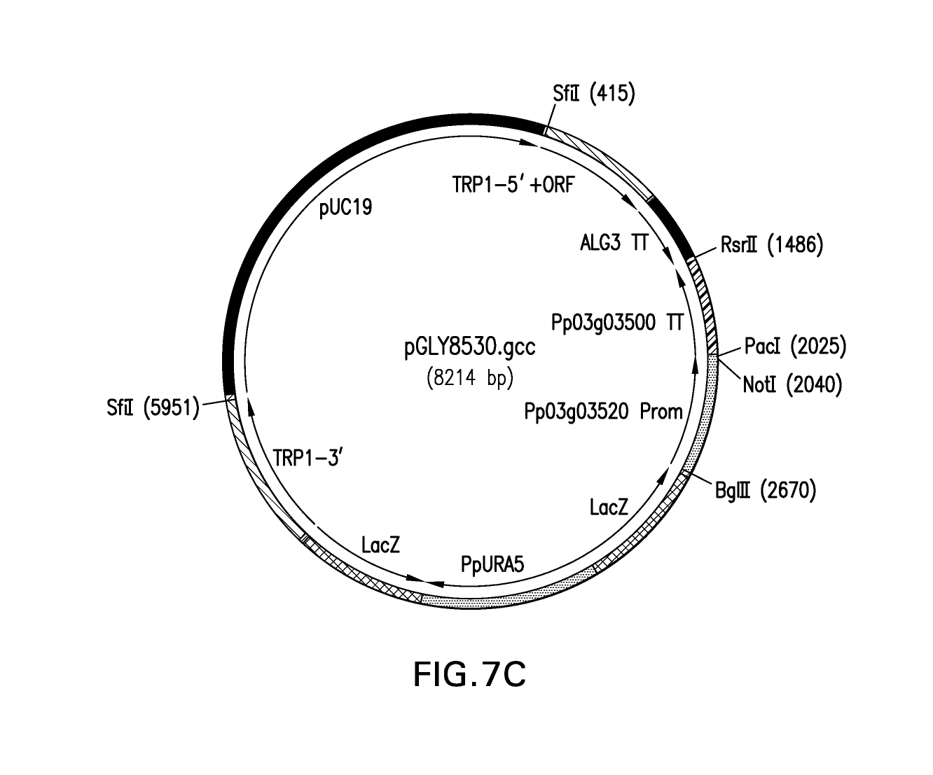

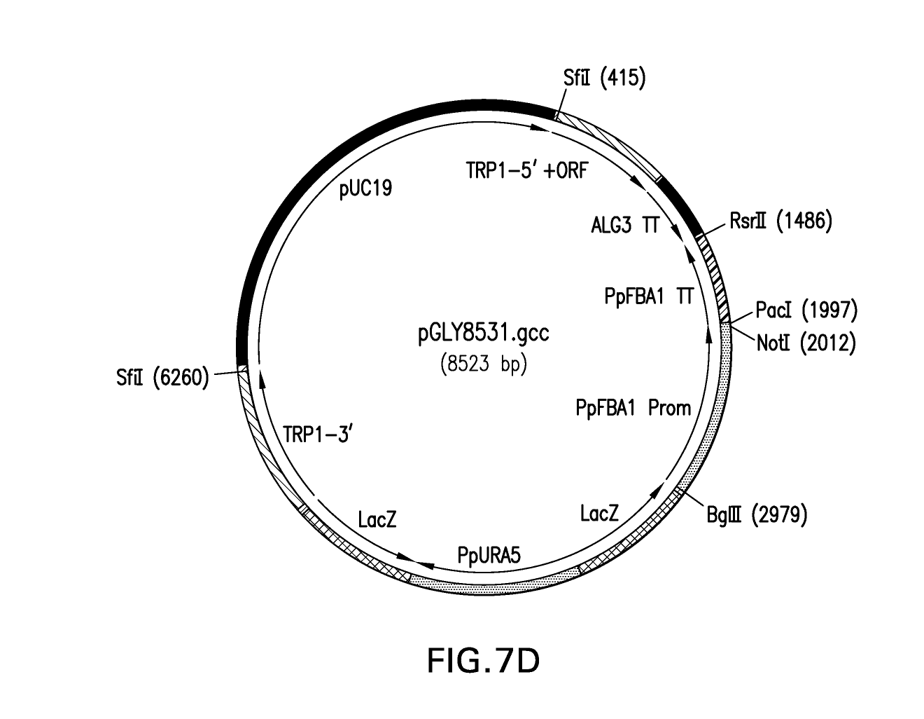

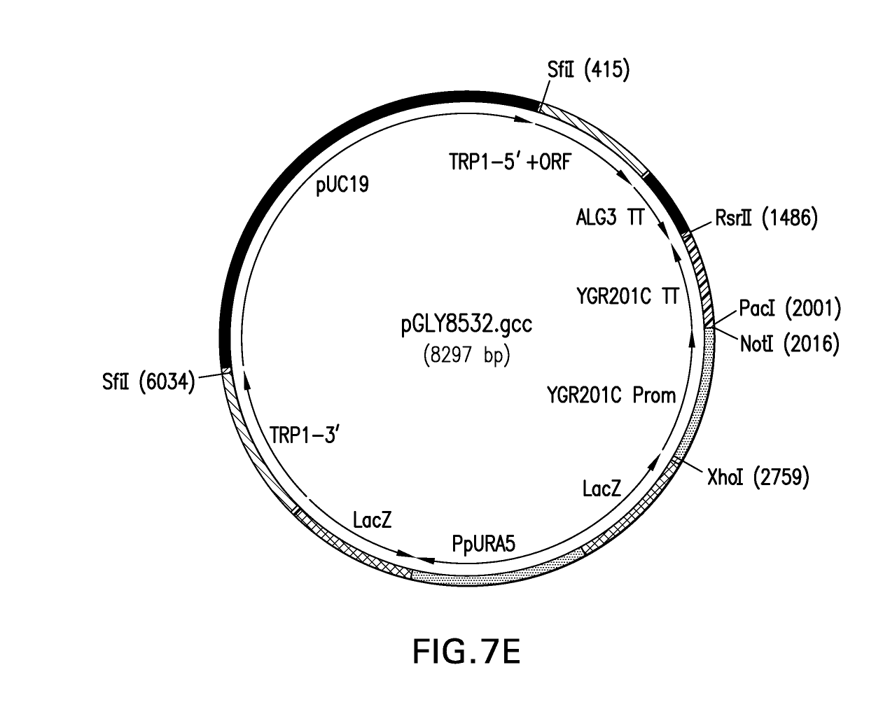

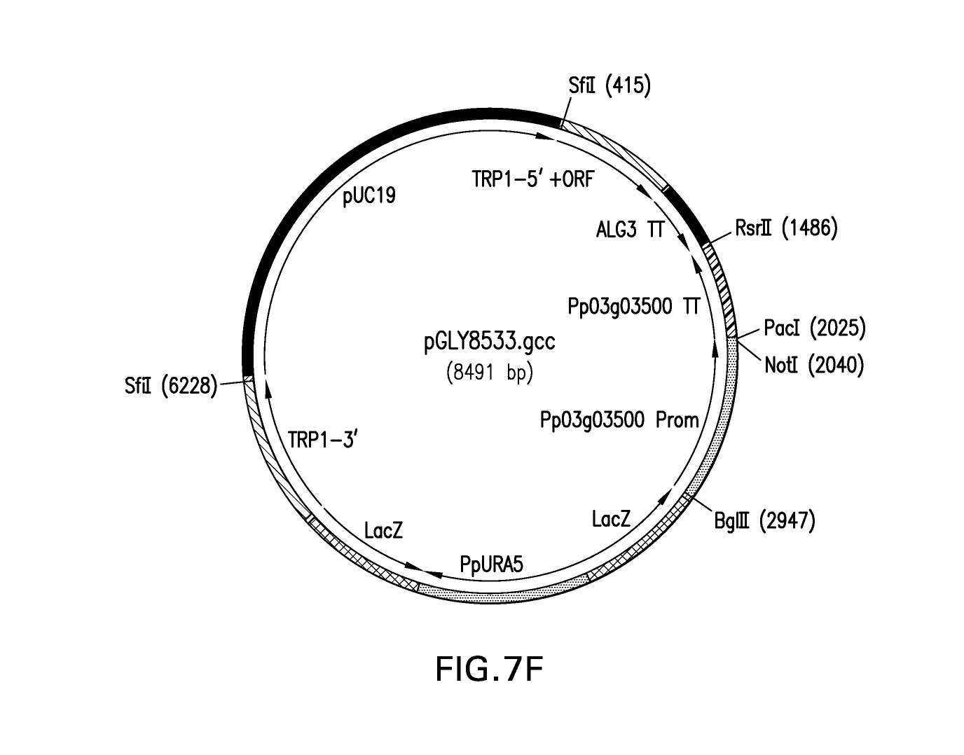

The extracted promoters of these 17 genes are contained herein as SEQ ID NOs: 47 through 63, respectively. The promoters and transcriptional terminators for several exemplary genes of this group were then in vitro synthesized (GeneArt, AG, Regensberg, Germany) as the 5′-proximal 1000 bp of genomic sequence to the ATG of each respective gene and the 500 bp of genomic sequence 3′ proximal to the stop codon of each respective gene. The promoters/terminators for these genes, Pp03g08760 (ScCWP1), Pp03g03520 (DAS2), Pp01g09290 (ScFBA1) and Pp03g00990 (ScYGR201C), as well as Pp03g03500 (DAS1) as a control, were subcloned into the AOX1 containing P. pastoris integration vector pGLY580 at the BglII/RsrII sites to generate plasmids pGLY8529-8533, respectively (These plasmids as well as pGLY580 are depicted in FIGS. 7A-7F).

### Example 6

### Identification of Strong Constitutive Promoters by Microarray Gene Expression Analysis

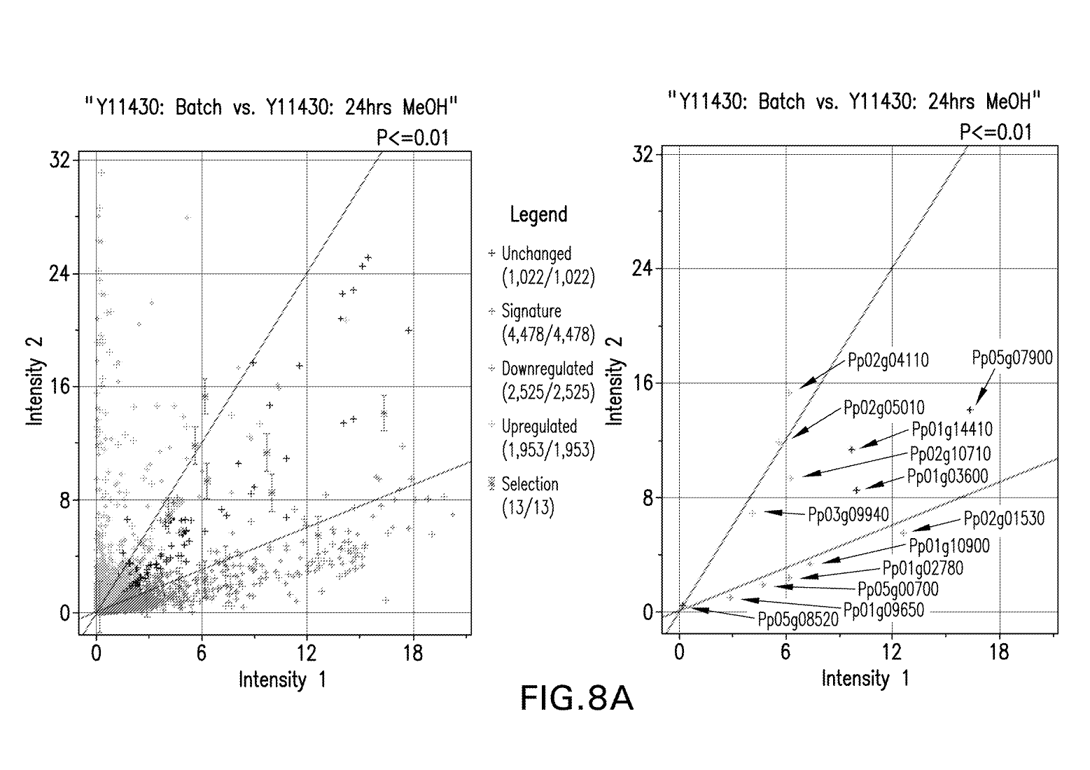

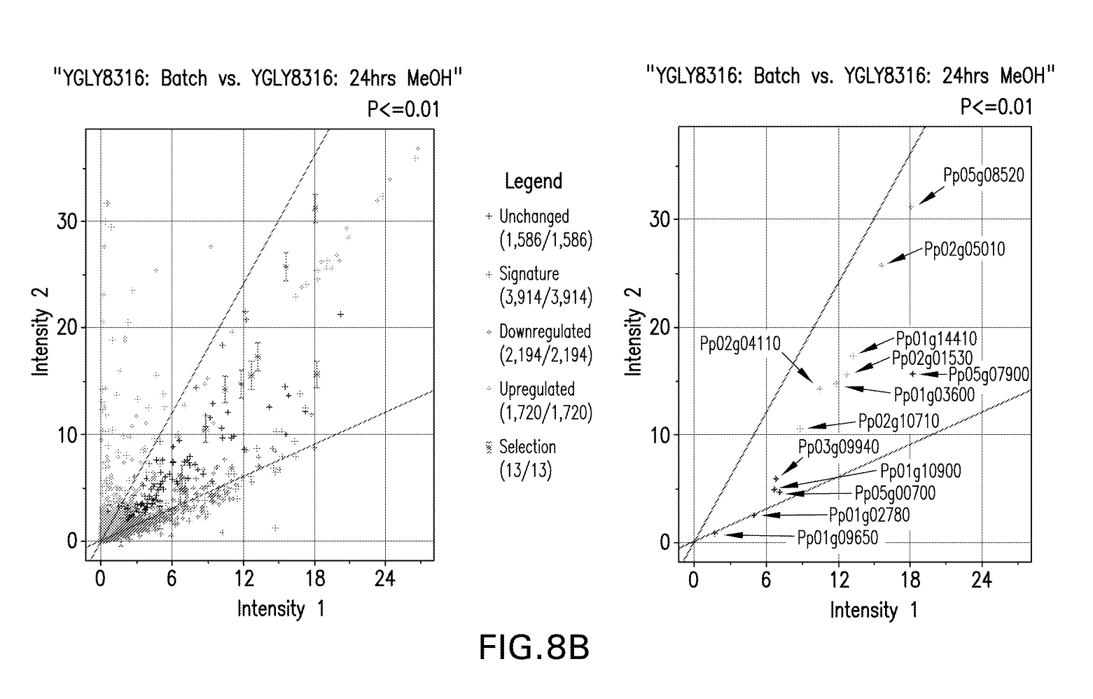

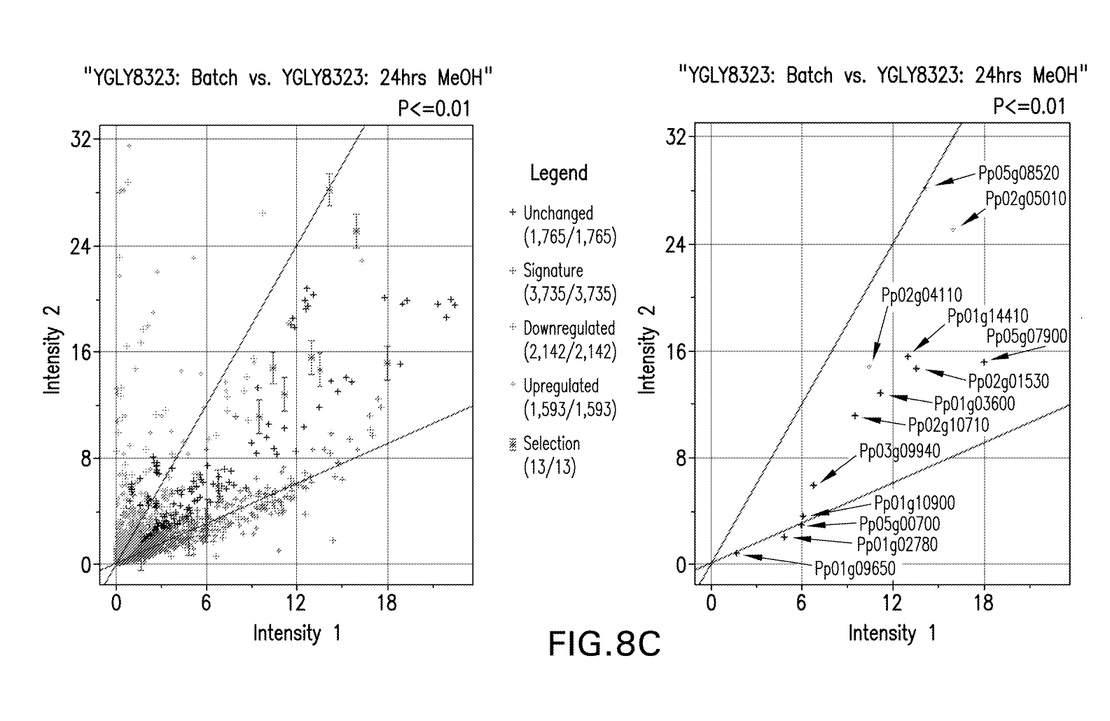

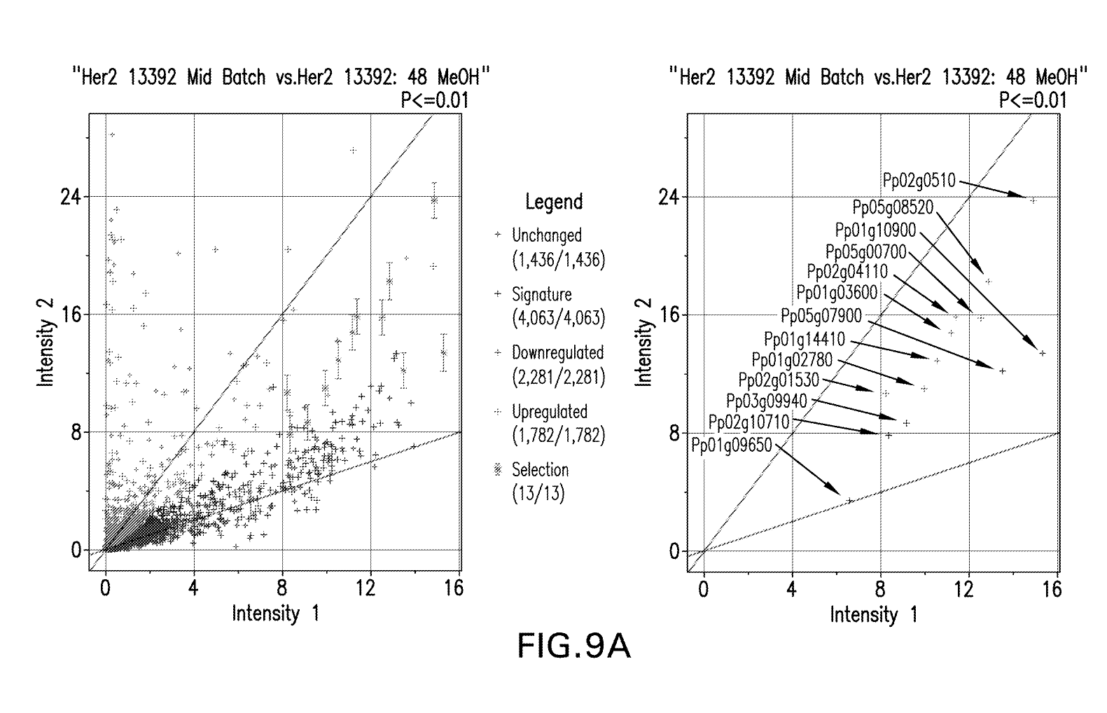

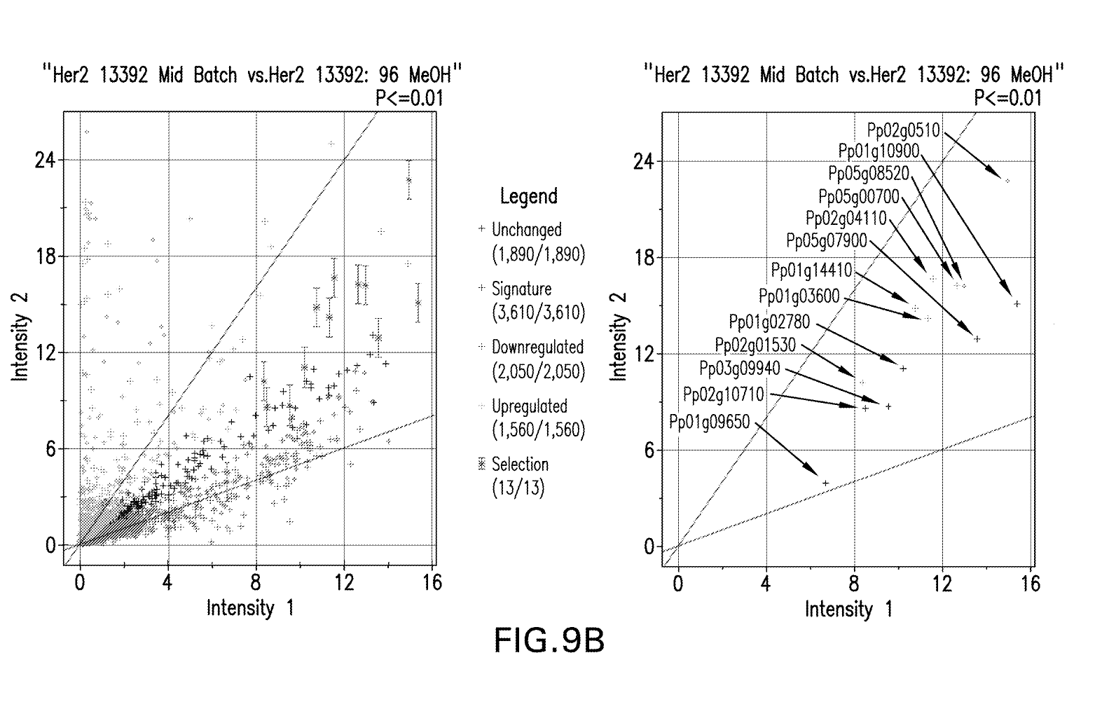

Gene expression data intensity profiles from the wild type/glycoengineered strains study with data ratioed to the Batch timepoint were analyzed to identify constitutive promoters. In particular, those genes were identified which maintain high intensity in the 4 hour of methanol induction vs. Batch and 24 hour of methanol induction vs. Batch samples. The intersection of the highest intensity genes was analyzed by individually comparing intensity profiles at each timepoint as plotted in dotplots for the glycerol (batch) versus methanol (24 hours MeOH) timepoints (FIG. 8A-C). The same analysis was repeated for the antibody expression comparison study data with exemplary data shown for the YGLY13992 (anti-HER2 expressing) strain comparing glycerol (batch) with 48 h and 96 h methanol (48 MeOH and 96 MeOH) timepionts (FIGS. 9A&B). From these data, 13 genes were identified as having desirable strong constitutive expression, in addition to the previously described GPD, TEF, and PMA1 genes. These genes are (predicted homolog identification in parentheses):

Pp02g05010 (ScPIR1, SEQ ID NO: 1),
Pp01g10900 (ScCHT2, SEQ ID NO: 2),
Pp05g07900 (ScAAC2/PET9, SEQ ID NO: 3),
Pp05g08520 (ScCCW12, SEQ ID NO: 4),
Pp02g01530 (ScPST1, SEQ ID NO: 5),
Pp05g00700 (unknown, SEQ ID NO: 6),
Pp02g04110 (ScPOR1, SEQ ID NO: 7),
Pp01g03600 (ScBGL2, SEQ ID NO: 8),
Pp01g14410 (ScACO1, SEQ ID NO: 9),
Pp01g09650 (ScYHR021C, SEQ ID NO: 10),
Pp01g02780 (ScYLR388W, SEQ ID NO: 11),
Pp03g09940 (ScPIL1, SEQ ID NO: 12),
Pp02g10710 (ScMDH1, SEQ ID NO: 13), and
Pp03g12300 (unknown).

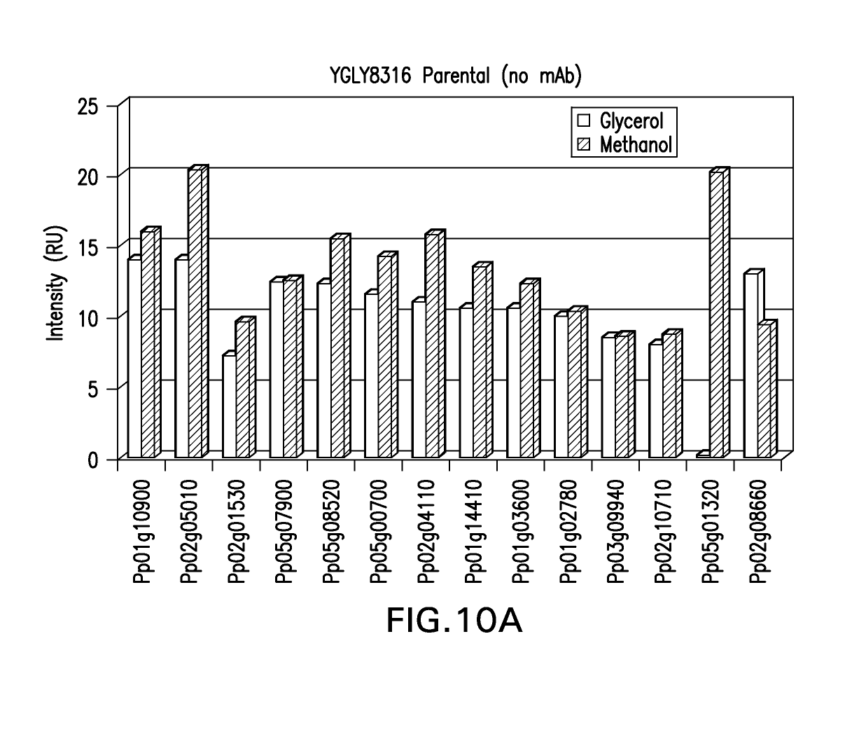

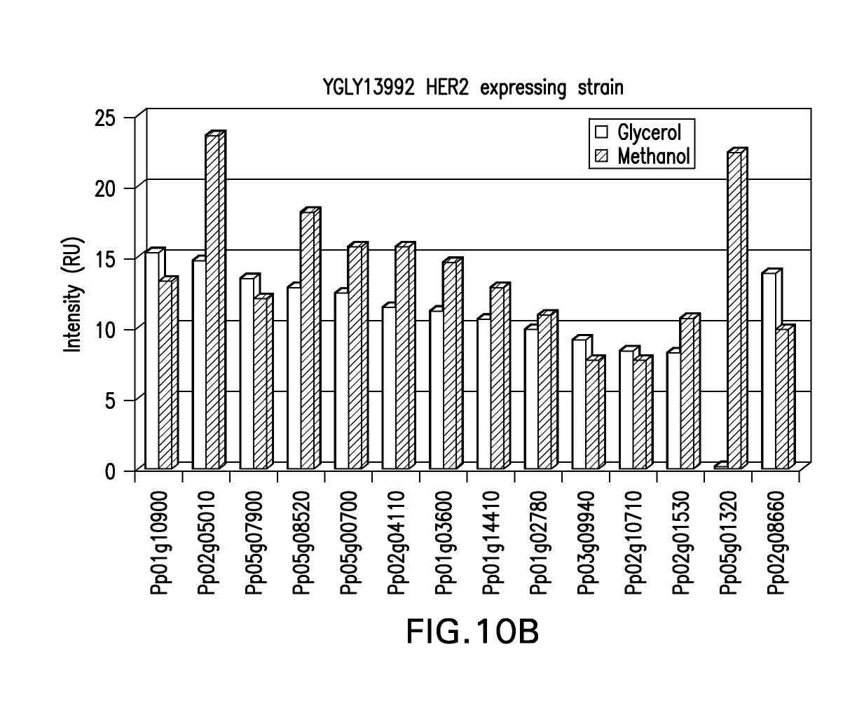

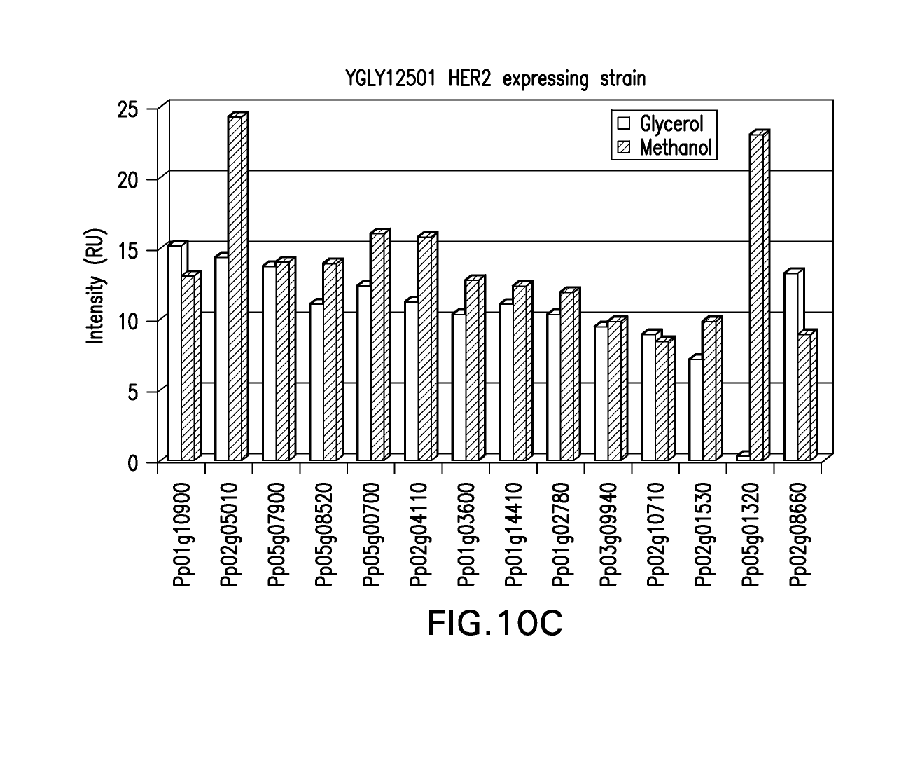

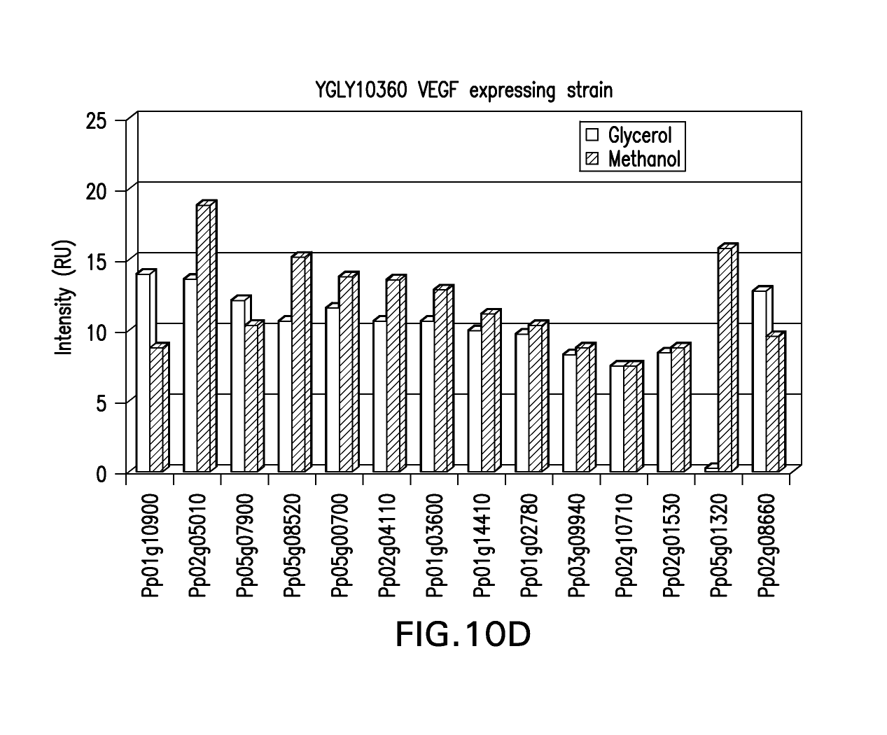

Surprisingly, despite the fact that the canonically constitutive housekeeping GPD gene shows significant regulation, along with nearly ⅔ of the genome, a number of these genes could be identified as having truly constitutive expression under these diverse carbon source conditions. The intensity data for the identified genes is plotted in comparison to AOX1 and GAPDH (FIG. 10).

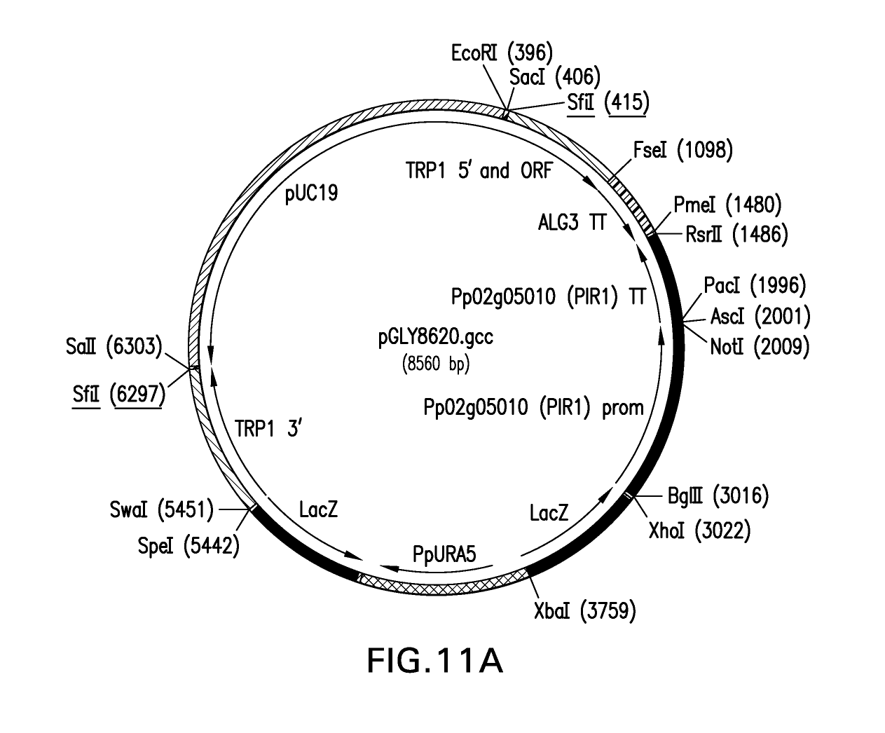

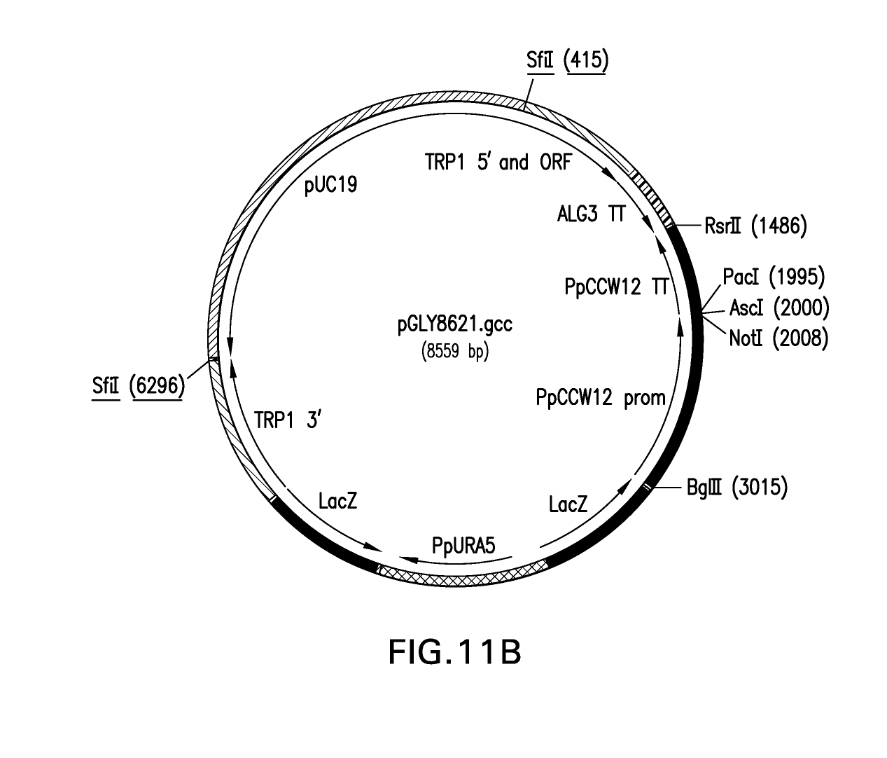

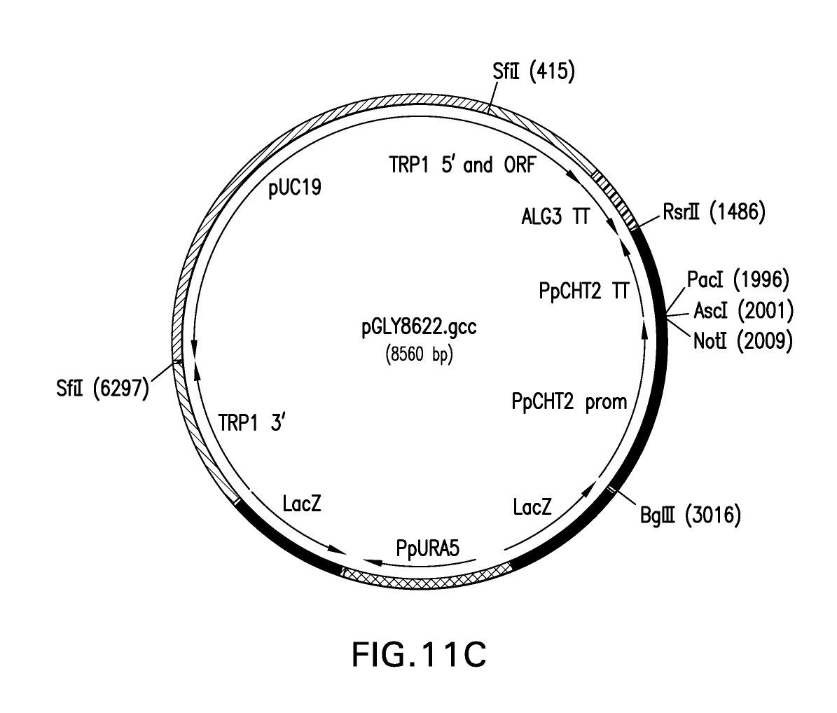

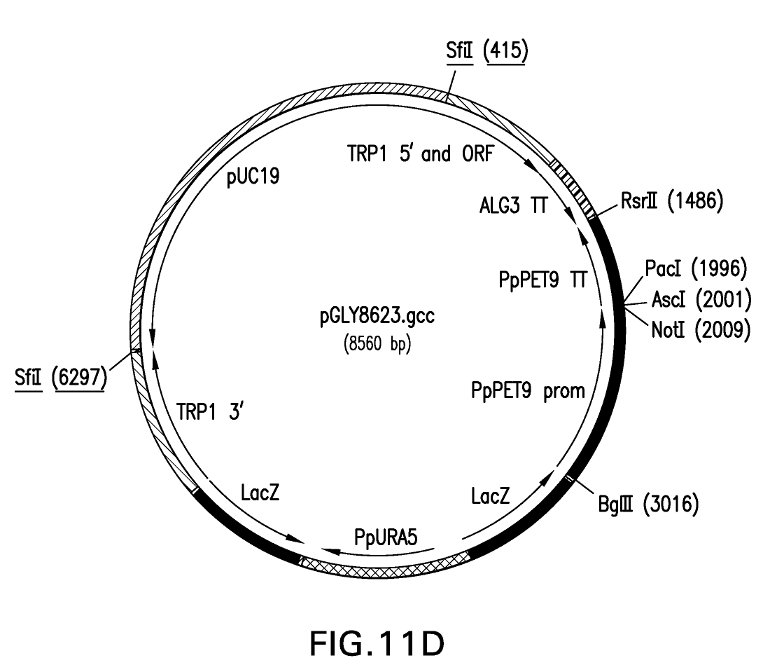

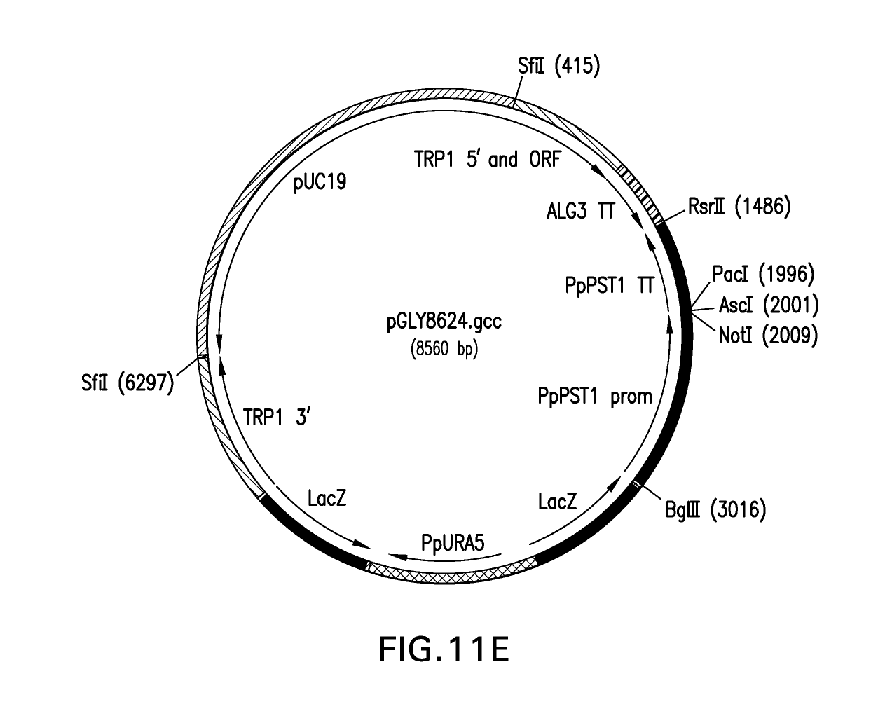

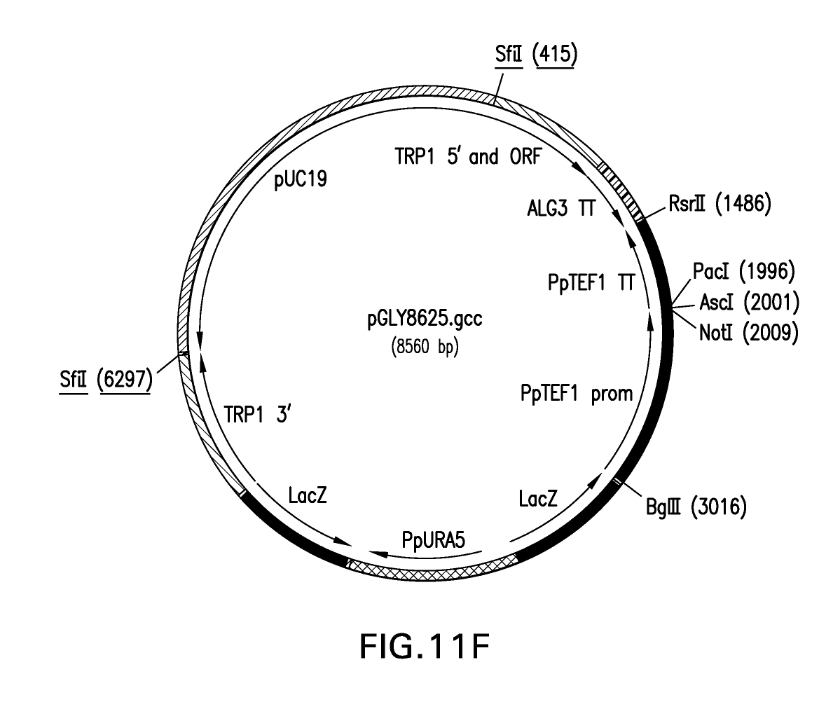

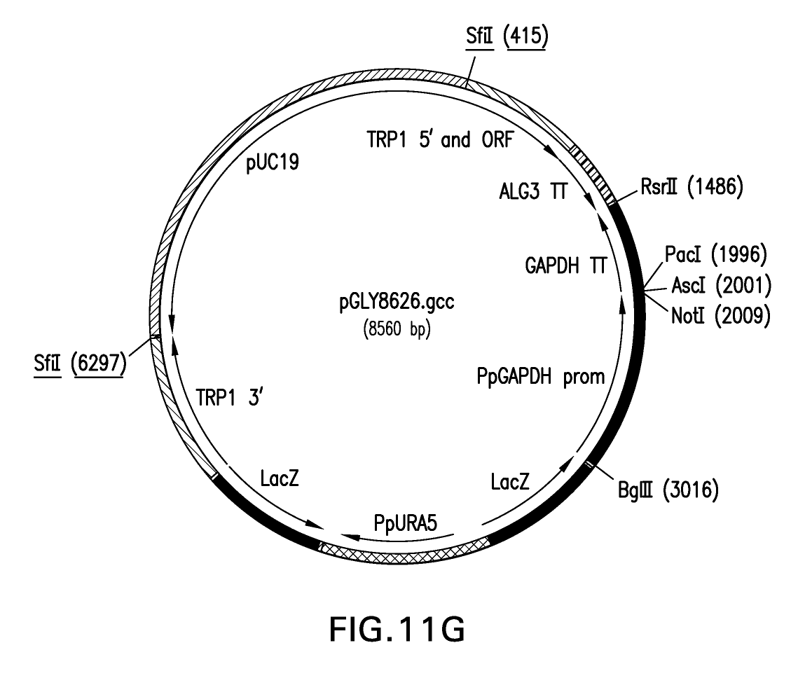

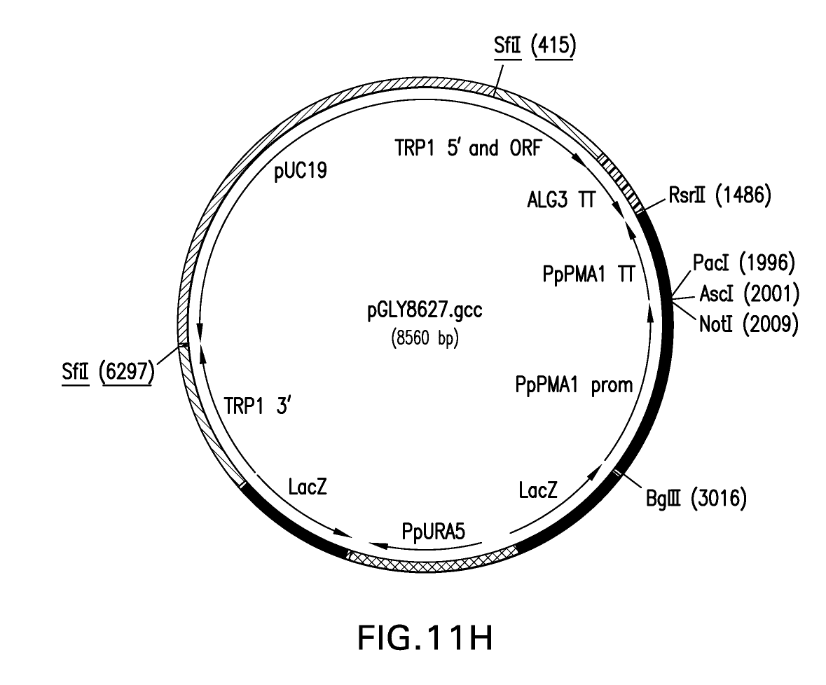

The gene regulatory regions (promoter/5′ untranslated region or UTR and transcriptional terminator/3′ UTR) for each of these genes was further identified by extracting the 1000 bp upstream of the start (ATG) codon and 500 bp downstream of the stop codon. These sequences were extracted and paired together as regulatory cassettes flanked around the sequences for recognition by restriction endonucleases NotI (GCGGCCGC), AscI (GGCGCGCC), and PacI (TTAATTAA) indicated in bold in the sequences and these regulatory cassettes are identified as SEQ ID NOs: 14-26. The sequence cassettes were then physically synthesized and cloned (GeneArt, AG, Regensberg, Germany) to be used as expression cassettes. As controls, the Pp TEF (Pp01g00550), PpGPD or GAPDH (Pp02g08660), PpPMA1 (Pp01g12610) gene regulatory elements were similarly generated and these cassette sequences are herein identified as SEQ ID NOs: 27-29, respectively. The cassettes for CCW12, CHT2, PET9, PST1, TEF, GPD, and PMA1 were then subcloned into a plasmid containing the P. pastoris URA5 gene and TRP1 integration sequences using the flanking BglII/RsrII restriction sites to generate the P. pastoris expression plasmids pGLY8620-8627, respectively (FIGS. 11A-H).

Interestingly, among both the induced and constitutive genes, we found that some genes that differed significantly in their expression from wild type to glycoengineered strains were among the strongest expressed genes by intensity profile. For example CWP1 (Pp03g08760) was strongly induced upon switch to methanol in the glycoengineered strains analyzed in both the wildtype/glycoengineered strain comparison study (YGLY8316 and YGLY8323) as well as in all of the strains in the mAb comparison study (YGLY8316, YGLY13992, YGLY12501, YGLY14401, and YGLY10360) but while methanol-induced, is only modestly expressed under either condition in the wild type strain. Similarly among the constitutive genes, Pp05g08520 (CCW12), Pp02g05010 (PIR1) and Pp05g00700 (unknown) are among the stronger constitutive genes in the engineered strains (YGLY8316, YGLY8323, YGLY13992, YGLY12501, YGLY14401, and YGLY10360), but are only expressed either moderately (PIR1, Pp05g00700) or very weakly (CCW12) in the wild type strains. All of these genes display unexpected high expression levels in the glycoengineered strains and this property allows their promoters to be exploited in the engineered strains as useful regulatory sequences.

### Example 7

### Identification of Methanol Repressible Promoters by Microarray Gene Expression Analysis

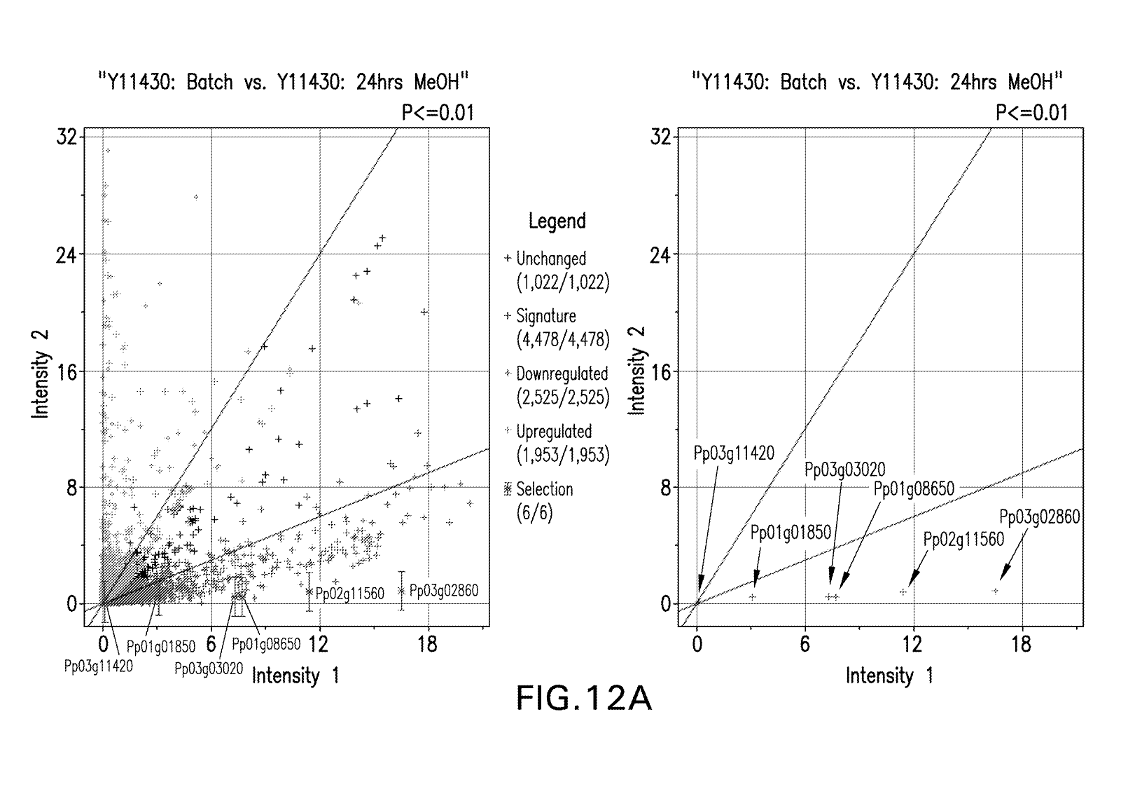

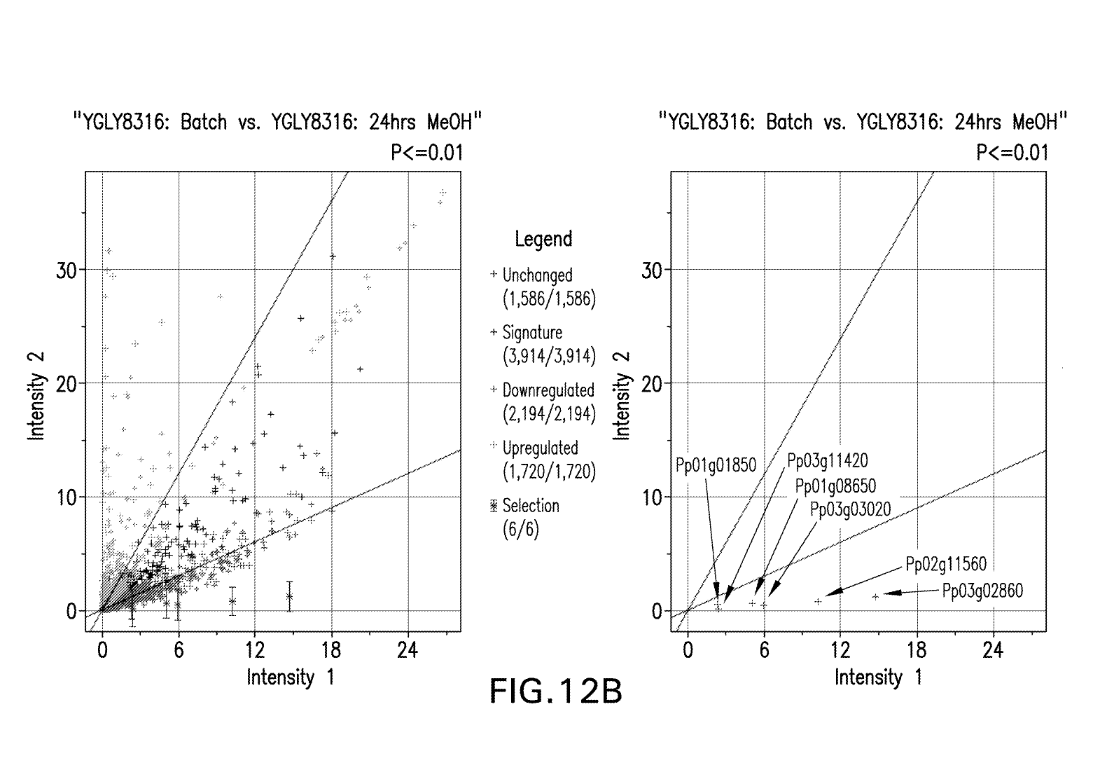

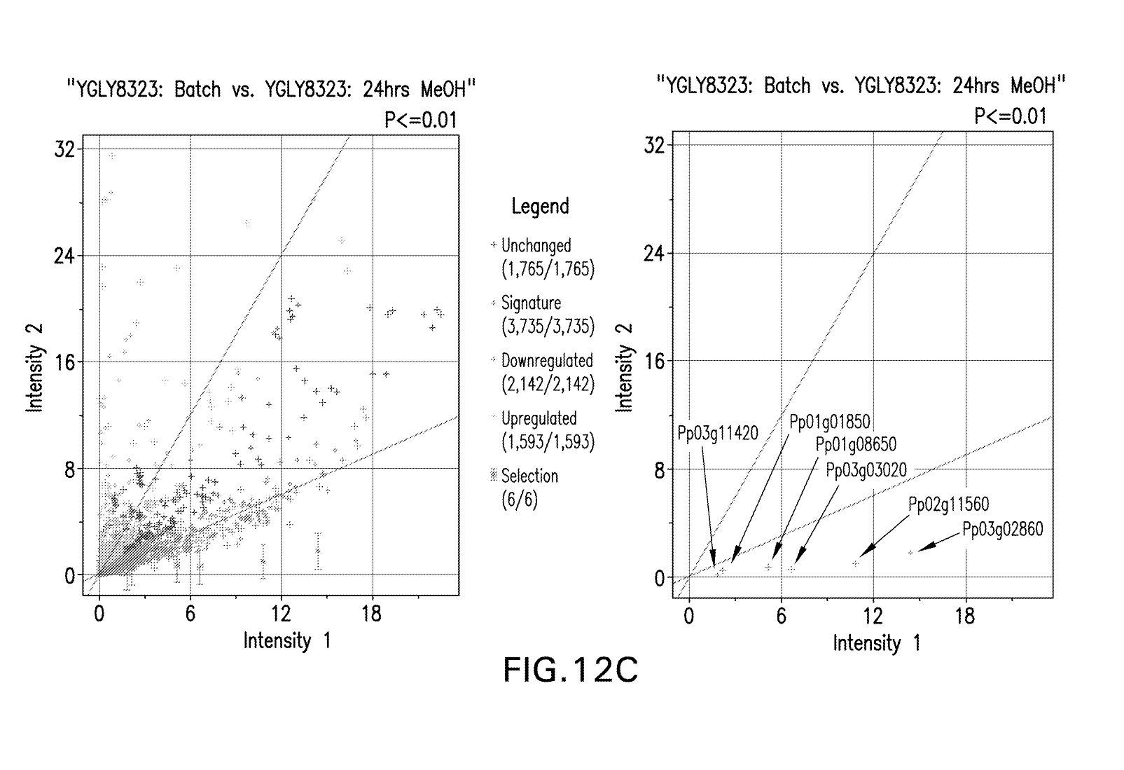

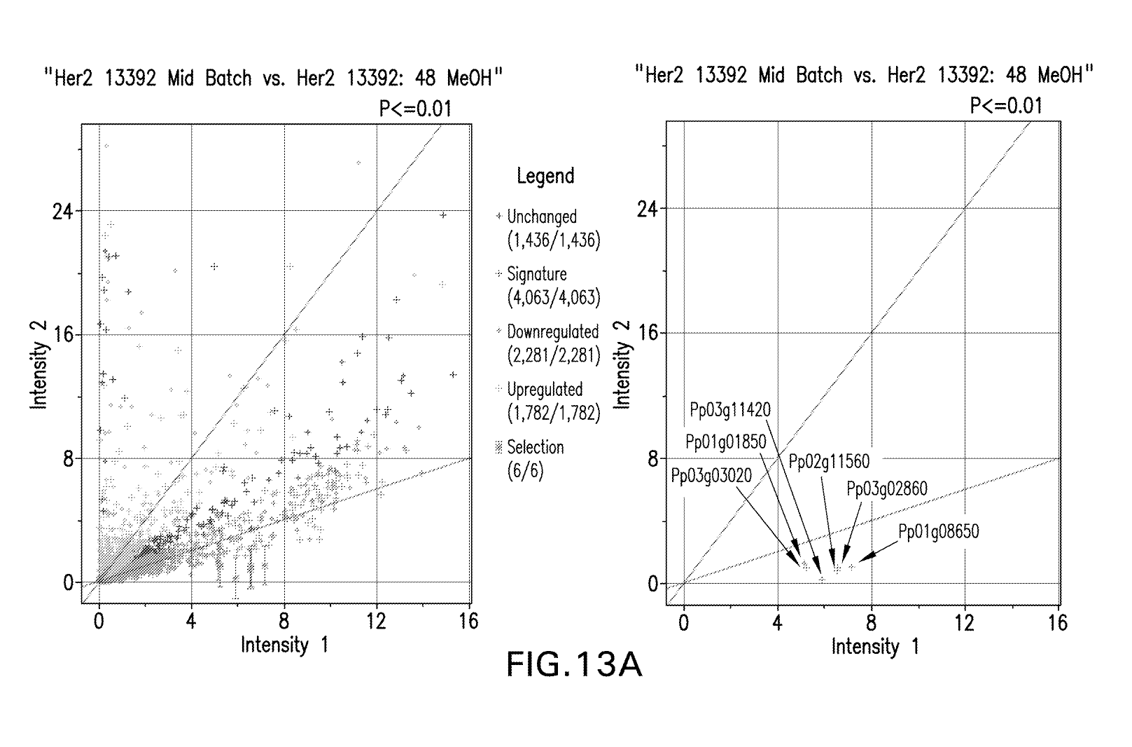

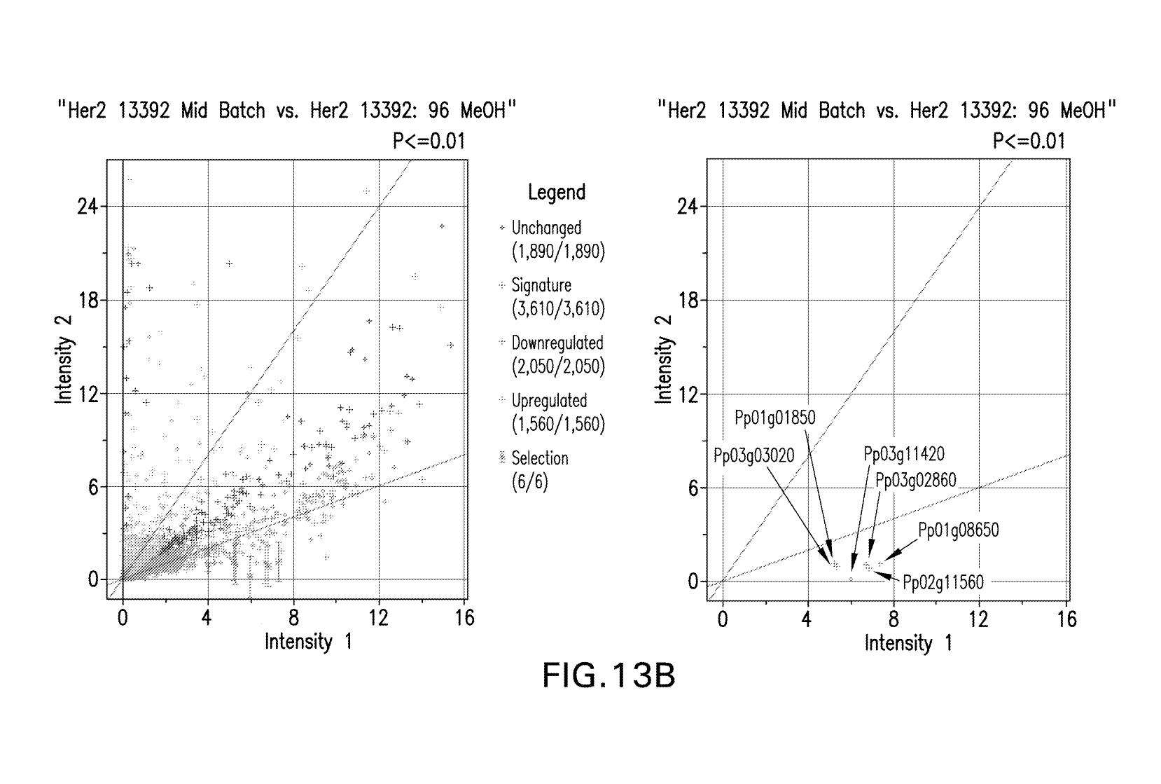

To identify methanol repressible promoters, gene expression data intensity profiles from the wild type/glycoengineered strains study were analyzed by first ratioing data to the Batch (50 mg/ml of wet cell weight; glycerol) timepoint. Similar to the inducible gene clusters, the number genes repressed by methanol (Clusters 3 and 6 in FIG. 2) is too large of a gene set to analyze individually. Therefore, the intersection of those genes with the highest intensity 1 (glycerol) but lowest intensity 2 (methanol) was analyzed by individually comparing intensity profiles at each timepoint as plotted in dotplots for the wild type (y11430) and glycoengineered (YGLY8316 and YGLY8323) strains comparing glycerol (batch) with methanol (24 hours MeOH) timepoints (FIGS. 12A-12C). The same analysis was repeated for the mAb expression comparison study data with exemplary data shown for the mAb expressing strain YGLY13992 (anti-HER2) comparing glycerol (batch) with 48 hour and 96 hour methanol (48 MeOH and 96 MeOH) timepoints (FIGS. 13A and 13B). From these data, 6 genes were identified as having desirable methanol repressible expression. The methanol repressible genes identified included (with common gene name or highest homolog in parentheses):

Pp03g11420 (ScARO10; SEQ ID NO: 64),
Pp02g11560 (ScMET6; SEQ ID NO: 65),
Pp01g08650 (ScYNL067W; SEQ ID NO: 66),
Pp01g01850 (PDHbeta1; SEQ ID NO: 67),
Pp03g03020 (ScSAM2; SEQ ID NO: 68),
Pp03g02860 (SAHH; SEQ ID NO: 69).

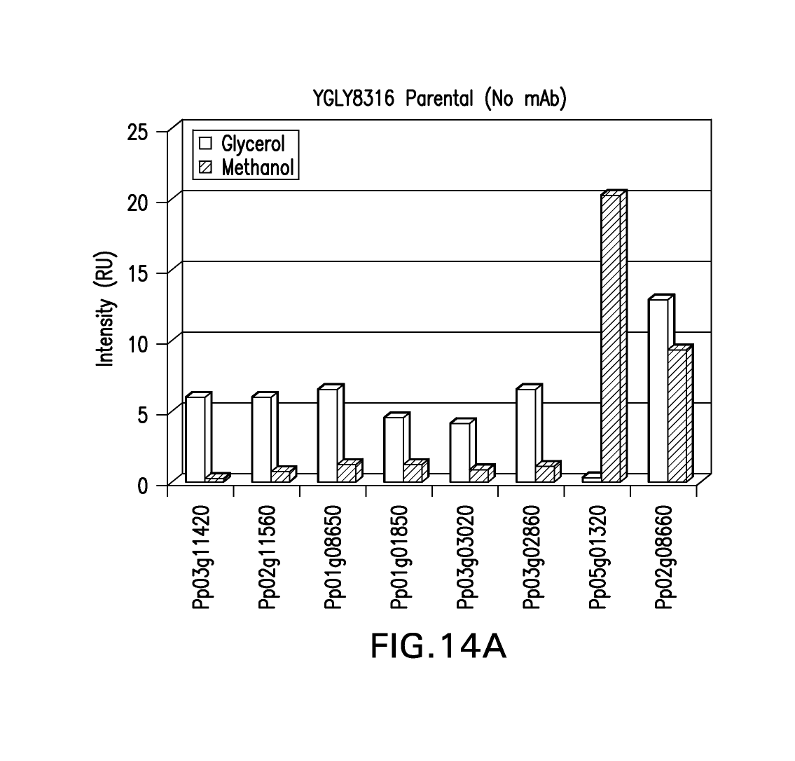

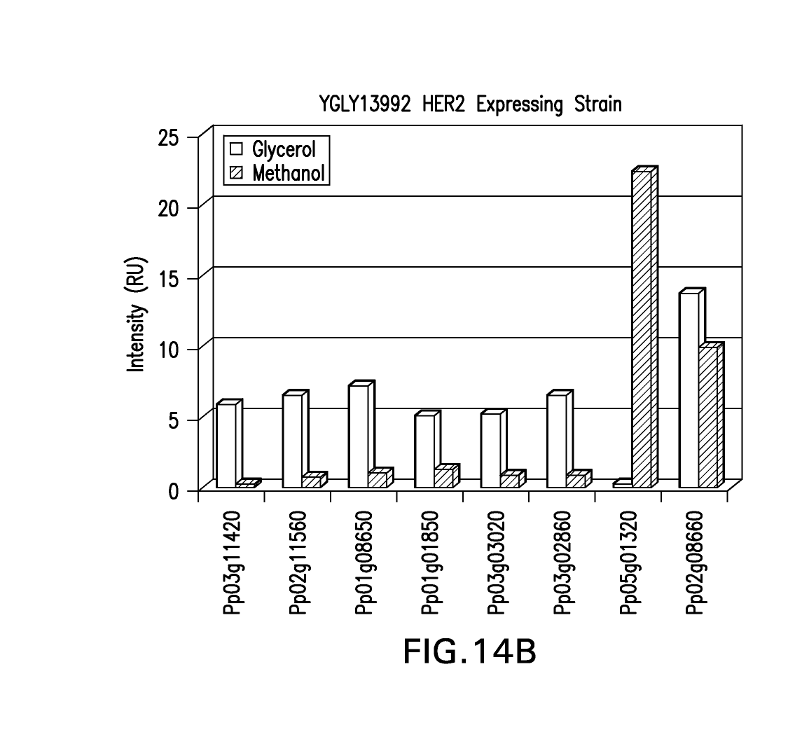

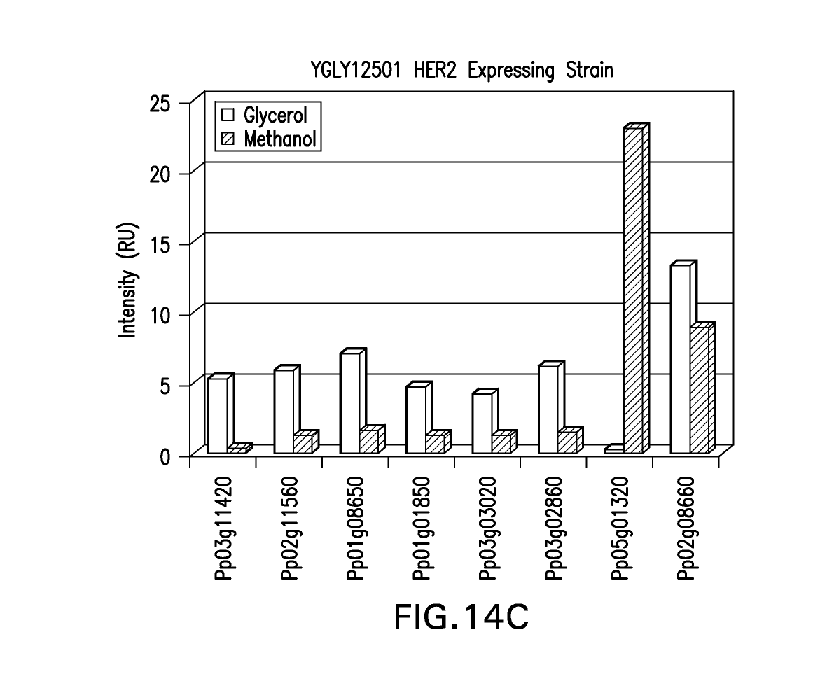

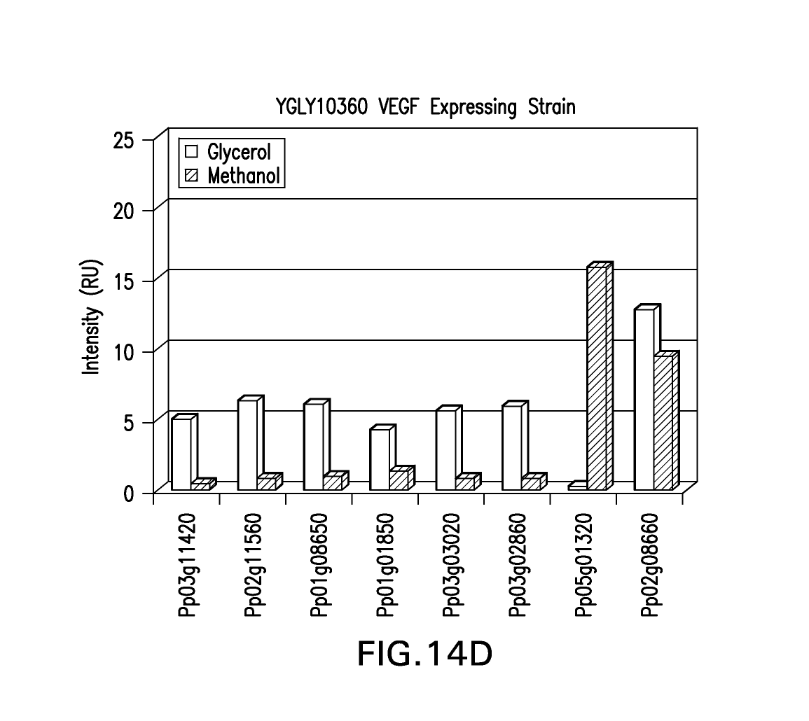

The intensity data for these genes is plotted in comparison to AOX1 and GAPDH (FIG. 14).

The promoters for these genes were extracted as the 5′-proximal 1000 bp of genomic sequence to the ATG of each respective gene. These sequences are contained herein as SEQ ID NOs.: 70-75, respectively.

### Example 8

### Reporter Gene Expression Analysis of Constitutive Promoters

Selected constitutive promoters were fused to the E. coli lacZ (β-galactosidase) gene by cloning a PCR amplified version of the lacZ gene into the NotI/PacI sites in the expression cassettes for promoters PIR1 (Pp02g05010, pGLY8620), CCW12 (Pp05g08520, pGLY8621), CHT2 (Pp01g10900, pGLY8622), PETS (Pp05g07900, pGLY8623), PST1 (Pp02g01530, pGLY8624), TEF (Pp01g00550, pGLY8625), GPD (Pp02g08660, pGLY8626), PMA1 (Pp02g12610, pGLY8627), to generate plasmids pGLY8640-pGLY8647, respectively.

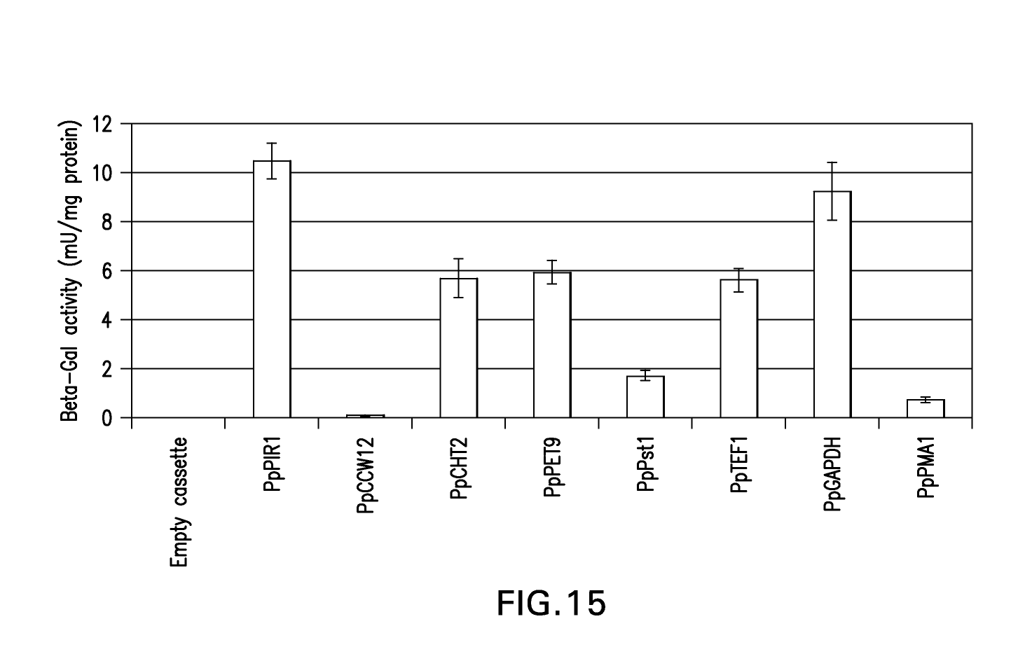

The lacZ containing expression plasmids pGLY8640-8647 were transformed into P. pastoris GFI5.0 strain (Bobrowicz et al., Glycobiol 2004; Davidson U.S. Pat. No. 7,795,002) YGLY8458 and clones were selected on media lacking uracil. Positive transformants were then cultivated in liquid culture in 96 deep well plates on media with glycerol as the sole carbon source for 72 hours and samples of the cells were harvested by centrifugation. The remainder of the culture was then cultivated for an additional 24 hours on media with methanol as the sole carbon source after which samples of the cells were again harvested. The harvested cell pellets were then subjected to a beta-galactosidase assay as previously described (Guarente Methods Emzymol 1983, 101: 181-191). The results of the assay are shown in FIG. 15. Here, the PIR1 promoter yielded higher beta-galactosidase activity than GPD or TEF while the CHT2 and PET9 promoters were stronger than PST1 and PMA1 but in the range of GPD and TEF. These results recapitulate what was observed with the microarray analysis with the exception that, in this experiment, the GPD promoter appeared stronger than that of TEF, whereas previous experiments (Ahn, Appl Microbiol Biotechnol, 2007, 74:601-608; and Lee WO2007058407) and the microarray analysis presented here indicated that TEF was slightly stronger. Together, these exemplary data confirm PIR1, CHT2, PET9 and PST1 as examples of a novel set of promoters useful for strong constitutive heterologous gene expression in yeast.

### Example 9

### Reporter Gene Expression Analysis of Inducible Promoters

Selected methanol-inducible promoters were fused to the E. coli LacZ (β-galactosidase) gene by cloning a PCR amplified version of the lacZ gene into the NotI/PacI sites in the expression cassettes for promoters Pp03g08760 (ScCWP1, pGLY8529), Pp03g03520 (DAS2, pGLY8530), Pp03g00990 (ScYGR201C, pGLY8532), Pp03g03500 (DAS1, pGLY8533), Pp01g09290 (ScFBA1, pGLY8531), to generate plasmids pGLY8549, pGLY8550, pGLY8552, pGLY8553, and pGLY8551, respectively.

### Example 10

### Expression of a Secreted Reporter Gene by Methanol-Inducible Promoters

Selected inducible promoters were also fused to the Human Fc gene by cloning a PCR amplified version of the Human Fc gene into the NotI/PacI sites in the expression cassettes for promoters CWP1 (Pp03g08760, pGLY8529), PpDAS2 (Pp03g03520, pGLY8530), FBA1 (Pp01g09290, pGLY8531), YGR201C (Pp03g00990, pGLY8532), as well as PpDAS1 (Pp03g03500, (pGLY8533), as a control to generate plasmids pGLY8539, pGLY8540, pGLY8548, pGLY8545, and pGLY8546, respectively. Also as a control, the AOX1 promoter (Pp05g01320) was inserted as a BglII/NotI fragment from plasmid pGLY4464 along with the hFc NotI/PacI PCR fragment, into pGLY580 digested with BglII/PacI to generate pGLY8547.

The hFc containing expression plasmids pGLY8539, pGLY8540, pGLY8545, pGLY8546, pGLY8547, and pGLY8548 were transformed into P. pastoris GFI5.0 strain (Bobrowicz et al., Glycobiol. 2004, 14(9):757-66; Davidson U.S. Pat. No. 7,795,002) YGLY8458 and clones were selected on media lacking uracil. Positive transformants were identified by PCR for the plasmid integration using standard methods.

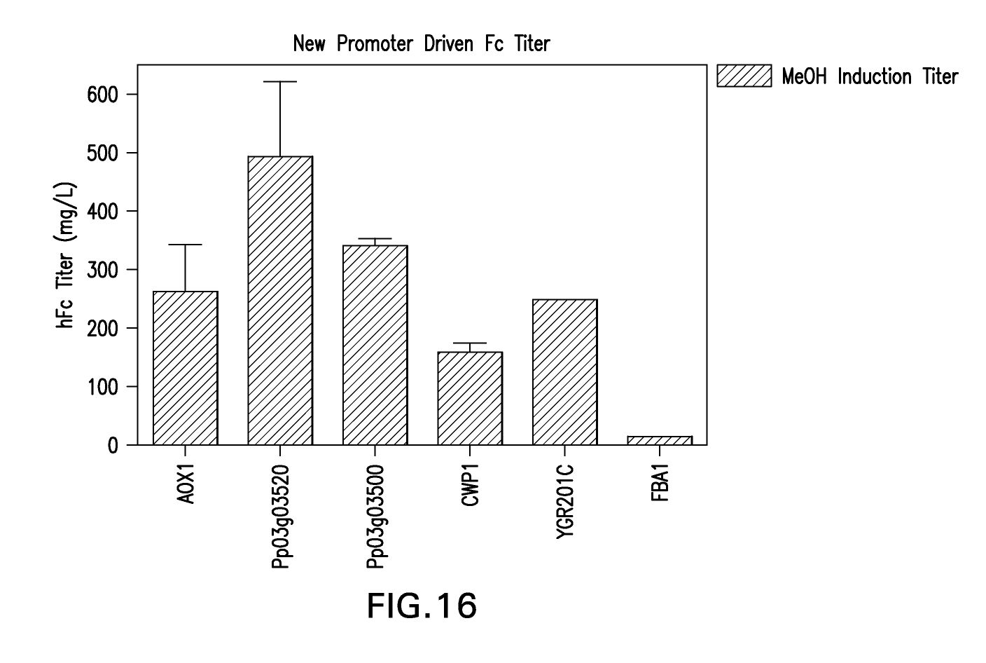

Positive transformants were then cultivated in liquid culture in an Applikon “micro24” 24 well 5 ml mini fermenter system in media with glycerol as the sole carbon source for 72 hours and sample supernatants were harvested by centrifugation. The remainder of the culture was then cultivated for an additional 72 hours on media with methanol as the sole carbon source after which sample supernatants of the cells were again harvested. The harvested supernatant was then subjected to a HPLC to determine Fc titer. The results of the assay for the 72 hour methanol samples are shown in FIG. 16. Not shown, but of note is that no measurable Fc titer was observed for any glycerol samples, consistent with the methanol inducible nature of these promoters. Here, interestingly the Pp03g03520 (DAS2) promoter yielded higher supernatant HPLC titer than the canonically strong AOX1 (Pp05g01320) and (DAS1) Pp03g03500 promoter controls. The CWP1 and YGR201C promoters displayed slightly weaker Fc expression than the AOX1 promoter, while the FBA1 promoter was determined to be much weaker than AOX1 in this assay, but still showed methanol-inducible activity. These results recapitulate what was observed with the microarray analysis, that both the previously identified Pp03g03500 (DAS1) and new Pp03g03520 (DAS2) promoters appear stronger than that of AOX1. Together, these data demonstrate that Pp03g03520, Pp03g03500, CWP1, FBA1, and YGR201C are examples of a novel set of promoters useful for tunable methanol-inducible heterologous gene expression in yeast.

### Example 11

### Construction of a Protein A-Sc SED1 Cell Surface Anchor Under Control of Methanol Repressible Promoters

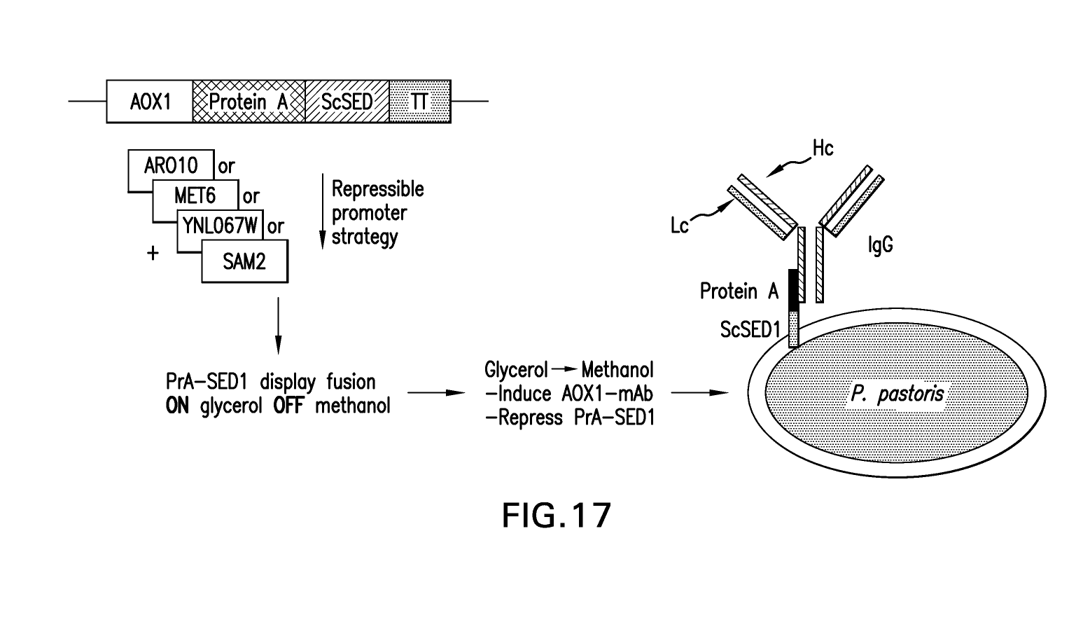

Whole antibodies can be displayed on the surface of P. pastoris cells by anchoring a protein-A/S. cerevisiae SED1 protein fusion and capturing secreted mAb provided that the protein-A anchor and the antibody are not co-expressed simultaneously (Prinz US2010/0009866). In that case, the GUT1 promoter (glucose-repressible) was used to drive the protein A-based anchor and GPD (GAPDH) promoter was used to drive the secreted/anchored monoclonal antibody. Increased expression of the GPD-mAb upon switch from glycerol to glucose, in combination with repression of the GUT1-SED1/ProteinA anchor, resulted in successful anchoring of monoclonal antibody on the cell surface. Here, several exemplary methanol repressible promoters were utilized for cell surface display of the protein A-based anchor to provide disparate expression of the anchor from the secreted AOX1-driven monoclonal antibody as depicted in the cartoon in FIG. 17.

Four methanol repressible promoters were chosen for the protein-A/SED1 anchor whole antibody cell surface display. The promoters of Pp03g11420 (Homolog to S. cerevisiae ARO10), Pp02g11560 (Homolog to S. cerevisiae METE), Pp01g08650 (Homolog to S. cerevisiae YNL067W, protein component of the large 60S ribosomal subunit), Pp03g03020 (Homolog to S. cerevisiae SAM2) showed strong transcription in the glycerol phase and strong repression in the methanol phase and were therefore chosen to express the protein-A/SED1 anchor.

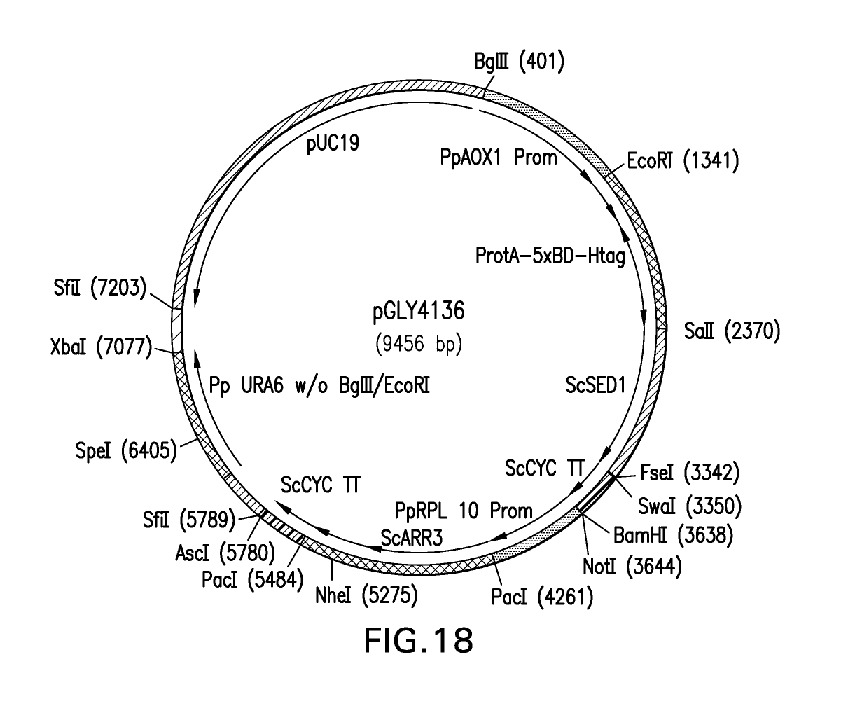

The sequences of the four promoters were in vitro synthesized (GeneArt, AG, Regensberg, Germany) and subcloned as BglII-EcoRI fragments into pGLY4136, in front of a gene encoding 5 IgG-binding domains of protein-A anchored to the S. cerevisiae SED1 protein, which anchored the protein-A onto the P. pastoris cell surface. The plasmid pGLY4136 also contained the Arsenite (Ars) resistance gene as a selection marker and the P. pastoris URA6 gene as integration site (FIG. 18). Cloning of the Pp03g11420 (PpARO10), Pp02g11560 (PpMET6), Pp01g08650 (PpYNL067W), and Pp03g03020 (PpSAM2) promoters into this plasmid at the BglII/EcoRI sites in place of AOX1 yielded pGLY9545, pGLY9546, pGLY9547, and pGLY9548, respectively.

Plasmids pGLY9545-9548 were transformed into the empty glycoengineered GS5.0 strain YGLY17108 that does not have a secreted monoclonal antibody construct, as well as glycoengineered GS5.0 strains YGLY13979 containing a secreted AOX1-driven anti-HER2 monoclonal antibody construct, along with YGLY18281 (AX132) and YGLY18483 (AX189), each expressing a distinct secreted AOX1-driven anti-PCSK9 monoclonal antibody construct. Clones were selected on plates containing 1 mM arsenite.

### Example 12

### Display of a Methanol Repressible Protein A-Sc SED1 Anchor on P. Pastoris Cell Surface

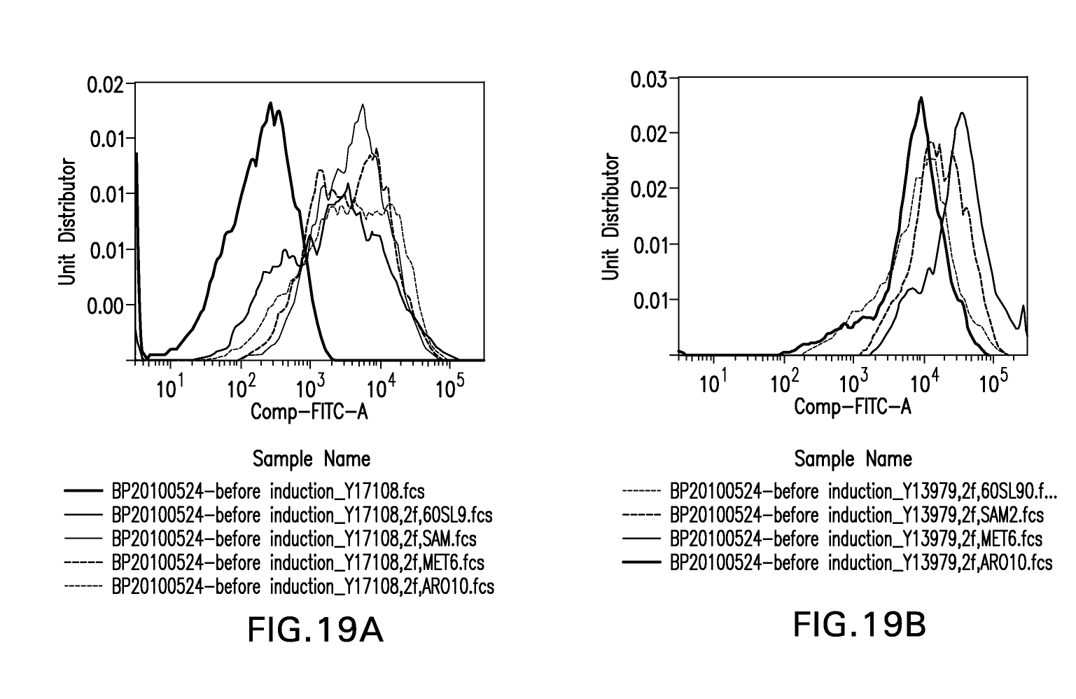

Transformants of the empty glycoengineered GS5.0 strain containing the protein-A/S. cerevisiae SED1 anchor under the four different repressible promoters were grown in glycerol media and then induced in methanol. Samples were taken in glycerol and after 24, 48 and 72 hours of induction in methanol and labeled with fluorescent rabbit IgG1-Alexa Fluor 488. The rabbit IgG1 bound to the protein-A on the yeast cell surface and can be monitored by FACS analysis (Lin et al, J. Immunol. Methods. 2010, 358(1-2):66-74). In glycerol phase, the protein-A was displayed on the cell surface under all four promoters (PpARO10; PpMET6, PpYNL067W, and PpSAM2) while the parental strain, without the protein A display construct, does not show any labeling (FIG. 19). Moreover, following methanol-induction, cell surface detection of protein-A decreased gradually over a 72 hour timecourse, suggesting that new protein-A was not being added to the cell surface while that which was produced during glycerol growth degraded and/or was diluted by cell division.

### Example 13

### Capture of Antibody by the Displayed Protein A-Sc SED1 Anchor on the P. Pastoris Cell Surface

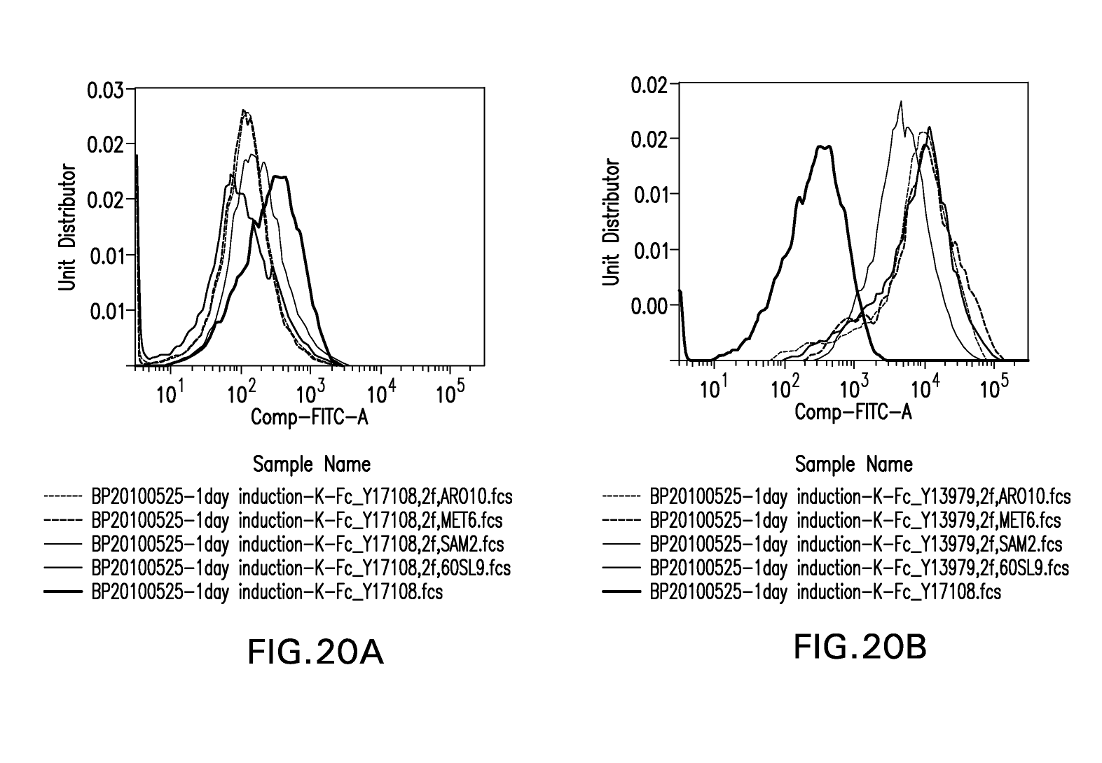

Transformants from the four antibody expressing glycoengineered GS5.0 strains containing the protein-A-S. cerevisiae SED1 anchor, under the four different methanol-repressible promoters, were grown in glycerol media and then induced in methanol for two days. Samples were taken after 24 and 48 hours of induction in methanol. YGLY13979 transformed anti-HER2 monoclonal antibody expressing strains were labeled with fluorescent Fab anti-Fc DyLight-488 and anti-human Kappa-APC conjugated to detect the light chain and the heavy chain of the displayed antibody. The displayed anti-Her2 antibody was efficiently captured on the cell surface at both timepoints as judged by the observed fluorescence shift of these cell populations, while the YGLY17108 strain without expressing an antibody or the strain with neither antibody nor protein A display do not show a fluorescence shift (FIG. 20).

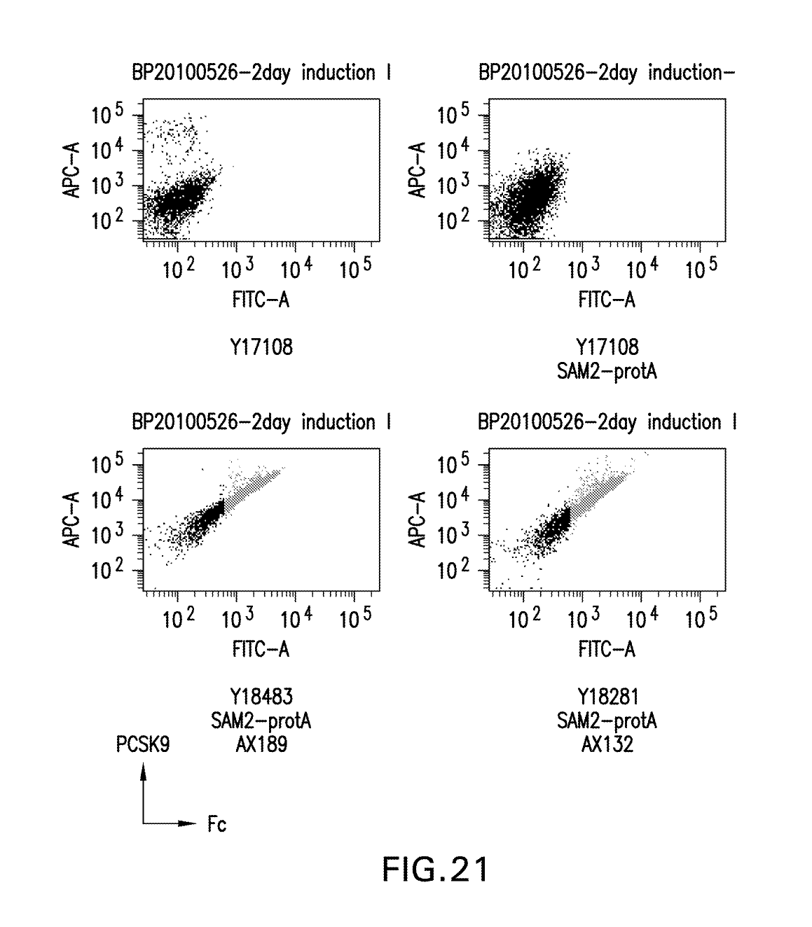

Two transformed anti-PCSK9 expressing strains, YGLY22299 and YGLY22301, were labeled with fluorescent Fab anti-Fc DyLight-488 to detect the antibody heavy chain and with biotinylated PCSK9 antigen and further labeled with streptavidin-Alexa Fluor 635 conjugate to detect the biotinylated PCSK9 antigen. FIG. 21 demonstrates that anchored antibody can be detected on the cell surface of each strain, as detected by both the antigen (PCSK9) and a molecule that detects the heavy chain of the antibody (anti-Fc Fab). Moreover, supernatants harvested from these cultivations revealed the presence of secreted antibody as judged by SDS-PAGE, Western immunoblot, bead assay, Caliper assay. Together, these data demonstrated that antibody was properly secreted in these strains upon methanol induction and furthermore, while some is captured by the protein-A anchored on the cell surface and can be used for FACS labeling and sorting, part of the antibody was secreted freely into the media. Importantly, this freely secreted antibody can then be purified and utilized for downstream biochemical and biophysical analyses allowing display/sorting and analysis of secreted full-length mAbs from the same strain.

### Example 14

### 40 L Scale-Fermentation of New Promoter Cassettes Driving lacZ

One unexpected aspect to the constitutive promoter analysis was the high level of expression obtained from the control strong GAP (Pp02g08660) promoter. The lacZ construct used to test this promoter included about 1 kb of the GAP promoter as well as 500 bb of the native GAP transcriptional terminator sequence (SEQ ID NO: 28). Previous reports have focused on fusing only 500 bp of the GAP promoter with either the S. cerevisiae CYC1 transcriptional terminator or the P. pastoris AOX1 transcriptional terminator. Here, the increase in expression levels for the GAP promoter (FIG. 15) far exceeded the expression level of the previous GAP-CYC1 cassette as well as the commonly used TEF promoter cassette. However, the new GAP cassette still maintained a similar expression profile (strong expression with a mild ˜20-30% decline under methanol feed conditions). Therefore, it was possible that this difference in promoter size and/or terminator identity may have resulted in the unexpected increase in expression GAP promoter-driven levels.

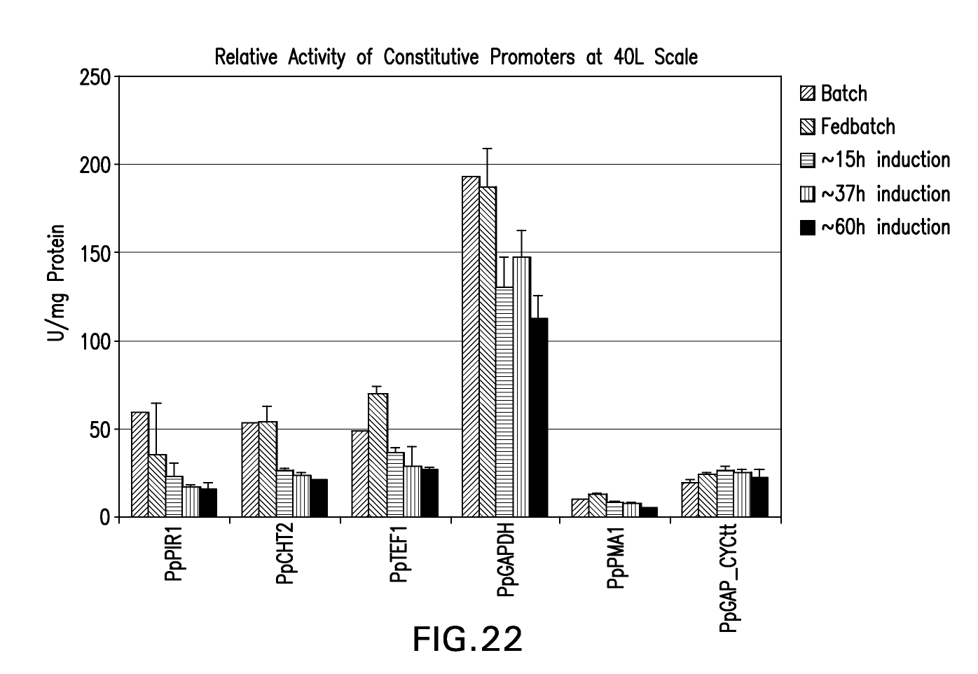

To this end, an additional control promoter-terminator combination was generated by fusing the traditional 500 bp of the P. pastoris GAP gene, Pp01g08620, promoter (nucleotides 7-492 of SEQ ID NO: 76) and ˜300 bp 3′ terminator region of the S. cerevisiae CYC1 gene (nucleotides 515-807 of SEQ ID NO: 76) from pGLY580 (FIG. 7A) with the E. coli lacZ gene to generate plasmid pGLY9747. The E. coli lacZ gene was cloned into this canonical GAP/CYC1 promoter/terminator fusion to generate plasmid pGLY9747 (FIG. 22). This pGLY9747 lacZ containing GAP-CYC1 expression plasmid was transformed into P. pastoris GFI5.0 strain YGLY8458 as previously and clones were selected on media lacking uracil. Positive transformants confirmed by PCR were then cultivated in liquid culture in 96 deep well plates on media with glycerol as the sole carbon source for 72 hours and samples of the cells were harvested by centrifugation. The remainder of the culture was then cultivated for an additional 24 hours on media with methanol as the sole carbon source after which samples of the cells were again harvested. The harvested cell pellets were then subjected to a beta-galactosidase assay as previously described to confirm expression (Guarente, Methods Emzymol 1983, 101: 181-191).

Strains containing the GAP-CYC1 fusion (YGLY23848), the PIR1 promoter/terminator fusion (YGLY23728), the CHT2 promoter/terminator fusion (YGLY23734), the TEF promoter/terminator fusion (YGLY23743), the PMA1 promoter/terminator fusion (YGLY23749), and the newly described GAP promoter/terminator fusion from this work (YGLY23747), were cultivated at 40 liter fermentation scale to confirm constitutive promoter activity during the course of a glycerol-to-methanol fermentation process at large scale. First, a Research Cell Bank (RCB) was generated for each strain by cultivating a loopful of cells from a YPD plate for 48 hours in 200 ml of BMGY media (Invitrogen, Carlsbad, Calif.) to a measured optical density of 20-80. Cells are then mixed with 80% glycerol (v/v) to generate a final concentration of 20% glycerol to cell suspension (v/v) and cells are frozen at −80° C. in 1 ml aliquots.

For fermentation, performed in a stainless steel 40 liter Applikon (Foster City, Calif.) bioreactor, a vial (1 mL) of a RCB was inoculated into 500 mL of BSGY medium (4% glycerol, 1% yeast extract, 2% Soytone, 1.34% YNB without amino acids, 0.23% K₂HPO₄, 1.19% KH₂PO₄, 8 μg/L biotin) in 2.8 liter-baffled flask. The culture incubated at 24° C., while shaking on an orbital shaker at 180 rpm for 48±4 hours. The bioreactor was inoculated with a 10% volumetric ratio of seed to initial modified BSGY medium containing 50 g/L of maltitol and no sorbitol. Cultivation conditions were as follows: temperature set at 24±0.5° C., pH controlled at 6.5±0.1 with 30% ammonium hydroxide, dissolved oxygen was maintained at 20% of saturation by cascading agitation rate on the addition of pure oxygen to the fixed airflow rate of 0.7 vvm. After depletion of the initial glycerol (4%) charge, a 50% glycerol solution containing 12.5 mL/L of PTM1 salts (6.5 g FeSO₄.7H₂O, 2.0 g ZnCl₂, 0.6 g CuSO₄.5H₂O, 3.0 g MnSO₄.7H₂O, 0.5 g CoCl₂.6H₂O, 0.2 g NaMoO₄.2H₂O, 0.2 g biotin, 80 mg NaI, 20 mg H₃BO₄ per L) was fed exponentially at a rate of 0.08 h⁻¹ for 8 hours. After a 30 minute starvation phase, induction was initiated where methanol was fed exponentially starting at 1.5 g/L/h increasing at a rate of 0.008 h⁻¹ and the entire induction phase was conducted under methanol-limited conditions.

Samples were harvested by removing 1 ml of broth and centrifuging for 30 seconds at top speed in a microcentrifuge, then flash freezing at −80° C. Samples were harvested during glycerol batch (˜50 mg/ml of wet cell weight), at the middle of glycerol fedbatch, and at 15+/−2 h, 37+/−2 h, and 60+/−2 hour of methanol induction. For lacZ assays, frozen cell pellets (100-200 ml) were washed twice in 1 ml PBS and resuspended in 200 ul complete protein inhibitor cocktail (Roche, cat #11 873 580 001) containing PBS. The cells were disrupted by vigorously vortexing cell suspension (100 ml) twice with 10 mg of 425-600 mesh glass beads (acid washed and air dried) for 2 minutes following addition of zymolyase (1 U/ml; AMS Biotechnology; Zymolyase®-20T). The mixture was placed at room temperature for 60 minutes with occasional brief vortexing. The protein content of the cell lysate was determined by BCA assay (Pierce, cat#23225). The unit of galactosidase activity was determined by the rate of 4-Methylumbelliferyl β-D-galactopyranoside hydrolysis in PBS per min per mg protein. β-Galactosidase from Kluyveromyces lactis (Sigma, Cat# G3665) was used as standard. The release of 4-Methylumbelliferone was measured by fluorescence detection (ex=355, em=460) for the duration of 5-60 minutes.

The 40 liter lacZ expression data demonstrated the scalability of each of the promoter cassettes tested. Similar to previous results, all promoters drove expression of lacZ under all conditions tested including the new PIR1 and CHT2 promoters and all promoters showed some level of expression reduction at later timepoints on methanol induction. Also, consistent with previous results, the PMA1 promoter, commonly used as a strong constitutive promoter, was quite weak compared to the other promoters tested and was especially reduced in expression on methanol compared to the other promoters. Again, the 1 kb GAP promoter paired with its native terminator was stronger than most of the other promoters, and here was significantly stronger than even the PIR1 or TEF promoters. However, the control 500 bp GAP promoter paired with the CYC1 terminator was significantly weaker than the 1 kb GAP promoter and in fact weaker than the TEF and PIR1 promoters as previously expected. These data demonstrated that the 1 kb GAP promoter paired with its native terminator established a new version of this promoter with a similar near constitutive nature (weaker on methanol than glycerol but still highly active) but much more active than the canonical 500 bp version previously reported. And the surprising identification of this new version of the GAP promoter will be a useful option as a highly active promoter useful for driving strong transcription of transgenes in P. pastoris.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

We claim:

1. An isolated hybrid polynucleotide comprising a promoter selected from the group consisting of:
Pichia pastoris GAPDH promoter;
Pichia pastoris Pp02g05010 (PpPIR1) promoter;
Pichia pastoris Pp05g08520 (ScCCW12) promoter;
Pichia pastoris Pp01g10900 (ScCHT2) promoter;
Pichia pastoris Pp05g07900 (ScAAC2/PET9) promoter;
Pichia pastoris Pp02g01530 (ScPST1) promoter;
Pichia pastoris Pp05g00700 promoter;
Pichia pastoris Pp02g04110 (ScPOR1) promoter;
Pichia pastoris Pp01g03600 (ScBGL2) promoter;
Pichia pastoris Pp01g14410 (ScACO1) promoter;
Pichia pastoris Pp01g09650 (ScYHR021C) promoter;
Pichia pastoris Pp01g02780 (ScYLR388W) promoter;
Pichia pastoris Pp03g09940 (ScPIL1) promoter;
Pichia pastoris Pp02g10710 (ScMDH1) promoter;
Pichia pastoris Pp01g09290 (ScFBA1) promoter;
Pichia pastoris Pp03g03520 (PpDAS2) promoter;
Pichia pastoris Pp03g08760 (ScCWP1) promoter;
Pichia pastoris Pp03g00990 (ScYGR201c) promoter;
Pichia pastoris Pp02g05270 (AN2948.2) promoter;
Pichia pastoris Pp02g12310 (ScDUR3) promoter;
Pichia pastoris Pp03g05430 (ScTHI4) promoter;
Pichia pastoris Pp03g03490 (AN2957.2) promoter;
Pichia pastoris Pp05g09410 (ScTHI13) promoter;
Pichia pastoris Pp02g07970 (ScPEX11/PMP27) promoter;
Pichia pastoris Pp01g12200 (AN7917.2) promoter;
Pichia pastoris Pp03g11380 (ScPMP47) promoter;
Pichia pastoris Pp03g08340 promoter;
Pichia pastoris Pp05g04390 (ScTIR3) promoter;
Pichia pastoris Pp01g08380 (ScYIL057c) promoter;
Pichia pastoris Pp01g05090 (ScSAY1) promoter;
Pichia pastoris Pp01g13950 (ScTPN1) promoter;
Pichia pastoris Pp03g11420 (ScARO10) promoter;
Pichia pastoris Pp02g11560 (ScMET6) promoter;
Pichia pastoris Pp01g08650 (ScYNL067W) promoter;
Pichia pastoris Pp01g01850 (PpPDHbeta1) promoter;
Pichia pastoris Pp03g03020 (ScSAM2) promoter; and
Pichia pastoris Pp03g02860 (PpSAHH) promoter; or a functional variant thereof; optionally, operably linked to a heterologous polynucleotide and/or to a transcriptional terminator.

2. The isolated hybrid polynucleotide of claim 1 wherein the heterologous polynucleotide encodes an immunoglobulin or interferon.

3. The isolated hybrid polynucleotide of claim 1 wherein the heterologous polynucleotide encodes:
an immunoglobulin heavy chain and/or immunoglobulin light chain optionally linked to an immunoglobulin constant domain;
an immunoglobulin chain of an antibody or antigen-binding fragment thereof that binds specifically to VEGF, HER1, HER2, HER3, glycoprotein IIb/IIIa, CD52, IL-2R alpha receptor (CD25), epidermal growth factor receptor (EGFR), Complement system protein C5, CD11a, TNF alpha, CD33, IGF1R, CD20, T cell CD3 Receptor, alpha-4 (alpha 4) integrin, PCSK9, immunoglobulin E (IgE), RSV F protein or ErbB2; or said heterologous polynucleotide encodes:
VEGF, HER1, HER2, HER3, glycoprotein IIb/IIIa, CD52, IL-2R alpha receptor (CD25), epidermal growth factor receptor (EGFR), Complement system protein C5, CD11a, TNF alpha, CD33, IGF1R, CD20T cell CD3 Receptor, alpha-4 (alpha 4) integrin, PCSK9, immunoglobulin E (IgE), RSV F protein or ErbB2; or an immunogenic fragment thereof;
or said heterologous polynucleotide encodes:
green fluorescent protein, Aequorea victoria GFP mutant 3, luciferase, Renilla luciferase, Photinus pyralis luciferase, Photinus pyralis luciferase slk mutant, Vibrio fischeri luxA, Vibrio fischeri luxB, Vibrio fischeri luxC, Vibrio fischeri luxD, Vibrio fischeri luxE, Vibrio fischeri luxAB, Vibrio fischeri luxCDABE, Vibrio harveyi luxA, Vibrio harveyi luxB, Vibrio harveyi luxC, Vibrio harveyi luxD, Vibrio harveyi luxE, Vibrio harveyi luxAB, Vibrio harveyi luxCDABE, Photorhabdus luminscens LuxA, Photorhabdus luminscens LuxB, Photorhabdus luminscens LuxC, Photorhabdus luminscens LuxD, Photorhabdus luminscens LuxE, Photorhabdus luminscens LuxCDABE, E. coli lacZ, the Aequorea victoria Aequorin, KanMX, pat1, nat1, hph, CAT, Sh Ble, GUS, CYH2 or CAN1.

4. The isolated hybrid polynucleotide of claim 1 wherein said promoter comprises a nucleotide sequence selected from the group consisting of:
nucleotides 1-1000 of SEQ ID NO: 14;
nucleotides 1-1000 of SEQ ID NO: 15;
nucleotides 1-1000 of SEQ ID NO: 16;
nucleotides 1-1000 of SEQ ID NO: 17;
nucleotides 1-1000 of SEQ ID NO: 18;
nucleotides 1-1001 of SEQ ID NO: 19;
nucleotides 1-1000 of SEQ ID NO: 20;
nucleotides 1-1000 of SEQ ID NO: 21;
nucleotides 1-1000 of SEQ ID NO: 22;
nucleotides 1-1000 of SEQ ID NO: 23;
nucleotides 1-1000 of SEQ ID NO: 24;
nucleotides 1-1000 of SEQ ID NO: 25;
nucleotides 1-1000 of SEQ ID NO: 26;
nucleotides 1-1000 of SEQ ID NO: 27;
nucleotides 1-1000 of SEQ ID NO: 28;
nucleotides 1-1000 of SEQ ID NO: 29; and
SEQ ID NOs: 47-63 and 70-76.

5. An isolated vector comprising a hybrid polynucleotide of claim 1.

6. An isolated host cell comprising a hybrid polynucleotide or vector of claim 1.

7. The host cell of claim 6 which is selected from the group consisting of a fungal cell, a Pichia cell, Pichia pastoris, Pichia flnlandica, Pichia trehalophila, Pichia koclamae, Pichia membranaefaciens, Pichia minuta, Ogataea minute, Pichia lindneri, Pichia opuntiae, Pichia thermotolerans, Pichia salictaria, Pichia guercuum, Pichia pijperi, Pichia stiptis, Pichia methanolica, Pichia, Saccharomyces cerevisiae, Saccharomyces, Hansenula polymorpha, Kluyveromyces, Kluyveromyces lactis, Candida albicans, Aspergillus nidulans, Aspergillus niger, Aspergillus oryzae, Trichoderma reesei, Chrysosporium lucknowense, Fusarium, Fusańum gramineum, Fusarium venenatum and Neuraspora crassa.

8. A composition comprising the host cell of claim 6 and growth culture medium.

9. The composition of claim 8 comprising methanol.

10. A method for making a polypeptide comprising introducing, into an isolated fungal host cell, an isolated hybrid polynucleotide comprising a promoter selected from the group consisting of:
Pichia pastoris GAPDH promoter;
Pichia pastoris Pp02g05010 (PpPIR1) promoter;
Pichia pastoris Pp05g08520 (ScCCW12) promoter;
Pichia pastoris Pp01g10900 (ScCHT2) promoter;
Pichia pastoris Pp05g07900 (ScAAC2/PET9) promoter;
Pichia pastoris Pp02g01530 (ScPST1) promoter;
Pichia pastoris Pp05g00700 promoter;
Pichia pastoris Pp02g04110 (ScPOR1) promoter;
Pichia pastoris Pp01g03600 (ScBGL2) promoter;
Pichia pastoris Pp01g14410 (ScACO1) promoter;
Pichia pastoris Pp01g09650 (ScYHR021C) promoter;
Pichia pastoris Pp01g02780 (ScYLR388W) promoter;
Pichia pastoris Pp03g09940 (ScPIL1) promoter;
Pichia pastoris Pp02g10710 (ScMDH1) promoter;
Pichia pastoris Pp01g09290 (ScFBA1) promoter;
Pichia pastoris Pp03g03520 (PpDAS2) promoter;
Pichia pastoris Pp03g08760 (ScCWP1) promoter;
Pichia pastoris Pp03g00990 (ScYGR201c) promoter;
Pichia pastoris Pp02g05270 (AN2948.2) promoter;
Pichia pastoris Pp02g12310 (ScDUR3) promoter;
Pichia pastoris Pp03g05430 (ScTHI4) promoter;
Pichia pastoris Pp03g03490 (AN2957.2) promoter;
Pichia pastoris Pp05g09410 (ScTHI13) promoter;
Pichia pastoris Pp02g07970 (ScPEX11/PMP27) promoter;
Pichia pastoris Pp01g12200 (AN7917.2) promoter;
Pichia pastoris Pp03g11380 (ScPMP47) promoter;
Pichia pastoris Pp03g08340 promoter;
Pichia pastoris Pp05g04390 (ScTIR3) promoter;
Pichia pastoris Pp01g08380 (ScYIL057c) promoter;
Pichia pastoris Pp01g05090 (ScSAY1) promoter;
Pichia pastoris Pp01g13950 (ScTPN1) promoter;
Pichia pastoris Pp03g11420 (ScARO10) promoter;
Pichia pastoris Pp02g11560 (ScMET6) promoter;
Pichia pastoris Pp01g08650 (ScYNL067W) promoter;
Pichia pastoris Pp01g01850 (PpPDHbeta1) promoter;
Pichia pastoris Pp03g03020 (ScSAM2) promoter; and
Pichia pastoris Pp03g02860 (PpSAHH) promoter; or a functional variant thereof;
operably linked to a heterologous polynucleotide encoding said polypeptide, and, optionally, to a transcriptional terminator; and
culturing the host cell under conditions wherein said polynucleotide is expressed; optionally wherein said host cell is cultured in the presence of methanol.

11. The method of claim 10 wherein the fungal host cell is selected from the group consisting of a fungal cell, a Pichia cell, Pichia pastoris, Pichia flnlandica, Pichia trehalophila, Pichia koclamae, Pichia membranaefaciens, Pichia minuta, Ogataea minuta, Pichia lindneri, Pichia opuntiae, Pichia thermotolerans, Pichia salictaria, Pichia guercuum, Pichia pijperi, Pichia stiptis, Pichia methanolica, Pichia, Saccharomyces cerevisiae, Saccharomyces, Hansenula polymorphs, Kluyveromyces, Kluyveromyces lactis, Candida albicans, Aspergillus nidulans, Aspergillus niger, Aspergillus oryzae, Trichoderma reesei, Chrysosporium lucknowense, Fusarium, Fusańum gramineum, Fusarium venenatum and Neuraspora crassa.

12. A method for methanol-inducing expression of a heterologous polynucleotide in a fungal host cell, wherein said host cell comprises a promoter selected from the group consisting of:
Pichia pastoris Pp01g09290 (ScFBA1) promoter;
Pichia pastoris Pp03g03520 (PpDAS2) promoter;
Pichia pastoris Pp03g08760 (ScCWP1) promoter;
Pichia pasterns Pp03g00990 (ScYGR201c) promoter;
Pichia pasterns Pp02g05270 (AN2948.2) promoter;
Pichia pasterns Pp02g12310 (ScDUR3) promoter;
Pichia pasterns Pp03g05430 (ScTHI4) promoter;
Pichia pastoris Pp03g03490 (AN2957.2) promoter;
Pichia pasterns Pp05g09410 (ScTHI13) promoter;
Pichia pastoris Pp02g07970 (ScPEX11/PMP27) promoter;
Pichia pastoris Pp01g12200 (AN7917.2) promoter;
Pichia pasterns Pp03g11380 (ScPMP47) promoter;
Pichia pastoris Pp03g08340 promoter;
Pichia pastoris Pp05g04390 (ScTIR3) promoter;
Pichia pastoris Pp01g08380 (ScYIL057c) promoter;
Pichia pasterns Pp01g05090 (ScSAY1) promoter; and
Pichia pastoris Pp01g13950 (ScTPN1) promoter;
operably linked to the heterologous polynucleotide, and, optionally, to a transcriptional terminator; comprising culturing the fungal host cell in a growth medium comprising methanol.

13. A method for methanol-repressing expression of a heterologous polynucleotide in a fungal host cell, wherein said host cell comprises a promoter selected from the group consisting of:
Pichia pasterns Pp03g11420 (ScARO10) promoter;
Pichia pasterns Pp02g11560 (ScMET6) promoter;
Pichia pasterns Pp01g08650 (ScYNL067W) promoter;
Pichia pastoris Pp01g01850 (PpPDHbeta1) promoter;
Pichia pastoris Pp03g03020 (ScSAM2) promoter; and
Pichia pasterns Pp03g02860 (PpSAHH) promoter;
operably linked to the heterologous polynucleotide, and, optionally, to a transcriptional terminator; comprising culturing the fungal host cell in a growth medium comprising methanol.


SEQUENCE LISTING (76 sequences)

SEQ ID NO: 1 - DNA, 1068 bp, Pichia pastoris
atgatgtacaggaacttaataattgctactgcccttacttgcggtgcatacagtgcctacgtgccttccgaaccatggagcacactgacacctgatgctagccttgaaagtgccctcaaagattactcacaaacttttggaatagctattaagtccttagatgccgacaagattaagagagaggctgtttcccagattggagatggacagattcaggcggctacaatcacctcatctgaaccgaaagtaactgcccaagtagtttcccaaataggggacggccaaattcaagccacgacctccacttcatcaaaatcgaaagaaaccgctcaagttgtttcccaaataggtgacggtcaaattcaagccacgacctccacttcatcaaaatcgaaagaaaccgctcaagttgtttcccaaataggtgacggtcaaattcaagccacgacctccacttcatcaaaatcgaaagaaaccgctcaagttgtttcccaaataggtgacggtcaaattcaagccacgacctccacttcatcaaaatcgaaagaaaccgctcaagttgtttcccaaataggtgacggtcaaattcaagctacgacctccacttcatcggaagtaaaacaaactacaggagttgtctcccaaataggagatggccagatccaagccactacagccactacatctgtcgcttctcagattggagacggccaagtgcaggagtcaaaaccaacggacacatcagaggataaagggacttctgatttagtgtcttgccttactgattcttctcttgctttggttcttgaaaagggtgtgctgacagacgctcagggtagaattggtgcgattgtggccaataggcaatttcaatttgatggaccgccaccccaagctggcaccatatatgcaggaggatggtcgattacagacgatgctaagctggcccttggtaacagtacaaccttctatcaatgtctttcgggtaccttctacaatctctatgacgaaaaaattggcgaacagtgtgaaccagtcgagttggacattgtagacctcatagagtgt

SEQ ID NO: 2 - DNA, 609 bp, Pichia pastoris
atgcaatacagatctctctttttaggttccgccttattggccgctgctaacgctgctgtttacaacaccaccgtcactgacgttgtttccgagttggagaccaccgttctgactatcacctcttgtgctgaggacaagtgtatcaccagtaagtccaccggattgatcactacctccaccctcaccaagcacggtgttgtcactgttgtcaccactgtctgtgacttgccaagcaccaccaagagctacgtcccacctgctaagactactactattcctcctccagagaagactaccaccactgtcccacctccagccaagactaccaccactgtcccacctccagccaagactactagtaccgtcccacctccagctaagaccagctctcaccatgagtctaccatcactgtgactgtcccatcctccacttctaccaagaagattgagactgagtctaccacttaccactttgttacccagaccactactgctagaaacattaccccaccagccatcaccacccaatctcacggtgccgctggtatgaacgccgccaacttcgtcggattaggtgctgccgctgttgccgccgctgctttggtcttg

SEQ ID NO: 3 - DNA, 912 bp, Pichia pastoris
atggctgacaacaacaagtctaacttcttcgtcgacttcatgatgggtggtgtttccgccgccgtctccaagaccgctgctgctccaattgagagagtcaagcttcttatccagaaccaagatgagatgcttaagcagggccgtcttgctaagaagtacgatggtatcgctgaatgtttcaagagaaccgctgctgatgagggtattgcttctttctggagaggtaacactgctaacgttattcgttatttcccaactcaagctctgaactttgctttcaaggacaaattcaaggctatgttcggtttcaagaaggatgagggatggtggaagtggttggccggtaaccttgcttccggtggtttggctggtgccacttctttgttcttcgtttactctttggattacgccagaaccagattggctaacgatgccaaggcttccaagggttccggtgagcgtcaattcaacggtttgatcgatgtctacaagaagactttggctactgatggtattgctggtttgtacagaggtttcttgccttccgttgttggtattgttgtttaccgtggtttgtacttcggtttgtacgactctttgaagccaatcgttttggttggtcctcttgaaggttctttcttggcttccttcctgttgggttggaccgttactactggtgcttctactgcttcttacccattggacactgttagaagaagaatgatgatgacctctggtcaagccgttaagtacaacggtgctttcgacgctttcagaaagatcgttgctgctgagggtatcaaatctttgttcaagggttgtggtgctaacatcctgagaggtgtcgctggtgccggtgttatctccatgtacgaccaattgcaaatgattatgttcggaaagaagttcaaa

SEQ ID NO: 4 - DNA, 1227 bp, Pichia pastoris
atgctaaccaaggttatttcactcgctattttaactgcttcagcctttgctgattctggagagttcactctttggaacttgtcacccggtgacccttacgactcaactttctggggggtatctgaaggtttaatcgtccctgtagaacctggtgtgacttttgttatcactgatgacctacagcttaagactactgatgatcaattcgttactgttggtgaggactccgctctaggtttaggagctgaaggttcggtagaattcagcatcatcaacgaggatggcattacctctctttactacaacggtgagcttgttactgcttacatttgtgagggtgcagaaccccagatttatctcacaggttcagaggaggaccccgaatgtgtttcttacactgtcgctgtgataggcgtagacggcgaagccccaccaactttcccagaggaagacgacgagacaacaacaaccgatgatccaaccgatgagccaaccgatgagccaactgatgagccaactgatgagccaactgatgagccaaccgatgagccaactgatgagccaaccgatgagccaactgatgaaccaactgatgagccaactgatgagccaactgatgagccaaccgatgagccaactgatgagccaaccgatgagccaaccgatgagccaactgaagagccaactgaagagccaactgaagagccaaccgatgaaccaactccacctcctcctcattggggaaatgaaactgtaactgctactaagactgagtatgaaactacaaaagttactatcacttcctgtgaggaaaccaaatgctatgagactacttctgatgcttgggtttctacttgcaccacagaaattggcggaaaggtaactaaaattgttacttggtgcccaatcccatctactccaggtcctaaaccacctaagcctaccaagccaaccgaaactaagccaaccactgttcctgcaccaaccactaagaagccagaaacaccaactactaagaagccagaaacgccagcccctgagaagccagaaaagacaaccactgttattcctccaccaaccactgagaagccatccactttgtctaccagtagtgttactggaagtgttaccattccaactataactgccactggcggtgctggttccaatttcaacttgggtggattaaccgtcggagttgctggtattgcaatggctttgtttgtg

SEQ ID NO: 5 - DNA, 1778 bp, Pichia pastoris
atgcagtttggaaaggttctatttgctatttctgccctggctgtcacagctctgggagaaacaacttcttcattgagtaagtattgatcatttgaaattttttcgggagttttttttgatgttctgcaaaggaatccaagaaacatttatgaggttaccaggtaggtttttgttcagaagtaactaggttgactcaagctttcaactgttcaatctaatagcctgaactgtttgagtttacctggtagtcctgagtctttcaattacgagagaatccctgtgatccgatccctgtcgccaaattcaaatcgcccaagacggtcagccaatttgttttctgatggcttgggcgctttatgcgtcagggacgaggaactacagttttgccatacatgatacgggactatccaatgcagctcaccctttgtggtgtgaatcttgcccgtttcctaaaacgaatcaaacgggcggagctcaatggcagttccacatctgtatggagctatttttagatttttggttcctttcgcgatttgttgatcatgacactcccctaacatttcattttcttctgagataacgtactaacaatatgccaaggtgccacattgacttcaaccacacgaatttcaattgcatctggatgtagtcttgaagacttcactgctactgctcagtctaatcttgatgagttgtccgattgtgaagctgtcagaggtgatatccacattgccggaagtctgggatcagctgctatcgctaatgtcaaggccatttacggttctttaattgtcaagaatgccacctctttggtttcattgaccgctgattctttgaccaccattaccgaacaattggctctttctgagttgaccattttggacacactctcttttgctcaattgacttccgttggttctatttacttcgtcaccttgccagctttggaagagaccggtttccacactggtgtctctgacactgagtctgtttacatttctgacactgctttaaccaacttggacggtatcgttgctaccgatgtcgatgttttcaatgtcaacaacaacttcaatttggacactgttgactcgcacttggaaactgtttcctctgctttggaggtttccttcaactcagacgatgttgaagtatcctttgacaatttgttgtgggctaacaacattaccttccgtaacgttgcttccgtctctttgaacaacctgaccactgttaacgcttctttgggtttcattgagtctggtttccaaaagttgagtttcccaggaatcaccagggttggtggatctttctccattgttgacaacgacgatttagaagaaatagacttttctaacgttcagtcgattggtggtggtttaatcatcgctaacaactctaagttgactgacttcagtgaatgggaggacttgcaaactgttggtggtgctcttgttttggagggttctttcgacaacggttccttcccatctttgagagctgttagaggtgccttctctttggagtctgatggagatatctcttgtgatgacttctctgatatcaggggtgacactgccggtgagtatcagtgctctgctgcttcgttctccacttctgctagtgctcagtcttcttctagttcaaccagtactggcggatcctctacccacaccggtagttccactgctaccagctcttctagtgaggatgctggtgtagctttggctcctgcttctctcttcactttgttagcctcaattgttctcggattcttg

SEQ ID NO: 6 - DNA, 195 bp, Pichia pastoris
atgcaattcaagtctatcgttttaactctagctgccgttaccgttgctcaagctaacaacctatcaaacgagagtaatggtactaatcactccaaccatacttcttccgtgccaactggagctgccgttcgtgcctctggtatgggagctggcttgttgggagctggtgttgtagccggtgttgctctattgatt

SEQ ID NO: 7 - DNA, 849 bp, Pichia pastoris
atggccgtaccagcattttccgacatctcaaaagcatctaatgatgtactaggcaaggacttttatcacttgaccccagtctctttggacgtgaagactgttgctgccaacggtgtaactttcactgccaaaggtaaatctgccggtgacaagctatctggaaacctggagaccaagtacgccgacaaaaagaacggtttgactttgactcagggctggaacactgccaatgccctagacaccaaggttgagctggctgacactttgactcccggtttgaaagctgaggttgtcggatctgttgttcccgacaagaagaaggacgctaagctgaatttgacctatgctcaccaagcattcactgcccgaacattcttggatttgttaaagggccctacagtgaatgctgactttaccgctggtaaagacggtgtcactttgggtggaactgcttcctatgacattaatgccgcttccgtcaccaaatatgctttcgctgtcggatacaaggccccagactactccatttctctttctgctttggataacgtcaaactgttttctgccggttattaccacaaagtttctcctctcgtcgaggtaggtggtaaagctacttacgactctaagtcctctattgcaaacccagttgctttggaagtagcaactaagtatcaggttgattcgacagctttcgttaaagccaaaatagctgattctggtattgcctcatttgcctactcccaagatttaagaaagggtgttaagttaggtttaggagctgctattgacgttctcaagttgaacgaggccactcataagctaggtgtatctctttctttctctgct

SEQ ID NO: 8 - DNA, 933 bp, Pichia pastoris
atgatctttaatcttaaaacactggctgcggttgcaatctccatttcacaagtgtctgcagtttcctctctgggttttgctctcggaaacaagaacgttgacggaacttgtaagtacttggccgactacgaggccgacttggatactattagaggcggctctgaagccgttgctattagagcttattccgctgaagactgtaacactttacaatacctcggtcctgctgttgaagagaagggcttcaaattagttctatcagtcagaccactggatgagagctactaccaggcagaaaagaatgcactaagtgaataccttccccaattatctgtttcgactttgcaatttttgtcagttggatccgaagctttgtacagagacgacttgccagcttcagatctggctgataaaattagagatatgaaggagtttttggctggcttgactgacaagaatggggactcctactcttccgtcccagtcggaaccatcgattcatggaacgtccttgtagacgcctccgctgcaccagctattgaagcatctgatgccgtttacgccaacgctttctcatactggcaaggtcaaggtccttccaactctacctattccttctttgacgatatcatgcaagcattgcaagtaatccagaccatcaagggatctactgatatcgatttctgggttggtgagaccggatggcctaccgacggtgacaactttggtgatgctgttccatctattgagaacgctgacaacttttggaaagaagctatctgcggtatcagaggttggggcattaacacattcgttttccaggcatttgacgaagactggaaggaagaggacgacgctgttgaaaaccacttcggtgtttgggacagttccagacagttaaagctggactcattaggttgcgacttttcttct

SEQ ID NO: 9 - DNA, 2340 bp, Pichia pastoris
atgttatccgccagaagagtacttgctaagataaacagccgtggattggccacggtgtcagggctcaccaaggattctctcgttgagatgaacctgttggaaaagggcaactacattaattacaagcaacaacttgacaacgtcaatattgtcaaggaaagactgggaagacctttgacctacgctgaaaagctgttgtacggtcacttggacaagcctcatgaacaagacattgagagaggtgtctcttacttgaaattgagaccagacagaatcgcttgtcaggatgctaccgctcagatggccattttgcaattcatgtccgctggtatgccttctgtcgctactccaactactgtgcactgtgatcacttgattcaggctcaaaagggtggtgctgccgatttggagcgtgccatcagactgaacaaggaagtctacgatttcttggcaaccgcttgtgccaaatacaacattggtttctggaagccaggttcaggtattattcatcaaattgttctggaaaactatgctttcccaggtgaattgctgatcggtaccgattcccacactcctaacgctggtggtttgggtcaattggccatcggtgttggtggtgctgatgccgtcgatgttatggctggtttgccatgggaattgaaggccccaaagattatcggtgttaagctgaccggtagaatgaatggatggacttctccaaaggatatcattctgaagttggctggtatcactaccgtcaagggtggtactggtgctatcgtcgagtacttcggtgatggtgttgacaccttctcttgtactggtatggctaccatctgtaatatgggtgctgaaattggtgccactacttctgtgttcccattcaacaactccatggttgacttcttggacgctactggaagatctgagattggtgagtttgccaaggtcttccaaaaggagtacttgtctgccgaccctggttgtgagtacgaccaggttatcgagatagacctgaacaccttagagccacacattaacggtcctttcaccccagatttggccactcctgtctccaagatgaaggaggttgccgttgccaatgactggcctctcgaggtcaaggttggtttgatcggttcttgtactaactcctcttatgaggacatgaccagagccgcttctatcattgaagatgctgcctcccatggtgtcaaggctaagtctttgtacactgtcactccaggttccgaacaaattcgtgctaccattgccagggatggtcaactgaagactttcactgacttcggtggatccgttttggctaacgcttgtggtccatgtattggacaatgggatcgtcaagatatcaagaagggtgacaagaacactattgtctcttctttcaacagaaacttcacttctagaaatgacggtaacccagctactcacgcttttgttgcttccccagagatggtcaccgcttatgctattgcaggtgatttgagattcaacccattgactgacaagcttaaggacaaggacggaaacgaattcttgcttaaggaccctgtgggagtcggtcttcctgtccgtggttacgaccctggtgaaaacacttaccaggctcctcctgaagacagagcctccgttgaagttgtcatttctccaagctcagaccgtctgcaaagactgactccattccagccatgggatggaaaggacgctgagagattgccaattctgattaagtccgttggtaagaccaccaccgatcatatttctatggctggtccttggttgaagtaccgtggtcacttgcagaacatttccaacaattacatgattggtgccataaacgctgaaaacggtgaagccaacaacgttaagaaccactacaccggtgtctactccggtgtcccagacactgccgccgcgtaccgtgacaatggtgttaagtgggtcgtcattggtggtgagaacttcggtgaaggttcctccagagaacacgcagccttggagccaagatacttgggtggtttcgctattatcaccaagtccttcgctcgtattcacgagaccaacttgaagaaacaaggtctgttgccattgaacttcactgatcctgcagcttacgacagaattcaacccgatgatgaggttgacattttgggattgaccgagttggctccaggtaagaatgtcactttgagagttcaccctgccgacggctccccaacttgggaaactccattgtctcacacctacaatgccgaacagatcgaatggttcaagtacggttctgctttgaacaacatggctgccgtcaaggcctctaaa

SEQ ID NO: 10 - DNA, 246 bp, Pichia pastoris
atggttttagtccaagatttattgaaccccaacccagtctccgaggccaagcaacacaaactaaagactttggtccaagctccaagatccttcttcatggatgtcaagtgcccaggttgttttgagatcaccactgtcttctctcatgctcaaaccgctgtaacctgtgattcatgcaccactgttttgtgtactccaaccggtggtaaggctagattgtcagagggatgtgccttcagaaggaag

SEQ ID NO: 11 - DNA, 168 bp, Pichia pastoris
atggctcacgaatcagtttggttttcacacccaagaaactacggaaagggttctagacagtgccgtgtctgtgcctctcaccaaggtttaatccgtaagtacggcctgaacatctgccgtcaatgctttagagagagagccaacgacattggattccacaagtaccgt

SEQ ID NO: 12 - DNA, 906 bp, Pichia pastoris
atgcatagaacttactccctacgttcaggccgtgctcctacggctgctgatgtcaacaaccctccaccacctacctctactaccaagagcaagttctttggtaaaaactccattgcttcttctcttcgaaaaaacgccgctggcgcttttggacctgaattgtccaagaagttggctcaattgatcaaaattgagaagaacttggagcgatccatcgagcttgttgccagagagagaagaactgttgctaagcagttgtctctgtggggtgaggaaaacgatgatgacgtgtctgatgttaccgacaaattgggtgttctcatttacgagattggtgaattagaagatcaatttgttgacaagtacgatcaatacagaatcactctgaaatccatccgtaacatagaatcttctgtccaaccatccagagacagaaagcaaaaaatcaccgatgagattgcccacctcaagtacaaagaccctcaatctccaaagattccagtcttggagcaagagctcgtccgtgctgaggccgaatctttggttgctgaggctcaattgtccaacattaccagagagaaactgaaggctgctttcaactaccaattcgacgcccttgctgaattgtctgagaagctagcattgattgctggttatggtaaggctttgctagagttgttggatgactctccagtcactcctggtgaaaccagacctgcctatgatggttacgaggcttccaaacaaattattatcgatgctgagaatgctctttcctcttggactcaggacaatgctgctgtcagaccgtcactttctatcagaaatagtgaatatgatgaggagcccaacgaagagtgggaagaaaaggaagagggtgacgaaactcgtgacgaaactcaagcc

SEQ ID NO: 13 - DNA, 1002 bp, Pichia pastoris
atgttgtccacaattgccaagcgtcaattctcctcctctgcctctactgcctacaaggttgccgttcttggtgccgctggtggaattggtcagcctttgtcgttgctgatgaagttgaaccacaaggttactgacttagccctgtatgacatccgtttggctccaggtgtagccgctgatgtatcccacatcccaaccaactccaccgtcaccggttacactcctgaagataatggtttggaaaagacactgacgggagctgatctggtcatcattccagctggtgtcccaagaaagccaggtatgaccagagacgatctgttcaacaccaatgcttctattgtcagagatttggccaaagctgttggtgactacagtcctagtgctgcggttgctattatttctaacccagttaactccactgttccaattgttgctgaggtcttgaagtccaagggtgtctacaacccaaagaagctattcggtgtcaccactttggatgttctgagagcctctcgtttcttgtctcaagtgcaaggtaccaacccagccagtgagccagtcactgttgttggtggtcactcaggtgtcactattgttcctctgctgtctcaatctaagcacaaggacttgccaaaggacacttacgacgctctggtccaccgtatccaatttggtggtgatgaggttgtcaaggccaaagatggtgctggatctgctacgctgtctatggctcaggccggtgctagatttgccagctccgtgttgaacggtttggccggtgagaatgatgtcgttgagccatctttcgtcgactctccattgttcaaggatgagggtattgaattcttctcctccaaggttactttgggtccagagggtgtcaagaccatccatggtttgggagaattgtctgctgctgaagaggagatgatcacaactgccaaggagactttggccaagaacatcgctaagggtcaagagtttgttaagcaaaaccca

SEQ ID NO: 14 - DNA, 1541 bp, Pichia pastoris
  misc_feature (1)..(1000): Promoter
ggcaaactgtttagagttgtgacgatgatcatgtccaataactattgatttaccgccagcttttgacatatagttggccagtccctgagatgcttgtattatcgtgagatcattcattagactaaaaccagcacccattctacctcgaagtcccgctgttccaaaagtaattcttcttcgtaagcgttcttcaagaactttaaacttttcattctcaagaagtttgagaatctctgatctagtggatggattacgatcaatgctaagccattgatgcgctaacgccttaattgatggggacatgatgtcagatgtaataccttgtttaccgaaatttagctatctcacgtagcctagaatggccgctgctaatcggcctatatctaaaacggcatatcgaggatcgatgaagtttacacgtgacgcattcagtgtaagtaaattacatgcagtgccggctacttgactaaagtaagaaaaaactcagaaatcaaagaaataacagaagctgtacgacacaacgaaataaatacaaactttcttaggatgcaatttaacaagaatcgtcttattgtctctcggaaacattcaagaatttcagcccattatccacattcagtcatggcgcattatttagattgtgttgtttgataaaaaaagaatacagcaagtaacaacgagtagtcagttactgagatattttttagtaccgtcgatccggttacggggaatttaactgcaatatatcaaacgttaaactggatcgagttcaaaaaaacaagctctaactcagtcaagctttatgtatagtttcctaatcaggtaattttttatgatcttgtctgtaggtttgattaaatttccattacatctaaaaatagtaacccagccatctgaaaaatggatctttcgggattcaatattcacagtataaaaggaagacttctcttaggattctgacccgttcatactcattgctaaacatattaaatactgagaagcggccgcggcgcgccttaattaaatgttgggtttatggtcatttcatgatattggctgttcttgtgtaaaattttaacttcttcgtataaaatgcatcttattttttgtatcatgaaatcgtagtgtcgattgagtggcatcaaactggactcttttcgcgttactaagcttttggatgtgcaattacatttggcctcaatactaagacccatctgcagtaaaagttgagaccttagtaaggaaaacacataacttgattactttgggtcgctaaactttgcaacatcaatctgcatggtcagaaaactcgtcttcgcagtgtactgcacaccataatagttcacatctttcgcgcttccgtctagtggtatgatttccccagaaaacacaccacatcagccaaagatagcatgattttgaactaaatgagatgaagattatataaatttgaccgcagtctgaagggctataaatagggcttagttcttctccccatttaatagtcagaataactactctactcggaccgatttaaatgg

SEQ ID NO: 15 - DNA, 1540 bp, Pichia pastoris
  misc_feature (1)..(1000): promoter
ttttcgtttgggttcattaactgacgtatgacatggcgtggacagcaagtcgtccaaaaataactaggcagctgcgacattttttcgtagagctagtttcattcgaacctgaatttcggtatgcatggcatggcgaacgtaaacaaaaacctttgcctctgttgaaattaaccaccaaaaattgatggtctcatgatgtggaactcacgatatttgttctagcccctcccccgtggtaatcacaagtcgaagtgtgcccactttttcagtgtaagtattgtgcctaacatatatcattattctatgtgataaacgccacaagtcaaaaagactgacacgatgatactctccagggtcctgttgactctactccttacaaccctcagggaccagagcagttctcggtaactactgtgacgtgagcagagtcgaacaagaccgaacgtgggaccagtaaaatgcaatctgcatgcacagaaacgaccgtgattgtgtccaatgtaagaagctggaattgtgcatgccatgactaggttttgaggctagtaccaaatggaaaagtacacaccaccaactgggttggtggtgacagtcagagatccagaaccattgcaggcagtttccagactctgctgagtcactttcgtgttcaatgaaagtaaatggaagtaaatcccttacaaattgtccaattgagaaataacatcttcaaactgctgttcgcatttgccgagaaaaagcttccctatttgggttctattcaaaggaaaacatcaatgcctgatttaccctggcaatgatgtgttcttatgtatgatcttatgcacacccctgtccagaaagatccacaaaaatcctattccattctgggtcgaacctaatcccaagccatccaaaaatggctaataagagatttaaaaatagtagatatcacggtatataaacagccaattagtcctttaattggcttgaagataattaatataactaccgttcaatagcggccgcggcgcgccttaattaaattgcttattattaacactgtattctattgtttctcattgtagccacattgcacagctctgttcttattattgccaatctgaaagttaagcctaagtccgtttttaagctattgatgtacaacatctttcacgtgatacagatgctgtctgcccatgcatgcctaaatctaatccctaatatactacatttcgcttattttaccctcactacttctcctagattatcggtgaataaaatgctgcggctagataaaatccgccaatctgctaacactgactattttggctaggttcgtctgtgtttatctttaaccaggttattgttcatctgtgttatttttgtattgtgttttgcaccatgcgcgtcattgcgcataccaagcattgagtaacaaagagagattcaatcatgaacaataagcaaaacctagttacaataagggagtagtataacagcttatgcgttatgcattaatcgactaaaaactcagcaaaccacggaccgatttaaatgg

SEQ ID NO: 16 - DNA, 1541 bp, Pichia pastoris
  misc_feature (1)..(1000): promoter
cttgccaggatctgaccactacaagaagcgtaattaagtatttattatgtagcttgaggaattgatttctaaaggccaaagatgatgtagagcaatagtagagtgattagcaagtttctgaacatgttttgtggtcgggtattaggcgccgtacctctgtattttttttttttggcagatgggatgaccactgtgcttacataattttcgatgggcccttttggaaacgaccactccttccgatcacgtaatactaggcgagtatcactacgcgtcggtatccgcattttagccgctttaacgacaaggtcagggtaaatactgtgttgcatgggcgatgccgtaggatctggccaacttggaacagagcagcccaccgcgtaggcagcgcttgggaccagtttgttagccaatcagttcccccgtctgagtccctacacgctcaggcttgcatggatatgcatgaaaaatgtcaactcagtcgcacacagggatgctgttgtcgtggtgagaaaaagctaaaatagcaaacccatagcaatcgagagactgtgattggaagcaaaagaaccaggcattgactaggaaagactgacaagaaaattagacggtgccagtggtatttcattgaattgcactgaacacgcctcccatttccttccctcaatccattcaaagggcaattaaaccttatctcggcctacattagttgtcatggaaacgtctcactttctctctgtgtcccacagtagcgagtaggcggagagaccaaaaatggtctctcggccctaatcaaaacatcctttgtcgcgttgttttcagatcgattcgcaatctgagagaaaaacaataacaccaaaataaaagcttggaaaaagaaaacatgattcacatccactagtaaaatataaatactcctgccgcctccccctttcctttcttcttattcctcaactcactgtttcagtttattccaactactttcactcacttatcaaaagcggccgcggcgcgccttaattaagtttgatcactgaattacctcacacggttgtattttagcaaaatttcttcgtaattaaccaatttttttatttacaaactttgtagaggtcgctttttcaagagataaagagcgatcggccttcggttttttattccgctttgggatatctccgaagacttaaggttaaggcttatatattgcgcgatgcaccgtttctatttctctcccgcttttcatccttaagtccaatcagtatggcttttttgtcgaagttcttggggggcaaatccggcgaagcccaggcggctgaggtgatctcagaaatagttcctctttcacagtgccccgctacttgttcggccgctgagtgcttcacaaggtatccaaacagttacaaagatgacccaggctccaaaaacgatctcctttggaaatcaactaaaccgtactctttacacattatcattccaacgggtaagtccgattggaagcgtgacgccactgatgttgatggcacgcggaccgatttaaatgg

SEQ ID NO: 17 - DNA, 1541 bp, Pichia pastoris
  misc_feature (1)..(1000): promoter
ttgttgttcctaaggctctgagagttctgagattgaagccaggtagaaaattcaccactgtcggaaagttgtctacttccgtcggttggaaatacgagtctgttgttgagaagttggaggagaagagaaaggctgaggaagctgagtaccaggagaagaagagagcttacacccagagattagacgcagctagtgccgagtttgcccaaaccgaggagggaaagcagttggctgcctttggttactaaatagtaaagtagggtatcttcaagtaatagtatactaaccatctgaaataaccaccgtcctgtagttttttttcgatatcgaagagcctatgctagtactgtggatttgcgctccatccaacatctgtgcgcaaactaaaacttccgagactgacatctaccatcgctagaccctaagtaaaaccaatctcgcgtccgaacttttaaatttcagtccttaaaacttcagagcattggttgtagtttccggatctgaggggtcgtattggagtcaagagacggagctgcctccacagcgcgaaacgtcaaccccaacaccaacctgaatttgcaatcaccatggggacaagtttcagcagtcaatgggcaattcagacgttgatacggtacccatttgctaagctcaatgacgatccatccaacttcagagaaaggcctttctctggtatgctctggtattcattcgtcttttatcactctcgttgcacaatgcccgggtactcccggaacaagggagtcttccagccaagctgtacagagtgaaaaatagaaatacacctttgcaatcaagacgcgcgttggccaatcacaagacttaatcggtgcaaagaaggattaccaaatttttttttcccaaaatcgctatatagaaataatggaggaaaaagggttaatataaaggagaattcccccgtttttctccccttttcttttcttcttcaggctttcttacaaatctataatattccaaagcggccgcggcgcgccttaattaagccaattagtttgaagtgagattttatttcattcctgttaatattatatactagagtatattttaaattaattgttcatgaacttgccaaattatgttagttttgtgtaaacaatcttaggctatccaatttagttctacttttggtagatttcctgttttggtaaattacaaacaacaatgatttgacttatattctattcggaattttacttatcaccttgtacagtttgtggggatttccggacatgaagaagtttggactggaaaaggacggagatggaatcttcaggctcaaacgaaggtcctcctctcccaataagaagggcaaaacaacattgcctcctttctcgtcacctccaaatctagcccatgacagtccaacagggcccccaatattacctactctcagaaccagagtttctgaaactgacactttggtagatggtatcagtgatacggtcaacattccctctccacaagctatgaatgcgagggccgcggaccgatttaaatgg

SEQ ID NO: 18 - DNA, 1541 bp, Pichia pastoris
  misc_feature (1)..(1000): promoter
cgtgggaagaagggttggatggcttatccaaattgatcacacctgaagagatatagtccttcaagtctcttttcaagggagagcacccgtgagggataggagtataatgcccacttctaaccaacaacttgtcgtagttcttcaagagcaaaggccagtccgaagtatcattggaaggagtcacggcctctggcttaattgtgtaatcagtttcacccatgataagttaacgtaaagaaaattttcagtagaaaagaaaaaaaaaacctaacctttaagtgtcgtgtactcaacaaacgatgttggatgaaccttttcacatagtcactggagttaggaagtgcgatgcggtattacattacagatggatgaaggcctcccgcaatgctttagtttgaacatctacgtgggatttaaggtgcaaccaacaaccggagtctgttaatctaaattgggaacaacggacattgacaaaaaagaagcggctatttgtttatgtttaccatgaggatttcagctcaatccgtcttatataatctgtcacctacttgagttttgtggccttttcaacaacggaattgagatacagtttgggatttttttcatgctaagtcgtatttctctttcaccctttgctgaactaatccaaagtttattttgatctttcaacgtctacacattttgagctacttctgatcttctcttcgagttttgggcttcggtacccgtaacaatagtgcaaactgctagtgtgtaaatattcgcgcatcgtgaataatcctcaaacctacatgttgatgcaactagtttaccctttttagtattattccccgatctggacaaacaaaaataacgtctcacaacaaatcaagcctcgattttttgctctggaaaatatcaaccgacgtactatattaggcctcctaaggccattttttttttccattttctcttcacatccttgcgcttatttttttttgctctaattaaaagtccaaaggcggccgcggcgcgccttaattaaacatagagaaataaaaagaagaagcagaaaagttgaatggaatatttgtattgtttagttttattttaaaatttagttcgtaatgaatgttttataagtatttctatacgtacttacaaccccttaccgtagtttccagactcaaaaaaggaaaagataatcttgacaatgttttctaaagaggccaaccattccaagagttgagcggtgaccttttcttggtcatctcccaattttttgaaaaagtccgctctatatggacaggctttcatagctaacttgaaaattggcttgatcacgaacgaatgatgcttacttaaggttttaccataggcattagtgaaagccttcgtgagttcatagtctggattgtctacagtctctctcatagctgctgcagtaaattgaaggcctctagatagccataataaaccttgggtagcggatttgcccttgatcgatgcgtctttctcatccaaaaccaagtcttgaagcgtagccggaccgatttaaatgg

SEQ ID NO: 19 - DNA, 1522 bp, Pichia pastoris
  misc_feature (1)..(1001): promoter
gtccagaaaacttatgaaacgaaaatttctcgttgacaaaagaatcaagtagttgcaaaatcactgtttacaattggacatttcgaatgtactatgaggcgatcgatcttccttgattgaatagtccaatacggaaaatgaaatcgtgtcgatttattacttaatcggtcgaggtatgttcgaacggtttcaatgaaagcaggagaagaaggttactctccaagaccacattgaccatccgagcccgccaggccctacttgacagccaggctactaaaactgaatgtaacaaggaaaagagagtttactacttttcgatctcctaaagctaaggagactggatggttttggttgtacctgcaggacaggggattcggtctgagttgaaaggccaaattgagattctgtgacaaacaaaaagagctggtcctatggaattccagattcttctatcttgcataaatacatgcttatttaggtaactcattttgcccaaataggaaatattggagtatcctttctataacagaattgtgtttaattcttatgtcatcctacgggcatcgctctatatcattcaccacgtacgttccaatgatagcgtaatagcgtgcgaagaaaagggaatagtaagccacttgtagaaaagtgttggctgatcactactcaaaatcagctccataccctgagttaatcagttcacgttgctcaagcgtctaaaaatagattgctattgcctgcgaattctcttaggcctggaacgttctactgggctcgcggaccgtgtttattcctgattctggaggtaaacggtggcttttgcagaaaacgcgcaggcagtgttaattgcaaattttttcttccgtcgtatttctttttcctgctggtgaaccttgattatttcccatataaataagggttgaagctccttgattttttttcttttcttgtatccaaaaaatcacactttgagcagtcaatatttataatattagccgaaggcggccgcggcgcgccttaattaagctgtttgtccttatacatcattccaaggttagaaaaggccggaaaacagacatttttatttcatagaatattgcagtaggatatatccgctacactcgcttatcagcccattttaatttaatattttagtttaattttttaactgatcagttagccttctttcagcaagggctgaaagatgtcgtgcgtaaactcaaatagttacttactttagcaatgatcatttgtttgctcttgttgtagccaagtttctcggcagacaaaagccgctcgttgaagtccattcggaaagtcaaaggtcatcgaaagtatttcgaaggggctgaatctcgaagggggctgaatctgcagggactttacgtgtcttatcgatcaaaagattaattctaaaacaggcaagaaatgcgcgaattaatcttacaactatagttgctgctgctttttagagcggtcaacgtttcttaccaggttgtttaatttattgattattcaattg

SEQ ID NO: 20 - DNA, 1524 bp, Pichia pastoris
  misc_feature (1)..(1000): promoter
ttatgccatcgtgaacacaaatatcacaggtgcatttggtgctatttcctggtgtctattagactggcgtttggagcgccgtttcagtactgttgctctatgctccggtgccatttcgggcctcgtggcagcaactccagcctcaggtatcattcctctttgggccagtgttattcttggtattgtatcaggagtggtttgtaactacgcaaccaagattaaagtcatttgtcgagtcgatgattccatggatgttctagcagagcacggtatcgctggtgttattggtctcgtcttcaacgcattatttgggtcggctactgtcattggttatgatggccttaccgagcacgaaggtggttggatagaccacaactggaaacagttgtacaaacagattgcattcatttttgcttgtattggatactcgatggccatcaccgctcttatctgtttcatcctcaaccgtattccatttttgcaactgcgagcttcagaagaggctgaggagaaaggtatggatgaggatcagattggagagttcgcttatgactacgtggaagtacgtcgtgattttttggcttggggatcaggcccaaacaatggcttcaaggagccggaagttctggatcaggtagttccggttaatgatttcagcagtgaccagaatgtgactaatgagaccaacgaatctgagaagcagtagagtaaatatagagatgatatttagtgtattctaatgcttatgtaatgtattaagcaaaaagttgtgtttatgagttagcatttgtcttagcaaacataaaattatgtcgacatttgcaacccgcatgtctagtgtttttagatcgatcttcgatgtgtagaataatgcctccacgtgatgccccgcgattttgttgggtctcaatgcctccaacataaacccatcacgtataaaaagccctcttaaccctcccccctgtttcgtttgcttcatcacttaacctgaactatcaaagcggccgcggcgcgccttaattaataagcgttctaggtagcaagttttttaaagatgaaaaattagtaatatgatgagtactcgtatattgctgctatgtctagcgtacttctgattaccccactcggacgaactctggtttggtgttcttgtcgatcagtaaatcggttttgaatgtctcgatgtactccacatcgccctccccttttcccccggcaaaacgtccatactcatcaaatatgccagaaatacaccacccctgaagcagttttctcatgatacccactaatacccccaccctatgtttgcccttattcgaatgaattaacattgggtagttttctttgtgaacaattagcttcagagcctgctgaataaccgagtcatccttgaacataaacggctctacacagctttgcatatttaggtaatgaaaatctataccttgatcgcggagccaccggtaatagtcatagttgtccgtcttgtcacccaaatatatgattgtttttaggttcagtttt

SEQ ID NO: 21 - DNA, 1524 bp, Pichia pastoris
  misc_feature (1)..(1000): promoter
tctaataatctgaaaactcgatttgttaatattggacgagacaccctttttgaactagaaaaaggccaggaagtccccatgaacgtaagggttaccgttgatttgaaaaagaaaatagttgtatctcccttggatgcttacggaatcacaggtttaaaatcaaacttcggctacagtgtcagagctgtcaaggaatttcaccaaatatatacagagagcggtaatccagatggttactctcgatcctgtttcgttgaagcaggggattttttcataaaccaatctcacacgcaggatcacaatatggaaaagttaccaatgattgataattccgaggaatttaaagatggtcaagagcatcttttactagtgatcacaagatggagagagctccaagaatttttcaaagctgacaattccgacatgctcaaagagatagagagctgcgagtcaatgtttgataaccgtcttgagataatcaacggaaccaaactagaagatgcgattctgatagcccttgccaaactagagagttagtatgttgatagcatcaaagttctaggatgtcagatgtctagaatcgttctgattcgaattgttcattttgaggcatatccaaaccattttgggcttgtttggatgcaagtttcttcgcgcgtgtattgctccctacgttatcaccacgacaactaaccgtctagatccgaaacagtgagtccttcaattggaagttcgtctacaggtgacgggaaaagaaccataaaatcatagtaaataaatgaaatcagtatcttaattatccctactaacccatccttgttgctaggtatgctctcgtatagtgtctcctcaaaaactcaccgaagctaaaaatagaaccgtatgatgtggtcgttccccacccgacaatctcgatatttcaaacccatctcccgccctttccttttcagtgttttcttttagattagcttcttctactgattacttctctgttgcaaaggcggccgcggcgcgccttaattaatgagctctatcaagtcattttattattaccctcaaataggtcatatagcttttatacctcagacttaagcgtattacgtgattgagagaagggcgttagcaactattttgacattttttgactctgccacatttgattacataatcaaagaggccacctctccgacggagtcccaaaattctagtatatgaaccgagaatcttatccacccaccaaaaaccttgcacctttagctgatacgatcaatgctttctacacgtgtagttagactaccaaaggttattagcgggggcaataagcaactggcacgagggttggcctccctttcggatgaaaagtcgttaaacgaagcaaacccaacggatcttgcaggacttaaaagaaaggccaaaagaagacctacgaaacttgctgatgaattgaagacaggtccatcgttcgctgatttcgtcacggggaaggctaaggacatgctagtagatccattggaactggcgaga

SEQ ID NO: 22 - DNA, 1524 bp, Pichia pastoris
  misc_feature (1)..(1000): promoter
ttcatcagttgcccattcactcttatctggcttcaaggctttactgtttcccctttagctatcctgatgccctaccaaatcgattgttgcccttacaccaatgtacgatcacaacaattcccatgttgttagtccctaccatttgcgaagctcggaggcctaggtcgttgaacgtcacacattacagctgctccgaaattcgcataaggaacactcgcaccaatcacaaggtgatggaaattgcttacggagtagaaactcccaatcccgaaggtaaatacttttctagtgcaccccgacattcgactcaaaaggcttaaactaaactcctaaaatgtccgtggttgaccaatagaaagtatcactcagctccctgattgttcatagcctaactgtttctgaatctctccaagtttattgctgtcgggtgagcctatgattatcccctttcacaataggctcattgtgtcttaggaagtacctgcccacttccccctgataaactttccaccatccccggtcattcgctcatgaccttgttattaccatgccaaaacatcccataatgaaagggtatcggcaacatgggagctaaatttcagaccctcgagatggagtcggtaatcgttcgagaatcacatggccaccccacatttcaattgtagatcagactgtcaatcttgacataaccgtcgataaatgacttagatttccttcagatactcagattatcagtatctggactctcctgacctttttctatttgctccaaagtcttcgaatctttcctatcttgttggagtctgacaaactactttaacgttattggctgaattccaccctcggatccaacttctccttttcgctaccgagccaaggcaaatgtctgatgcagctagtttttactggcataccaggagatcgcattcgggacatatattatggaagttcccttcttttttttccttcttctttttcccttttgttcccgttatacagtgcggccgcggcgcgccttaattaataagttttagccacctacaaattccaataatcggctgtttgttctgattaggtaatatcatgaattatttattacatattttatttatttgtatccttatccaaaacaaggtttcagatctgcttagtactacgtatccgttgttgtaccaaccttgttaccactacgaccaagaatctcactgcggctcattcttttgtcttcagatctccacaatttgagagatatcactgcttttgtgtttccttgttcgaaacatcgactgagatatataaatagacaatgctgtccctcctacggcttgtttcctcagtttctttagcacttccaatagctttgtataaagatgaatactgaggatgtttactcgccagtcatcgctgacaatgggtctgaagatctcctaactactaccagcaaagaagtagtcaccccaggagaccttggtgctaagcttgaagagatcaaattagaatattctgaaaatgagacaccatccg

SEQ ID NO: 23 - DNA, 1524 bp, Pichia pastoris
  misc_feature (1)..(1000): promoter
ttggtataccacattactgaaatagagaccagtccaggtacaacgacagcttccaacgcacccaatagagctctgattaccatcaaactggcgtaggtgtcgcatgcagcatggcaagccaccacgattccccaaagagtgacacaagtagcaattgttgtagcgggagggaatttctgtaatgcccagcagactggggcttcaaaagcaacttgagctacgtagaagattgtactcagattgttgaactgatttcctaccagattatctttgatacccatcacagcagcatagttcaataagaacttgtctataaactgcaagaaatatattgctgacaggattgtcatgactcgaatgtcaatcttaaacttcagtttggatggaaggtcagttatcgactcagtcaggttgatattctttttcttgagatacttgaaggcctcatcttgtttaatcaaagactcatcatcggatgagctgttgcgtttctcgtggtccatttcgctcgctgaagaacgagcttagctgaaatgcaccctcttatattaaatcaagaaaattatccaacacctatcatggggtaacctgagccgtaggcatctcgtattgccctatttggacttaagggtgagtcagctacatatagccgaaaaagaaaaaggggaaaaaagcaagcatctgctacgaatagccgggcagactgaatcaaaaatgcattgaatggagcctacattacttttaacttttccagtccacactagcgatagattcaaattgatgattgcattgctctttactacatgatgaatcaaacgactcaaccccttttaggcaactcaccagtcagaaactcttggtagggagaggaaaaaatgaaaaaccgtttgtcacgtgatagggctgaagcctactttgtgccgggtaacttttttggatgttctcccacttaattctttttcagaggcaaaaatcttttctgcatcaagtatcctacaccgcggccgcggcgcgccttaattaataaaagaattattgaagatggttgaaagaagtggaacgatcaagaggataacgtgtaaatggtcaaaatcactatcgttatttttgcatagtcatgtacattaatgttcaagtgaagtgtaatattagagatgaactgtaggaaagttggacgtagccagacttttgtctgttctagttgtaagaagttgctattgtagttttctttggtcgtgtgagtattaatagttgatccttttgtctgtggtttagttgtaaaaatgtcggattatgcgttcgagtccaagatactgcattaattatcaagcaccgtttaatcctgcctcgtttgtcgtaaagcttatccacccctggatctcgccttttttcgcaccagcccaattcgttaatggaaccgtccgttccatttagagacgtcccgaaccagcagttggttatatgaattcgaacccctggcataaatgtcacatgaattcgtcatcacactccgatatcgtagct

SEQ ID NO: 24 - DNA, 1524 bp, Pichia pastoris
  misc_feature (1)..(1000): promoter
actgaaaggaagttggacatgtctggcaaaacaaatttaatattcgatttaaaacgttcaattctgaaggagttgatgctgggctggtctgtgattttcaaatcctgagagtttgctgtggagtcattaagagatactaacagcaaattggcaatatcgacgccatcaatatcatctgccaatgccaagatggtgacaactttctctgcaccattggagccagcaaacaattgtctttccaaagacgtctgaaggattttattttgtcgtaactgttgagcaatattctttctctcctgtttggttgttatcttgagtttcttattagttcctgtctccgatttctcaactttacccttgtaacttgccttcaaggagccctttgaggcatgtctcgacttgaagggcttatggttattcttaagagtggacctatgggaatgaccagccattgcttaggagaaattgtataattaaagtacactgtaggtctaaaattttcagatcctttaatacataattttttttttctcatctccctaagctcatcacgtgtaatataccgaaagtaagccgtatgtcatgtgcacaattccacggaaaggatgccaaatacatcccatcatcactaggtttgagccctagaatgacacgtgagatacagagtttttttttcccactgttcgcgcaaactggactgggttcctggagtcaatttgttcgttcctattcacttcgtacaccaaaaacatcaaagagatcaattattgtaagtttgaaccaccatgatattttaactggagtggagaggctgatgtattggatgtttatgtgaagccaaacaacaggtcacaccaaagaaattcaaaattcatgttgaaagggaaactatcaagaccatcagagagtaccagttgaaaaaaaaaaaatcctgcaaactcaagccagctcattcacttcagtaaaatcctaaaggacgcctactaacattactagcatcgcggccgcggcgcgccttaattaataaacaaattttaggtccttttcaaaaattcacatgtataatttaatctgaaattatctatcttttatttcctcaaatcgagagtcgatttgtgcctgttggcgaatgtattccagagtccgttggaagtgtttcgttggagtatcgatgcttgtattctcctcaccgattgtcaggatactgttttggatgagttcaatcaagccattgagatacttgatggtgatgagttcgttgccatgtatgaaaaagtaaagagatctacttagaatctccacaaacagttccagtgagacatttgtctccaagcaggagtctgctatccgaagggacttttgcaggcattctagaactttcttgttatcagttttaatcacaacctgcagttcatcctcactagttttggctgtctctgactctatctcgtcttcctcatccagctcttcgataatccaccacaaatgactagccagataaactgcacggcattgatctgtctt

SEQ ID NO: 25 - DNA, 1524 bp, Pichia pastoris
  misc_feature (1)..(1000): promoter
atgtgggagatttatgttgtttacaaagaggacaagacgttcattggatttgccaccggatactcgtattggaagtatcctggccatgaaatcttcgactccgatgcgaaatacctttggagaaagaagatctcacagtttgttatcttacctccataccaaggacaatctcacggaagccaactttacaagacgatatttgaacaatggttcaaggatgaccaagtggcagaaattactgtcgaagaccctagtgaggcatttgacgatctaagggacagatgtgatctggagagactttatcaaagagggttgttggaaacattaccggaaccagtaatctctcccgaatggtttcaaacacagcaagcaaaagagaaaatagagaagagacagttccagcggtgtgttgaaatgctcttattacacgccttgaaatcaaagaaaaacacccgtcttcaaataaaaaagagacttttcatcaagaacagagacgcactcttggacctcgacgaagccaccagaaaggacaaactgcaagttgcttatgaacgcttagaagaagactaccagagaatattggaaaaagttagatttgtcaaaaaacgacctcatcaggacacttaatatataattccgacctacgtaataacctaacaatatgaataataagatagaaacaaccctcccgcaaacataagcaagaacaattggaaacaatgccatacttgctatattgggttattttgattacctcattgggatatcgaatttctagttccaattcgaaattacggtcggaaatttctcggtttggcttttgatgcagcaatattccatcctcggaatggcgtaatgcaggaagcaccgaggatgacggcctcagtcatgttctttcgttttgaattgcttccttctcccaggagactgctataaaaatcaaacaatctctcccccatttcaagttcactttcatcaattgaaaacaaatcatcggaatgcggccgcggcgcgccttaattaataagatgtcccatttagtattagcattgaacgatgttgatgttgtctgagtatatggttaatacgtaatagctccggggagctaatttaagtctcccttaataattgattctagccaacaattagtgttattgtaatatcttttccttccaaaattccatagttctgtagaggctcatgatcgatagctaccaggttatgagtcgcaagcgtcaatatgccataaaagacactagctgcgatgctcttattacacccagcctcggtgatattgctggaaactagctcttgaaaggtgacttttgaaaacaccttgtttgaaatggggttaacttttcgttgaccaacaattgaagatttattacgcaaaaaatggtagaatcgctcatctctgggattcgaaggagctttgtcttccaaatgattgtacagagagtcatgggttgtaaactcagcatcaaaactctgttcgtaaagtgcttcatcaatgatattgtgtct

SEQ ID NO: 26 - DNA, 1524 bp, Pichia pastoris
  misc_feature (1)..(1000): promoter
ttgacggttatgcggaaggttcttgaactcataaatactccagtcaagtttgaatctggcattagaaaatagatttgctgtgtagctctgaactcgttggtctggcttgattattttttgggatagactgtgtttcggattgtatttcaagttgaggtcattagttagtgatttgggagaggcaatagattttttggaggcacttttagattgtttggtcgggcctttataaagaggtttggttgtggactgctttgaagagctcttagctggctggcttaaaatacgagagatattgacattgcccacatatttttccttcatgaaggagatgaggtgaaatattctttagtaatctagcagggaattcttggaagggtaatttagacggaagacgtcatcttgcggaagggtgaaacattccaagcagagtgctggttccgagattctccgctcctgtagaaaccttcaaccttccaacattagaaccttcgcgaatagactccctatccaatcacgtttctccgtttcaaggaagtagcctgggtgtccggtacgccaaaaaaaaaaaaggcctcaccggaacttgacaatttcattcattttagctcagccttacaacagattcaccagtccatactgctctgccatctgccaattatagtctccgagaaaaaaaaaacatcatgcttttcactgtatttcttccttttatccacacccccaaccgatcaaccctgcttcacgtcctcctcggaagtggtgctgcattgggcccgtttttagtttgttcccccacactgagatgaacaacccgactctcggttctcaattgcgcataaacgtcactttgaatagtctcgctaaagtaccggatatagctcggctacacctcgtattaccccgtaactatctgtcggagccgaccccttgattcacattatatatatcgtcgacaacttccactattctccaaccctcaaccattataatcccttcagcggccgcggcgcgccttaattaataagtttgattatatgtacttagattatttttcaatgaaatgaatgagatttggagatcctgggtgacgatattgaacgtagtcaaacagctaattttttttcttttttcccttcaaaatgagcacagtaaccatagttccttgcagagcactcacaaaagtgttgagtagttggttaccaagtacatttctggggcaaccggtttagtatctagtgggagattagcccactcgttaacagcactagggtatttttagaccccctatctgggctatgattcgggttagctccagcagcttccccatagagcgcagtctagcaagaagtcttgtcatgtagacccctctgctccgtgctgtaattatgcggtgaaagtgctttatccgatagactcctcagtcctttcaccctcactcatgctcctcctagtaacaacgaatctaaaatcctcatctgaaatttcccaatcaggaaatcttggtctcattttgctctcatc

SEQ ID NO: 27 - DNA, 1541 bp, Pichia pastoris
  misc_feature (1)..(1000): promoter
gtatttgacaggttggggagcaaataagtgatgatgtcccatgaaagtagaaaatggctagtagaaggcaaaaatttgaaattcttagagtcaaatagttagactccaagttctaatccacatttggtcagtttcatagcatccagagcttttgccactggtgaacatatctacccattgcgatgcaacaagtcactgaaagcctaaaacggagattcccctatcttacagcctcgttcaaaaaaactgctaccgtttatctgctatggccgatgtgaggatgcgctcatgcccaagagtccaactttatcaaaaacttgacccgtcatacaggctctagatcaagaagcaaacttaatctcagcatctggttacgtaactctggcaaccagtaacacgcttaaggtttggaacaacactaaactaccttgcggtactaccattgacactacacatccttaattccaatcctgtctggcctccttcaccttttaaccatcttgcccattccaactcgtgtcagattgcgtatcaagtgaaaaaaaaaaaattttaaatctttaacccaatcaggtaataactgtcgcctcttttatctgccgcactgcatgaggtgtccccttagtgggaaagagtactgagccaaccctggaggacagcaagggaaaaatacctacaacttgcttcataatggtcgtaaaaacaatccttgtcggatataagtgttgtagactgtcccttatcctctgcgatgttcttcctctcaaagtttgcgatttctctctatcagaattgccatcaagagactcaggactaatttcgcagtcccacacgcactcgtacatgattggctgaaatttccctaaagaatttctttttcacgaaaattttttttttacacaagattttcagcagatataaaatggagagcaggacctccgctgtgactcttcttttttttcttttattctcactacatacattttagttattcgccaacgcggccgcggcgcgccttaattaaattgcttgaagctttaatttattttattaacataataataatacaagcatgatatatttgtattttgttcgttaacattgatgttttcttcatttactgttattgtttgtaactttgatcgatttatcttttctactttactgtaatatggctggcgggtgagccttgaactccctgtattactttaccttgctattacttaatctattgactagcagcgacctcttcaaccgaagggcaagtacacagcaagttcatgtctccgtaagtgtcatcaaccctggaaacagtgggccatgtcttttgctccttcaaaaatggcaatgggtaggctgcctcctctcttgtgtatcctctctgggaccactcagcgtcacttgtgctaataatatcttttaggttgtgtggggagttgtgcaagattgcaccatctgtttctccgttttctactttacggatttcttctctaatagagatcatagagtcaatgaatctgtctacggaccgatttaaatgg

SEQ ID NO: 28 - DNA, 1541 bp, Pichia pastoris
  misc_feature (1)..(1000): promoter
ctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacagcggccgcggcgcgccttaattaaatcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaagacttttgggtcggaggactggctctttccgatcaaattggaatggaaaattgctcctctaagaaagggtgccaacactctttgtaacacaggacaccgtttattgctaactcgattgcattctttcctttcccacaccgggatctggtcttggtgaacaatctctcctgtccttatctaaatatatcatcgcactgtaaccttccttattacttttcgagcgtccgtcctgtattatcttcaacctgaaaccaaactctaaccaggcttcactcgtggatctataattgaacatgaaaaactcggaccgatttaaatgg

SEQ ID NO: 29 - DNA, 1541 bp, Pichia pastoris
  misc_feature (1)..(1000): promoter
aatgagaataatgtaatatgcaagatcagaaagaatgaaaggagttgaaaaaaaaaaccgttgcgttttgaccttgaatggggtggaggtttccattcaaagtaaagcctgtgtcttggtattttcggcggcacaagaaatcgtaattttcatcttctaaacgatgaagatcgcagcccaacctgtatgtagttaaccggtcggaattataagaaagattttcgatcaacaaaccctagcaaatagaaagcagggttacaactttaaaccgaagtcacaaacgataaaccactcagctcccacccaaattcattcccactagcagaaaggaattatttaatccctcaggaaacctcgatgattctcccgttcttccatgggcgggtatcgcaaaatgaggaatttttcaaatttctctattgtcaagactgtttattatctaagaaatagcccaatccgaagctcagttttgaaaaaatcacttccgcgtttcttttttacagcccgatgaatatccaaatttggaatatggattactctatcgggactgcagataatatgacaacaacgcagattacattttaggtaaggcataaacaccagccagaaatgaaacgcccactagccatggtcgaatagtccaatgaattcagatagctatggtctaaaagctgatgttttttattgggtaatggcgaagagtccagtacgacttccagcagagctgagatggccatttttgggggtattagtaactttttgagctcttttcacttcgatgaagtgtcccattcgggatataatcggatcgcgtcgttttctcgaaaatacagcttagcgtcgtccgcttgttgtaaaagcagcaccacattcctaatctcttatataaacaaaacaacccaaattatcagtgctgttttcccaccagatataagtttcttttctcttccgctttttgattttttatctctttcctttaaaaacttctttaccttaaagggcggccgcggcgcgccttaattaagcttcacgatttgtgttccagtttatcccccctttatataccgttaaccctttccctgttgagctgactgttgttgtattaccgcaatttttccaagtttgccatgcttttcgtgttatttgaccgatgtcttttttcccaaatcaaactatatttgttaccatttaaaccaagttatcttttgtattaagagtctaagtttgttcccaggcttcatgtgagagtgataaccatccagactatgattcttgttttttattgggtttgtttgtgtgatacatctgagttgtgattcgtaaagtatgtcagtctatctagatttttaatagttaattggtaatcaatgacttgtttgttttaacttttaaattgtgggtcgtatccacgcgtttagtttagctgttcatggctgttagaggagggcgatgtttatatacagaggacaagaatgaggaggcggcgtgtatttttaaaatggagacgcgactcctgtacaccttcggaccgatttaaatgg

SEQ ID NO: 30 - DNA, 1101 bp, Pichia pastoris
atgtctacatttgatttcctttccagaaaaagcggtgtcatcgttggagatgacgttagaaagctattcgaatatgcaagagaaaggaaatttgccattccttccatcaatgtaacgtcctcttctacagcggttgctgtattggaggctgctagagacaacaaatcaccagttatgctgcaagtatctcagggaggtgctgcttttttccagggaaaaggcgtcaataataaagaccttagcgcttcagtgactggatctattgctgctgctctattgatcagaactattgcaccttcttatggcatacctgtcattctgcacactgaccactgtcaaaaaaaatggctcccttggtttgacggaatgttagatgcagatgaagaatatttcaagacccatggagagcctttgttctcctcccacatgttagacctgtcggaggaaacagacgatgaaaacattgctatttgtgtgaaatatttcaagagaatgacaaaaatgaaccagtggttagaaatggagattgggataactggtggggaggaagatggagtgaataacgaaaatgctgataaagactcactctataccagtcccgaaactgtttttgcagttcataaagcactggctcctatttctccaaacttcgccattgctgcagcctttggcaatgttcatggggtctacaaacctggcaatgtggagttgagaccatcaattttgggtgaacatcaagcttacgcagctcaacagttaggccttaaaaatggatcaaaaccacttttcctagttttccatggcggttcgggatcttctcaacaagagttcaacactgctattaaccatggagttgttaaggttaatttagacactgattgtcaatatgcatacaccattggttcaagagactatatcctgaaaaacaaagactatcttcaatccatggttggaaatccgcagggagctgataaacccaacaagaaatactttgatccaagagtgtggattagagagagtgagaaaaccatgagtggccgtgttaaagaggctctggaggtgttccatgctgctggtaccttcaagtctgagtcaaaactg

SEQ ID NO: 31 - DNA, 2121 bp, Pichia pastoris
atggctagaattcccaaagcagtttcttacaatgatgacatccatgacttggtcatcaaaaccttccgttgttacgttctcgacttagtcgaacagtatggtggtggtcaccctggttctgccatgggtatggtcgccattggtatcgctctgtggaagtaccagatgaagtacgctccaaatgatccagactacttcaacagagatcgttttgtcttgtcaaacggtcacgtctgtttgttccaatacttgttccagcacttaactggtttgaaggagatgactgtcaagcaacttcaatcttaccactcttccgattatcactcattgactcctggacaccctgaaattgagaaccctgctgttgaggttaccactggtcccctgggacaaggtatctctaacgctgtcggtatggccattggttcaaagaacctggccgctacttacaacagacctggcttccctgtcgttgacaacactatctatgctattgttggtgatgcttgtttgcaagagggacctgctttggaatcgatttccttagccggtcacttggccttggacaaccttattgtgatctacgacaacaaccaggtttgttgtgatggttccgtcgatgttaacaacaccgaagacatctccgcaaagttcagagctcagaactggaatgttatcgacattgtagacggttctagagatgtcgctaccattgtcaaggctatcgattgggccaaggctgagactgagagaccaactctgatcaacgttagaactgaaattggacaggattctgctttcggtaaccaccacgctgctcacggttctgctctaggtgaggaaggtatccgggagttgaagactaagtacggttttaaccctgcccaaaagttctggttccctaaagaagtatacgacttctttgctgagaaaccagctaaaggtgacgagttagtaaagaactggaaaaagttagttgatagctatgtcaaagagtaccctcgtgagggacaagagttcctttctcgtgttagaggtgagcttccaaagaactggagaacttacattcctcaagacaagcctaccgaaccaaccgccaccagaacctctgctagagaaattgttagggcccttggaaagaaccttcctcaagttattgccggttccggtgacttatctgtctcaattcttttgaactgggacggagtgaagtacttcttcaaccctaagttacagactttctgtggattaggtggtgactactctggtagatatattgagtttggtatcagagaacactctatgtgtgctattgccaacggtttggctgcatacaacaagggtactttcttgcctattacctctaccttctacatgttctacctgtatgcagcacctgccttgcgtatggctgctcttcaagagttgaaagcgattcacattgctacacacgactctattggagctggtgaagatggtccaacccaccagcctattgctttgtcttcattattcagagctatgcccaacttctactacatgagaccagccgatgctaccgaagttgcagctctgtttgaagtggctgttgagcttgaacactccacattgctttctctgtccagacacgaggttgaccaatacccaggtaagacttctgcccaaggagccaaaagaggtggttacgttgttgaagactgcgaaggaaagccagatgtgcaactgatcggaactggttccgagttggaattcgctattaagactgctcgtttgctaagacaacagaagggatggaaggtcagagttctgtcattcccatgtcagagattgtttgacgagcagtctattacttacagacgttccgtccttagaagaggagaagttccaactgtcgttgttgaggcctatgtcgcatacggatgggagagatacgccactgctggttacaccatgaacaccttcggtaagtctcttcctgttgaggatgtctacaaatacttcggatacactcctgagaagattggtgagagagtggttcaatatgtcaactctatcaaggctagtcctcaaatcctttacgaattccacgacttgaagggaaaaccaaagcatgacaagttg

SEQ ID NO: 32 - DNA, 867 bp, Pichia pastoris
atgttcaacctgaaaactattctcatctcaacacttgcatcgatcgctgttgccgaccaaaccttcggtgtccttctaatccggagtggatccccatatcactattcgactctcactaatagagacgaaaagattgttgctggaggtggcaacaaaaaagtgaccctcacagatgagggagctctgaagtatgatggtggtaaatggataggtcttgatgatgatggctatgcggtacagaccgacaaaccagttacaggttggagcactaacggtggatacctctattttgaccaaggcttaattgtttgcacggaggactatatcggatatgtgaagaaacatggtgaatgcaaaggtgacagctatggtatggcttggaaggtactcccagccgacgatgacaaggatgatgacaaggatgatgataaagatgatgacaaggattatgacgatgacaatgaccacggtgatggtgattactattgctcgatcacaggaacctatgccatcaaatccaaaggcagtaagcatcaatacgaggccatcaaaaaagttgatgcacatcctcatgtcttctctgtaggaggagatcagggaaacgatctgattgtgactttccaaaaggattgttcgctggtagatcaagataacagaggcgtatatgttgaccctaattctggagaagtcggaaacgttgacccttggggagaactcacgccatctgttaaatgggatattgacgacggatacctgatctttaatggtgagtccaatttcaggtcatgtccatctggtaatggatattcattgtctatcaaggattgtgttgggggaactgacattggccttaaagtatgggagaaa

SEQ ID NO: 33 - DNA, 648 bp, Pichia pastoris
atgtcacaaggaacaatttacttagtactcgcctccccaagatcatctttattcaaggatctgattgaatattacggtctcgatatcaagatctctgacacttccgacccagagtttgcgaagacatttcccttgaagagaactccctccttcaaaggtcctgatttcgtactacatgaagctttggcaatatttgtgtatatcacttcgttgattcctcaaaaccatgggctgtacggttcttccaacttggattacgcccaaaccatcaagtggctatccttcactgcgtctgagatcatatctggcttagttcaagcactgtatccgctcatcggaaacctgccatactccaaagatggcgttgaccaggcactcaaagatttggagtcgtatgttgctgtttatgaaacccagttaaagcaaacgaagtatcttgttggcgataagattactttggctgatctgtttgctgttcagtctatcaggtggggattacaatatatttgggacgtcaattggttgaaacaacatccattgattgatgcctggttcaaggatgttactcaacatccaatcatcgtcaaatctcttggagatttcaagccgttgcagaaggctcttcctaatgctcctcctaagggcaat

SEQ ID NO: 34 - DNA, 1206 bp, Pichia pastoris
atgagcaaacaaactccgtcaggaatgacttttatttccctagtggatgattcaaatgattctcctgtaactcaatctacttcagccgataaggacgagaagcatacagctgtgtcgcaggagcagataaacgctgctggaaagaagcagaaccaagttgatgacgaggaagaagaggagcagaaggaggaatacaaacgttctcttgaagaaattgaaatcaaagatgctgcttattacaagatgacagatcctgttctcctgtatgatattccggcccaggttaattcttctgatgagtacatcaaatggtctcccaatatcgtacagacgagaatgttgctcaaccatttgcaaattccatttgaagtacaatggttaacgtaccctcaaataaagccaaccttacaaaagttaggtgtcaagccttgggattcagatcctgagtatactcttccagctatttcatataaggggactactgtgatgggcagatccgagattgaggaatttgttaagcaaaactttgaccccttatcaactccaaattttacagattattttctggacatcgaagaccgtaaatttaaccgagctatttccaactattcagatcagattcttatggggaagctttctttcccactgttggctttttcggaaagtattgtggtaaaagatgagaatgaccctgaatatttccaaagaacaaaaaatgaaagattcggtgttgactgccaggctttggtaaaggataaagaaaagattcatgaaatggtggaagccatttgcaatgaattgcctggatttatggagctatatgattactcggaagctgagaacgctgatttactatttctgaaagtaaacaagtatttgatgggctttcacattactcgtgctgatcttattattgcatcatatgttttctggatcaagactgctgttaaggctggaatggaagaacactcatttcacaattattggtttgatgcttggtataacagaatctccaaacttcttgactcccccttcgatggctcaaaggttgaacaagaagcgcttgatgatttgaacaagaggatagagggctctcatcagaatcgtgtcagagagtctggggagcaaattgatgaaaatgacaagaccgagaatgtcaatgaactcactgaacctgccagttcgactgttgagcagccccccgcg

SEQ ID NO: 35 - DNA, 1617 bp, Pichia pastoris
atgactctcagctcacaggcttctaatgccatcatttatgtgacttatgggctgactttaatattctgtgtggggctagcttggtatcataatgacaagtcaaaattcttatcgtcaaaccagacaaaaacagggataccactggcccttaacttcatagcctcggcgatggggtgtggtattcttacaacatacacacaagtggctaacttggcaggtatacatggattaatgacttatactattgttggagctttgcccatattctttttctccttctggggacctcttatcagaagaaaatgtccagaaggatttgttctgactgaatggactttccaaagatttggaagtgtcacaggatattatctgtctcttgccacaattttgacaatgttcttgttcatggtggcagaacttagtgctattaagttcgctgtcgaagctttgacaggtttggacggccttcctgtggtcatcgttgagtgtattgtcacgaccatttatacttctattggaggcttccaggttagtttttctacagataattatcaggcctgtgttgttctagtcttggcagttattggtgtcattggctttgctctgaacgtcaacattgacccagaacttaaaagagaaactgaaagctacctattgggtgccaacaagttaggatggcaattactttacatcttgtttattgctattgctacttgcgattgtttcatatcaggattttggttaagaacatttgcagcaaaaacaaacaaggaccttatgattggaaccggtattgcgtctattgttgccatgaccatctgcactttggtaggtttgccaggcatttttggtgtgtggacaggagatgtggttatcggaagtcccgaaggttacctgtcttttttcatcatggtatcaaccatgggcaactggatgataggattaatcctgatcttttccattgttctaagtacttgtacctttgactcgcttcaaagtggattgacctccactattgtcaacgattttggaagaggacgtatgcctctttgggcagctagagtaatcactattttggttatggtcccttccattgtcgttgctgtcaaagttgcagatgatgttttaaagatatacttcattgccgatttgatttcttcctcggtcattccttctctgttcatcggtctctccactcgcttctacttctggtctggttgggaagtagtcggtggtggatttgctggtctttttttcgtgtgggtcttcggaaccgtttactacggtgatgccgctgaaggaggcaagttgttactgatttggaatggtatctacgattctgaagattggggtccatttggtgcgtttgttgttgccccaggtgtgtctttggtaggtggtttgatcatctgtggtgtacgattagcagttctcaaggtatacagcaacattaagggtactccatttactgctcttgatagacctgaaaaacttggctttggtggtgtcgcaattggtgactcttccgatattgaagaggtctacgaagagactttaaacgaatctcaatcaaaaaaatctactgattacgtcaaggaagaagataatttccgtgct

SEQ ID NO: 36 - DNA, 1035 bp, Pichia pastoris
atgacgctttccttattattgagaagtactaacatttttagggctcctaccgcaattgaaactcaaactcaaaccactccagctttcaccgaaccccaggttctgaagctcaagcagaatgttagaaacccagactctctggttgctaacgctgttaccccagcttttgactggaacacctttgagttcgctcccatccgtgagtcaaccgtttctcgcgctatgaccaagcgttactttgctgatttggacaagtacgctgaatcggatgttgtgattgtcggtgctggttctgctggtttgtctgctgcttatactttgggtaaggctagacctgatttgaagattgccatcattgaatcaaacgtcgccgttggtggtggatgcttccttggtggtcagttgttctctgctatggtcttgagaaagcctgctcatttgttcttaaacgatcttggattggagtacgaggacgagggagattatgttgttgttaagcacgcagcttactttatcactactctttgttccaaggttcttgctcttcctaacgtcaagctgttcaatgccaccgctgttgaggacttgcttaccagaaaggacgagaacggccagattcgtattgctggtgttgttaccaactggactttggtcacaatgcaccaccacgaccaatcttgtatggaccccaacactatcaacgctaatgttgttttgtcagctactggccacgatggtccgttcggtgctttctgcatcaagcgtggtgtcgagattggtgccgttaaaaaaatggacggtatgcgtggtcttgacatgaacaaagctgaggatgctgttgttaagggtgccagtgaaattgctccaggattggttgttgccggtatggaggttgctgagcactctggttccaacagaatgggtcccactttcggtgccatggctctttctggagtcaaggccgcagaggaagttctgaaggtcttcgatgagagaaagaagcagaaccagcaatgctatggtggactttccgct

SEQ ID NO: 37 - DNA, 1464 bp, Pichia pastoris
atgacccttagttcgtctcatctgaatagtcaacactccgacactttggcaaatggcactaacggtaactattctagcaccgtttccaacaacttgagcttaagtttgaactccttctctttctctgacaagttctcattgagtccaccaacaatcactgacgccgaaaagttttcattgatgagaaacttcattgacaacatctcgccatggtttgacacttttgacaataccaaacagtttggaacaaaaattccagttctggccaaaaaatgttcttcattgtactatgccattctggctatatcttctcgtcaaagagaaaggataaagaaagagcacaatgaaaaaacattgcaatgctaccaatactcactacaacagctcatccctactgttcaaagctcaaataatattgagtacattatcacatgtattctcctgagtgtgttccacatcatgtctagtgaaccttcaacccagagggacatcattgtgtcattggcaaaatacattcaagcatgcaacataaacggatttacatctaatgacaaactggaaaagagtattttctggaactatgtcaatttggatttggctacttgtgcaatcggtgaagagtcaatggtcattccttttagctactgggttaaagagacaactgactacaagaccattcaagatgtgaagccatttttcaccaagaagactagcacgacaactgacgatgacttggacgatatgtatgccatctacatgctgtacattagtggtagaatcattaacctgttgaactgcagagatgcgaagctcaattttgagcccaagtgggagtttttgtggaatgaactcaatgaatgggaattgaacaaacccttgacctttcaaagtattgttcagttcaaggccaatgacgaatcgcagggcggatcaacttttccaactgttctattctccaactctcgaagctgttacagtaaccagctgtatcatatgagctacatcatcttagtgcagaataaaccacgattatacaaaatcccctttactacagtttctgcttcaatgtcatctccatcggacaacaaagctgggatgtctgcttccagcacacctgcttcagaccaccacgcttctggtgatcatttgtctccaagaagtgtagagccctctctttcgacaacgttgagccctccgcctaatgcaaacggtgcaggtaacaagttccgctctacgctctggcatgccaagcagatctgtgggatttctatcaacaacaaccacaacagcaatctagcagccaaagtgaactcattgcaaccattgtggcacgctggaaagctaattagttccaagtctgaacatacacagttgctgaaactgttgaacaaccttgagtgtgcaacaggctggcctatgaactggaagggcaaggagttaattgactactggaatgttgaagaa

SEQ ID NO: 38 - DNA, 1020 bp, Pichia pastoris
atgtctactaacaagatcactttctgtcttaactggcaagctgccccataccatgccccaatctacctggcccaaaaattgggctacttcaaggatgagggtcttgatattgctatcttggaaccaggtaacccatctgatgtcactgaactaattggctctggaaaggttgacatgggtcttaaggctatgattcatactttggctgccaaggctcgtggtttccccgttacatctgtcggttctcttttggatgagcccttcaccggaattttgtatctcgagagctccggtatcactgatttccaatccctcaagggaaagagaattggttacgttggtgaattcggaaaaattcaattagatgagctgaccaagcattatggaatgactcctgatgactacactgctgttagaagcgggatgaacgttgctagagagataatcaacggtaacattgatgctggtattggtattgaatgtgttcagcaagttgagttggaggagtatctgagatcccaaggaaaggacgttgatggggctaaaatgctcagaattgacaagctggccgagctaggatgctgctgtttctgtaccgtcttgtacattgtcaacgacaagttcttggctgctaacccagataaggtcagaaagtttatgagtgctgttaagagagctactgactatgttattcaaaagccagctgaggcatacgctgatttcattgagattaagccacttatgggaactcctctgaactacaagattttccaaagaagttatgcctacttttctgagtctctttacaacgtccacagagactggaacaaggtcaatgcctacggtaagagattgatggttttgccagaagactttaaggccaattataccaacgagtacctatcttggccagaacccaaggaagtctcggatccactggaggctcaaagaagaatgaacatccaccaagaacaatgcaagtgtaatccatctttcaaaagactggctctcactggtctt

SEQ ID NO: 39 - DNA, 747 bp, Pichia pastoris
atggttttagacactgttgtttaccaccccacactggataaggttatccagtatctggacagctcggcaggtagagacaaattgctccgtctgttgcaatatttaaccaagtttgtctctttctacctgatcaagaatggacattcaattgttactgcccagacagtacgccgaatagaggctattgcaaccttgaacaggaaggctctgagattcctcaagcccctgaaccacctcaaatctgcttcggcaacttttgacaataaactaaccgacaaggtcaccagatattcacaggtgttgcgagatctcggctacgctgtctacctggcattggactctgtttcctggttcaagcagctcggtatctcctccactaaaagactgcctcaagttcaaaaactggcctcattattttggttcgtcgccgttgtcggtggagcagtcaatgatctgagaaagatcagattgtctcaacagaaagtggcctctctcaaacaagagctggttgtcacttcagacaaggagggagaacaaacagtctccaaggagaccatcaacttaatcgagtcagaatccaaactgattggtagcacgacaataactcttatcagggatctgttggatggttatatcgctcttaacggctttgccttacaaaacaaagatgagaaggttggattggccggtgtcatttcctccttgataggaattagggatgtctggcagggaaaatacatcaatgaa

SEQ ID NO: 40 - DNA, 1290 bp, Pichia pastoris
atgacatcaaatataaatggttctctgcctcaaagcgctaatgtggtggttattggttctggatttgctggaaccgctgtttcttactatttacagaatgaaattgctggtgaccaatcgattgtgatgctagaagcaagagcagccgtctctggagccacttcccgaaattctggacttataaaacctgagtaccatagaaatcatggtgaatatgttgacaagtttgggtcgaaagttgcggggcaattggtcaattttgaggttgacaacatgaaggagctggctagaattctaactcttgattccgatcttaatgaaaaggcagacttccaagagcggatacatcttgatgcttactctaaccctcgttcgtcggagactgccatcaatgatttctatgcttttatggaaaatgaagaagtctccgtagacctgaaaagaaaagtgcaaatattatttggggatttggccaagcaactgagtaatgtgcctactaccccttttgtcattcattatcctaatggatctgttaattcgtatgatttcgtcactgctatgttaaccagagctattcaaaaaggcctagccctatataccaatactctagtggaagaagtgcagcaattggattctggtgtatggaaagtatctacttctagaggtgaattatttgcggacaaagtggtttttaccaccaacgcctacacacaaggactcctgcctgagttttcgaaatcaattatacctatacgaggcgtttcatcacaagttaacgttcgtaatacgggtaatgagcactctctgtccattggctccgacatttatgttccacaatccaagaatctgctctacagttcactgggtgagctggaaaagcagaccaatttcatggctaattacaacactgtggacgattcaactgtatcgtcccaatcgttagaatatttgcaaaaaaatttaggtaaagtgaactcacttgctactgccactgctacttcatcatcgtctggaatcatggcctacactgatgatcattttccttacgttgggcaattgagcgagctgggaaaactaaatgcatatattttggctggttgcaactgttcgggtctttccagaatgctcctttgtgctaaagaacttgccaagtccgttgcattggggtcggagttaagcgacagagtcccagctccctacaaggtaactagggaaagaatgggactcgttgacaaattgatgcaatcggtattgaatgaaagagaaattagagagactagagcaagactc

SEQ ID NO: 41 - DNA, 1158 bp, Pichia pastoris
atgtcaaaaaacgctcaggttgacgatcttgcccatgggcttgcaggtgccggagggggaattctctcaatgattatcacttatccccttctgaccctatcaacccatgcccaatcttcaaaaacccagaaaccactagatggctctgtagatgagaaggaattggagcccaagaagtcttcaacttacggtactctaaaacggattttgaaaaagcagggagttcggggtctctacaacgggctggaaagtgctatccttgggatagcagtcaacaacttcatttattactacttctatgagctaactggaaacactttggaaggcttgtcccgtggtagaaagagaggttccagagttggtggtctctctgcattccaaagtattgttgctggagctattgctggtgtgatttcccgtattgccacaaatcctatctgggtagcaaataccagaatgactgttctttccagagaacagagagatctgaagagggtaaacacattgcaggcaattttgtacatcttcaagactgagggattcaaaacgctgttcagtggcctcattccggcattgtttttggtcctgaacccgatcattcattacacgatttttgagcaactgaaaacactactggtaaagacaagaaagagagcgttgactcccttggatgctttactgttgggtgcttttggaaaacttatttcaacggtgatcacctacccctacgtcaccctgagaactagaatgcatttgcaaaacgctgagaatgctaggaattcttccggtgagagctcagtcagcaattccgctgttctttcggccacgtcagacgaagacttgggcaaagagggcgacaacgagaaaaagattgcccaggaacaacctaccaacactatttggggcctttccacgaaaatgttaaaagaagaaggaatatcaagtttctacagtggaatgtctgtcaagttatcgcagtcaatcttgtcagctgcctttttgttctttttcaaagaagagttggtatcagccagtgatgtggcaattaaggcggtgaaaaaggtcgactacaagtctttcttgaataaccccgctcaagtagtgcctgatttgaaccctgcaccatctcccaatgctgacggctctccagtggatatt

SEQ ID NO: 42 - DNA, 1830 bp, Pichia pastoris
atgacgacattatcgttgaagcaaactgcatatgaattcgtcgaaggcatgacaggtgactaccactacttaaagaaccttgatgcttcagattcaaatctacgataccttaaagattatagttttccaaccttgcaagagttgaatctttccaatttatcctacctgaagaaacttgtgtctgatggagatcaattgagcgggttggaaaagttaactgctgatgggtccaatatcgaacttatcgatattaagggagatgtcctggcaagtatctcctgttcgaacacaaaagagctaaaagcaatcaaaggtgaactgccggcgattaactatatctatgcagacaactccagcttggaatcggtatccttattgccatatgaagacgaaaaatttcagttcaagagaatgttctttctatatttggtcaactgtcccaatctcatatcctttcaaagagggcagtatcccaagcttcggactgtcgatttaagtaattccacgatacaagttcttcattcatctcttataaagcatcttggtgctttatacttgggcaacgcaactattaaagagtttgtaattgacgacaacgacaatgaagatgagctcacccttcggcaactaaaaactttgaatttatcaaattccaggatcccaacaaatatgacagattttcttttaagacatatttttagaaagaagagcaatccctgggataaagaatggtcgtcattgatttggcaatcagtcaatacagatcaagaggtgcaagaggtccagagttttattaagagaattccacagctcaaaaaactggatatatccaattcatcaaaagcaaactcattgctgtccaatctagtgtctgacattccagagttttggaacagcttagaagatttggatgtcttcaattccaacactgagggttttaacttttcaaacagcaaatcattgattcacctgggttgcagcgatcccgatgcaactgttttgaaagttgacgacatgcccgccttgaaggcgctggatgtcttgggaccctctaacattgaaactgtaaagttatccaactgcattagactcgaaaaggttgacttcactggctgctacgctttgaaacgctttgagtcagactctttgagtcttctgcaattgaagttttcaaataactcattggaaacatggtcccttaatgactcctctatcagaagcatcacgttagagaacaatacaggtttcagaaggctagaaggaaatttcccgaatttggaaacgttagatatcagtaatagcgctgttgaagttatcaatattcatgatggtagcaaattgaagaagctcagcattttgagaactcgaagtgttgaagagattcaaattgataatctttcaacactacaagaattgcgctatgacaccaaagacgaaaagaatacgaaagcagtaaaggagtttgcagataagattttatcaaagagggaaccaagttttacagccaaggtactccaaagagtccgctttgaacacaggggcttattcacaggtgggttcaggttttttgatagaagaactgaggaactggtttgctgctttaaagatatcaacatttccgaaaataagatcaatgcaaaatgtggcatctgtgaggaaaactacgaggaaggtcagaaatgtcagattctctattgcaagcatgcttttcatactgattgcgtagaacagatgctacaaatgggtaacgataggtgcaggcattgcaacgatgaaattgacattgcctcagtaggtctaagtagcgatccattgccctttagatggttg

SEQ ID NO: 43 - DNA, 582 bp, Pichia pastoris
atgagattttctaacgtcgttttaactgcaattgccgctgccggcgtacaggcagatgaagccctttacactgtgttctacaatgatgtcactgagaacgcccaagagtatctgtcttacatccaggccaatactgcggctggtttcactgacctcttgagtctgtacactgaactggccacttacaccgacgattcttacacaagtatctttactgaggaggatttccctgcgagcgaactttcatcgttcgttgttaacctgccatggtattcctccagaattgagccacaagttgcggctgctgaaactggtgaaagtgaggaggaatcagagactggtgaaagtgaggaagaatcagagactggtgaggagacagaaactgagactggatctgagtctgaatctgagtctgaatcggagacctccgctactggcactggcactggcacctccgcctctgagagcgcggagactgaaacttctaccgacgctgctgtgtctatcgatcacccaaagtccaccttattgatgggtttgactgccgcagttgtcagtatcactttcggagtctttgccttg

SEQ ID NO: 44 - DNA, 531 bp, Pichia pastoris
atgggtagaagaaagtcacaagctgctgcagagagaaatcttgaaccaattaaaattagtaccgactcaattaagaaaaggcctcgtcgagattccaatgaacctccattcaaaaagtttgatgatctagaaatgtttgaaacttacttgaagggtgaatcttgggataacgattttgatttcctccacgctcgtttggattattatcctccatttattcgtaatgaaattcatgacgatccggaaaagattaaaccaacaatgaacaataagtccaagaagtttgtgagaaacttgcatcatcacgttgacaaacatctgttaaagcaaattaatgacatggttggaatcgagtacaaattcaaacgggaggaagagaagttaccagatggccgactaatctggcgctacaaagacgaatcagatcatggatttgaaggtcttgaccggaaatggacagtcgaagttgatgtcgagtgtagtcccaacgatccaactgtagttgtcgatatgaggtccattcctattgac

SEQ ID NO: 45 - DNA, 969 bp, Pichia pastoris
atgaccttgacgattaacccgagtttaaagaacgtcttagaaaaccagaagggcggcgcacctgtggagaatgatcttaaggcgctgagaaagcatgctgacaagatagaggaacactactacaaagaggtgataaccaaaccatctgacgttgcaggcatgcaatttactatcactgctcatgacggagatcaaattgagtgccgtttctactctaaagatgctgatcctaaccaagtaaggaccagtaagctaccgtgcattattcacttccatgggggtggatacataaccggtgacgttagccgttacagtcatttgacctctcaatatgtctcagcgactggcgtacctgttctgagtgtggactatagactggctccagaatatccagctccaattcctcaagaagatggctttagtgcgctacagtacctttatgaacattgtgataacctaatgattgatcccaacaagattattctaatgggagattcatcaggcgctgggttggctttatctgtggctgcattggcagcagaaaggggcattccaatagccaagcagattctcatttatccaatgttggactcaaacaataccgaagaacccgattctgaagaccttaaggagaaaaaaccttatctaacatggactcatagaatgaacaagctggcctttgattctcttctatcagaaaaagaagaatatgagccatcattattcccattcaacgatcctaatgttgattggtccaaatttcccaaaacttatttggaaaccgtgacagttgacatcttaaacgatgacggccggcagctgcatagaaatttgactgagaacaatgtacaagttgaatttcatgagtgggaaggactttgccatggcttcgaacctcttctttggaccgaggaaaatgatctacttcaaaacattttagagaagcgttaccaagcaatcctggatgta

SEQ ID NO: 46 - DNA, 1590 bp, Pichia pastoris
atggaaaaaaaagcttccagtgatctggagttagaaacggatagcaaaatctcggagacagagaaaaggggtccagtatcaaagttactgaacggcctcaactatgtctccaagaagctagattcctttggtggtgaatccaccggtatcgaaagagtttctccagatcaaaggcggaccaatatgacgaggattatcattcacgttatgggtctgtggttatcaggatgtggtggtataacttctatgtccagtttttttttaggacctttgatttttggactggggttcaaagatagtatgataagtggattagtatcttgcacattggggtgtctcttagccgcctattgtagtacgatgggaccaaggtcaggactacgtcaaatcgtcagtgctaggttattttttggtccttgggctgtcaggttcccagctttaatcagtgctatcggtttcgttggttggagtgtgactaattgcgtccttgggggacaaatactgtacagcgtttccaatgacaaactgcccatagaaataggcattgtgattatctcaatgatatcgttggtgattgccattttcggtatcagaattctcttgcatactgagacgcttatctccattccagtgttcacggtgttaattctcctgtacatcattggctccaatgaataccccaattacttgaacactgtatccattggggacactatgacgatcagaggaaacttcttatcatttttcgctttaggtttttctgtgacagctacttggggaggatgcgctagtgattattatacactattaccagaaaacaccaaccagctattcgtatttttcatgaccttctttgccatttggataccttcgtttactggagcggtgctgtcgatcttacttggcaatgcggccactgttcatgagccgtggttggaagcttatacgaacaactcactgggtggattattgcatgaaattttttcccgttggaatgggtttggaatgttccttttggtgatattcttcctttcacttatcacaaacaatattattaatacctactctggagcattggaactgcaactgataggtggaccactttcatattttccaagatggttcttgagtgtggtcatggttgtaatattcatggtttgttctctggctggtagagaccaatttgccacaattctttccaatttcttgccaatgctggggtattggatttccatatacttcactttactgttagaagagaatgtcatcttccggtccaacaaaactctaattaaactttatcaatatgagttctctaccattcctggagagaagcaagatcttctggagggcaaatccagaccatattacaactttgacatctatctttctcgggataggcttactcacggatttgcttcttcattggcgttttgttttggtgtagtcggagcaatttgtggaatgtgccaagtctactacattggacctatagcatccaagattggaagccatggagctgatcttggtatgtggttagctattggatttactgctgttacatatccggtattcagatatattgaacta

SEQ ID NO: 47 - DNA, 1000 bp, Pichia pastoris
actctacccaggattatttttcttctgcgaatacaaaactgcttatatgtcacacggataactcctcttttaacgagatagttgacttctattaaaaagtccgcatagttagatttacctccatcttgagttagaagatgaaccttttcattatagggtggatcataccaattccaaccatctggggccaaggttgaaaaactgggggttcttggaaagttatagaagaacaaagttcctattctggcaaaattatgaaggtcattggtgacattggtaacacttgtcagatccataaattaatccataagataaggcaaatgtgcttaagtaattgaaaacagtgttgtgattatataagcatggtatttgaatagaactactggggttaacttatctagtaggatggaagttgagggagatcaagatgcttaaagaaaaggattggccaatatgaaagccataattagcaatacttatttaatcagataattgtggggcattgtgacttgacttttaccaggacttcaaacctcaaccatttaaacagttatagaagacgtaccgtcacttttgcttttaatgtgatctaaatgtgatcacatgaactcaaactaaaatgatatcttttactggacaaaaatgttatcctgcaaacagaaagctttcttctattctaagaagaacatttacattggtgggaaacctgaaaacagaaaataaatactccccagtgaccctatgagcaggatttttgcatccctattgtaggcctttcaaactcacacctaatatttcccgccactcacactatcaatgatcacttcccagttctcttcttcccctattcgtaccatgcaacccttacacgccttttccatttcggttcggatgcgacttccagtctgtggggtacgtagcctattctcttagccggtatttaaacatacaaattcacccaaattctaccttgataaggtaattgattaatttcataaat

SEQ ID NO: 48 - DNA, 1000 bp, Pichia pastoris
aataaaaaaacgttatagaaagaaattggactacgatatgctccaatccaaattgtcaaaattgaccaccgaaaaagaacaattggaatttgacaagaggaacaactcactagattctcaaacggagcgtcacctagagtcagtttccaagtcaattacagaaagtttggaaacagaagaggagtatctacaattgaattccaaacttaaagtcgagctgtccgaattcatgtcgctaaggctttcttacttggaccccatttttgaaagtttcattaaagttcagtcaaaaattttcatggacatttatgacacattaaagagcggactaccttatgttgattctctatccaaagaggattatcagtccaagatcttggactctagaatagataacattctgtcgaaaatggaagcgctgaaccttcaagcttacattgatgattagagcaatgatataaacaacaattgagtgacaggtctactttgttctcaaaaggccataaccatctgtttgcatctcttatcaccacaccatcctcctcatctggccttcaattgtggggaacaactagcatcccaacaccagactaactccacccagatgaaaccagttgtcgcttaccagtcaatgaatgttgagctaacgttccttgaaactcgaatgatcccagccttgctgcgtatcatccctccgctattccgccgcttgctccaaccatgtttccgcctttttcgaacaagttcaaatacctatctttggcaggacttttcctcctgccttttttagcctcaggtctcggttagcctctaggcaaattctggtcttcatacctatatcaacttttcatcagatagcctttgggttcaaaaaagaactaaagcaggatgcctgatatataaatcccagatgatctgcttttgaaactattttcagtatcttgattcgtttacttacaaacaactattgttgattttatctggagaataatcgaacaaa

SEQ ID NO: 49 - DNA, 1000 bp, Pichia pastoris
aattgtcaccttgaacagcacaatcttaaacaactagatctacaccatgataacattcaacaaacgaatagcaacattagcggcaacgttattttcattcattgtgctttatactctctttaacagtggagctcaattttccaaccaactagatcagcctgttcccctcaaaactccagagctcatcataccgaatcagagtactgagaatgatccccctcttccattcatgccaaaaatggctaacgaaactttgaaagcagaacttggaaatgcttcctggaaactctttcacactattcttgctagatatcctgaatccccatcggagaatcaaaaatcaaccttaaatgactacatttatttgtttgcacaggtttatccatgtggagactgtgcaagacatttcaatttattgctgcagaaataccctccacaattgtcctcaagacaggtggctgcagtgtggggatgtcatattcacaatcaggtcaataagagattggagaaaccacaatacgactgctccaatattctagaggattacgattgtggatgtggctctgatgaaaaggaagtagatgacactctgaataacgaaacaatagaacacttgcaaagtatcaaaattactgaaaaagagagtgaacaatttggtcgatgattacctgattagggcaaatggccaagaccaaattatggagaatcctcaacataccacactttaacaatgctattttaatcttttaagctcaaaccgatgctcatcgccctttcaaggtcatacttaaactcctgtgggttatacttagaactcatctctcaaatgcgtgacgtcatcgcgcatgtcaggtaccctcaaacggtctttggggtaaactggtagacccttggagggtacctatatttggtatcactggcattgatgagttagatttgggtataaataggtcattgaatatcccttcatgaagtatcaattaagaaatcacaaacacaa

SEQ ID NO: 50 - DNA, 1000 bp, Pichia pastoris
attgttgtgaatactctccttcatttggatttcttggacttcggactctcttgatctctcttcgaaagttttaactctgttcatgtataattttacccgctgtaggtcgctcataataccatgagtatgcacatcttttactccattaactttcaggtatgcaaaatacaatgaagatagtatatagctcaaagaatttagcattttgcattgatctaattgtgacattttctctatgatatcatctagcttcttaaactcgagaatctcgtccaacgaggcagaaacattgtccagtcttacgtcaagattattcacgagtttctggaccgtatcaacgttttccatcttaagattacagtaagtatcgtccttttgaactgcaaaggtagaaaagttaatttttgatttggtagtacactatgaaacttgctcaccccaatctttcctcctgacaggttgatctttatccctctactaaattgccccaagtgtatcaagtagactagatctcgcgaaagaacagcctaataaactccgaagcatgatggcctctatccggaaaacgttaagagatgtggcaacaggagggcacatagaatttttaaagacgctgaagaatgctatcatagtccgtaaaaatgtgatagtactttgtttagtgcgtacgccacttattcggggccaatagctaaacccaggtttgctggcagcaaattcaactgtagattgaatctctctaacaataatggtgttcaatcccctggctggtcacggggaggactatcttgcgtgatccgcttggaaaatgttgtgtatccctttctcaattgcggaaagcatctgctacttcccataggcaccagttacccaattgatatttccaaaaaagattaccatatgttcatctagaagtataaatacaagtggacattcaatgaatatttcattcaattagtcattgacactttcatcaacttactacgtcttattcaacaat

SEQ ID NO: 51 - DNA, 1000 bp, Pichia pastoris
cctgatttgatctaatcctggctcatagttaccttgtctcgcttgctgatgttgtttatgttcaaacttggactcaactccaaattcagcttgcacttcatttgtgtcgaccaacgaaactgcagccggttcagaaaggctgaaaccctctcttctcaatagtcctaagatctggctaactccagcttctaaagtcttcaattttctgtctatattcaagttcatttttgctatcggtgacagcttgaacacttggctttctggttgaatggtttgcttgatatcaaacgaaccgttttggtccaaattgggtaagcatccaattatattctcctttgaattatcaggatctgtcgcccgtttgagcaaatcttcttcaaaggaacagacttggctcatcgaggcacatcgaaggcatgcgatagaatctgaagaaggaaaacaacgagtcttctgtctccgacaggatgagcaagcctttgaagctctagaatacaaaggtttcttgggtttggtcactttgtttgactttgtggagtcatttcctggaagcacaggagccatttctcagcttctctctcagtttttaaagtttgtcgaaatattttgataaatgctctgtacttgataagaactgctatctttgatttcgcgacagaggtgaaagagaacagaatccatcacctccttacatcagaaatgagagtgagaattcatagcctaccaaaacaaggactcagggaattgataagtttaccccacaatttcgcttactccgcggtcatgtatgatggatgatccagtggtatagtccaaacgaaacattcgacaaagttctttttgcttctacctagaggggtctcaatttacgttaaagccagtcgggcatatactcgatcgtataaatagaaacgttttagccctcgttttcaccataccattcctttttcctgggtattctactaagaccaaaagagtacctctccacactaccaaaacc

SEQ ID NO: 52 - DNA, 1000 bp, Pichia pastoris
tatttagatatcaagcatgtgaatagcgtagctggtatactaacaatcatttaggccccaaaagttaagaactcagacactatcaaccagaagctggccctgaccatcaagtcaggaaagtacaccttgggattcaagtccaccgtcaaggctatcagacagggtaaggccaagttgattattatctcttccaacaccccagtcttgagaaagtccgagttggagtactacgccatgttgtccaagaccaacatctactacttccaaggtggtaacaacgagttaggaactgcttgtggtaagctcttcagagttggtaccctggctattcttgatgctggtgactctgacctgctttccgttgttggaaactaaactaattagtcgaagaattttttttcaaccaacttatttcctgtaatagttaatagagatttttcattggacattgcgaccttaatatttgcggggagcatctacttcctcattggtaaccctgctatttcttgaaaaacgatttttcgtttgtagactccaccatatgaacgcagatctgtcgcccatggtcttgcttgaaatgattgcgcgaaagatttgtacctgtcaacactagcccatatctcatcacctcgtaggactagaaataggtaaatgggtcgaccttgggcatggccctagcatctgcaaaaggtgcatccttcaacactcttctgcccaagaaaagttagctcaatttaaagcttcctgcttacaattgctgccattttccgttttccaatgagcctggattgttgatgagcatcttagaaatgccgcattgggaagtctcagtcgactagcgactcgagctcatcagccatttgaatctcccatgcagcctaagaattagtcgtttcttttctttttcatttttccccttcattttttttggtcactgaccaatactatataaacttgtgttcacccacttatagttggcgattatttccaggcacaagta

SEQ ID NO: 53 - DNA, 1000 bp, Pichia pastoris
tgctgttttgggctcgtacggatgtttcttaggtccgatgattggtgttatgacttgtgactactacttcgtacgtcatcagaaactcaagctgacagacctctacaaagccgacaagagttctatttactggttctacaaaggattcaactggagaggtttcgttgcctggatttgcggttttactccaggtattacagggtttcctagcgtcaaccccaacttgactggggttcctacagcctgtatcaagatgttctacatttcgtttatcattggttacccgatcggattcttagttcatctggcactcaataagctattccctccaccaggtcttggtgaagtcgatgagtatgactactaccactctttcaccgaaaaggaagcactgaaattaggaatggcccctagttccgagttggacagagtcagcaccgatgacccgatcaatattccttacgacgagaagtctttaggctaatgtagttaaatagttaatcgaaacaatcgtgtatcctctttatcgtaccagcgggattcgctgcttggatgggtgactcctgtccagttgactcaaagtagtcaaaataggcctggagacccttaacaggtcgatgagtagcctactatgagaaaacccctcaccacaactggactataaaagggcacgtcaatccccaaagcaactcttttctttcatccctactttattactttatcctttgatcttcattgaagaaaatctgaaacaattgtaagcagcaatccacacctccccagcaatgactcaatttactaaccccattgacagaaaatgtgagcatcttttttagatgtcatgatgataggtggagtattcttaattattgctttcagcaaaccggtgcccataaagtgtttcccattaaatcaatgagaggcattaaggctgagattaaacggttgaacttgaactagataattctagcggaaagaattgctctttttattacgtcgt

SEQ ID NO: 54 - DNA, 1000 bp, Pichia pastoris
ctgccgaaagaagcacaagaaatgtgacgagaacagaaacccaaaatgtgacttttgcactttgaaaggcttggaatgtgtctggccagagaacaataagaagaatatcttcgttaacaactccatgaaggatttcttaggcaagaaaacggtggatggagctgatagtctcaatttggccgtgaatctgcaacaacagcagagttcaaacacaattgccaatcaatcgctttcctcaattggattggaaagttttggttacggctctggtatcaaaaacgagtttaacttccaagacttgataggttcaaactctggcagttcagatccgacattttcagtagacgctgacgaggcccaaaaactcgacatttccaacaagaacagtcgtaagagacagaaactaggtttgctgccggtcagcaatgcaacttcccatttgaacggtttcaatggaatgtccaatggaaagtcacactctttctcttcaccgtctgggactaatgacgatgaactaagtggcttgatgttcaactcaccaagcttcaaccccctcacagttaacgattctaccaacaacagcaaccacaatataggtttgtctccgatgtcatgcttattttctacagttcaagaagcatctcaaaaaaagcatggaaattccagtagacacttttcatacccatctgggccggaggacctttggttcaatgagttccaaaaacaggccctcacagccaatggagaaaatgctgtccaacagggagatgatgcttctaagaacaacacagccattcctaaggaccagtcttcgaactcatcgattttcagttcacgttctagtgcagcttctagcaactcaggagacgatattggaaggatgggcccattctccaaaggaccagagattgagttcaactacgattcttttttggaatcgttgaaggcagagtcaccctcttcttcaaagtacaatctgccggaaactttgaaagagtac

SEQ ID NO: 55 - DNA, 1000 bp, Pichia pastoris
atcttttcagcttcatcgtcagtgatatttctcagcccacagaccaagtcaactttggaatctaacaaccttgttcttacaatgttagaactcttaagtcgcatgccatgatcttcaagctgaattttgtgaaggaggtcaaaccccacaatggcatctagttgtttagaatacatgccttcgacaagtgtttgagtgtccaaaatcaagagctcaaaattattgaatttgtctgccaataacgccgtaaattgattagtgtccagcccaccaacaataggagcacctatagttaatttttcagataaatttaagttatcaaggtaaaggagctctaagtttaccccttccaacagggttatttgagaactcaataaattgttgaattcaaaaccaattgtctttgaattctccactggagcttccttgctgaaattgattttgataccattggcatcaaagagacccgtatgataactccataaaaaggggagatgataggccttaaattcatcgttaatctgcaaatttattcctgacatgtctttgtaaatagttatagttcagaaactggaattgagctcaaaaaactggaatcgagcggatatttgaagattgatgccttactcatgaattgattgataagagctccgtgattcactctgtcaatgattacccctctcctacccgatttgggactttttcttcagtcttggggactttttttcatatgacttgaccttgctttcccaatagggaaggactcacccatggatgattaagtttggattactcgtttaggaaatagtagccatgaatcaatttgaatcataccatcatgaaatagggttaggctgtaaatgcctcaaaaatggctcttgaggctggatttttgggtattggaatgttggtagcaattggtataaaaggccatttgtatttcacttttttgtccttcatactttactcttctcaactttggaaacttcaataaatcatc

SEQ ID NO: 56 - DNA, 1000 bp, Pichia pastoris
caaacttaaccgaccgttcttccatccgtttattaatatacacacctatgaactgagccaggttttcaggtctctgtgactctctatacattgacggaacaacatccgttcagtctcatccaattgcagcccaaactctgagtttagcaattgcaaatggttattatctgacgagtaatcgttgatggcacatgccctctgtttgaacatctcttgaacaatagccatcagttctgtgtcattaaacatgcttccccatttcactgacagtttgtagaaatagggcaacaattgatgcaaatcgattttcaacgcattggttttgatagcattgatgatcttggagctgtaaaagtccggctggataagctcaatgaaataggttggttgatctggatcttcttttgggtcattttgttcgctctgtatttcacaaattgccagaatctctgccaaccacagtggtaggtccaacttggtgttctgaatcacaggcttccccgggttgttctctaaataaccgaggcccggcacagaaatcgtaaaccgacacggtatcttttgtccgtccgccagtatctcatcaaggtcgtagtagcccatgatgagtatcaaaggggatttggttatgcgatgcaacgagagattgtttatcccagatgctgatgtaaaaaccttaaccagcgtgacagtagaaataagacacgttaaaattacccgcgcttccctaacaattggctctgcctttcggcaagtttctaactgccctcccctctcacatgcaccacgaacttaccgttcgctcctagcagaaccaccccaaagtttaatcaggaccgcattttagcctattgctgtagaaccccacaacataacctggtccagagccagccctttatatatggtaaatcccgtttgaacttcgaagtggaatcggaatttttacatcaaagaaactgatactgaaacttttggcttcgacttggactttctcttaatc

SEQ ID NO: 57 - DNA, 1000 bp, Pichia pastoris
acaattgtttaacgccttgttggacagttgggatttcaactgaagttttcgaaacagttccggtgatgaggatataattgagttccattttgttgagaccaataaacaggcatacatgtctgagaaattcaacaggcgaaagatcagcttcactagctctatcggcaaaaaaatggaaggatctattgttcttggagtgactaatcttagtttctttgtacaaggcactggtgcgtcaaactttaaaactttgaagtgtagctgatttttcttgtccaatttgattttcagtgcatttttcagttcctttaattgttccaaaagaaccttggaatatctaaattgattatcctgatctcttctagtttctattatggagatcccttctcttgctgtagacaaagcctcactgatcttcccttgtaggtctaaaattttgcaagttctcaaataacccttgcagttgtggcactctctcttgatcattttaagaccatctttcaaggccatattcaattgatctagcttttccaaacaggctgccctacaatctaacagagatatcagttgagatacacagctggcggccaactcgggatcattctcaatgaaattgatagtagaagtaaatgccttgtagcagcgcctatactctttttgtttgaaaagctcaatacctgtcttgagtccctgatcaaccaattgacggtccatttgctgcctccttcagggccccacgatagtatactctttgccagttcaatgcacaggcatgtaaaaacgcgctaccatcagataccaccgataccgccccaaatggcccaactcggtaatatctggggaggctatcttagactaaaaagattaaaaaaatacccgggaaccacaactaaaatggtgtcaccctcttgcaaacttatataacctcttcatgtcgtttgggaaccactattacttccccgtttcaactacatcgtttccaaagtggatagaccgtata

SEQ ID NO: 58 - DNA, 1000 bp, Pichia pastoris
agctcagattggaaatgatttttgatcctaccaagaagcctttgatttccagaatctccgctaagtaagtaacccccgcaaacgcatgcatccatgcaaacaaaatactaacaattttagccccgttgttgagaaacccagaaaattgaatgttcaaccaatccagacgatcaataagaaaaaaggcccaaaggctacttccaaacctgctgccgccaaacctgctccttcaaaagccggtcccaagggaggtaagaaggtgagaaagccaaagaagacagttgaagaattggatcaggaaatggctgactactttgaaaataagaattagcccaacaaaatatgtacaagtattatataaatgaatctacatggtgtgttttatttagatcctccaaaccaaggaaagaaactaaacttatctccggacttacgagtcaaataactatccgcagttccttggaactcagactttcttccataagcggtcatatcatctttggactgtgggaatcctggacgaatctttgaaatgtcataatcttgctctctatctccaagcacagcgtccggtaaatgctggttcttctttctcagatgaatcttggatttaacaaataaagccgtgcctatggctaatgtactcaaaaacaaagtctgcttccagaatttcgcaaacgatggaatgccatttcctgtaaatgtactcattgaacctatgtttgattaaagttggtgtgaagtcatcaaacgagagtaaaatcagatactcgtgcaccggccaaaattgactgagctaatctctgcaggcttgacatccgaacacaacaaataggcgacaaatcttaactatctaatcgtaggctatggtagaactttgtgggggtagaggaagactacaacagcaagacaaaacaaaagagtcatagtttgactctctgcttttttcttctttctcttctttttcttcctccatattcgttatttatttcgaactgga

SEQ ID NO: 59 - DNA, 1000 bp, Pichia pastoris
gtaaataagttaaagtttaaaaaggaaaggatgcaaaaaatatccttgaaggcaacgaatattttgaaatccccgatgccaaataaatgctatcacttaaagagcacaatagaggtggaaaaagaaaaacttggtcaagctaagggttagcaagtttctgtttgtgataatcagggagaaggtgtcagaaaaaacatgattatgtaagtggtgttaggagccgttaaagcattctgtcggccaatagcaagcccgccttttgtcatctttttgcggtttctctgtgtgagacactaatcacctttgtaagacatcgggaaaaccgttgcgcaaaatgagatagagattgttctcgatagaggagcgtagtagcctctccagcctgctttagcaacataatagaaaagaaatatgcgttgcctagggaggctacgtatgcccagcataaacgagtgtttaccttacttcgcacgagcagtagccactaagatcattataaactcacctattgtcttcatgctgtgctccgcgtatttgtctgttcagggtgtcatttctcgtcatgagaatctgattgatgactatgcgagattacccctggattttttttgatcccgtaacgcgaacttgaacattgactttgatatggcaatgggccctaatatgccctaatatgccctaagcttaacaattgacttctgttctctggcagactccacagaaaactggttgacaggtctaatttctttttgaatcatttccggtgattcattttgatgcttagagtgagtcatgggttctttatccgcattcttcttcgcgtctgctgtgcttaataatagcctactaaaaatgtggggagcctcttaccttatgtctataaaaacaagcacatgactatgccatcgccttcatagttgttctgcgggtttttgcttgttttatgaccgtagagaccaaccaatttacatatctacagggtagcacattcgataagaaa

SEQ ID NO: 60 - DNA, 1000 bp, Pichia pastoris
gtgaacgatggcgtatatttgagcggccattgaattattgttgagttgtacaatgattgcaaatggtccatgataagggatgactactagctgttgaaaggggggatgaaaatataagcctaggggattccgggcggaaacgagggcgggggtgacgattattcttgaaggccgctgaccggggtgagaagaacaatcccttggacgggcaaaacgctagatcgaagattttcagactcgaacgatcggccatacctgagatcgatcggtcaaaaagattcacttgctgggtgcctctgtcgtattggcttcaggccccacaaacttaaccttaccttaatggttctttggtcctttggttctatcctgaaacatattgccacatttcccccaccaaaactctaatcaaaccgagaagctttattgctattttccccatagactaatccatatcccctttggaaacgccaactgcatctgcaccctaatgccttcaaacccatgaccctagcacaagaaccctcggaattataccatccccatcccatatgacgaccatagttcgcgtattcttctccatcatgtcctaaaagggcaaatcagttttgtggagaagtcatagggtaggaaagaaggttgcttcaaatggttgcgaccggtcccacatggggcatgcgtgaggtaaaagaactccatcgcactagggatcaacggtagcagattagtaatttacctgaacagtaatcaactagttatcacagtcaatccgtagcttattgcacctatcaatttgcctaagccaaggacggacgacattactaaggtgcaccaactaagccctctgcgctaaaggagaaaatctatatttgtcttgcatgtgccatgcagtcgtgatttaattgacgaaagcaagtttttccgtttaaaagggacataaattcactgcaattgaggccctttacattgctcagattcaccaacttcctaccgttctaatata

SEQ ID NO: 61 - DNA, 1000 bp, Pichia pastoris
ttatccgatgcgcttcaaagctggaattgtaaatatagagaaaaagaaggatgttgttttattcttgaaagagtataattttacttctagcaactctcccacttcgcttgacttcatttatttcttgggcacataggcgtagtaatctagaccaacagataatttgccggaatgatatagcgattggaaaatgaactgaaattttttgctgtctttcaatttgacgggcagttcatcagtgaccgaccatataaatacgttgagaatgttattcttcctcgtagttgaagtggcttcataatttcagaactcaatagataaactaggatgttttaaagcaattaatgctcacaagtaaggagcgactctcttgcttttcgaatactaaaagtatcgtcccaacccagaaaaaaagacctcttaactgcaaaataaactctatatatttcttctaaaacagtttcaggttggatagtatcgcattctcatcacttctaactagtaggccatgagatatattaacgtttacttgagttctaagttctccgaattagatgcacagcacaaacaagattaggtttcacttggtacaaaatacgaacagagtttaaggtcgtaatttcatttcgttattgatccccacaatctattcttatcacagtcatcagatagtcgcgaaaaagcatgcagaaaagggggtcgtccctatctaagttgtagcattacaacaaatatgactacactcagtgtcgcaatcggtatagccaacgctgcaaaatggattctactgagaatggtatgatgatcccaggatcaatttcccaaaaattaaaaaaagtaaaataaaaagcatcagatattagggaggtggtaagattgctctgcaagcgatcacgagattttaggttttcctttatgtactatataaagcgcagattggatgccgcttttccctcctgggctatgataatatagcgaacgaaatacacgccaaaataaa

SEQ ID NO: 62 - DNA, 1000 bp, Pichia pastoris
gagcaggctttttggctgctaacgggtcctcaaattcatttccatcttcgtcttcctcaatttgttccactgcctcaacttcctctatataatgcttcaacattgattcaattccattttttagggtgatggaacttgaagagcaacttctgcaagctcctcttaatcttagatatactgttcctgtctcgtactcaaatctaacaaattcgatgtcaccaccatcatcctggatggcaggtcttattctggtaaatattagctctttaaccatgctgacgacttcatcttcgtcatcctcttccagcaaagcctggtcatttgcgtctgactggtgctgttcgttcaagacaggagtaccgttattcaaagactctgtgaggactgcaaatatctctggtttcaacaaagaccaatcatcttgagtcttcttctctacagtgataaaatcgtgacctatcatgatggttttcactccatctatcccaaacaacttgagagctaaaggtgatttgaatgcttgtcttccgttaagaaactctatagtggtttgctcaggtaaaatcttcatggatggcaaaaacttgagggcatcatcgttgggggtcgtttgtgtttgaataaataggctcctcaaaaaagtcctgttgataggcactatcttttgtctatttaatagtcgtagcattgctgtgtgtattgtgatgggattgtagaggaaaagaaaaatgagaaactagcaccctttagatagtgcgcattggttggcatcttagtgggggaggccacaaggagaaagcatcttctccgcctggtcgtggtgctgacaaataaatcgatcattaacggtacagtcatctttattatttataatataatatcatagatactatgttataattataatttagaaaagataaagtttgactagccgttccccagaggctatataaggaaggtggttaagtcctccaggttttactgtttcttccttattgct

SEQ ID NO: 63 - DNA, 1000 bp, Pichia pastoris
ccactaacattaaccaacagcgaaatgcacgacattattcaaaatgtcgtaccgtctgagggtgcagagatgtagtagtgtgaaaagagggagaaaacagtggatgctggatgttcgatgtcggacctgtgagtttgatcaataggtcaattactgggaagatgaaatggatgcttgtgtagcgtttaggtgctcaaagaagattgatgttggggcgagagcctaatttcaaacgccaacgagattgtttgttggtgagcgggtccaacttccaattggatcattcctctgctcgagccaaagagacgttattttttgacgcctgctaagctttttttaagctttttaaggatctttcgtagtgaagagttttgagtttttttcctgaaccaagctagataacctgacttcatctgagaaaaacagaccacaacggttaatcaaaagttgggagatcaaatcaggagatttctccagttaccatgcgcatacacaacagataaccgatgattatgagtcccctttttctttctttgacaggtcttttgatcgtaaaatcaaatggacagtcataagtgaaactttgatagagtggtggcgtacctattgcactcactaatttgcagtacagtttactcgcagacacccgtagcctatcaagtccctttcccttacttatttcaaaaaatatgcctctttcaaacctaccatgactcgttcgttgtaaaaaggaacccctttagtcgggaaaaaagtctcgcaatgagttagatcggggtagtcataaccgtagccatgactcgctttaatcattgcctgtttaatcaccgggtattaagcaaattgcgttcactaaccaatatttatccgttttagtcgcaagaaaatttcaaatccgatctgcaaggtgagatgagtcgtcgctagatgcggttatataataagaggtctttccccatactacaatcacttgacccaaaagtagtagaatttcactata

SEQ ID NO: 64 - DNA, 1758 bp, Pichia pastoris
atggcaccaagtgcctcaaccattccaatgggtgaatacattttcagaagaatccaatcattaggcgtatccagtgtgtttggggttcccggagatttcaatctgaacctattggagcatctctactcagtggaaggcatgtcgtgggtaggttgtgccaacgaactaaactctgcctatgctgcagacggttactctagagcttcaaataaaatgggatgtgtgataacaacttttggtgttggagagttgagtgcaatcaatggaatatcaggtgctttctcagagtacgtgcctattcttcatattgttggaacaactcctctctctgccaagattgctgaaaacaatcatacccatcatttggtcccaaagttgtctgtctttgagccgtcagaccatttcacgtacgaaaaaatggtagcacctgtttcttgtcatcaggaaactgttgtaaatgctagtgatgcaccaggacagattgacacattgataagacagatcttgaagtacaaaaggccaggatatctgtttttaccttctgatttggccgatatcaatgtagatggggattttttaattcaaagaactactcaacaattctatcaatccgtcgacacaaatcaatctctaacaagagaggttgcaaccaaggtactggataagatatacaattgttcaaatcctgccgtgctgggagatatactttgcgatcgtttccaagtaactgaacatgttagagcatttgtcaaaaatgcatgtatcaagagtttttccactttcatgggtaaatcagtcctggatgagagtgactcaagatatatcggaacctacaatggcgtggagtcaaataacgaggtgattggatacttccaggcttctgatctcatattgcatattggaaactactacaatgaaatcaactctggacatgacactttgtacaacaacatcgacgaagagcaattgatccttatgcatccagagtacatcaaaattggaactgagttgttcaggaacgtcaacttcgtgcacgtcctagacgtaatgcttcaaatgatggatgtgtctcaaatcccccgaggcattagccccactttatcaaagaaagagattaaccacatcgaacacatttcagcttccactccaatttctcaaacacaccttctgcataaattgcaagacttcattaaggaagatgattttgttgtagtggaaacaggatctattatgttcggacttccggatttagtccttccaaagggtgcccgtttgtttggacagcatttctacttgtccattggctacgctttacctgctgccctaggagtaggagttgctatgaaagatggaaacagtaagggaagactcatccttttagagggagacggatccgcccaaatgactattcaagaattcggaaactatgtctaccagcaaatcactcctatcatttttctcttaaacaacagcggatacacggtggaaagaataattaaggggcctcaaagggaatataatgatattttgccaaactggaattggaccgaaatttttaagacatttggagacagatatgaatctaaaagtgaaacaaaaaagatccaaactgtcgaggagttggaccaagttatgctgtacaccaataataacaattccaagctgaagctttttgaagtaatacttgatcaaatggatgttccttggagatttagttatatgactgctgccagcaagaacaaagccaaaatcgtaggt

SEQ ID NO: 65 - DNA, 2307 bp, Pichia pastoris
aaaatggttcaatcatctgtcttaggtttcccacgtatcggtgcctttagagaattaaagaagaccaccgaggcctactggtctggtaaggtcggaaaagacgagcttttcaaagtcggaaaggagatcagagagaacaactggaagctgcaaaaggctgctggtgtcgatgtcattgcttccaacgacttctcctactacgaccaagttcttgacctgtctcttctgtttaacgctattccagagagatacactaagtacgagttggacccaattgacaccctattcgccatgggtagaggtttacaaagaaaggccaccgactccgagaaggctgttgatgtcaccgctttggagatggttaaatggtttgattctaactaccactacgtcagacccactttctctcactccactgagttcaagctgaatggtcaaaagccagttgacgagtacttagaggccaagaaacttggaattgagactagaccagttgttgttggtccagtttcttacctgttcttgggtaaggctgacaaagactctcttgacttggagccaatctctcttttggagaagattttgcctgtctacgctgaactactggccaagctgtccgctgctggtgccacttccgtgcaaatcgatgagccaatcctggttttagatctcccagagaaggttcaagctgctttcaagactgcttatgaataccttgccaatgctaagaacattccaaagttggttgttgcctcctacttcggtgatgtcagaccaaacttggcttctatcaagggtttaccagtccacggtttccactttgactttgtcagagctccagagcaattcgacgaagttgttgccgcattgacagctgagcaagttttgtccgtcggtatcattgacggtagaaacatctggaaagctgatttctccgaggctgttgctttcgttgaaaaggctattgctgctttgggtaaggacagagttattgttgccacctcttcctctttgttgcacacaccagttgacttgaccaacgaaaagaagctggactccgagatcaagaactggttttcgtttgctacccaaaagttggatgaggttgttgtcgtcgccaaggctgtatctggtgaggatgtcaaggaggctttgtctgtaaatgccgctgccatcaagtctagaaaggactctgctatcactaacgatgctgatgttcaaaagaaggttgactccatcaatgagaagttatcttccagagctgctgctttccctgaaagattggctgctcaaaagggcaagttcaacttgcctttgttcccaaccaccaccattggttctttcccacagactaaggatatcagaatcaacagaaacaagttcaccaagggtgaaatcactgctgagcaatatgacactttcatcaaatctgagattgagaaagtcgtcagattccaggaggagattggtttggatgttcttgtccacggtgaaccagagagaaacgatatggttcaatactttggtgagcagctgaagggttttgccttcaccaccaatggttgggtccaatcttacggttctcgttacgttagaccacctgtggttgtcggtgacgtttctagacctcatgccatgtctgtcaaggagtctgtttacgctcagtccatcactaagaagcctatgaagggtatgttgactggtcctatcaccgtcttgagatggtctttcccaagaaacgacgtttcccaaaaggttcaagctctgcaattgggtcttgctctgagagatgaagttaacgacttagaggccgcaagtgtcgaagttattcaagttgacgagccagctattagagaaggtttgccattgagaagcggtcaagaaagatctgactacttgaaatacgctgctgaatctttcagaattgctacttccggtgtcaagaacactactcagatccactctcacttctgttactctgatttggatcctaaccatatcaaggctttggacgctgacgttgtctctattgagttctctaagaaagatgatcctaactacattcaagagttctctaactaccctaaccacatcggattgggtttgtttgacatccactctccaagaattccttccaaggaggagttcattgccagaattggtgagattcttaaggtgtacccagctgacaagttctgggtcaaccctgactgtggtttgaagaccagaggctgggaggaggtcagagcctctttgactaatatggttgaagctgctaagacctaccgtgaaaagtacgctcagaat

SEQ ID NO: 66 - DNA, 573 bp, Pichia pastoris
atgaaatacgttttatctgagcaagtccttacagtcccagaagatgtgtctgtgtctattaaggccagaattatcaaggtgactggaccaagaggtgaactgaccaaggatctgaagcacataaacgttgcttttgagaaatctggcgacaacgagattaagatcattgtgcatcacggtaacagaaagcacgttgctgctttgagaactgtcaagtcattaatttctaacatgatcactggtgtcaccaagggttacaagtacaagatgagattggtttatgcgcatttcccaattaatgtcaacttcctcgagagagacggtaatcagtacgttgagatcagaaacttcttgggtgagaagagagtcagagaggtcaaagtttacgagggtgtcactgcatccaactcttctgctttgaaggatgagctaatctttgagggtaactccattgagaacgtctcccaaacttgtgccgatgtccaacagatttgccgtgttagaaacaaggatattcgtaaattcttggacggtatctacgtctccgagaaaggaaccattgtccaagacgaa

SEQ ID NO: 67 - DNA, 1041 bp, Pichia pastoris
atgagtgtaaactcattgagagcagcttcttcctcggcgggtccaacaaagttgtctgtcagagacgctttgaattcagccatggccgaagaattggacagagaccctgaggtgttcttgatcggtgaggaggtcgcacagtacaacggtgcttacaaggtttccagaggactgctagacaaatacgggcccaaacgaatcgttgataccccaattaccgaaatgggtttcactggtcttgctgtgggtgcttcgttggcaggcttgaagccaatctgcgaattcatgacatttaactttgccatgcagtcaatcgatcacattatcaattccgctgccaagaccctctacatgtctggtggtaagcaaccctgtaacatcactttccgtggtcctaacggagctgctgctggtgttgcagcccaacattcccaggactactctgcttggtacggatctatcccaggtctgaaagttatctctccctactctgccgaggactataagggtctgttcaagagcgccatcagagacccaaaccctaccatctttttggaaaatgaactgttgtacaacgaagagttcgaagtttctcctgaggttctgtcccctgatttcactgttccaattggtaaagccaagatcgagcgtgaaggtaccgatatcacgattgtatcccacagcagaaatttgcagttctgtttggaggcagccaccattttgaaggaaaagtatggtgtctcatctgaggttctcaaccttcgttccatcaagccattggatgttcctgccattgttgaatctgtcaagaagaccaaccatctgataactgttgaagccggtttcccagcctttggtgttggttccgagatttgcgctcaggtcatggaatccgaagcttttgactacctagatgcccctgttgaaagagtgaccggatgtgaagttccaaccccctacgccaaggaattagaagactttgccttcccagacaccccaactattataagagctgtcgagaaggttctttcgttgaaagag

SEQ ID NO: 68 - DNA, 1152 bp, Pichia pastoris
atgtctaaaaacgaaacattctttttcacttctgaatccgtcggtgaaggtcatccagacaagctttgtgatcaggtctccgatgctgttttggatgcttgtttgaccgtcgaccctctaagtaaggtcgcctgtgaaaccgctgctaagaccggtatggtcatggttttcggtgaaattaccaccaaagctcaactggacttccaaaaaattatcagagacactgtcaagcacattggttacgaccactctgacaagggtctggactacaagaccatgagcgttcttgtcgccatcgagcaccaatctcctgatatcgctcaaggtcttcactacgagaaggctttggaggagttgggagccggtgaccaaggtatcatgtttggttatgctactgatgaaactgatgagaagttgcctttgaccttgcttttggctcatcaactgaaccacgagctggcttcttgcagaagatctggatctcttccatggttgagaccagacaccaagacccaagttactattgagtacaaatacgacaacggtgctgttattcctctgagagttgacactgtcgtcatctccgcccaacactccgaagagatcaccactgctgacattagagtgcaattgactgagcacgtgatcaagaaggttattccaagccacctgctggatgagaagacaaagtaccacattcaaccatctggcaagtttatcattggtggtatcgccggtgacgctggtttgactggtagaaagattattgtcgacacttacggtggatggggtgctcacggaggaggagccttctccggtaaggatttctccaaggtcgaccgttccgctgcttacgctgccagatgggttgccaagtctcttgtacacgccaagctggccagaagatgtttggtccaattctcttacgctattggtgttccagagcctctttccatctacgttgacacatacggtacctctacctactcatctgacgaattggtcaagattatcaacaagaacttcgacctgagacctggtgttatcgtgaaggagctagaccttgccagaccaatctacttcaagactgcttcctacggtcacttcaccaaccaagaaaacccatgggagcagccaaaggttcttaagctt

SEQ ID NO: 69 - DNA, 1335 bp, Pichia pastoris
atgtctaactacaaagtcgccgacatttcacttgctgccttcggtagaaaggacattgaactcagtgagaatgagatgccaggtctcatttacatcagagagaagtacggacctgcccaacctttgaaaggtgccagaatcgccggatgtctgcacatgactattcaaaccgccgtcctcattgagactttggtcgccttgggtgctgaggtcacctggtcctcatgtaacattttctccacccaggaccacgctgccgctgctattgctgctaccggtgttccagtctttgcctggaagggagagaccgaggaggagtacttgtggtgtatcgagcaacaattatttgccttcaaggacaacaagaagctgaacttgattttggacgacggtggtgatttgacttctttggtccacgagaagtaccctgaaatgttggatgactgtttcggtctgtccgaggagaccaccactggtgtccaccacttgtacaagatggtcaaggatgctaccttgaaggttcctgccatcaacgtcaacgactccgtcaccaagtccaagtttgacaacttgtacggttgtcgtgaatctttgatcgacggtatcaagcgtgccaccgatgttatgatcgcaggtaaggttgccgttgtcgctggtttcggtgacgttggtaaaggttgtgccatggctcttagaggtatgggtgccagagttatcatcagtgagattgaccctatcaacgctctgcaagctgctgttgaaggttaccaagttgcccctcttgatgacgttgtctccattggtcaaatctttgttaccaccactggttgcagagacatcatcaccggtaagcacttcgagcaaatgccagaagatgccattgtctccaacattggtcacttcgacattgagattgacgttgcttggttgaaggccaacgctcaggacgtcagcaacatcaagcctcaagttgacagatacttaatgaagaatggtcgtcacgttattcttttggctgacggtagattggtcaacttgggttgtgccactggtcactcttctttcgtcatgtcctgttctttctctaaccaggtcctggctcaaattgctctgttcaagtctaacgacagtgagttcagaaagcaattcgttgagttcgaaaagtctggtccattcgatgttggtgtccacgttttaccaaaaatcttggatgaaactgttgccagatgccatttggctcacttaggtgctaagctgaccaagttgtccagtgttcaatctgagtacttaggtatcccagttgagggacctttcaaggttgatcactaccgttac

SEQ ID NO: 70 - DNA, 1000 bp, Pichia pastoris
aaagtaatccggaagttgaagctctgaacaacgacttagacaacatgagcgactttgaccctgcggactttgattactcagatagtgacgaggaagaaaagaaaaaggacgataatgttccagttcagataccatcccatttggctgctatagctgcccaagaaccctaccctgaggacagtgacaacgataatgacaataaattctcatctgatgaagaggccaagatctttggaccagactctgaagacgaattgagcagtgaagatgaaaaacctaagaagaagagagccagaaccgacacgtcggatgatgtttctaaaaagtccaagagccttaagggtttaccgatgttcgcctcgatagaagactacgctgatctcttggaggatgatggcgaggacgaagagtgatggcctagaacattttcatagcattatctatctaatttcaatcaatgtaagaaggtatcttttaacctggcatttttttatattaccgtctctttccgtcctttcggttaaccatggattggtgtggcatttttgaagtgccacaagtgcggatatattcggttattcggagtctgccacaatattttcgcacactttttcccacaagcagtcgccatactccttcctgaataaccgaaaatttacaaggaaatttctggcacgaaaaattcccttttatagagggaacctacccaccggcgttcccgaaagggatcagggctccttccaacgatcccatttcaaatatcgtagatttgtggaccagtcttatctcggaaaacgataaccattgcagactacaaaagacacaacactgttgaatattctagtgtagattcccttgctcggtgtgtaggcatattttcctataagtacagtctcatgttacctaaaatctctctaaacaacttgtagaagcaaccataatggccccagttccagatatagcataccatactttgaacgacctggatcattcatggtcc

SEQ ID NO: 71 - DNA, 1000 bp, Pichia pastoris
gatgacgttgatgccgggcatttgcgatactgattcagaaattctggacgcagcattgttcaaaatgtgtcgttgtctagaaaattcgtcccgagtttcgtacgcctgtgatagtaatctgtcagcaaacgagttaacattatccacccgattgcgctcttgtctcatgtactcttcttcattcaggctatcgacgttgcttgtgtgagagcgtttcttgtgctcctgtatgtcagacctaacagagaatagaaggtttagtttgttccgctcctgttgaattgactcctgaatgcggccaaaatcacgtttatggtcattaagtatctctttatgtctgtgtaattgctgtagtttggaagcggcaagagtgtcatccgaatctgcgattcggctcaaggcattgaccaattcctgccttcgttccaggatatcctttaacgccttctccaacgacaattcttcgcctgtggcagaactagacgatgattggccaaaagacgcatattgagacagtagcgactctgtcttgttctccaattgcaacgcttgggaccttgtttgggagtagttcgacattgggttcctctgagatgtttgacaagtgagagctaaatgataacgaaatgcctacctggcaggacgtgtactgatcaaacctcccaggttcacatcggtcacttgctcgattccagcaagctacgccctttaagttttgtccaccagctttgcgcactctcttgcctctttcgaaccccgagcgcgcttcagatgcagatcaaagcacgagatgccacgtgacagtccatgtattctttcgtttatcttcgtatagacaataatatttcattgactctgtcaatggtcgatgttcacgtgcaaaaattttcaattcgtttgttgggcgacacctccactacgtatataaaaggatccgaccgcccacttgtccttgcttcctgtaattgtttcccaaacaactagtagttcaattattact

SEQ ID NO: 72 - DNA, 1000 bp, Pichia pastoris
atgcttacgcgtctgtttcagtcttttcagctaagtcttgtgaaccatgtgagtcgtgtgcatatcactagaacatattctagtagttcttgttcatgctttgtgctgttgactaccgttacccctgttgattgtttatggaaggtagttcctgcagaacgtgcatttacaaagagctattgctttgagaatcttctatgtacaacttactgcaggtgtgcatacgctatttatgccaagctctttacaggttagagtagtcacctcatggtaataatttcctgagctacaagtccacacgggtctttacaaacaggaaagctaaacgaaaaatgggaagcattgaaatttgggtttctgtcaaccaatgtggtctttacttcccatactccgtctagggaatgtctctaggattgcatagaaacttttcaaagttgaaccagaggtattgcaaaaaaatgttggccaaacaaacaccaaagggtgtaaacatgcctcgttcgattttaccccccctcccaattgcgcatataaaagatggagcacaggtaatctttacatttgggtaccctcccgacatgcgcgttaaaggcagtagagtcttgggcagtactcgtcaacaggaccccaattgctgacattcaactacatacaattcttagtgattcttggagtacagttgattgatctggatagacatccctgcgagtagtcttcgggaattacagtgataataataaaggtgtactcctttgccaagacaggtaagcttcttcaagatctcaaactttgttgtacatatgtacgtttccatcaggggactttcgtttcgcagcaacttcgctagtagtggcaaggaaaagaaaactacactgtgactgcgatttcctgtttttacacactattaacaaagtcgtgtattcaactgaaaaattcgctttctatcgcttcaggcccgttagagtgtgaaaaatttagaaacagtaccaatcaatacgag

SEQ ID NO: 73 - DNA, 1000 bp, Pichia pastoris
agttaagaactatagtttctacagagctactccaagtcaacagcagttacagcagcagcagcaatctcagcaggtgcaacaggcacagcaagttcaacaggctcaacaagctcagcaggcccagcaggctcagcaggttcaacaacaggctcagcaggcccaacagcagcaggcacagcaacagcaacaacagcaacaattgcagcagcagcacatccaacaattgcaacaaatacagcaactacaacttgcgcaacagcagcagcaacagcaacagcagcgtcaacaacaggaacaccttcttcagcaattgcagcagcagcagcagcaacaatatcaccaacctcaaacacaaggccaaaatttcccacaacagtacttgatgcaacaggcccaagctcaggcccaagctcaagctcaagttttagcccatgctcagcatgacaactcatccaatccaatgttcactaacatcagagaaggatctggggtctcagctacacctccacccccaggattgtttagtggagtgaccaatcacactacggaaactcataacactccatcatctgaactattgaaccagctgatgaacggggagggtagaaactcgatcaatgcttagtcggtgattatctgttatataagaagtacctattagttgaataaagtaataatattggatgtctgatgttccgaggcttccctagtccgagtcgattgcgcgcgtaaattggtgcttttcccccaccgaaacaataatgaggggatctccatatcacgtgatgcattcggtgtaacttttagtggtataaaccgcggtcggatgcactccgcctaacaaacttctgtgaggtgcgaaacaaggaacccgtaaaggaaaacctcattgattacctgttagttcctactttctcttttacccacaaggttcactctcaccacaatgttgccatttcaagcagtagccaaagcaggcctcaagccccaattgacccgc

SEQ ID NO: 74 - DNA, 1000 bp, Pichia pastoris
cagtatgcaacatggccgatgttggtttgcgtttctgggcgcaccctgatggaccacgctgagcatgtttctcttcttcctgtctttgttgggcaatgttgagctgattacaccgtcaggagttaatatgggccgctcgtgactttagctctatatacgacgcttggtatcgcacaagctcgttttccctgcagaattggaagcagtagactacagctacagtaatcagttgaccggggtaatacaggcacttcctcagaaatccagattaaactccaaaggctgcctcaaagattccaacaaccagaatgatcaagtaaactacgacacgtccaaaagagaagatgaccacgttctttcgaagaggcgcatagaagttggtgactggcacgaggagttttcatccaaggctgggaaaaattcgttgttgaatcgtgggataatcgcactttaggcaaaccttacagcctcaaacgcagctgttatcaccgccagagaccaattaagcgctctactttcattccttctggacccgcttgcatgaccctctccaaaaaaaatatgtccgaacactcgcccttggtactttcctttaaatccagagatgcatgaagaaaaccaagatattgagatcacgtgagtcgtcgcaattgcctttaaacgatcctatacgaatttttcattaagcaaagtttcacatgcgacgacggctcagcgtacaagcacggcaattgggcgaattgtgccaacataaaccatcagactcagcgtgtctgtcctggcaataccaccattggtggtttgacgccgaatcatccacgattgcccttttaattgaccctctagctaaagtttggctcaaaaacacgttggaggaattaatatgtggcacgtgatgtccaacaaatttttcaagacttacataaatactccatgtctgctccttcatcattacaacccaccgtttcctttacctcaacctcaaaacactgtttataca

SEQ ID NO: 75 - DNA, 1000 bp, Pichia pastoris
gagacaagtatactgatcttctttctacctattttggttgctacccatccactatacagggagcccagcagacccccaaaggacaacatggtggtaatcatagcccgttgactagatttcatcgggatacattgttcaaacaactccggatgctgggcccaccaagacatggggccttcctgatgcagcctacaagatataattctctggggtgcgttcaactcactcagatgatagccaaacaggaatgaacctaagcagatcactaatatgttgaagcccagcgtgggtgtaacttgagcttgggtgtcgaaatgcatttttcagtttcaatgttgctataaacaaaccaaagttgaggggaaacaatctataaaataggatttttgaagtacgtgaattgttgctgtctgccagattcgggatgccgatttgataagagttttcagatttgatagggatgtaagcaaagcgggttgtttccaacggagaacgaacgataataacgcaggaactcatgacgatatgcaaagaatcccctgattatttcttctaaaacgtagcccggttgttcttgtgtcgggtttccttcaaaagggaccctgtgtactatgtgttatccgatccttagagggaatctccgtgtaagggacatccagcatcttgccaatttttcaaaagcaattctcagatctttggcacctgacgtcctagtccacgacaagagatacgttcttcctgggggtggtttcaacggcctgagcttgcacttattacaattggcaatcgagctggtattatttctcgcatgtagcaggatcatacaacatccagacaccccaacccatatccctaattaccacgttctcacatgcgctcaacaaaaaactcgtaaccctcgtttatttttttaaggaacctcaaaagattttcacacctataaatatcgtgaaaaactatcctccattttcctctctactacactaaaccaaataatacc

SEQ ID NO: 76 - DNA, 814 bp, Pichia pastoris
agatcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacagcggccgcactagcttaattaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgccggctcttaagcggaccg